(19) United States
(12) Patent Application Publication
(10) Pub. No.: US 2024/0067758 A1
(43) Pub. Date: Feb. 29, 2024

(54) MULTI-SPECIFIC ANTIBODIES AND ANTIBODY COMBINATIONS

(71) Applicant: UCB BIOPHARMA SRL, Brussels (BE)

(72) Inventors: Joseph Michael David RASTRICK, Slough, Berkshire (GB); John Paul SILVA, Slough, Berkshire (GB); Daniel John LIGHTWOOD, Slough, Berkshire (GB); Ralph ADAMS, Slough, Berkshire (GB); Roger Thomas PALFRAMAN, Slough, Berkshire (GB); Kerry Louise TYSON, Slough, Berkshire (GB); Peter Charles ELLIOTT, Slough, Berkshire (GB); Seema MAYANK, Slough, Berkshire (GB); Andrea Julie CROSBY, Slough, Berkshire (GB); Emily Mary Cairistine BARRY, Slough, Berkshire (GB); Seppe Frans Roman LEYSEN, Slough, Berkshire (GB); Zainab AHDASH, Slough, Berkshire (GB)

(21) Appl. No.: 18/255,213

(22) PCT Filed: Dec. 6, 2021
(86) PCT No.: PCT/EP2021/084402

§ 371 Date: May 31, 2023

(30) Foreign Application Priority Data
Dec. 7, 2020 (EP) 20212128.1

(51) Int. Cl. C07K 16/46; C12N 15/63; A61P 17/00

(52) U.S. Cl. CPC C07K 16/468; C12N 15/63; A61P 17/00; C07K 2317/31; C07K 2317/76; C07K 2317/565; C07K 2317/55; C07K 2317/622; C07K 2317/14

(57) ABSTRACT

The present invention relates to antibodies binding to IL13 and IL22. The invention provides novel multi-specific antibodies that bind to both IL13 and IL22 and compositions comprising an antibody that binds to IL13 and an antibody that binds to IL22. The invention further relates to therapeutic uses of the combination of anti-IL13 and anti-IL22 antibodies and multi-specific antibodies that bind to both IL13 and IL22.

### FIELD OF THE INVENTION

The present invention relates to antibodies that bind to IL13 and IL22. Such antibodies provided herein are useful in the treatment of inflammatory conditions, in particular, skin inflammation.

### BACKGROUND

Atopic dermatitis (AD), also known as atopic eczema, is an inflammatory condition which results in epidermal dysfunction and thickening, eczematous lesions and pruritis. The condition is prevalent in people of all ages and ethnicities and has the greatest disease burden of all skin diseases measured by disability-adjusted life years (Laughter et al, Br. J. Dermatol. 2020; Epub ahead of print). AD is a complex condition and its pathophysiology is influenced by multiple factors, such as genetics, environmental and immunological factors. Although type-2 immune mechanisms are important in the pathology of atopic dermatitis, increasing evidence supports a role for several immune pathways.

Treatments used for AD include systemic immunosuppressants such as cyclosporin, methotrexate, mycophenolate mofetil and azathioprine. Antidepressants and naltrexone may be used to control pruritus. In 2016, crisaborole, a topical phosphodiesterase-4 inhibitor, was approved for mild-to-moderate eczema, and in 2017, dupilumab, a monoclonal antibody antagonist of IL-4Rα was approved to treat moderate-to-severe eczema. Current treatment options however, offer only temporary, incomplete, symptomatic relief.

IL22 is a member of the IL10 cytokine family with multiple functions in various inflammatory and tissue responses, depending on the environmental context. IL22 is mainly produced by lymphoid cells such as T helper 1 (Th1) cells, Th17 cells, and Th22 cells, γδ T cells, Natural Killer (NK) cells and innate lymphoid cells (ILCs) 3 as well as non-lymphoid cells such as fibroblasts, neutrophils, macrophages and mast cells (For an overview see: Lanfranca M P et al. J. Mol. Med. (Berl) (2016) 94(5):523-534). IL22 signals through a heterodimeric transmembrane receptor complex composed of IL22 receptor 1 (IL22R1, also known as IL22RA1, or as Interleukin-22 receptor subunit alpha-1) and IL10 receptor 2 (IL10R2), whereas IL10 signals through IL10R1 and IL10R2. Similar to other members of the IL-10 family, IL22 mediates its effects through the IL22R1/IL10R2 complex and subsequent JAK-signal transducer and activator of transcription (STAT) signaling pathways, including Jak1, Tyk2, and STAT3. In contrast to IL10, IL22 is also reported to signal through a number of MAPK pathways such as ERK1/2, JNK and p38. In the skin, IL22 acts on keratinocytes through binding to IL22R1 expressed on these cells.

Unlike other members of the IL10 cytokine family, IL22 has a soluble-secreted receptor, known as IL22 binding protein (IL22BP, also known as IL22RA2, or as Interleukin-22 Receptor Subunit Alpha 2). Although IL22BP shares the highest structural homology with the IL22R1 chain, IL22BP exhibits a much higher affinity for IL22 than IL22R1 and therefore prevents the binding of IL22 to IL22R1.

It has been shown that IL22BP, which is specific for IL22, blocks its activity. Inhibition of total IL22 has shown efficacious signals in patients with severe atopic dermatitis or in patients with high baseline IL22 expression (Guttman-Yassky E et al. J Am Acad Dermatol. 2018; 78(5): 872-881 and Brunner P M et al, J Allergy Clin Immunol. 2019; 143(1): 142-154). Inhibition of the IL22R1 has also been proposed as a potential therapeutic option to inhibit IL22 which would also partially block the effect of IL-20 and IL-24. To date, no therapeutic option exists that is designed to specifically target biologically active IL22 that is not bound to IL22BP, thus not impacting on the normal biological function of IL22BP.

Increased expression of Th22 cytokine IL22 is a characteristic finding in atopic dermatitis (AD). However, the specific role of IL22 in the pathogenesis of AD in vivo is not completely understood. The role of IL22 in the development and maintenance of AD has not been specifically explored but it has been hypothesized that IL22 plays an important role in the development of AD by impairing skin barrier function, immune dysregulation, and pruritus.

U.S. Pat. Nos. 8,906,375 and 7,901,684 disclose antibodies binding IL22 and usefulness of such antibodies in the treatment of AD. U.S. Pat. No. 7,737,259 discloses specific anti-IL22 antibodies useful in the treatment of psoriasis.

IL13 is a short-chain cytokine sharing 25% sequence identity with IL4. It comprises approximately 132 amino acids, forming a secondary structure of four helices spanning residues 10-21 (helix A), 43-52 (helix B), 61-69 (helix C), and 92-110 (helix D), along with two β strands spanning residues 33-36 and 87-90. The solution structure of IL13 has been solved, revealing the predicted up-up-down-down four-helix-bundle conformation that is also observed with IL4.

Human IL13 is a 17 kDa glycoprotein and is produced by activated T-cells of the Th2 lineage, although Th0 and Th1 CD4+ T cells, CD8+ T cells, and several non-T cell populations such as mast cells also produce IL13. The functions of IL13 include immunoglobulin isotype switching to IgE in human B cells and suppressing inflammatory cytokine production in both humans and mice. IL13 has been shown to play a role in epidermal thickening.

IL13 binds its cell surface receptors, IL13R-alpha1 and IL13R-alpha2. IL13R-alpha1 interacts with IL13 with a low affinity (KD˜10 nM), followed by recruitment of IL4R-alpha to form a high affinity (KD˜0.4 nM) heterodimeric receptor signaling complex.

The IL4R/IL13Ralpha1 complex is expressed on many cell types, such as B cells, monocytes/macrophages, dendritic cells, eosinophils, basophils, fibroblasts, endothelial cells, airway epithelial cells, and airway smooth muscle cells. Ligation of the IL13R-alpha/IL4R receptor complex results in activation of a variety of signal-transduction pathways, including signal transducer and activator of transcription 6 (STATE) and insulin receptor substrate 2 (IRS2) pathways.

The IL13R-alpha2 chain alone has a high affinity for IL13 (KD˜0.25-0.4 nM). It functions both as a decoy receptor that negatively regulates IL13 binding, and as a signaling receptor that induces TGF-β synthesis and fibrosis via the AP-1 pathway in macrophages and possibly other cell types.

### SUMMARY OF THE INVENTION

The present invention addresses the need for new treatments of inflammatory conditions, such as inflammatory skin conditions, in particular atopic dermatitis, by providing antibodies that bind both IL13 and IL22. The invention for the first time demonstrates that inhibiting both IL13 and IL22 restores normal skin phenotype.

The present invention provides a multi-specific antibody comprising at least two antigen binding domains, wherein one antigen binding domain binds to IL13 and the second antigen-binding domain binds to IL22.

The present invention also provide a pharmaceutical composition comprising an antibody that binds to IL13 and an antibody that binds to IL22.

The present invention also provides a combination of an antibody that binds to and neutralizes IL13 and an antibody that binds to and neutralizes IL22 for use in the treatment of an inflammatory skin condition.

### BRIEF DESCRIPTION OF THE DRAWINGS

The present invention is described below by reference to the following drawings, in which:

FIG. 1 shows a schematic representation of two examples of multi-specific antibodies according to the present invention: (A) TrYbe molecule having IL13-, IL22- and albumin-binding domains; (B) Knobs-into-Holes molecule having IL13- and IL22-binding domains (in 2 possible orientations).

FIG. 2 shows Ab650 humanization alignments. (A) Light chain graft; (B) heavy chain graft

FIG. 3 shows humanization of antibody 11041 light chain. Different variants generated for that chain are also shown. CDR sequences are underlined.

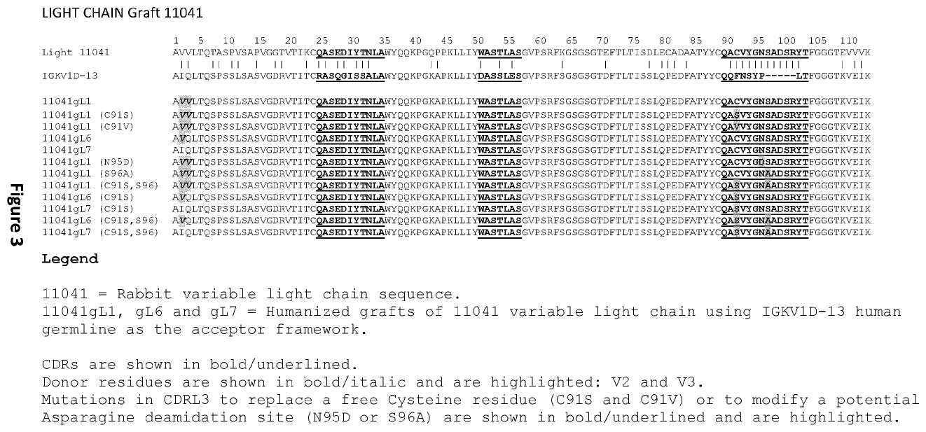

FIG. 4 shows humanization of antibody 11041 heavy chain. Different variants generated for that chain are also shown. CDR sequences are underlined.

FIG. 5 shows humanization of antibody 11070 light chain (A) and heavy chain (B). Different variants generated for that chain are also shown. CDR sequences are underlined.

FIG. 6 shows IL22 peptide coverage map of the HDX-MS experiment.

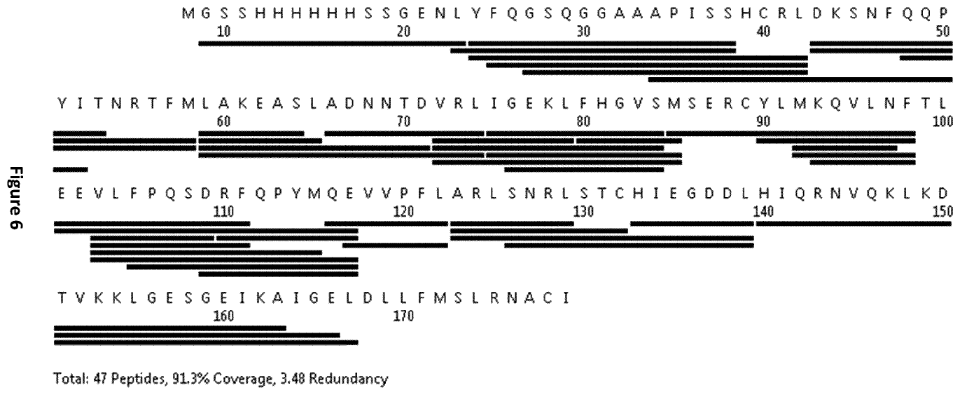

FIG. 7 shows the results of HDX-MS analysis for 11041gL13gH14 Fab. (A) peptides showing significant reduced deuterium incorporation upon antibody binding are listed. Peptides showing a similar exchange pattern in the presence and absence of the antibody have a non-significant deuterium incorporation and are displayed in light grey. (B) determined 11041gL13gH14 Fab epitope is projected onto the IL22 3D structure and highlighted in black. Relative 11041gL13gH14 Fab binding to IL22 from X-ray data is displayed for reference.

FIG. 8 shows the results of HDX-MS analysis for 11070gL7gH16 Fab. (A) peptides showing significant reduced deuterium incorporation upon antibody binding are listed. Peptides showing a similar exchange pattern in the presence and absence of the antibody have a non-significant deuterium incorporation and are displayed in light grey. (B) determined 11070gL7gH16 Fab epitope is projected onto the IL22 3D structure and highlighted in black.

FIG. 9 shows the results of X-ray analysis of 11041gL13gH14 Fab binding to IL22. (A) cartoon representation of 11041gL13gH14 Fab binding to IL-22. (B) a detailed view on the interaction interface between IL-22 and of 11041gL13gH14 Fab.

FIG. 10 shows that 11041gL13gH14 Fab molecule prevents the interaction of IL22 with the IL22R1 receptor (A) IL22 (surface representation) in complex with its receptor IL22R1 (PDB: 3DLQ). (B) The 11041gL13gH14 Fab light chain blocks the interaction site between IL22 and IL22R1.

FIG. 11 shows in (A) Cryo-EM structure of IL22 in complex with Fezakinumab and 11070gL7gH16 Fab (VR11070) in Fab format; and in (B) Model of IL22 in complex with Fezakinumab and 11041gL13gH14 Fab (VR11041). The model was created by superposing the IL22/11041gL13gH14 Fab crystal structure on the cryo-EM structure in panel (A). This reveals that 11070gL7gH16 Fab and 11041gL13gH14 Fab have a similar epitope on IL22.

FIG. 12 shows in (A) superposition of the crystal structure of IL22R1 bound to IL22, on the cryo-EM structure of IL-22 in complex with 11070gL7gH16 Fab and Fezakinumab Fab; and in (B) The sidechains of IL-22 residues known to contribute to the interaction with IL-10R2 are shown as sticks. This site is occupied by the Fezakinumab Fab molecule.

FIG. 13 shows the SDS-PAGE results for the IL13/IL22 TrYbe under reducing (lane 1) or non-reducing (lane 2) conditions. Mark 12 protein markers (Life Technologies) were used as standards (M). Molecular weights (MW) were measured in kilo daltons (kDa).

FIG. 14 shows IL13/IL22 TrYbe (TRYBE) activity in the in vitro human primary keratinocyte assay. (A). Example of eotaxin-3 response in the assay. Geometric mean n=4-6, stimulation: IL-13 and IL-22 at 100 ng/ml, Lebrikizumab (Leb) and Fezakinumab (Fez) at 100 nM and IL13/IL22 TrYbe (TRYBE) at 25 nM. (B). Example of S100A7 response in the assay. Geometric mean n=5-6, stimulation: IL-13 and IL-22 at 100 ng/ml, Lebrikizumab (Leb) and Fezakinumab (Fez) at 100 nM and IL13/IL22 TrYbe (TRYBE) at 25 nM.

FIG. 15 shows IL13/IL22 TrYbe and IL13/IL22 KiH molecules activity in the in vitro human primary keratinocyte assay. (A) Percentage inhibition of eotaxin-3 and S100A7 by IL13/IL22 TrYbe in the assay. Mean±SD, n=3 donors, Statistic: log(inhibitor) vs. response (three parameters), Stimulation: IL-13 and IL-22 at 100 ng/ml. (B) Percentage inhibition of eotaxin-3 and S100A7 by IL13/IL22 bispecific in the KiH formats (IL13 K/IL 22 H and IL13 H/IL 22 K) in the assay. Mean±SD, n=2 donors, Statistic: log(inhibitor) vs. response (three parameters), Stimulation: IL-13 and IL-22 at 100 ng/ml.

FIG. 16 shows the effect of IL-13, IL-22 or their combination (100 ng/ml each) on reconstituted human epidermis (EpiDerm FT from MatTek) after 7 day culture (treated every other day).

FIG. 17 shows the titration of IL13/IL22 TrYbe (TRYBE) from 66 nM to 0.2 nM, with combination of 100 ng/ml IL-13 and IL-22, in EpiDermFT model, or 66 nM IL13/IL22 TrYbe (TRYBE) with IL-13 or IL-22 alone.

FIG. 18 shows the effect of Molar equivalent Lebrikizumab, Fezakinumab (alone or in combination) and IL13/IL22 TrYbe, on combination of 100 ng/ml IL-13 and IL-22 in EpiDermFT model.

### DETAILED DESCRIPTION OF THE INVENTION

### Abbreviations

TABLE 1
Abbreviations used throughout the specification

| | |
|---|---|
| ADCC | antibody-dependent cellular cytotoxicity |
| CDC | complement dependent cytotoxicity |
| CDR | complementarity-determining region |
| CH1, CH2, CH3 | constant heavy domain |
| CL | constant light |
| dsscFv | disulphide stabilised scFv |
| Fab | fragment antigen-binding |
| Fc | fragment crystallizable |
| FR1, FR2, FR3, FR4 | framework region |
| Fv | variable domain |
| HVR | hyper-variable region |
| KD | constant of dissociation |
| mAb | monoclonal antibody |
| scFv | single chain variable-fragment |
| VH | variable heavy region |
| VHH | single domain antibody |
| VL | variable light region |
| VNAR | variable domain of IgNAR |

TABLE 2
Amino acids abbreviations

| Abbreviation | 1 letter abbreviation | Amino acid name |
|---|---|---|
| Ala | A | Alanine |
| Arg | R | Arginine |
| Asn | N | Asparagine |
| Asp | D | Aspartic acid |
| Cys | C | Cysteine |
| Gln | Q | Glutamine |
| Glu | E | Glutamic acid |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Pyl | O | Pyrrolysine |
| Ser | S | Serine |
| Sec | U | Selenocysteine |
| Thr | T | Threonine |
| Trp | W | Tryptophan |
| Tyr | Y | Tyrosine |
| Val | V | Valine |

### Definitions

The following terms are used throughout the specification.

The term “acceptor human framework” is used herein is a framework comprising the amino acid sequence of a light chain variable domain (VL) framework or a heavy chain variable domain (VH) framework derived from a human immunoglobulin framework or a human consensus framework. An acceptor human framework derived from a human immunoglobulin framework or a human consensus framework may comprise the same amino acid sequence thereof, or it may contain amino acid sequence changes.

The term “affinity” refers to the strength of all noncovalent interactions between an antibody thereof and the target protein. Unless indicated otherwise, as used herein, the term “binding affinity” refers to intrinsic binding affinity which reflects a 1:1 interaction between members of a binding pair (e.g., antibody and antigen). The affinity of a molecule for its binding partner can be generally represented by the dissociation constant (KD). Affinity can be measured by common methods known in the art, including those described herein.

The term “affinity matured” in the context of antibody refers to an antibody with one or more alterations in the hypervariable regions, compared to a parent antibody which does not possess such alterations, where such alterations resulting in an improvement in the affinity of the antibody for antigen.

The term “antibody” herein is used in the broadest sense and encompasses various antibody structures, including but not limited to monoclonal antibodies, polyclonal antibodies, and multi-specific antibodies as long as they exhibit the desired antigen-binding activity. The term antibody as used herein relates to whole (full-length) antibodies (i.e. comprising the elements of two heavy chains and two light chains) and functionally active fragments thereof (i.e., molecules that contain an antigen binding domain that specifically binds to an antigen, also termed antibody fragments or antigen-binding fragments). Features described herein with respect to antibodies also apply to antibody fragments unless context dictates otherwise. An antibody may comprise a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibodies are described in WO2015/197772. The term “antibody” encompasses monovalent, i.e., antibodies comprising only one antigen binding domain (e.g. one-armed antibodies comprising a full-length heavy chain and a full-length light chain interconnected, also termed “half-antibody”), and multivalent antibodies, i.e. antibodies comprising more than one antigen binding domain, e.g bivalent.

The term “antibody binding to the same epitope as a reference antibody” refers to an antibody that blocks binding of the reference antibody to its antigen in a competition assay by 50% or more, and conversely, the reference antibody blocks binding of the antibody to its antigen in a competition assay by 50% or more.

The term “antibody-dependent cellular cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells.

The term “antigen-binding domain” as employed herein refers to a portion of the antibody, which comprises a part or the whole of one or more variable domains, for example a part or the whole of a pair of variable domains VH and VL, that interact specifically with a target antigen. In the context of the present invention the term is used in relation to three different antigens: IL13, IL22, and albumin. Hence, such antigen-binding domains are referred to as “IL13-binding domain”, “IL22-binding domain”, and “albumin-binding domain”. A binding domain may comprise a single domain antibody. Each binding domain may be monovalent. Each binding domain may comprise no more than one VH and one VL. Antigen-binding domain may comprise or consist of an antibody or antigen-binding fragment of an antibody. An example of an antigen-binding domain is a VH/VL unit comprised of a heavy chain variable domain (VH) and a light chain variable domain (VL).

The term “antigen-binding fragment” as employed herein refer to functionally active antibody binding fragments including but not limited to Fab, modified Fab, Fab′, modified Fab′, F(ab′)2, Fv, single domain antibodies, scFv, Fv, bi, tri or tetra-valent antibodies, Bis-scFv, diabodies, triabodies, tetrabodies and epitope-binding fragments of any of the above (see for example Holliger and Hudson, 2005, Nature Biotech. 23(9): 1126-1136; Adair and Lawson, 2005, Drug Design Reviews—Online 2(3), 209-217). A “binding fragment” as employed herein refers to a fragment capable of binding a target peptide or antigen with sufficient affinity to characterize the fragment as specific for the peptide or antigen.

The term “antibody variant” refers to a polypeptide, for example, an antibody possessing the desired characteristics described herein and comprising a VH and/or a VL that has at least about 80% amino acid sequence identity with a VH and/or a VL of the reference antibody. Such antibody variants include, for instance, antibodies wherein one or more amino acid residues are added to or deleted from the VH and/or a VL domain. Ordinarily, an antibody variant will have at least about 80% amino acid sequence identity, alternatively at least about any of 85%, 90%, 95%, 96%, 97%, 98%, or 99% amino acid sequence identity, to an antibody described herein. Optionally, variant antibodies will have no more than one conservative amino acid substitution as compared to an antibody sequence provided herein, alternatively no more than about any of 2, 3, 4, 5, 6, 7, 8, 9, or 10 conservative amino acid substitution as compared to an antibody sequence provided herein.

The term “bispecific” or “bispecific antibody” as employed herein refers to an antibody with two antigen specificities.

The term “complementarity determining regions” or “CDRs” refers to generally, antibodies comprise six CDRs: three in the VH (H1, H2, H3), and three in the VL (L1, L2, L3). The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system. Unless indicated otherwise, CDR residues and other residues in the variable domain (e.g., FR residues) are numbered herein according to Kabat.

The term “chimeric” antibody refers to an antibody in which the variable domain (or at least a portion thereof) of the heavy and/or light chain is derived from a particular source or species, while the remainder of the heavy and/or light chain (i.e. the constant domains) is derived from a different source or species. (Morrison; PNAS 81, 6851 (1984)). Chimeric antibodies can for instance comprise non-human variable domains and human constant domains. Chimeric antibodies are typically produced using recombinant DNA methods. A subcategory of “chimeric antibodies” is “humanized antibodies”.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “combination” or “antibody combination” in the context of multiple antibodies refers to multiple (2 or more) antibodies that are not physically mixed (being a part of a composition), but are provided a separate antibodies or each in a form of a composition in combination with other ingredients.

The term “complement-dependent cytotoxicity”, or “CDC” refers to a mechanism for inducing cell death in which an Fc effector domain of a target-bound antibody binds to and activates complement component C1q which in turn activates the complement cascade leading to target cell death.

The terms “constant domain(s)” or “constant region”, as used herein are used interchangeably to refer to the domain(s) of an antibody which is outside the variable regions. The constant domains are identical in all antibodies of the same isotype but are different from one isotype to another. Typically, the constant region of a heavy chain is formed, from N to C terminal, by CH1-hinge —CH2-CH3-, optionally CH4, comprising three or four constant domains.

The term “competing antibody” or “cross-competing antibody” shall be interpreted as meaning that the claimed antibody binds to either (i) the same position on the antigen to which the reference antibody binds, or (ii) a position on the antigen where the antibody sterically hinders the binding of the reference antibody to the antigen.

The term “Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The term “derived from” in the context of generating variable sequences refers to the fact that the sequence employed or a sequence highly similar to the sequence employed was obtained from the original genetic material, such as the light or heavy chain of an antibody.

The term “diabody” as employed herein refers to two Fv pairs, a first VH/VL pair and a further VH/VL pair which have two inter-Fv linkers, such that the VH of a first Fv is linked to the VL of the second Fv and the VL of the first Fv is linked to the VH of the second Fv.

The term “DiFab” as employed herein refers to two Fab molecules linked via their C-terminus of the heavy chains.

The term “DiFab′” as employed herein refers to two Fab′ molecules linked via one or more disulfide bonds in the hinge region thereof.

The term “dsFab” as employed herein refers to a Fab with an intra-variable region disulfide bond.

The term “dsscFv” or “disulphide-stabilised single chain variable fragment” as employed herein refer to a single chain variable fragment which is stabilised by a peptide linker between the VH and VL variable domain and also includes an inter-domain disulphide bond between VH and VL. (see for example, Weatherill et al., Protein Engineering, Design & Selection, 25 (321-329), 2012, WO2007109254.

The term “DVD-Ig” (also known as dual V domain IgG) refers to a full-length antibody with 4 additional variable domains, one on the N-terminus of each heavy and each light chain.

The term “effector functions” refer to those biological activities attributable to the Fc region of an antibody, which vary with the antibody isotype. Examples of antibody effector functions include: Clq binding and complement dependent cytotoxicity (CDC), Fc receptor binding, antibody-dependent cell-mediated cytotoxicity (ADCC), phagocytosis, down regulation of cell surface receptors (e.g. B cell receptor), and B cell activation.

The term “effector molecule” as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodine, radioisotopes, chelated metals, nanoparticles and reporter groups such as fluorescent compounds or compounds which may be detected by NMR or ESR spectroscopy.

The term “epitope” or “binding site” in the context of antibodies refer to a site (or a part) on an antigen to which the paratope of an antibody binds or recognizes. Epitopes can be formed both from contiguous amino acids (also often called “linear epitopes”) or noncontiguous amino acids formed by tertiary folding of a protein (often called “conformational epitopes”). Epitopes formed from contiguous amino acids are typically retained on exposure to denaturing solvents whereas epitopes formed by folding are typically lost on treatment with denaturing solvents. An epitope typically includes at least 3, and more usually, at least 5-10 amino acids in a unique spatial conformation. Epitopes usually consist of chemically active surface groups of molecules such as amino acids, sugar side chains and usually have specific 3D structural and charge characteristics.

The “EU index” or “EU index as in Kabat” or “EU numbering scheme” refers to the numbering of the EU antibody (Edelman et al., 1969, Proc Natl Acad Sci USA 63:78-85). Such is generally used when referring to a residue in an antibody heavy chain constant region (e.g., as reported in Kabat et al.). Unless stated otherwise, the EU numbering scheme is used to refer to residues in antibody heavy chain constant regions described herein.

The term “Fab” refers to as used herein refers to an antibody fragment comprising a light chain fragment comprising a VL (variable light) domain and a constant domain of a light chain (CL), and a VH (variable heavy) domain and a first constant domain (CH1) of a heavy chain. Dimers of a Fab′ according to the present disclosure create a F(ab′)2 where, for example, dimerization may be through the hinge.

The term “Fab′-Fv” as employed herein is similar to FabFv, wherein the Fab portion is replaced by a Fab′. The format may be provided as a PEGylated version thereof.

The term “Fab′-scFv” as employed herein is a Fab′ molecule with a scFv appended on the C-terminal of the light or heavy chain.

The term “Fab-dsFv” as employed herein refers to a FabFv wherein an intra-Fv disulfide bond stabilises the appended C-terminal variable regions. The format may be provided as a PEGylated version thereof.

The term “Fab-Fv” as employed herein refers to a Fab fragment with a variable region appended to the C-terminal of each of the following, the CH1 of the heavy chain and CL of the light chain. The format may be provided as a PEGylated version thereof.

The term “Fab-scFv” as employed herein is a Fab molecule with a scFv appended on the C-terminal of the light or heavy chain.

The term “Fc”, “Fc fragment”, and “Fc region” are used interchangeably to refer to the C-terminal region of an antibody comprising the constant region of an antibody excluding the first constant region immunoglobulin domain. Thus, Fc refers to the last two constant domains, CH2 and CH3, of IgA, IgD, and IgG, or the last three constant domains of IgE and IgM, and the flexible hinge N-terminal to these domains. The human IgG1 heavy chain Fc region is defined herein to comprise residues C226 to its carboxyl-terminus, wherein the numbering is according to the EU index. In the context of human IgG1, the lower hinge refers to positions 226-236, the CH2 domain refers to positions 237-340 and the CH3 domain refers to positions 341-447 according to the EU index. The corresponding Fc region of other immunoglobulins can be identified by sequence alignments.

The term “Framework” or “FR” refers to variable domain residues other than hypervariable region residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The term “full length antibody” used herein to refer to an antibody having a structure substantially similar to a native antibody structure or having heavy chains that contain an Fc region as defined herein. Each light chain is comprised of a light chain variable region (abbreviated herein as VL) and a light chain constant region (CL). Each heavy chain is comprised of a heavy variable region (abbreviated herein as VH) and a heavy chain constant region (CH) constituted of three constant domains CH1, CH2 and CH3, or four constant domains CH1, CH2, CH3 and CH4, depending on the Ig class. The constant regions of the antibodies may mediate the binding of the immunoglobulin to host tissues or factors, including various cells of the immune system (e.g., effector cells) and the first component (Clq) of the classical complement system.

The term “Fv” refers to two variable domains of full length antibodies, for example co-operative variable domains, such as a cognate pair or affinity matured variable domains, i.e. a VH and VL pair.

The term “highly similar” as employed in the context of amino-acid sequences is intended to refer to an amino acid sequence which over its full length is 95% similar or more, such as 96, 97, 98 or 99% similar.

The term “human antibody” refers to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “human consensus framework” refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup I, III or IV as in Kabat et al.

The term “humanized” antibody refers to an antibody comprising amino acid residues from non-human HVRs and amino acid residues from human FRs. Typically the heavy and/or light chain contains one or more CDRs (including, if desired, one or more modified CDRs) from a donor antibody (e.g. a non-human antibody such as a murine or rabbit monoclonal antibody) and is grafted into a heavy and/or light chain variable region framework of an acceptor antibody (a human antibody)(see e.g. Vaughan et al, Nature Biotechnology, 16, 535-539, 1998). The advantage of such humanized antibodies is to reduce immunogenicity to humans, while retaining the specificity and affinity of the parental non-human antibody. Rather than the entire CDR being transferred, only one or more of the specificity determining residues from any one of the CDRs described herein above can be transferred to the human antibody framework (see e.g., Kashmiri et al., 2005, Methods, 36, 25-34). A “humanized” antibody refers to a chimeric antibody comprising amino acid residues from non-human HVRs and amino acid residues from human FRs. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”).

The term “IC50” as used herein refers to the half maximal inhibitory concentration which is a measure of the effectiveness of a substance, such as an antibody, in inhibiting a specific biological or biochemical function. The IC50 is a quantitative measure which indicates how much of a particular substance is needed to inhibit a given biological process by 50%.

The “identity” between amino-acids in the sequence indicates that at any particular position in the aligned sequences, the amino acid residue is identical between the sequences.

The term “IgG-scFv” as employed herein is a full-length antibody with a scFv on the C-terminal of each of the heavy chains or each of the light chains.

The term “IgG-V” as employed herein is a full-length antibody with a variable domain on the C-terminal of each of the heavy chains or each of the light chains

The term “isolated” means, throughout this specification, that the antibody, or polynucleotide, as the case may be, exists in a physical milieu distinct from that in which it may occur in nature. The term “isolated” nucleic acid refers to a nucleic acid molecule that has been isolated from its natural environment or that has been synthetically created. An isolated nucleic acid may comprise synthetic DNA, for instance produced by chemical processing, cDNA, genomic DNA or any combination thereof.

The term “Kabat residue designations” or “Kabat” refer to the residue numbering scheme commonly used for antibodies. Such do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. For details see Kabat et al., Sequences of Proteins of Immunological Interest, 5th Ed. Public Health Service, National Institutes of Health, Bethesda, MD (1991). Unless indicated otherwise, Kabat numbering is used throughout the specification

The term “KD” as used herein refers to the constant of dissociation which is obtained from the ratio of Kd to Ka (i.e. Kd/Ka) and is expressed as a molar concentration (M). Kd and Ka refers to the dissociation rate and association rate, respectively, of a particular antigen-antibody interaction. KD values for antibodies can be determined using methods well established in the art.

The term “monoclonal antibody” (or “mAb”) refers to an antibody obtained from a population of substantially homogeneous antibodies, i.e. each individual of a monoclonal antibody preparation are identical except for possible mutations (e.g., naturally occurring mutations), that may be present in minor amounts. Certain differences in the protein sequences linked to post-translational modifications (for example, cleavage of the heavy chain C-terminal lysine, deamidation of asparagine residues and/or isomerisation of aspartate residues) may nevertheless exist between the various different antibody molecules present in the composition. Contrary to polyclonal antibody preparations, each monoclonal antibody of a monoclonal antibody preparation is directed against a single determinant on an antigen.

The term “multi-paratopic antibody” as employed herein refers to an antibody as described herein which comprises two or more distinct paratopes, which interact with different epitopes either from the same antigen or from two different antigens. Multi-paratopic antibodies described herein may be biparatopic, triparatopic, tetraparatopic.

The term “multi-specific” or “multi-specific antibody” as employed herein refers to an antibody as described herein which has at least two binding domains, i.e. two or more binding domains, for example two or three binding domains, wherein the at least two binding domains independently bind two different antigens or two different epitopes on the same antigen. Multi-specific antibodies are generally monovalent for each specificity (antigen). Multi-specific antibodies described herein encompass monovalent and multivalent, e.g. bivalent, trivalent, tetravalent multi-specific antibodies.

The term “neutralizing” (or “neutralize”) in the context of antibodies and antigen-binding domains describes an antibody (or an antigen-binding domain) that is capable of inhibiting or attenuating the biological signaling activity of its target (target protein).

The term “paratope” refers to a region of an antibody which recognizes and binds to an antigen.

The term “percent (%) sequence identity (or similarity)” with respect to the polypeptide and antibody sequences is defined as the percentage of amino acid residues in a candidate sequence that are identical (or similar) to the amino acid residues in the polypeptide being compared, after aligning the sequences and introducing gaps, if necessary, to achieve the maximum percent sequence identity, and not considering any conservative substitutions as part of the sequence identity.

A “pharmaceutically acceptable carrier” refers to an ingredient in a pharmaceutical formulation, other than an active ingredient, which is nontoxic to a subject. Pharmaceutically acceptable carriers include, but are not limited to, a buffer, excipient, stabilizer, or preservative.

The term “polyclonal antibody” refers to a mixture of different antibody molecules which bind to (or otherwise interact with) more than one epitope of an antigen

The term “prevent” in the context of antibodies is used herein interchangeably with the term “inhibit” and indicates the effect the antibodies according to the present invention have with respect to a particular biological process or molecular interaction.

The term “scDiabody” refers to a diabody comprising an intra-Fv linker, such that the molecule comprises three linkers and forms a normal scFv whose VH and VL terminals are each linked to a one of the variable regions of a further Fv pair.

The term “Scdiabody-CH3” as employed herein refers to two scdiabody molecules each linked, for example via a hinge to a CH3 domain.

The term “ScDiabody-Fc” as employed herein is two scdiabodies, wherein each one is appended to the N-terminus of a CH2 domain, for example via a hinge, of constant region fragment —CH2CH3.

The term “single chain variable fragment” or “scFv” as employed herein refers to a single chain variable fragment which is stabilised by a peptide linker between the VH and VL variable domains.

The term “ScFv-Fc-scFv” as employed herein refers to four scFvs, wherein one of each is appended to the N-terminus and the C-terminus of both the heavy chains of a —CH2CH3 fragment.

The term “scFv-IgG” as employed herein is a full-length antibody with a scFv on the N-terminal of each of the heavy chains or each of the light chains.

The term “similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

The term “single domain antibody” as used herein refers to an antibody fragment consisting of a single monomeric variable domain. Examples of single domain antibodies include VH or VL or VHH or V-NAR.

The term “specific” as employed herein in the context of antibodies is intended to refer to an antibody that only recognizes the antigen to which it is specific or an antibody that has significantly higher binding affinity to the antigen to which it is specific compared to binding to antigens to which it is non-specific, for example at least 5, 6, 7, 8, 9, 10 times higher binding affinity.

The term “sterically blocking” or “sterically preventing” as employed herein is intended to refer to the means of blocking an interaction between first and second proteins by a third protein's binding to the first protein. The binding between the first and the third proteins prevents the second protein from binding to the first protein due to unfavorable van der Waals or electrostatic interactions between the second and third proteins.

The terms “subject” or “individual in the context of the treatments and diagnosis generally refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). More specifically, the individual or subject is a human

The term “Tandem scFv” as employed herein refers to at least two scFvs linked via a single linker such that there is a single inter-Fv linker.

The term “Tandem scFv-Fc” as employed herein refers to at least two tandem scFvs, wherein each one is appended to the N-terminus of a CH2 domain, for example via a hinge, of constant region fragment —CH2CH3.

The term “target” or “antibody target” as used herein refers to target antigen to which the antibody binds.

The term “Tetrabody” as employed herein refers to a format similar to the diabody comprising fours Fvs and four inter-Fv linkers.

The term “therapeutically effective amount” refers to the amount of an antibody thereof that, when administered to a subject for treating a disease, is sufficient to produce such treatment for the disease. The therapeutically effective amount will vary depending on the antibody, the disease and its severity and the age, weight, etc., of the subject to be treated.

The term “tribody” (also referred to a Fab(scFv)₂) as employed herein refers to a Fab fragment with a first scFv appended to the C-terminal of the light chain and a second scFv appended to the C-terminal of the heavy the chain.

The term “trispecific or trispecific antibody” as employed herein refers to an antibody with three antigen binding specificities. For example, the antibody is an antibody with three antigen binding domains (trivalent), which independently bind three different antigens or three different epitopes on the same antigen, i.e. each binding domain is monovalent for each antigen. One of the examples of a trispecific antibody format is TrYbe.

The terms “prevent”, or “preventing” and the like, refer to obtaining a prophylactic effect in terms of completely or partially preventing a disease or symptom thereof. Preventing thus encompasses stopping the disease from occurring in a subject who may be predisposed to the disease but has not yet been diagnosed as having the disease.

The terms “treatment”, “treating” and the like, refer to obtaining a desired pharmacologic and/or physiologic effect. The effect may be therapeutic in terms of a partial or complete cure for a disease and/or adverse effect attributable to the disease. Treatment thus encompasses (a) inhibiting the disease, i.e., arresting its development; and (b) relieving the disease, i.e., causing regression of the disease.

The term “TrYbe” as employed herein refers to a tribody comprising two dsscFvs (Fab(dsscFv)₂). The term “IL13/IL22 TrYbe” as employed herein refers to a TrYbe that comprises IL13- and IL22-binding domains, and an albumin-binding domain.

The term “variable region” or “variable domain” refers to the domain of an antibody heavy or light chain that is involved in binding the antibody to antigen. The variable domains of the heavy chain (VH) and light chain (VL) of a full length generally have similar structures, with each domain comprising four conserved framework regions (FRs) and three CDRs. (See, e.g., Kindt et al. Kuby Immunology, 6th ed., W.H. Freeman and Co., page 91 (2007).) A single VH or VL domain may be sufficient to confer antigen-binding specificity. Each VH and VL is composed of three CDRs and four FRs, arranged from amino-terminus to carboxy-terminus in the following order: FR1, CDR1, FR2, CDR2, FR3, CDR3, FR4. The CDRs and the FR together form a variable region. By convention, the CDRs in the heavy chain variable region of an antibody are referred as CDR-H1, CDR-H2 and CDR-H3 and in the light chain variable regions as CDR-L1, CDR-L2 and CDR-L3. They are numbered sequentially in the direction from the N-terminus to the C-terminus of each chain. CDRs are conventionally numbered according to a system devised by Kabat.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” The term “vector” includes “expression vectors”.

The term “VH” refers to the variable domain (or the sequence) of the heavy chain.

The term “V-IgG” as employed herein is a full-length antibody with a variable domain on the N-terminal of each of the heavy chains or each of the light chains.

The term “VL” refers to the variable domain (or the sequence) of the light chain.

### Interleukin 22 (IL22)

The term “interleukin-22” or “IL22” refers to a class II cytokine capable of binding to IL22R1 (also known as IL22RA1, IL22 receptor 1, or Interleukin-22 receptor subunit alpha-1) and/or a receptor complex of IL22R1 and IL10RA2 (also known as IL22BP, IL22 binding protein, or Interleukin-22 Receptor Subunit Alpha 2). IL22 is also known as interleukin-10 related T cell-derived inducible factor (IL-TIF). The term refers to naturally occurring or endogenous mammalian IL22 proteins and to proteins having an amino acid sequence which is the same as that of a naturally occurring or endogenous corresponding mammalian IL22 protein (e.g., recombinant proteins, synthetic proteins). Accordingly, as defined herein, the term includes mature IL22 protein, polymorphic or allelic variants, and other isoforms of IL22 (e.g., produced by alternative splicing or other cellular processes), and modified or unmodified forms of the foregoing (e.g., Lipidated, glycosylated). Naturally occurring or endogenous IL22 include wild type proteins such as mature IL22, polymorphic or allelic variants and other isoforms and mutant forms which occur naturally in mammals (e.g., humans, non-human primates). These proteins and proteins having the same amino acid sequence as a naturally occurring or endogenous corresponding IL22 are referred to by the name of the corresponding mammal.

The amino acid sequence of mature human IL22 corresponds to amino acids 34-179 of SEQ ID NO:1. Analysis of recombinant human IL22 reveals many structural domains. (Nagem et al. (2002) Structure, 10:1051-62; US2002/0187512).

### Interleukin 13 (IL13)

“Interleukin-13” or “IL13” refers to naturally occurring or endogenous mammalian IL13 proteins and to proteins having an amino acid sequence which is the same as that of a naturally occurring or endogenous corresponding mammalian IL13 protein (e.g., recombinant proteins, synthetic proteins). Accordingly, as defined herein, the term includes mature IL13 protein, polymorphic or allelic variants, and other isoforms of IL13 (e.g., produced by alternative splicing or other cellular processes), and modified or unmodified forms of the foregoing (e.g., Lipidated, glycosylated). Naturally occurring or endogenous IL13 include wild type proteins such as mature IL13, polymorphic or allelic variants and other isoforms and mutant forms which occur naturally in mammals (e.g., humans, non-human primates). These proteins and proteins having the same amino acid sequence as a naturally occurring or endogenous corresponding IL13 are referred to by the name of the corresponding mammal. For example, where the corresponding mammal is a human, the protein is designated as a human IL13. Several mutant IL13 proteins are known in the art, such as those disclosed in WO 03/035847.

The term “human IL13”, as used herein, includes a human IL13 cytokine. The term includes a monomeric protein of 13 kDa polypeptide. The structure of human IL13 is described further in, for example, (Moy, Diblasio et al. 2001 J Mol Biol 310 219-30). The term human IL13 is intended to include recombinant human IL13 (which can be prepared by standard recombinant expression methods). The amino acid sequence of mature human IL13 corresponds to amino acids 25-146 of SEQ ID NO:5.

Antibodies and Antigen Binding Domains that Bind to IL22 and IL13

The present invention provides an antibody that comprises an antigen binding domain that binds to IL13 (“IL13-binding domain”) and an antigen binding domain that binds to IL22 (“IL22-binding domain”) (multi-specific antibody).

Alternatively, the IL13 and IL22 antigen-binding domains might also be present on different antibodies. Hence, the invention in some embodiments utilizes an antibody that comprises an IL22-binding domain, and an antibody that comprises an IL13-binding domain. Such antibodies might be a part of a composition. Alternatively, they might be provided individually, or each in a form of a composition. The antibody might be a full-length antibody or a fragment of a full-length antibody.

The antibodies may be (or derived from) polyclonal, monoclonal, fully human, humanized or chimeric.

The antibodies described further in for purposes of reference and example only and do not limit the scope of invention.

An antibody used according to the invention may be a monoclonal antibody or a polyclonal antibody, and is preferably a monoclonal antibody. An antibody used according to the invention may be a chimeric antibody, a CDR-grafted antibody, a nanobody, a human or humanized antibody. For the production of both monoclonal and polyclonal antibodies, the animal used to raise such antibodies is typically a non-human mammal such as a goat, rabbit, rat or mouse but the antibody may also be raised in other species.

Polyclonal antibodies may be produced by routine methods such as immunization of a suitable animal with an antigen of interest. Blood may be subsequently removed from such animal and the produced antibodies purified.

Monoclonal antibodies may be made by a variety of techniques, including but not limited to, the hybridoma method, recombinant DNA methods, phage-display methods, and methods utilizing transgenic animals containing all or a part of the human immunoglobulin loci. Some exemplary methods for making monoclonal antibodies are described herein.

For example, monoclonal antibodies may be prepared using the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Monoclonal antibodies may also be generated using single lymphocyte antibody methods by cloning and expressing immunoglobulin variable region cDNAs generated from single lymphocytes selected for the production of specific antibodies by for example the methods described in WO9202551, WO2004051268 and WO2004106377.

Antibodies generated against the target polypeptide may be obtained, where immunization of an animal is necessary, by administering the polypeptide to an animal, preferably a non-human animal, using well-known and routine protocols, see for example Handbook of Experimental Immunology, D. M. Weir (ed.), Vol 4, Blackwell Scientific Publishers, Oxford, England, 1986). Many animals, such as rabbits, mice, rats, sheep, cows, camels or pigs may be immunized. However, mice, rabbits, pigs and rats are generally used.

Monoclonal antibodies can also be generated using various. phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280). In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol, 227: 381-388 (1992). Patent publications describing human antibody phage libraries include, for example: U.S. Pat. No. 5,750,373, and US 2005/0079574, U52005/0119455, US2005/0266000, US2007/0117126, US2007/0160598, US2007/0237764, US2007/0292936, and US2009/0002360.

Screening for antibodies can be performed using assays to measure binding to the target polypeptide and/or assays to measure the ability of the antibody to block a particular interaction. An example of a binding assay is an ELISA, for example, using a fusion protein of the target polypeptide, which is immobilized on plates, and employing a conjugated secondary antibody to detect the antibody bound to the target. An example of a blocking assay is a flow cytometry based assay measuring the blocking of a ligand protein binding to the target polypeptide. A fluorescently labelled secondary antibody is used to detect the amount of such ligand protein binding to the target polypeptide.

Antibodies may be isolated by screening combinatorial libraries for antibodies with the desired activity or activities. For example, a variety of methods are known in the art for generating phage display libraries and screening such libraries for antibodies possessing the desired binding characteristics.

Antibodies or antibody fragments isolated from human antibody libraries are considered human antibodies or human antibody fragments.

The antibody may be a full length antibody. More particularly the antibody may be of the IgG isotype. More particularly the antibody may be an IgG1 or IgG4.

The constant region domains of the antibody, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. It will also be known to the person skilled in the art that antibodies may undergo a variety of posttranslational modifications. The type and extent of these modifications often depends on the host cell line used to express the antibody as well as the cell culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Accordingly, the C-terminal lysine of the antibody heavy chain may be absent.

Alternatively, the antibody is an antigen-binding fragment.

For a review of certain antigen-binding fragments, see Hudson et al. Nat. Med. 9: 129-134 (2003). For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. Fab and F(ab′)2 fragments comprising salvage receptor binding epitope residues and having increased in vivo half-life are disclosed in U.S. Pat. No. 5,869,046.

Antigen-binding fragments and methods of producing them are well known in the art, see for example Verma et al., 1998, Journal of Immunological Methods, 216, 165-181; Adair and Lawson, 2005. Therapeutic antibodies. Drug Design Reviews—Online 2(3):209-217. The Fab-Fv format was first disclosed in WO2009/040562 and the disulphide stabilized version thereof, the Fab-dsFv, was first disclosed in WO2010/035012, and TrYbe format is disclosed in WO2015/197772.

Various techniques have been developed for the production of antibody fragments. Such fragments might be derived via proteolytic digestion of intact antibodies (see, e.g., Morimoto et al., Journal of Biochemical and Biophysical Methods 24: 107-117 (1992) and Brennan et al, Science 229:81 (1985)). However, antibody fragments can also be produced directly by recombinant host cells. For example, antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)).

F(ab′)₂ fragments can be isolated directly from recombinant host cell culture. The antibody may be a single chain Fv fragment (scFv). Such are described in WO 93/16185; U.S. Pat. Nos. 5,571,894; and 5,587,458. The antibody fragment may also be a “linear antibody,” e.g., as described in U.S. Pat. No. 5,641,870. Such linear antibody fragments may be monospecific or bispecific.

The antibody may be a Fab, Fab′, F(ab′)₂, Fv, dsFv, scFv, or dsscFv. The antibody may be a single domain antibody or a nanobody, for example VH or VL or VHH or VNAR. The antibody may be Fab or Fab′ fragment described in WO2011/117648, WO2005/003169, WO2005/003170 and WO2005/003171.

The antibody may be a Disulphide—stabilized single chain variable fragment (dsscFv).

The disulfide bond between the variable domains VH and VL may be between two of the residues listed below:
V_{H}37+V_{L}95 see for example Protein Science 6, 781-788 Zhu et al (1997);
V_{H}44+V_{L}100 see for example Weatherill et al., Protein Engineering, Design & Selection, 25 (321-329), 2012;
V_{H}44+V_{L}105 see for example J Biochem. 118, 825-831 Luo et al (1995);
V_{H}45+V_{L}87 see for example Protein Science 6, 781-788 Zhu et al (1997);
V_{H}55+V_{L}101 see for example FEBS Letters 377 135-139 Young et al (1995);
V_{H}100+V_{L}50 see for example Biochemistry 29 1362-1367 Glockshuber et al (1990);
V_{H}100b+V_{L}49; see for example Biochemistry 29 1362-1367 Glockshuber et al (1990);
V_{H}98+V_{L} 46 see for example Protein Science 6, 781-788 Zhu et al (1997);
V_{H}101+V_{L}46; see for example Protein Science 6, 781-788 Zhu et al (1997);
V_{H}105+V_{L}43 see for example; Proc. Natl. Acad. Sci. USA Vol. 90 pp. 7538-7542 Brinkmann et al (1993); or Proteins 19, 35-47 Jung et al (1994),
V_{H}106+V_{L}57 see for example FEBS Letters 377 135-139 Young et al (1995)
and a position or positions corresponding thereto in a variable region pair located in the molecule.

The disulphide bond may be formed between positions VH44 and VL100.

It will be appreciated by the skilled person that antigen-binding fragments described herein may also be characterized as monoclonal, chimeric, humanized, fully human, multispecific, bispecific etc., and that discussion of these terms also relate to such fragments.

### Multi-Specific Antibodies Binding to IL22 and IL13

The present invention provides a multi-specific antibody comprising at least two antigen

binding domains, wherein at least one antigen binding domain binds to IL13 (“IL13-binding domain”) and at least one antigen-binding domain binds to IL22 (“IL22-binding domain”). In particular, such antigen-binding domains bind specifically to their corresponding targets.

Examples of multi-specific antibodies, which also are contemplated for use in the context of the disclosure, include bi, tri or tetra-valent antibodies, Bis-scFv, diabodies, triabodies, tetrabodies, bibodies and tribodies (see for example Holliger and Hudson, 2005, Nature Biotech 23(9): 1126-1136; Schoonjans et al. 2001, Biomolecular Engineering, 17(6), 193-202).

In one embodiment the multi-specific antibody is a bispecific antibody. In one embodiment, the antibody comprises two antigen binding domains wherein one binding domain binds to IL13 and the other binding domain binds to IL22, i.e. each binding domain is monovalent for each antigen. In one embodiment, the antibody is a tetravalent bispecific antibody, i.e. the antibody comprises four antigen binding domains, wherein for example two binding domains bind to IL13 and the other two binding domains bind to IL22. In one embodiment, the antibody is a trivalent bispecific antibody.

In one embodiment the multi-specific antibody is a trispecific antibody.

The multi-specific antibody of the invention may be a multi-paratopic antibody.

In one embodiment, each binding domain is monovalent. Preferably each binding domain comprises two antibody variable domains. More preferably each binding domain comprises no more than one VH and one VL.

More particularly the binding domain which binds to IL13 and the binding domain which binds to IL22 are independently selected from a Fab, scFv, Fv, dsFv and dsscFv

A variety of different multi-specific antibody formats are known in the art. Different classifications have been proposed, but multi-specific IgG antibody formats generally include bispecific IgG, appended IgG, multi-specific (e.g. bispecific) antibody fragments, multi-specific (e.g. bispecific) fusion proteins, and multi-specific (e.g. bispecific) antibody conjugates, as described for example in Spiess et al., Alternative molecular formats and therapeutic applications for bispecific antibodies. Mol Immunol. 67(2015):95-106.

Techniques for making bispecific antibodies include, but are not limited to, CrossMab technology (Klein et al. Engineering therapeutic bispecific antibodies using CrossMab technology, Methods 154 (2019) 21-31), Knobs-in-holes engineering (e.g. WO1996027011, WO1998050431), DuoBody technology (e.g. WO2011131746), Azymetric technology (e.g. WO2012058768). Further technologies for making bispecific antibodies have been described for example in Godar et al., 2018, Therapeutic bispecific antibody formats: a patent applications review (1994-2017), Expert Opinion on Therapeutic Patents, 28:3, 251-276. Bispecific antibodies include in particular CrossMab antibodies, DAF (two-in-one), DAF (four-in-one), DutaMab, DT-IgG, Knobs-in-holes common LC, Knobs-in-holes assembly, Charge pair, Fab-arm exchange, SEEDbody, Triomab, LUZ-Y, Fcab, κλ-body and orthogonal Fab.

Appended IgG classically comprise full-length IgG engineered by appending additional antigen-binding fragment to the N- and/or C-terminus of the heavy and/or light chain of the IgG. Examples of such additional antigen-binding fragments include sdAb antibodies (e.g. VH or VL), Fv, scFv, dsscFv, Fab, scFab. Appended IgG antibody formats include in particular DVD-IgG, IgG(H)-scFv, scFv-(H)IgG, IgG(L)-scFv, scFv-(L)IgG, IgG(L,H)-Fv, IgG(H)-V, V(H)-IgG, IgC(L)-V, V(L)-IgG, KIH IgG-scFab, 2scFv-IgG, IgG-2scFv, scFv4-Ig, Zybody and DVI-IgG (four-in-one), for example as described in Spiess et al., Mol Immunol. 67(2015):95-106.

Multi-specific antibodies include nanobody, nanobody-HSA, BiTEs, diabody, DART, TandAb, scDiabody, sc-Diabody-CH3, Diabody-CH3, Triple Body, Miniantibody; Minibody, Tri Bi minibody, scFv-CH3 KIH, Fab-scFv, scFv-CH-CL-scFv, F(ab)₂, F(ab′)₂-scFv₂, scFv-KIH, Fab-scFv-Fc, Tetravalent HCAb, scDiabody-Fc, Diabody-Fc, Tandem scFv-Fc; and intrabody, as described, for example, Spiess et al., Mol Immunol. 67(2015):95-106.

Multi-specific fusion proteins include Dock and Lock, ImmTAC, HSAbody, scDiabody-HSA, and Tandem scFv-Toxin.

Multi-specific antibody conjugates include IgG-IgG; Cov-X-Body; and scFv1-PEG-scFv2.

Additional multi-specific antibody formats have been described for example in Brinkmann and Kontermann, mAbs, 9:2, 182-212 (2017), for example tandem scFv, triplebody, Fab-VHH, taFv-Fc, scFv4-Ig, scFv₂-Fcab, scFv4-IgG. Bibodies, tribodies and methods for producing the same are disclosed for example in WO99/37791.

A preferred multi-specific antibody for use in the present invention comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such multi-specific antibodies are described in WO2015/197772. In a preferred embodiment the multi-specific antibody comprises a Fab binding to human IL22 linked to two scFv or dsscFv, where one scFv or dsscFv binds to IL13 and one scFv or dsscFv binds to albumin. Another preferred antibody for use in the present invention fragment comprises a Fab linked to only one scFv or dsscFv, as described for example in WO2013/068571, and Dave et al., Mabs, 8(7) 1319-1335 (2016).

Another preferred multi specific antibody for use in the present invention is a Knobs-into-holes antibody (“KiH”). Generally, such technology involves introducing a protuberance (“knob”) at the interface of a first polypeptide (such as a first CH3 domain in a first antibody heavy chain) and a corresponding cavity (“hole”) in the interface of a second polypeptide (such as a second CH3 domain in a second antibody heavy chain), such that the protuberance can be positioned in the cavity so as to assist the formation bispecific antibody. Protuberances are constructed by replacing small amino acid side chains from the interface of the first polypeptide (such as a first CH3 domain in a first antibody heavy chain) with larger side chains (e.g. arginine, phenylalanine, tyrosine or tryptophan). Compensatory cavities of identical or similar size to the protuberances are created in the interface of the second polypeptide (such as a second CH3 domain in a second antibody heavy chain) by replacing large amino acid side chains with smaller ones (e.g. alanine, serine, valine, or threonine). The protuberance and cavity can be made by altering the nucleic acid encoding the polypeptides, e.g. by site-specific mutagenesis, or by peptide synthesis. Further details regarding “knobs-into-holes” technology is described in, e.g., U.S. Pat. Nos. 5,731,168; 7,695,936; WO2009/089004; US2009/0182127; Marvin and Z u, Acta Pharmacologica Sincia (2005) 26(6):649-658; Kontermann Acta Pharmacologica Sincia (2005) 26: 1-9; Ridgway et al, Prot Eng 9, 617-621 (1996); and Carter, J Immunol Meth 248, 7-15 (2001).

### Antibodies Binding to Albumin

The high specificity and affinity of antibodies make them ideal diagnostic and therapeutic agents, particularly for modulating protein:protein interactions. However, antibodies may suffer from an increased rate of clearance from serum, especially when they lack the Fc domain that imparts a long lifetime in vivo (Medasan et al., 1997, J. Immunol. 158:2211-2217).

Means to improve the half-life of antibodies are known. One approach has been to conjugate the fragment to polymer molecules. Thus, the short circulating half-life of Fab′, F(ab′)2 fragments in animals has been improved by conjugation to polyethylene glycol (PEG; see, for example, WO98/25791, WO99/64460 and WO98/37200). Another approach has been to modify the antibody fragment by conjugation to an agent that interacts with the FcRn receptor (see, for example, WO97/34631). Yet another approach to extend half-life has been to use polypeptides that bind serum albumin (see, for example, Smith et al., 2001, Bioconjugate Chem. 12:750-756; EP0486525; U.S. Pat. No. 6,267,964; WO04/001064; WO02/076489; and WO01/45746).

Serum albumin is an abundant protein in both vascular and extravascular compartments with a half-life in man of about 19 days (Peters, 1985, Adv Protein Chem. 37:161-245). This is similar to the half-life of IgG1, which is about 21 days (Waldeman & Strober, 1969, Progr. Allergy, 13:1-110).

Anti-serum albumin binding single variable domains have been described along with their use as conjugates to increase the half-life of drugs, including NCE (chemical entity) drugs, proteins and peptides, see for example, Holt et al., Protein Engineering, Design & Selection, vol 21, 5, pp 283-288, WO04003019, WO2008/096158, WO05118642, WO2006/0591056 and WO2011/006915. Other anti-serum albumin antibodies and their use in multi-specific antibody formats have been described in WO2009/040562, WO2010/035012 and WO2011/086091. In particular, the present inventors have previously described an anti-albumin antibody with improved humanization in WO2013/068571.

In some embodiments the multi-specific antibodies of the present invention have been engineered to bind to human serum albumin (e.g. contain an albumin-binding domain), in order to extend their in vivo serum half-life, resulting in improved pharmacokinetic profiles.

### Humanized, Human, and Chimeric Antibodies and Methods of Producing Such

The antibodies of the present invention may be, but are not limited to, humanized, fully human or chimeric antibodies.

In one embodiment the antibodies are humanized. More particularly the antibody is a chimeric, human, or humanized antibody.

In certain embodiments, an antibody provided herein is a chimeric antibody. Examples of chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In one embodiment, the antibody is a humanized antibody.

Humanized antibodies may optionally further comprise one or more framework residues derived from the non-human species from which the CDRs were derived. It will be appreciated that it may only be necessary to transfer the specificity determining residues of the CDRs rather than the entire CDR (see for example, Kashmiri et al., 2005, Methods, 36, 25-34).

Suitably, the humanized antibody according to the present invention has a variable domain comprising human acceptor framework regions as well as one or more of the CDRs and optionally further including one or more donor framework residues.

Thus, provided in one embodiment is a humanized antibody wherein the variable domain comprises human acceptor framework regions and non-human donor CDRs.

When the CDRs or specificity determining residues are grafted, any appropriate acceptor variable region framework sequence may be used having regard to the class/type of the donor antibody from which the CDRs are derived, including mouse, primate and human framework regions.

Examples of human frameworks which can be used in the present invention are KOL, NEWM, REI, EU, TUR, TEI, LAY and POM (Kabat et al). For example, KOL and NEWM can be used for the heavy chain, REI can be used for the light chain and EU, LAY and POM can be used for both the heavy chain and the light chain. Alternatively, human germline sequences may be used; these are available at: www.imgt.org. In embodiments, the acceptor framework is IGHV1-69 human germline, IGKV1D-13 human germline, IGHV3-66 human germline, IGKV1-12 human germline, IGKV1-39 human germline and/or IGHV4-31 human germline. In embodiments, the human framework contains 1-5, 1-4, 1-3 or 1-2 donor antibody amino acid residues.

In a humanized antibody of the present invention, the acceptor heavy and light chains do not necessarily need to be derived from the same antibody and may, if desired, comprise composite chains having framework regions derived from different chains.

In certain embodiments, an antibody provided herein is a human antibody. Human antibodies can be produced using various techniques known in the art.

Human antibodies comprise heavy or light chain variable regions or full length heavy or light chains that are “the product of” or “derived from” a particular germline sequence if the variable regions or full-length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody or fragment thereof that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acid sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Antigen-Binding Domains and their Sequences

An antigen binding domain will generally comprise 6 CDRs, three from a heavy chain and three from a light chain. In one embodiment, the CDRs are in a framework and together form a variable region. Thus, in one embodiment, the binding domain specific for antigen comprises a light chain variable region and a heavy chain variable region.

In the context of the antibodies of the present invention tree types of antigen-binding domains are being referred to: an IL22-binding domain, an IL13-binding domain, and an albumin-binding domain.

TABLE 3
Summary of sequences for IL22 and IL13
antigen-binding domains (b.d.)

| | IL22 b.d. | IL22 b.d | |
|---|---|---|---|
| | (11041) | (11070) | IL13 b.d. |
| | SEQ IDs | SEQ IDs | SEQ IDs |
| CDR-L1 | 8 | 70 | 22 |
| CDR-L2 | 9 | 71 | 23 |
| CDR-L3 | 10 | 72 | 24 |
| CDR-H1 | 11 | 73 | 25 |
| CDR-H2 | 12 | 74 | 26 |
| CDR-H3 | 13 | 75 | 27 |
| VL of Fab | 14 | 76 | — |
| VH of Fab | 16 | 78 | — |
| VL of scFv | — | — | 28 |
| VH of scFv | — | — | 29 |
| VL of dsscFv | — | — | 32 |
| VH of dsscFv | — | — | 33 |
| LC Fab | 18 | 80 | — |
| HC Fab | 20 | 82 | — |
| scFv (VH/VL) | — | — | 36 |
| dsscFv (VH/VL) | — | — | 38 |

In one embodiment, the multi-specific antibody comprises an antigen-binding domain that binds to IL22 comprising
a light chain variable region comprising:
   a CDR-L1 comprising SEQ ID NO:8,
   a CDR-L2 comprising SEQ ID NO:9, and
   a CDR-L3 comprising SEQ ID NO:10;
and a heavy chain variable region comprising:
   a CDR-H1 comprising SEQ ID NO:11,
   a CDR-H2 comprising SEQ ID NO:12, and
   a CDR-H3 comprising SEQ ID NO:13.

In another embodiment, the multi-specific antibody comprises an antigen-binding domain that binds to IL22 comprising
a light chain variable region comprising:
   a CDR-L1 comprising SEQ ID NO:70,
   a CDR-L2 comprising SEQ ID NO:71, and
   a CDR-L3 comprising SEQ ID NO:72;
and a heavy chain variable region comprising:
   a CDR-H1 comprising SEQ ID NO:73,
   a CDR-H2 comprising SEQ ID NO: 74, and
   a CDR-H3 comprising SEQ ID NO:75.

In one embodiment, the multi-specific antibody comprises an antigen-binding domain that binds to IL13 comprising
a light chain variable region comprising:
   a CDR-L1 comprising SEQ ID NO:22,
   a CDR-L2 comprising SEQ ID NO:23, and
   a CDR-L3 comprising SEQ ID NO:24;
and a heavy chain variable region comprising:
   a CDR-H1 comprising SEQ ID NO:25,
   a CDR-H2 comprising SEQ ID NO:26, and
   a CDR-H3 comprising SEQ ID NO:27.

In one embodiment, the antigen-binding domain that binds to IL22 comprises a light chain variable region comprising the sequence given in SEQ ID NO:14 and heavy chain variable region comprising the sequence given in SEQ ID NO:16.

Alternatively, the antigen-binding domain that binds to IL22 comprises a light chain variable region comprising the sequence given in SEQ ID NO:76 and heavy chain variable region comprising the sequence given in SEQ ID NO:78.

In one embodiment, the antigen-binding domain that binds to IL13 comprises a light chain variable region comprising the sequence given in SEQ ID NO:28 and a heavy chain variable region comprising the sequence given in SEQ ID NO:29.

In an alternative embodiment, the antigen-binding domain that binds to IL13 comprises a light chain variable region comprising the sequence given in SEQ ID NO:32 and a heavy chain variable region comprising the sequence given in SEQ ID NO:33.

In one embodiment, the antigen-binding domain that binds to IL13 is a scFv comprising the sequence given in SEQ ID NO:36 or a dsscFv comprising the sequence given in SEQ ID NO:38.

In one embodiment, the antigen-binding domain that binds to IL22 is a Fab comprising a light chain comprising the sequence given in SEQ ID NO: 18 and a heavy chain comprising the sequence given in SEQ ID NO: 20

Alternatively in one embodiment, an alternative antigen-binding domain that binds to IL22 is a Fab comprising a light chain comprising the sequence given in SEQ ID NO: 80 and a heavy chain comprising the sequence given in SEQ ID NO: 82

In one embodiment, the present invention provides a multi-specific antibody comprising an antigen-binding domain that binds to IL22 comprising
a light chain variable region comprising one or more of:
   a CDR-L1 comprising SEQ ID NO:8,
   a CDR-L2 comprising SEQ ID NO:9, and
   a CDR-L3 comprising SEQ ID NO:10;
and a heavy chain variable region comprising one or more of:
   a CDR-H1 comprising SEQ ID NO:11,
   a CDR-H2 comprising SEQ ID NO:12, and
   a CDR-H3 comprising SEQ ID NO:13;
      and an antigen-binding domain that binds to IL13 comprising
a light chain variable region comprising one or more of:
   a CDR-L1 comprising SEQ ID NO:22,
   a CDR-L2 comprising SEQ ID NO:23, and
   a CDR-L3 comprising SEQ ID NO:24;
and a heavy chain variable region comprising one or more of:
   a CDR-H1 comprising SEQ ID NO:25,
   a CDR-H2 comprising SEQ ID NO:26, and
   a CDR-H3 comprising SEQ ID NO:27.

Preferably, both of the antigen-binding domains comprise at least a CDR-H3 comprising the sequences provided above.

In one embodiment, the present invention provides a multi-specific antibody comprising an antigen-binding domain that binds to IL22 comprising
a light chain variable region comprising:
   a CDR-L1 comprising SEQ ID NO:8,
   a CDR-L2 comprising SEQ ID NO:9, and
   a CDR-L3 comprising SEQ ID NO:10;
and a heavy chain variable region comprising:
   a CDR-H1 comprising SEQ ID NO:11,
   a CDR-H2 comprising SEQ ID NO:12, and
   a CDR-H3 comprising SEQ ID NO:13;
      and an antigen-binding domain that binds to IL13 comprising
a light chain variable region comprising:
   a CDR-L1 comprising SEQ ID NO: 22,
   a CDR-L2 comprising SEQ ID NO:23, and
   a CDR-L3 comprising SEQ ID NO:24;
and a heavy chain variable region comprising:
   a CDR-H1 comprising SEQ ID NO:25,
   a CDR-H2 comprising SEQ ID NO:26, and
   a CDR-H3 comprising SEQ ID NO:27.

Alternatively, the present invention provides a multi-specific antibody comprising an antigen-binding domain that binds to IL22 comprising a light chain variable region comprising:
a CDR-L1 comprising SEQ ID NO:70,
a CDR-L2 comprising SEQ ID NO:71, and
a CDR-L3 comprising SEQ ID NO:72;
and a heavy chain variable region comprising:
a CDR-H1 comprising SEQ ID NO:73,
a CDR-H2 comprising SEQ ID NO:74, and
a CDR-H3 comprising SEQ ID NO:75;
   and an antigen-binding domain that binds to IL13 comprising a light chain variable region comprising:
a CDR-L1 comprising SEQ ID NO:22,
a CDR-L2 comprising SEQ ID NO:23, and
a CDR-L3 comprising SEQ ID NO:24;
and a heavy chain variable region comprising:
a CDR-H1 comprising SEQ ID NO:25,
a CDR-H2 comprising SEQ ID NO:26, and
a CDR-H3 comprising SEQ ID NO:27.

In one embodiment, the present invention provides a multi-specific antibody comprising an antigen-binding domain that binds to IL22 comprising
a light chain variable region comprising:
   a CDR-L1 comprising SEQ ID NO:8 or SEQ ID NO:70,
   a CDR-L2 comprising SEQ ID NO:9 or SEQ ID NO:71, and
   a CDR-L3 comprising SEQ ID NO:10 or SEQ ID NO:72;
and a heavy chain variable region comprising:
   a CDR-H1 comprising SEQ ID NO:11 or SEQ ID NO:73,
   a CDR-H2 comprising SEQ ID NO:12 or SEQ ID NO:74, and
   a CDR-H3 comprising SEQ ID NO:13 or SEQ ID NO:75;
      and an antigen-binding domain that binds to IL13 comprising
a light chain variable region comprising:
   a CDR-L1 comprising SEQ ID NO: 22,
   a CDR-L2 comprising SEQ ID NO:23, and
   a CDR-L3 comprising SEQ ID NO:24;
and a heavy chain variable region comprising:
   a CDR-H1 comprising SEQ ID NO:25,
   a CDR-H2 comprising SEQ ID NO:26, and
   a CDR-H3 comprising SEQ ID NO:27.

In one embodiment, the present invention provides a multi-specific antibody comprising:
(i) an antigen-binding domain that binds to IL22 comprising
   a light chain variable region comprising the sequence given in SEQ ID NO:14 and
   a heavy chain variable region comprising the sequence given in SEQ ID NO:16; and
(ii) an antigen-binding domain that binds to IL13 comprising
   a light chain variable region comprising the sequence given in SEQ ID NO:28 or 32 and
   a heavy chain variable region comprising the sequence given in SEQ ID NO: 29 or 33.

In a particular embodiment, the present invention provides a multi-specific antibody comprising:
(i) an antigen-binding domain that binds to IL22, wherein said antigen-binding domain is a Fab comprising
   a light chain comprising the sequence given in SEQ ID NO: 18 and
   a heavy chain comprising the sequence given in SEQ ID NO: 20; and
(ii) an antigen-binding domain that binds to IL13, wherein said antigen-binding domain is
   a scFv comprising the sequence given in SEQ ID NO:36 or
   a dsscFv comprising the sequence given in SEQ ID NO:38.

### Multi-Specific Antibody Formats

The present invention also provides a multi-specific antibody which binds to IL13 and IL22, comprising or consisting of:
a) a polypeptide chain of formula (I):

V_{H}—CH₁—(CH₂)ₛ—(CH₃)ₜ—X—(V₁)ₚ; and
b) a polypeptide chain of formula (II):

(V₃)ᵣ—Z—V_{L}—C_{L}—Y—(V₂)_{q};
wherein:
   V_{H} represents a heavy chain variable domain;
   CH₁ represents domain 1 of a heavy chain constant region;
   CH₂ represents domain 2 of a heavy chain constant region;
   CH₃ represents domain 3 of a heavy chain constant region;
   X represents a bond or linker;
   V₁ represents a dsscFv, a dsFv, a scFv, a VH, a VL or a WM;
   V₃ represents a dsscFv, a dsFv, a scFv, a VH, a VL or a WM;
   Z represents a bond or linker;
   V_{L} represents a light chain variable domain;
   C_{L} represents a domain from a light chain constant region, such as Ckappa;
   Y represents a bond or linker;
   V₂ represents a dsscFv, a dsFv, a scFv, a VH, a VL or a WM;
   p represents 0 or 1;
   q represents 0 or 1;
   r represents 0 or 1;
   s represents 0 or 1;
   t represents 0 or 1;
      wherein when p is 0, X is absent and when q is 0, Y is absent and when r is 0, Z is absent; and
      wherein when q is 0, r is 1 and when r is 0, q is 1; and
      wherein when both q and r are 1, and one of V₂ and V₃ is a V_{L}, only one of V₂ or V₃ is a V_{L}.

In one embodiment, the polypeptide chain of formula (I) comprises a protein A binding domain, and the polypeptide chain of formula (II) does not bind protein A.

In one embodiment when s is 0 and t is 0, the multi-specific antibody according to the present disclosure is provided as a dimer of a heavy and light chain of Formula (I) and (II) respectively, wherein the V_{H}—CH₁ portion together with the V_{L}—C_{L} portion form a functional Fab or Fab′ fragment.

In one embodiment when s is 1 and t is 1, the multi-specific antibody according to the present disclosure is provided as a dimer of two heavy chains and two light chains of: Formula (I) and (II) respectively, wherein the two heavy chains are connected by interchain interactions, notably at the level of CH₂—CH₃, and wherein the V_{H}—CH₁ portion of each heavy chain together with the V_{L}—C_{L} portion of each light chain, form a functional Fab or Fab′ fragment. In such embodiment, the two V_{H}—CH₁—CH₂— CH₃ portions together with the two V_{L}—C_{L} portions form a functional full-length antibody. In such embodiment, the full-length antibody may comprise a functional Fc region.

V_{H} represents a heavy chain variable domain. In one embodiment V_{H} is humanized. In one embodiment the V_{H} is fully human.

V_{L} represents a light chain variable domain. In one embodiment V_{L} is humanized. In one embodiment the V_{L} is fully human.

Generally, V_{H} and V_{L} together form an antigen binding domain. In one embodiment V_{H} and V_{L} form a cognate pair. In one example, the cognate pair bind the antigen co-operatively.

Variable regions for use in the present disclosure will generally be derived from an antibody, which may be generated by any method known in the art.

Variable regions for use in the present invention, as described herein above for V_{H} and V_{L} may be from any suitable source and may be for example, fully human or humanized.

In one embodiment, the binding domain formed by V_{H} and V_{L} are specific to a first antigen.

In one embodiment, the binding domain of V₁ is specific to a second antigen.

In one embodiment, the binding domain of V₂ is specific to a second or third antigen.

In one embodiment, the binding domain of V₃ is specific to a third or fourth antigen.

In one embodiment, each one of V_{H}-V_{L}, V₁, V₂ and V₃, as present, separately binds to its respective antigen.

In one embodiment, the CH₁ domain is a naturally occurring domain 1 from an antibody heavy chain or a derivative thereof. In one embodiment, the CH₂ domain is a naturally occurring domain 2 from an antibody heavy chain or a derivative thereof. In one embodiment, the CH₃ domain is a naturally occurring domain 3 from an antibody heavy chain or a derivative thereof.

In one embodiment, the C_{L} fragment, in the light chain, is a constant kappa sequence or a derivative thereof. In one embodiment, the C_{L} fragment, in the light chain, is a constant lambda sequence or a derivative thereof.

A derivative of a naturally occurring domain as employed herein is intended to refer to where at least one amino acid in a naturally occurring sequence have been replaced or deleted, for example to optimize the properties of the domain such as by eliminating undesirable properties but wherein the characterizing feature(s) of the domain is/are retained. In one embodiment, a derivative of a naturally occurring domain comprises two, three, four, five, six, seven, eight, nine, ten, eleven or twelve amino acid substitutions or deletions compared to a naturally occurring sequence.

In one embodiment, one or more natural or engineered inter chain (i.e. inter light and heavy chain) disulphide bonds are present in the functional Fab or Fab′ fragment.

In one embodiment, a “natural” disulfide bond is present between a CH₁ and C_{L} in the polypeptide chains of Formula (I) and (II).

When the CL domain is derived from either Kappa or Lambda, the natural position for a bond forming cysteine is 214 in human cKappa and cLambda (Kabat numbering 4th edition 1987).

The exact location of the disulfide bond forming cysteine in CH1 depends on the particular domain actually employed. Thus, for example in human gamma-1 the natural position of the disulfide bond is located at position 233 (Kabat numbering). The position of the bond forming cysteine for other human isotypes such as gamma 2, 3, 4, IgM and IgD are known, for example position 127 for human IgM, IgE, IgG2, IgG3, IgG4 and 128 of the heavy chain of human IgD and IgA2B.

Optionally, there may be a disulfide bond between the VH and VL of the polypeptides of formula I and II.

In one embodiment, the multi-specific antibody according to the disclosure has a disulfide bond in a position equivalent or corresponding to that naturally occurring between CH₁ and C_{L}.

In one embodiment, a constant region comprising CH₁ and a constant region such as C_{L} has a disulfide bond which is in a non-naturally occurring position. This may be engineered into the molecule by introducing cysteine(s) into the amino acid chain at the position or positions required. This non-natural disulfide bond is in addition to or as an alternative to the natural disulfide bond present between CH₁ and C_{L}. The cysteine(s) in natural positions can be replaced by an amino acid such as serine which is incapable of forming a disulfide bridge.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, N Y, 1989; Ausbel et al., Current Protocols in Molecular Biology, Greene Publishing & Wiley-Interscience, N Y, 1993). Site-directed mutagenesis kits are commercially available, e.g. QuikChange® Site-Directed Mutagenesis kit (Stratagene, La Jolla, CA). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

In one embodiment, a disulfide bond between CH₁ and C_{L} is completely absent, for example the interchain cysteines may be replaced by another amino acid, such as serine. Thus, in one embodiment there are no interchain disulphide bonds in the functional Fab fragment of the molecule. Disclosures such as WO2005/003170, incorporated herein by reference, describe how to provide Fab fragments without an inter chain disulphide bond.

Preferred antibody formats for use in the present invention include appended IgG and appended Fab, wherein a whole IgG or a Fab fragment, respectively, is engineered by appending at least one additional antigen-binding domain (e.g. one, two, three or four additional antigen-binding domains), for example a single domain antibody (such as VH or VL, or VHH), a scFv, a dsscFv, a dsFv to the N- and/or C-terminus of the light chain of said IgG or Fab, and optionally to the heavy chain of said IgG or Fab, for example as described in WO2009/040562, WO2010035012, WO2011/030107, WO2011/061492, WO2011/061246 and WO2011/086091 all incorporated herein by reference. In particular, the Fab-Fv format was first disclosed in WO2009/040562 and the disulphide stabilized version thereof, the Fab-dsFv, was first disclosed in WO2010/035012. A single linker Fab-dsFv, wherein the dsFv is connected to the Fab via a single linker between either the VL or VH domain of the Fv, and the C terminal of the LC of the Fab, was first disclosed in WO2014/096390, incorporated herein by reference. An appended IgG comprising a full-length IgG engineered by appending a dsFv to the C-terminus of the light chain (and optionally to the heavy chain) of the IgG, was first disclosed in WO2015/197789, incorporated herein by reference.

Another preferred antibody format for use in the present invention comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in WO2015/197772. Another preferred antibody for use in the present invention fragment comprises a Fab linked to only one scFv or dsscFv, as described for example in WO2013/068571 incorporated herein by reference, and Dave et al., 2016, Mabs, 8(7) 1319-1335.

V₁, when present, represents a dsscFv, a dsFv, a scFv, a VH, a VL or a VHH, for example a dsscFv, a dsFv, or a scFv.

V₂ when present, represents a dsscFv, a dsFv, a scFv, a VH, a VL or a VHH, for example a dsscFv, a dsFv, or a scFv.

V₃, when present, represents a dsscFv, a dsFv, a scFv, a VH, a VL or a VHH, for example a

dsscFv, a dsFv, or a scFv.

When both V₂ and V₃ are present, only one of V₂ and V₃ may represent a VL.

In one embodiment, when V₁ and/or V₂ and/or V₃ are a dsFv or a dsscFv, the disulfide bond between the variable domains VH and VL of V₁ and/or V₂ and/or V₃ is between two of the residues listed below (unless the context indicates otherwise Kabat numbering is employed in the list below). Wherever reference is made to Kabat numbering the relevant reference is Kabat et al., 1991 (5th edition, Bethesda, Md.), in Sequences of Proteins of Immunological Interest, US Department of Health and Human Services, NIH, USA.

In one embodiment the disulfide bond is in a position selected from the group comprising:
V_{H}37+V_{L}95 see for example Protein Science 6, 781-788 Zhu et al (1997);
V_{H}44+V_{L}100 see for example; for example, Weatherill et al., Protein Engineering, Design & Selection, 25 (321-329), 2012);
V_{H}44+V_{L}105 see for example J Biochem. 118, 825-831 Luo et al (1995);
V_{H}45+V_{L}87 see for example Protein Science 6, 781-788 Zhu et al (1997);
V_{H}55+V_{L}101 see for example FEBS Letters 377 135-139 Young et al (1995);
V_{H}100+V_{L}50 see for example Biochemistry 29 1362-1367 Glockshuber et al (1990);
V_{H}100b+V_{L}49; see for example Biochemistry 29 1362-1367 Glockshuber et al (1990);
V_{H}98+V_{L}46; see for example Protein Science 6, 781-788 Zhu et al (1997);
V_{H}101+V_{L}46; see for example Protein Science 6, 781-788 Zhu et al (1997);
V_{H}105+V_{L}43 see for example; Proc. Natl. Acad. Sci. USA Vol. 90 pp. 7538-7542 Brinkmann et al (1993); or Proteins 19, 35-47 Jung et al (1994),
V_{H}106+V_{L}57 see for example FEBS Letters 377 135-139 Young et al (1995)
   and a position corresponding thereto in a variable region pair located in the molecule.

In one embodiment, the disulphide bond is formed between positions V_{H}44 and V_{L}100.

The amino acid pairs listed above are in the positions conducive to replacement by cysteines such that disulfide bonds can be formed. Cysteines can be engineered into these desired positions by known techniques. In one embodiment, therefore, an engineered cysteine according to the present disclosure refers to where the naturally occurring residue at a given amino acid position has been replaced with a cysteine residue.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, N Y, 1989; Ausbel et al., Current Protocols in Molecular Biology, Greene Publishing & Wiley-Interscience, N Y, 1993). Site-directed mutagenesis kits are commercially available, e.g. QuikChange® Site-Directed Mutagenesis kit (Stratagen, La Jolla, CA). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

Accordingly, in one embodiment when V₁ and/or V₂ and/or V₃ are a dsFv or a dsscFv, the variable domains VH and VL of V₁ and/or the variable domains VH and VL of V₂, and/or the variable domains VH and VL of V₃, may be linked by a disulfide bond between two cysteine residues, wherein the position of the pair of cysteine residues is selected from the group consisting of: VH37 and VL95, VH44 and VL100, VH44 and VL105, VH45 and VL87, VH100 and VL50, VH100b and VL49, VH98 and VL46, VH101 and VL46, VH105 and VL43 and VH106 and VL57.

In one embodiment when V₁ and/or V₂ and/or V₃ are a dsFv or a dsscFv, the variable domains VH and VL of V₁ and/or the variable domains VH and VL of V₂, and/or the variable domains VH and VL of V₃, may be linked by a disulfide bond between two cysteine residues, one in VH and one in VL, which are outside of the CDRs wherein the position of the pair of cysteine residues is selected from the group consisting of VH37 and VL95, VH44 and VL100, VH44 and VL105, VH45 and VL87, VH100 and VL50, VH98 and VL46, VH105 and VL43 and VH106 and VL57.

In one embodiment when V₁ is a dsFv or a dsscFv, the variable domains VH and VL of V₁ are linked by a disulphide bond between two engineered cysteine residues, one at position VH44 and the other at VL100. In one embodiment when V₂ is a dsFv or a dsscFv, the variable domains VH and VL of V₂ are linked by a disulphide bond between two engineered cysteine residues, one at position VH44 and the other at VL100. In one embodiment when V3 is a dsFv or a dsscFv, the variable domains VH and VL of V₃ are linked by a disulphide bond between two engineered cysteine residues, one at position VH44 and the other at VL100.

In one embodiment when V₁ is a dsscFv, a dsFv, or a scFv, the VH domain of V₁ is attached to X.

In one embodiment when V₁ is a dsscFv, a dsFv, or a scFv, the VL domain of V₁ is attached to X.

In one embodiment when V₂ is a dsscFv, a dsFv, or a scFv, the VH domain of V₂ is attached to Y.

In one embodiment when V₂ is a dsscFv, a dsFv, or a scFv, the VL domain of V₂ is attached to Y.

In one embodiment when V₃ is a dsscFv, a dsFv, or a scFv, the VH domain of V₃ is attached to Z.

In one embodiment when V₃ is a dsscFv, a dsFv, or a scFv, the VL domain of V₃ is attached to Z.

The skilled person will appreciate that when V₁ and/or V₂ and/or V₃ represents a dsFv, the multi-specific antibody will comprise a third polypeptide encoding the corresponding free VH or VL domain which is not attached to X or Y or Z. When V₁ and V₂, V₂ and V₃, or V₁ and V₂ and V₃ are a dsFv then the “free variable domain” (i.e. the domain linked to via a disulphide bond to the remainder of the polypeptide) will be common to both chains. Thus, whilst the actual variable domain fused or linked via X or Y or Z to the polypeptide may be different in each polypeptide chain, the free variable domains paired therewith will generally be identical to each other.

In some embodiments, p is 1. In some embodiments, p is 0. In some embodiments, q is 1. In some embodiments, q is 0, and r is 1. In some embodiments, r is 1. In some embodiments, q is 1 and r is 0. In some embodiments, q is 1 and r is 1. In some embodiments, s is 1. In some embodiments, s is 0. In some embodiments, t is 1. In some embodiments, t is 0. In some embodiments, s is 1 and t is 1. In some embodiments, s is 0 and t is 0.

In one embodiment, p is 1, q is 1, r is 0, s is 0 and t is 0, and V1 and V2 both represent a dsscFv.

Thus, in one aspect, there is provided a multi-specific antibody which binds to IL22 and IL13, comprising or consisting of:
a) a polypeptide chain of formula (Ia):

V_{H}—CH₁—X—V₁; and
b) a polypeptide chain of formula (IIa):

V_{L}—C_{L}—Y—V₂;
wherein:
   V_{H} represents a heavy chain variable domain;
   CH₁ represents domain 1 of a heavy chain constant region;
   X represents a bond or linker;
   Y represents a bond or linker;
   V₁ represents a scFv, a dsscFv, or a dsFv;
   V_{L} represents a light chain variable domain;
   C_{L} represents a domain from a light chain constant region, such as Ckappa;
   V₂ represents a scFv, a dsscFv or a dsFv;
      wherein at least one of V₁ or V₂ is a dsscFv or a dsFv.

In one embodiment, the polypeptide chain of formula (Ia) comprises a protein A binding domain, and the polypeptide chain of formula (IIa) does not bind protein A.

In such embodiment, V₂ does not bind protein A, i.e. the scFv, dsscFv or dsFv of V₂ does not comprise a protein A binding domain. In one embodiment, V₂, i.e. the scFv, dsscFv or dsFv of V₂, comprises a VH1 domain. In another embodiment, V₂, i.e. the scFv, dsscFv or dsFv of V2, comprises a VH3 domain which does not bind protein A. In one embodiment, V₂, i.e. the scFv, dsscFv or dsFv of V₂, comprises a VH2 domain. In one embodiment, V2, i.e. the scFv, dsscFv or dsFv of V2, comprises a VH4 domain. In one embodiment, V₂, i.e. the scFv, dsscFv or dsFv of V₂, comprises a VH5 domain. In one embodiment, V₂ i.e. the scFv, dsscFv or dsFv of V₂, comprises a VH6 domain. In one embodiment, the polypeptide chain of formula (Ia) comprises only one protein A binding domain present in V_{H} or V₁. In one embodiment, the polypeptide chain of formula (Ia) comprises only one protein A binding domain present in V₁. In another embodiment, the polypeptide chain of formula (Ia) comprises two protein A binding domains present in V_{H} and V₁ respectively.

In another embodiment, p is 0, q is 1, r is 0, s is 1, t is 1, and V₂ is a dsscFv. Thus, in one aspect, there is provided a multi-specific antibody which binds to IL22 and IL13 comprising or consisting of:
a) a polypeptide chain of formula (Ib):

V_{H}—CH₁—CH₂—CH₃; and
b) a polypeptide chain of formula (IIb):

V_{L}—C_{L}—Y—V₂;
wherein:
   V_{H} represents a heavy chain variable domain;
   CH₁ represents domain 1 of a heavy chain constant region;
   CH₂ represents domain 2 of a heavy chain constant region;
   CH₃ represents domain 3 of a heavy chain constant region;
   Y represents a bond or linker;
   V_{L} represents a light chain variable domain;
   C_{L} represents a domain from a light chain constant region, such as Ckappa;
   V₂ represents a dsscFv.

In one embodiment, the polypeptide chain of formula (Ib) comprises a protein A binding domain, and the polypeptide chain of formula (IIb) does not bind protein A.

In such embodiment, V₂ does not bind protein A, i.e. the dsscFv of V₂ does not comprise a protein A binding domain. In one embodiment, V₂ i.e. the dsscFv of V₂, comprises a VH1 domain. In another embodiment, V₂, i.e. the dsscFv of V₂, comprises a VH3 domain which does not bind protein A. In one embodiment, the polypeptide chain of formula (Ib) comprises only one protein A binding domain present in V_{H} or CH₂—CH₃. In another embodiment, the polypeptide chain of formula (Ib) comprises two protein A binding domains present in V_{H} and CH₂—CH₃ respectively.

In another embodiment, p is 0, q is 1, r is 0, s is 1, t is 1, and V₂ is a dsFv. Thus, in one aspect, there is provided a multi-specific antibody which binds to IL22 and IL13 comprising or consisting of:
a) a polypeptide chain of formula (Ic):

V_{H}—CH₁—CH₂—CH₃; and
b) a polypeptide chain of formula (IIc):

V_{L}—C_{L}—Y—V₂;
wherein:
   V_{H} represents a heavy chain variable domain;
   CH₁ represents domain 1 of a heavy chain constant region;
   CH₂ represents domain 2 of a heavy chain constant region;
   CH₃ represents domain 3 of a heavy chain constant region;
   Y represents a bond or linker;
   V_{L} represents a light chain variable domain;
   C_{L} represents a domain from a light chain constant region, such as Ckappa;
   V₂ represents a dsFv.

In one embodiment, the polypeptide chain of formula (Ic) comprises a protein A binding domain, and the polypeptide chain of formula (IIc) does not bind protein A.

In such embodiment, V₂ i.e. the dsFv of V₂, does not bind protein A. In one embodiment, the polypeptide chain of formula (Ic) comprises only one protein A binding domain present in V_{H} or CH₂—CH₃. In another embodiment, the polypeptide chain of formula (Ic) comprises two protein A binding domains present in V_{H} and CH₂—CH₃ respectively.

In another embodiment, p is 0, q is 0, r is 1, s is 1, t is 1, and V₃ is a dsscFv.

Thus, in one aspect, there is provided a multi-specific antibody which binds to IL22 and IL13 comprising or consisting of:
a) a polypeptide chain of formula (Id):

V_{H}—CH₁—CH₂—CH₃; and
b) a polypeptide chain of formula (IId):

V₃—Z—V_{L}—C_{L};
wherein:
   V_{H} represents a heavy chain variable domain;
   CH₁ represents domain 1 of a heavy chain constant region;
   CH₂ represents domain 2 of a heavy chain constant region;
   CH₃ represents domain 3 of a heavy chain constant region;
   Z represents a bond or linker;
   V_{L} represents a light chain variable domain;
   C_{L} represents a domain from a light chain constant region, such as Ckappa;
   V₃ represents a dsscFv.

In one embodiment, the polypeptide chain of formula (Id) comprises a protein A binding domain, and the polypeptide chain of formula (IId) does not bind protein A.

In such embodiment, V₃, i.e. the dsscFv of V₃ does not bind protein A. In one embodiment, the polypeptide chain of formula (Id) comprises only one protein A binding domain present in VH or CH₂—CH₃. In another embodiment, the polypeptide chain of formula (Id) comprises two protein A binding domains present in V_{H} and CH₂—CH₃ respectively.

In one embodiment of the multi-specific antibody of the invention,
V_{L} and V_{H} comprise an antigen binding domain that binds to IL22, and
V₂ comprises an antigen binding domain that binds to IL13.

In another embodiment of the multi-specific antibody of the invention,
V_{L} and V_{H} comprise an antigen binding domain that binds to IL22,
V₁ comprises an antigen binding domain that binds to serum albumin, and
V₂ comprises an antigen binding domain that binds to IL13.

TABLE 4
Summary of sequences of IL22, IL13 and albumin
antigen-binding domains

| | V_{L} and V_{H} | V₁ | V₂ |
|---|---|---|---|
| | SEQ ID NO | SEQ ID NO | SEQ ID NO |
| CDR-L1 | 8 | 40 | 22 |
| CDR-L2 | 9 | 41 | 23 |
| CDR-L3 | 10 | 42 | 24 |
| CDR-H1 | 11 | 43 | 25 |
| CDR-H2 | 12 | 44 | 26 |
| CDR-H3 | 13 | 45 | 27 |
| VL Fab | 14 | — | — |
| VH Fab | 16 | — | — |
| VL scFv | — | 46 | 28 |
| VH scFv | — | 47 | 29 |
| VL dsscFv | — | 50 | 32 |
| VH dsscFv | — | 51 | 33 |
| LC Fab | 18 | — | — |
| HC Fab | 20 | — | — |
| scFv (VH/VL) | — | 54 | 36 |
| dsscFv (VH/VL) | — | 56 | 38 |

In one embodiment, VL comprises
a CDR-L1 comprising SEQ ID NO:8,
a CDR-L2 comprising SEQ ID NO:9, and
a CDR-L3 comprising SEQ ID NO:10;
   and V_{H} comprises
a CDR-H1 comprising SEQ ID NO:11,
a CDR-H2 comprising SEQ ID NO:12, and
a CDR-H3 comprising SEQ ID NO:13.

In one embodiment, V₁ comprises
a light chain variable region comprising:
   a CDR-L1 comprising SEQ ID NO:40,
   a CDR-L2 comprising SEQ ID NO:41, and
   a CDR-L3 comprising SEQ ID NO:42;
and a heavy chain variable region comprising:
   a CDR-H1 comprising SEQ ID NO:43,
   a CDR-H2 comprising SEQ ID NO:44, and
   a CDR-H3 comprising SEQ ID NO:45.

In one embodiment, V₂ comprises
a light chain variable region comprising:
   a CDR-L1 comprising SEQ ID NO:22,
   a CDR-L2 comprising SEQ ID NO:23, and
   a CDR-L3 comprising SEQ ID NO:24;
and a heavy chain variable region comprising:
   a CDR-H1 comprising SEQ ID NO:25,
   a CDR-H2 comprising SEQ ID NO:26, and
   a CDR-H3 comprising SEQ ID NO:27.

In one embodiment, V_{L} comprises the sequence given in SEQ ID NO:14 and V_{H} comprises the sequence given in SEQ ID NO:16.

In one embodiment, V₁ comprises a light chain variable region comprising the sequence given in SEQ ID NO: 46 and a heavy chain variable region comprising the sequence given in SEQ ID NO: 47.

In an alternative embodiment, V₁ comprises a light chain variable region comprising the sequence given in SEQ ID NO: 50 and a heavy chain variable region comprising the sequence given in SEQ ID NO: 51.

In one embodiment, the light chain variable region and heavy chain variable region of V₁ are connected by a linker, said linker comprising the sequence given in SEQ ID NO:69.

In one embodiment, V₁ is a scFv comprising the sequence given in SEQ ID NO:54 or a dsscFv comprising the sequence given in SEQ ID NO: 56.

In one embodiment, V₂ comprises a light chain variable region comprising the sequence given in SEQ ID NO:28 and a heavy chain variable region comprising the sequence given in SEQ ID NO:29.

In an alternative embodiment, V₂ comprises a light chain variable region comprising the sequence given in SEQ ID NO:28 or 32 and a heavy chain variable region comprising the sequence given in SEQ ID NO:29 or 33.

In one embodiment, the light chain variable region and heavy chain variable region of V₂ are connected by a linker, said linker comprising the sequence given in SEQ ID NO:67.

In one embodiment, V₂ is a scFv comprising the sequence given in SEQ ID NO:36 or a dsscFv comprising the sequence given in SEQ ID NO:38.

In one embodiment, X is a linker comprising the sequence given in SEQ ID NO:68.

In one embodiment, Y is a linker comprising the sequence given in SEQ ID NO: 66.

In one embodiment, the polypeptide chain of formula (Ia) comprises the sequence given in SEQ ID NO:58 or SEQ ID NO: 60.

In one embodiment, the polypeptide chain of formula (IIa) comprises the sequence given in SEQ ID NO:62 or SEQ ID NO: 64.

In one embodiment, the polypeptide chain of formula (Ia) comprises the sequence given in SEQ ID NO: 60 and the polypeptide chain of formula (IIa) comprises the sequence given in SEQ ID NO: 64.

### Knobs-In-Holes Bi-Specific Format

In one aspect, the invention provides a multi-specific antibody that binds to IL22 and IL13 comprising at least two polypeptides, each polypeptide comprising a CH3 domain (“CH3 polypeptide”), engineered with the Knobs-in-holes technology.

The Knobs-in-holes technology relies on modifications of the interface between the two CH3 domains of two CH3 polypeptides, e.g. two heavy chains of an antibody. A bulky residue is introduced into the CH3 domain of one CH3 polypeptide and forms a protuberance (“knob”) and a cavity (or “hole”) is formed in a second CH3 polypeptide that is able to accommodate this bulky residue. The engineering of knob and hole mutations at the interface of the two CH3 polypeptides thus promote interaction between the first and the second CH3 polypeptides.

“Protuberances” are constructed by replacing small amino acid side chains from the interface of the first CH3 polypeptide with larger side chains (e.g. tyrosine or tryptophan). Compensatory “cavities” of identical or similar size to the protuberances are optionally created on the interface of the second CH3 polypeptide by replacing large amino acid side chains with smaller ones (e.g. alanine or threonine). Where a suitably positioned and dimensioned protuberance or cavity exists at the interface of either the first or second CH3 polypeptide, it is only necessary to engineer a corresponding cavity or protuberance, respectively, at the adjacent interface.

The resulting heterodimeric Fc-region can be further stabilized by the introduction/formation of artificial disulphide bridges. Non-naturally occurring disulphide bonds are constructed by replacing on the first CH3 polypeptide a naturally occurring amino acid with a free thiol-containing residue, such as cysteine, such that the free thiol interacts with another free thiol-containing residue on the second CH3 polypeptide such that a disulfide bond is formed between the first and second CH3 polypeptides.

The following substitutions resulting in appropriately spaced apart cysteine residues for the formation of new intra-chain disulphide bonds in the individual heavy chains of an Fc-region of an IgG antibody of subclass IgG1 have been found to increase heterodimer formation: Y349C in one chain and S354C in the other; Y349C in one chain and E356C in the other; Y349C in one chain and E357C in the other; L351C in one chain and S354C in the other; T394C in one chain and E397C in the other; or D399C in one chain and K392C in the other (numbering of the residues according to the Kabat EU index numbering system).

In one embodiment, the CH3 polypeptide is a heavy chain of an antibody. In one embodiment, the multi-specific antibody is a bispecific full-length immunoglobulin (Ig), e.g. an IgG, comprising two heavy chains, wherein the CH3 domain of at least one of the two heavy chains is engineered with the Knobs-in-holes technology, and wherein each heavy chain is paired with a light chain to form an antigen binding domain. In such embodiment, each antigen binding domain formed by a pair of heavy and light chain binds to a separate epitope on the same or different antigen. The heavy chain engineered to introduce a knob may be termed the “knob chain”. The heavy chain engineered to introduce the complementary hole may be termed the “hole chain”.

In one aspect, the multi-specific antibody of the present invention comprises one of the combinations of knobs and holes mutations (substitutions) as described in Table 5 (numbering of the residues according to the EU index numbering system). Alternatively, a knob and a hole mutation can be introduced to one heavy chain and a complementary knob and hole mutation can be introduced in the second heavy chain.

TABLE 5
Exemplary knobs and holes mutations (substitutions).
The numbering is according to EU.

| Knob | Hole |
|---|---|
| T366W | T366S, L368A, Y407V |
| T366W, S354C | T366S, L368A, Y407V, Y349C |
| T366W, Y349C | T366S, L368A, Y407V, E356C |
| T366Y | Y407T |
| T366W | Y407A |
| T394W | F405A |
| F405W | T394S |
| T366Y | Y407T |

In one embodiment, the antibody for use in the present invention comprises the knob substitutions T366W and Y349C in the heavy chain (i.e. the knob chain) and the hole substitutions T366S, L368A, Y407V and E356C in the second heavy chain (i.e. the hole chain).

Mutations may be introduced into the constant domain of a heavy chain or light chain by methods well known in the art, for example by site-directed mutagenesis.

In one embodiment, the light chains of the multi-specific antibody are identical to each other, and a first heavy chain and a first light chain form a binding domain that binds to a first antigen, and the second heavy chain and the second light chain form a binding domain that binds to a different antigen. In such embodiment, a host cell may be co-transfected with one or more vectors comprising the nucleic acids coding for the hole heavy chain, the knob heavy chain, and the common light chain. Methods of preparing a bispecific antibody comprising two common light chains have been described for example in U.S. Pat. No. 9,409,989.

In another embodiment, the multi-specific antibody engineered with the Knobs-in-holes technology comprises two light chains that are different.

Methods of preparing a bispecific antibody engineered with the Knobs-in-holes technology comprising two antibody heavy chains and two different light chains, each heavy chain pairing with a light chain to form a distinct antigen binding domain, have been described for example in WO11133886, WO2013/055958 and WO2015/171822.

More specifically, the present invention provides a multi-specific antibody which binds to IL22 and IL13 comprising or consisting of:
a) a polypeptide chain of formula (III):

VH₁—CH₁—CH₂—CH₃;
b) a polypeptide chain of formula (IV):

VL₁-C_{L};
c) a polypeptide chain of formula (V):

VH₂—CH₁—CH₂—CH₃; and
d) a polypeptide chain of formula (VI):
   VL₂-C_{L};
wherein:
   VH₁ and VH₂ represent a heavy chain variable domain;
   CH₁ represents domain 1 of a heavy chain constant region;
   CH₂ represents domain 2 of a heavy chain constant region;
   CH₃ represents domain 3 of a heavy chain constant region;
   VL₁ and VL₁ represent a light chain variable domain;
   C_{L} represents a domain from a light chain constant region, such as Ckappa;
      and wherein VH₁ and VL₁ comprise the IL22-binding domain, and VH₂ and VL₂ comprise the IL13-binding domain, and wherein CH₃ domains of the polypeptides of Formula III and V comprise one or more substitutions listed in Table 5.

In one embodiment, the antibody of the present invention comprises the knob substitutions T366W in the heavy chain (i.e. polypeptide of Formula III or V) and the hole substitutions T366S, L368A, Y407V in the second heavy chain (i.e. polypeptide of Formula V or III respectively).

In one embodiment, VL₁ comprises
a CDR-L1 comprising SEQ ID NO:8,
a CDR-L2 comprising SEQ ID NO:9, and
a CDR-L3 comprising SEQ ID NO:10;
   and VH₁ comprises
a CDR-H1 comprising SEQ ID NO:11,
a CDR-H2 comprising SEQ ID NO:12, and
a CDR-H3 comprising SEQ ID NO:13.

In one embodiment, VL2 comprises
a CDR-L1 comprising SEQ ID NO:22,
a CDR-L2 comprising SEQ ID NO:23, and
a CDR-L3 comprising SEQ ID NO:24;
   and VH₂ comprises
a CDR-H1 comprising SEQ ID NO:25,
a CDR-H2 comprising SEQ ID NO:26, and
a CDR-H3 comprising SEQ ID NO:27.

In one embodiment, the polypeptide chain of formula (III) comprises the sequence given in SEQ ID NO:144, the polypeptide chain of formula (IV) comprises the sequence given in SEQ ID NO:142, the polypeptide chain of formula (V) comprises the sequence given in SEQ ID NO:152, the polypeptide chain of formula (VI) comprises the sequence given in SEQ ID NO:148.

In one embodiment, the polypeptide chain of formula (III) comprises the sequence given in SEQ ID NO:146, the polypeptide chain of formula (IV) comprises the sequence given in SEQ ID NO:142, the polypeptide chain of formula (V) comprises the sequence given in SEQ ID NO:150, the polypeptide chain of formula (VI) comprises the sequence given in SEQ ID NO:148.

### Functional Properties of the Antibodies

A multi-specific antibody according to the present invention comprises at least two antigen binding domain domains, wherein one antigen binding domain that binds to IL13 and the second antigen-binding domain that binds to IL22. More specifically such multi-specific antibody is capable of binding to human and cynomolgus IL22 and IL13.

A composition according to the present invention comprises an antibody that comprises an antigen-binding domain that binds to IL22 and an antibody that comprises an antigen binding-domain that binds to IL13. More specifically the antigen-binding domain that binds to IL22 is capable of binding human and cynomolgus IL22 and the antigen-binding domain that binds to IL13 is capable of binding human and cynomolgus IL13.

The IL13-binding domain may:
i. bind to IL13 and prevent binding of IL13Rα1 and as a result also block subsequent interaction with IL4R; or
ii. bind to IL13 in such a way that allows binding to IL13Rα1 but prevents recruitment of IL4R into the complex.

The IL22-binding domain may:
i. bind to IL22 and prevent IL22 binding to IL22R1; or
ii. bind to IL22 but allow IL22R1 binding to IL22.

Preferably the antibodies are specific for their antigens.

The properties described here in relation to antigen-binding domains also apply to antibodies,

including multi-specific antibodies, that contain those domains.

The antibodies of the present invention are neutralizing antibodies.

Preferably the IL22-binding domain is neutralizing one or more IL22 activities. In particular, the IL22-binding domain is capable of neutralizing IL22 binding to IL22 receptor 1 (IL22R1). The IL22-binding domain binds to IL22 and inhibits IL22 binding to IL22 binding protein (IL22RA2 or IL22BP). Preferably, the IL22-binding domain is capable of neutralizing IL22 binding to IL22 receptor 1 (IL22R1) and IL22 binding protein (L22RA2).

The IL22-binding domain binds to the same region on IL22 as IL22R1. In one particular embodiment of the IL22-binding domain, the present invention provides an IL22-binding domain that binds to a region on IL22 such that the binding sterically blocks the interaction between IL22 and IL22R1.

In one embodiment, the IL22-binding domain according to the present invention binds to IL22 that is not bound to IL22 binding protein (“free IL22”). In another embodiment, the IL22-binding domain binds to IL22 and prevents IL22 from binding to IL22 binding protein.

Preferably the IL13-binding domain is neutralizing one or more of IL13 activities. The IL13-binding domain also inhibits IL13 interaction with IL13R-alpha1 and IL13R-alpha2. The IL13-binding domain binds to IL13 and prevents binding of IL13Rα1 and as a result also blocks IL-4R from binding. In one example, the IL13-binding domain binds to IL13 and blocks IL13 interaction with IL13R-alpha1 and/or IL13R-alpha2. Inhibition of IL13 binding to IL13R-alpha1 prevents the formation of the IL13/IL13R-alpha1/IL4R-alpha receptor complex. In one example, the IL13-binding domain allows the binding of IL13 to IL13R-alpha1 but blocks the binding of IL4R-alpha, so preventing the formation of the receptor complex.

In one embodiment, IL22-binding domain has a stronger binding affinity for IL22 compared to IL22R1. This is characterized by a constant of dissociation (KD) at least 10-fold higher for IL22 than for IL22R1 or IL22BP. Specifically such is measured using BIACore technique.

IL22-binding domain binds to IL22 with sufficient affinity and specificity. In certain embodiments, the IL22-binding domain binds to human IL22 with a K_{D} of about any one of 1 μM, 100 nM, 50 nM, 40 nM, 30 nM, 20 nM, 10 nM, 5 nM, 1 nM, 0.5 nM, 0.1 nM, 0.05 nM, or 0.001 nM (e.g. 10⁻⁸ M or less, e.g. from 10⁻⁸ M to 10⁻¹³ M, e.g., from 10⁻⁹ M to 10⁻¹³ M), including any range in between these values. In one embodiment, the IL22-binding domain according to the present invention binds to human IL22 with a KD of less than 100 pM.

In certain embodiments, the IL22-binding domain binds to cynomolgus IL22 with a KD of about any one of 1 μM, 100 nM, 50 nM, 40 nM, 30 nM, 20 nM, 10 nM, 5 nM, 1 nM, 0.5 nM, 0.1 nM, 0.05 nM, or 0.001 nM (e.g. 10⁻⁸ M or less, e.g. from 10⁻⁸ M to 10⁻¹³ M, e.g., from 10⁻⁹ M to 10⁻¹³ M), including any range in between these values. In one embodiment, the IL22-binding domain binds to cynomolgus IL22 with a KD of less than 100 pM.

In certain embodiments, the IL13-binding domain binds to human IL13 with a KD of about any one of 1 μM, 100 nM, 50 nM, 40 nM, 30 nM, 20 nM, 10 nM, 5 nM, 1 nM, 0.5 nM, 0.1 nM, 0.05 nM, or 0.001 nM (e.g. 10⁻⁸ M or less, e.g. from 10⁻⁸ M to 10⁻¹³ M, e.g., from 10⁻⁹ M to 10⁻¹³ M), including any range in between these values. In one embodiment, the IL13-binding domain according to the present invention binds to human IL13 with a KD of less than 100 pM.

In certain embodiments, the IL13-binding domain binds to cynomolgus IL13 with a KD of about any one of 1 μM, 100 nM, 50 nM, 40 nM, 30 nM, 20 nM, 10 nM, 5 nM, 1 nM, 0.5 nM, 0.1 nM, 0.05 nM, or 0.001 nM (e.g. 10⁻⁸ M or less, e.g. from 10⁻⁸ M to 10⁻¹³ M, e.g., from 10⁻⁹ M to 10⁻¹³ M), including any range in between these values. In one embodiment, the IL13-binding domain binds to cynomolgus IL13 with a KD of less than 250 pM.

It will be appreciated by the skilled person that KD value may differ depending on the format and overall structure of the antibody. For example, the KD of an antigen-binding domain might differ in the context of the multi-specific antibodies

The multi-specific antibody and the compositions of the present invention is also capable of inhibiting IL10 release in cells.

As demonstrated by the Examples, the IL22-binding domain is capable of inhibiting IL22-mediated keratinocyte proliferation and differentiation.

The multi-specific antibodies of the present invention show dosed dependent inhibition of IL13 biomarker (eotaxin-3) and IL22 dependent biomarker (S100A7).

The multi-specific antibodies of the present invention are capable of simultaneous binding to either human or cynomolgus IL22 and IL13. In one embodiment, the multi-specific antibody comprises an additional antigen-binding domain that binds to albumin and is capable of simultaneously binding either human or cynomolgus IL22, IL13 and albumin.

Consequently, the multi-specific antibodies of the present invention act in a similar way as a composition comprising an antibody that binds to IL13 and an antibody that binds to IL22.

The affinity of an antibody, as well as the extent to which an antibody inhibits binding, can be determined by the skilled person using conventional techniques, for example those described by Scatchard et al. (Ann. KY. Acad. Sci. 51:660-672 (1949)) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

### Epitopes and Antibodies Binding to the Same Epitope

Antibodies may compete for binding to IL22 and/or IL13 with, or bind to the same epitope as, those defined above in terms of light-chain, heavy-chain, light chain variable region (LCVR), heavy chain variable region (HCVR) or CDR sequences.

Antibodies may compete for binding to IL22 with, or bind to the same epitope as a multi-specific antibody which comprises a CDR-L1/CDR-L2/CDR-L3/CDR-H1/CDR-H2/CDR-H3 sequence combination of SEQ ID NOs: 8/9/10/11/12/13.

Antibodies may compete for binding to IL13 with, or bind to the same epitope a, a multi-specific antibody which comprises a CDR-L1/CDR-L2/CDR-L3/CDR-H1/CDR-H2/CDR-H3 sequence combination of SEQ ID NOs:22/23/24/25/26/27.

Antibodies may compete for binding to serum albumin with, or bind to the same epitope as a multi-specific antibody which comprises a CDR-L1/CDR-L2/CDR-L3/CDR-H1/CDR-H2/CDR-H3 sequence combination of SEQ ID NOs: 40/41/42/43/44/45.

An antibody may compete for binding to IL22 with, or bind to the same epitope as, a multi-specific antibody which comprises a LCVR and HCVR sequence pair of SEQ ID NOs: 14/16 or 76/78.

An antibody may compete for binding to IL22 with, or bind to the same epitope as a Fab comprising a light chain comprising the sequence given in SEQ ID NO:18 and a heavy chain comprising the sequence given in SEQ ID NO:20.

Thus in one embodiment, a IL22-binding domain binds to an epitope on IL22, said epitope comprising one or more residues of the polypeptide VRLIGEKLFHGVSM (SEQ ID NO: 155) corresponding to residues 72-85 of the amino-acid sequence of IL22 defined by SEQ ID NO: 1. More specifically an antibody binds to at least 1, at least 2, at least 3, at least 5, at least 8, at least 10, or all of residues selected from the residues 72-85 of the amino-acid sequence of IL22 defined by SEQ ID NO: 1. More particularly, the IL22-binding domain binds to the polypeptide VRLIGEKLFHGVSM (SEQ ID NO: 155) corresponding to residues 72-85 of the amino-acid sequence of IL22 defined by SEQ ID NO: 1.

In one embodiment, the IL22-binding domain binds to an epitope on IL22, said epitope comprising at least 1, at least 2, at least 3, at least 5, at least 8, at least 10, or all of residues selected from the list consisting of Gln48, Glu77, Phe80, His81, Gly82, Val83, Ser84, Met85, Arg88, Leu169, Met172, Ser173, Arg175, Asn176 and Ile179 of human IL22 (SEQ ID NO: 1) as determined at less than 4 Å contact distance. More specifically, the IL22-binding domain binds to an epitope on IL22, said epitope comprising at least 1, at least 2, at least 3, at least 5, at least 8, at least 10, or all of residues selected from the list consisting of Lys44, Phe47, Gln48, Ile75, Gly76, Glu77, Phe80, His81, Gly82, Val83, Ser84, Met85, Ser86, Arg88, Leu169, Met172, Ser173, Arg175, Asn176 and Ile179 of human IL22 (SEQ ID NO: 1) as determined at the distance of less than 5 Å contact distance between the antibody and IL22.

In particular, an antibody may compete for binding to IL22 with, or bind to the same epitope as, an antibody which comprises the residues of the heavy and light chains listed in tables 6 or 7 below. More particularly, an antibody comprises CDR-H3 sequence comprising residues defined in table 7, preferably, and binds to an epitope on IL22 as defined above. More specifically, an antibody of the invention comprises CDR-H1, CDR-H2 and CDR-H3 residues as defined in the tables 6 or 7 and binds to an epitope on IL22 as defined above.

TABLE 6
Amino acids of the light and heavy chains of the IL22-binding
domain of the present invention (11041) involved in
interactions with IL22 which have ≤4 Å contact distance
between the antibody and IL22. The positions of the residues
correspond to SEQ ID NO: 14 for the light chain and
SEQ ID NO 16 for the heavy chain (sequential numbering)

| light chain | heavy chain |
|---|---|
| Tyr30 (CDR1) | Ser31 (CDR1) |
| Thr31 (CDR1) | Tyr32 (CDR1) |
| Asn32 (CDR1) | Ala33 (CDR1) |
| Trp50 (CDR2) | Asp52 (CDR2) |
| Tyr93 (CDR3) | Ile53 (CDR2 |
| | Arg99 (CDR3) |
| | Phe100 (CDR3) |
| | Val101 (CDR3) |
| | Gly102 (CDR3) |
| | Val103 (CDR3) |
| | Asp104 (CDR3) |

TABLE 7
Amino acids of the light and heavy chains of the IL22-binding
domain of the present invention (11041) involved in
interactions with IL22 which have ≤5 Å contact distance
between the antibody and IL22. The positions of the residues
correspond to SEQ ID NO: 14 for the light chain and
SEQ ID NO 16 for the heavy chain (sequential numbering).

| light chain | heavy chain |
|---|---|
| Tyr30 (CDR1) | Ser30 (CDR1) |
| Thr31 (CDR1) | Ser31 (CDR1) |
| Asn32 (CDR1) | Tyr32 (CDR1) |
| Trp50 (CDR2) | Ala33 (CDR1) |
| Tyr93 (CDR3) | Ile50 (CDR2) |
| Gly94 (CDR3) | Asp52 (CDR2) |
| Tyr101 (CDR3) | Ile53 (CDR2 |
| | Glu54 (CDR2) |
| | Tyr58 (CDR2) |
| | Arg97 (CDR3) |
| | Asp98 (CDR3) |
| | Arg99 (CDR3) |
| | Phe100 (CDR3) |
| | Val101 (CDR3) |
| | Gly102 (CDR3) |
| | Val103 (CDR3) |
| | Asp104 (CDR3) |

More particularly, a multi-specific antibody of the present invention binds to an epitope on human IL22 as defined above, and wherein said antibody prevents binding of IL22 to IL22R1 and IL22RA2. More specifically the light chain of the antibody sterically prevents binding of IL22R1 to IL22.

The epitope can be identified by any suitable binding site mapping method known in the art in combination with any one of the antibodies provided by the present invention. Examples of such methods include screening peptides of varying lengths derived from full length target protein for binding to the antibody of the present invention and identify a fragment that can specifically bind to the antibody containing the sequence of the epitope recognized by the antibody. Target peptides may be produced synthetically. Peptides that bind the antibody can be identified by, for example, mass spectrometric analysis. In another example, NMR spectroscopy or X-ray crystallography can be used to identify the epitope bound by an antibody of the present invention. Typically, when the epitope determination is performed by X-ray crystallography, amino acid residues of the antigen within 4 Å from CDRs are considered to be amino acid residues part of the epitope. Once identified, the epitope may serve for preparing fragments which bind an antibody of the present invention and, if required, used as an immunogen to obtain additional antibodies which bind the same epitope.

In one embodiment the epitope of antibody is determined by X-ray crystallography.

One can easily determine whether an antibody binds to the same epitope as, or competes for binding with, a reference antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention or the reference antibody causes a conformation change in the antigen and hence preventing the binding of the test antibody.

To determine if an antibody competes for binding with a reference antibody, the above-described binding methodology is performed in two different experimental setups. In a first setup, the reference antibody is allowed to bind to the antigen under saturating conditions followed by assessment of binding of the test antibody to the antigen. In a second setup, the test antibody is allowed to bind to the antigen under saturating conditions followed by assessment of binding of the reference antibody to the protein/peptide. If, in both experimental setups, only the first (saturating) antibody is capable of binding to the protein/peptide, then it is concluded that the test antibody and the reference antibody compete for binding to the antigen. As will be appreciated by the skilled person, an antibody that competes for binding with a reference antibody may not necessarily bind to the identical epitope as the reference antibody, but may sterically block binding of the reference antibody by binding an overlapping or adjacent epitope or cause a conformational change leading to the lack of binding

Two antibodies bind to the same or overlapping epitope if each competitively inhibits (blocks) binding of the other to the antigen. Two antibodies have overlapping epitopes if some amino acid mutations that reduce or eliminate binding of one antibody reduce or eliminate binding of the other.

Alternatively, two antibodies have the same epitope if essentially all amino acid mutations in the antigen that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same part of the antigen as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Antibody Variants

In certain embodiments, antibody variants having one or more amino acid substitutions, insertions, and/or deletions are provided. Sites of interest for substitutional mutagenesis include the CDRs and FRs. Amino acid substitutions may be introduced into an antibody of interest and the products screened for a desired activity, e.g., retained/improved antigen binding, decreased immunogenicity, or improved ADCC or CDC.

In certain embodiments, amino acid sequence variants of the antibodies described herein are contemplated. For example, it may be desirable to improve the binding affinity and/or other biological properties of the antibody. Amino acid sequence variants of the antibody may be prepared by introducing appropriate modifications into the nucleotide sequence encoding the protein, or by peptide synthesis. Such modifications include, for example, deletions from, and/or insertions into and/or substitutions of residues within the amino acid sequences (such as in one or more CDRs and/or framework sequences or in a VH and/or a VL domain) of the antibody. Any combination of deletion, insertion, and substitution can be made to arrive at the final construct, provided that the final construct possesses the desired characteristics.

In certain embodiments of the variant VH and VL sequences provided herein, each HVR either is unaltered, or contains no more than one, two or three amino acid substitutions.

### IL22-Binding Domain Variants

It will be appreciated that one or more amino acid substitutions, additions and/or deletions may be made to the CDRs of the IL22-binding domain provided by the present invention without significantly altering the ability of the antibody to bind to IL22 and to attenuate IL22 activity. The effect of any amino acid substitutions, additions and/or deletions can be readily tested by one skilled in the art, for example by using the methods described herein, particularly those illustrated in the Examples, to determine IL22 binding and inhibition of the IL22 interactions with its receptor IL22R1 and IL22 binding protein.

Consequently, in certain embodiments of the variant VH and VL sequences of IL22-binding domain, each CDR either contains no more than one, two or three amino acid substitutions, wherein such amino-acid substitutions are conservative, and wherein the IL22-binding domain retains its binding properties to IL22 and blocks IL22 binding to IL22R1 and IL22 binding protein.

Accordingly, in one embodiment, the -IL22-binding domain comprises CDRs as defined by the sequences given in SEQ ID NO: 8/9/10/11/12/13 in which one or more amino acids in one or more of the CDRs has been substituted with another amino acid, for example a similar amino acid as defined herein below.

In one embodiment, the -IL22-binding domain comprises light chain variable domain which comprises a sequence which has at least 70%, 80%, 90%, 91%, 92%, 93%, 94%, 95%, 96%, 97%, 98%, or 99% identity or similarity to the sequence given in SEQ ID NO:14 and heavy chain variable domain which comprises a sequence which has at least 70%, 80%, 90%, 95% or 98% identity or similarity to the sequence given in SEQ ID NO:16.

In embodiments, one or more amino acid substitutions in one or more CDRs replaces a free Cysteine residue or modifies a potential Asparagine deamidation site. In embodiments, one or more amino acid substitutions in one or more CDRs modifies a potential Aspartic acid isomerization site. In embodiments, one or more amino acid substitutions in one or more CDRs removes a potential DP hydrolysis site. In embodiments of the IL22-binding domain, one or more amino acid substitutions in one or more CDRs replaces a free Cysteine residue or modifies a potential Asparagine deamidation site. In embodiments, one or more amino acid substitutions in one or more CDRs modifies a potential Aspartic acid isomerization site. In embodiments of the IL22-binding domain, one or more amino acid substitutions in one or more CDRs removes a potential DP hydrolysis site.

In embodiments, with reference to CDR-L3 (SEQ ID NO: 10) the substitutions are C91S or C91V; N95D; S96A; or a combination thereof; with reference to CDR-H2 (SEQ ID NO:12), the substitutions are D54E, G55A, or a combination thereof; with reference to CDR-H3 (SEQ ID NO:13), the substitutions is D107E, or a combination of the recited substitutions, wherein the positions within the light chain are according to SEQ ID NO: 14 and the positions within the heavy chain are according to SEQ ID NO: 16.

In one embodiment, an antibody of the present invention comprises a light chain variable region and a heavy chain variable region, wherein the light chain variable region comprises the sequence given in SEQ ID NO:14, wherein one or more residues at the positions 91, 95, and/or 96 have been substituted by another amino-acid; and the heavy chain variable region comprises the sequence given in SEQ ID NO:16, wherein one or more residues at the positions 54, 55, and/or 107 have been substituted by another amino-acid.

In some embodiments, IL22-binding domain is a Fab comprising a light chain comprising a sequence which has at least 70%, 80%, 90%, 91%, 92%, 93%, 94%, 95%, 96%, 97%, 98%, or 99% identity or similarity to the sequence given in SEQ ID NO:18 and/or a heavy chain comprising a sequence which has at least 70%, 80%, 90%, 91%, 92%, 93%, 94%, 95%, 96%, 97%, 98%, or 99% identity or similarity to the sequence given in SEQ ID NO:20.

In some embodiments, the IL22-binding domain comprises CDR-L1/CDR-L2/CDR-L3/CDR-H1/CDR-H2/CDR-H3 sequences comprising SEQ ID NOs:8/9/10/11/12/13 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO: 14 and 16 respectively.

### IL13-Binding Domain Variants

It will be also appreciated that one or more amino acid substitutions, additions and/or deletions may be made to the CDRs of the IL13-binding domain provided by the present invention without significantly altering the ability of the antibody to bind to IL13 and to neutralize IL13 activity. The effect of any amino acid substitutions, additions and/or deletions can be readily tested by one skilled in the art to determine IL13 binding to IL13R-alpha1 and IL13R-alpha2.

Consequently, in certain embodiments of the variant VH and VL sequences of IL13-binding domain, each CDR either contains no more than one, two or three amino acid substitutions, wherein such amino-acid substitutions are conservative, and wherein the IL13-binding domain retains its binding properties to IL13 and blocks IL13 binding to IL13R-alpha1 and IL13R-alpha2, and prevents binding of IL13Rα1 and blocks IL-4R from binding.

Accordingly, in one embodiment, the -IL13-binding domain comprises CDRs as defined by the sequences given in SEQ ID NO: 22/23/24/25/26/27 in which one or more amino acids in one or more of the CDRs has been substituted with another amino acid, for example a similar amino acid as defined herein below.

In some embodiments, IL13-binding domain comprises a light chain variable region comprising a sequence which has at least 70%, 80%, 90%, 91%, 92%, 93%, 94%, 95%, 96%, 97%, 98%, or 99% identity or similarity to the sequence given in SEQ ID NO:28 and/or a heavy chain variable region comprising a sequence which has at least 70%, 80%, 90%, 91%, 92%, 93%, 94%, 95%, 96%, 97%, 98%, or 99% identity or similarity to the sequence given in SEQ ID NO:29.

In some embodiments, IL13-binding domain comprises a light chain variable region comprising a sequence which has at least 70%, 80%, 90%, 91%, 92%, 93%, 94%, 95%, 96%, 97%, 98%, or 99% identity or similarity to the sequence given in SEQ ID NO:32 and/or a heavy chain variable region comprising a sequence which has at least 70%, 80%, 90%, 91%, 92%, 93%, 94%, 95%, 96%, 97%, 98%, or 99% identity or similarity to the sequence given in SEQ ID NO:33.

In some embodiments, IL13 binding domain is a scFv comprising a sequence which has at least 70%, 80%, 90%, 91%, 92%, 93%, 94%, 95%, 96%, 97%, 98%, or 99% identity or similarity to the sequence given in SEQ ID NO:36 or a dsscFv comprising a sequence which has at least 70%, 80%, 90%, 91%, 92%, 93%, 94%, 95%, 96%, 97%, 98%, or 99% identity or similarity to the sequence given in SEQ ID NO:38.

In some embodiments, the IL13-binding domain comprises CDR-L1/CDR-L2/CDR-L3/CDR-H1/CDR-H2/CDR-H3 sequences comprising SEQ ID NOs:22/23/24/25/26/27 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO: 28 and 29 respectively.

In some embodiments, the IL13-binding domain comprises CDR-L1/CDR-L2/CDR-L3/CDR-H1/CDR-H2/CDR-H3 sequences comprising SEQ ID NOs:22/23/24/25/26/27 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO: 32 and 33 respectively.

### Albumin-Binding Domain Variants

In one embodiment, the -albumin-binding domain comprises CDRs as defined by the sequences given in SEQ ID NO: 40/41/42/43/44/45 in which one or more amino acids in one or more of the CDRs has been substituted with another amino acid, for example a similar amino acid as defined herein below.

In some embodiments, the albumin-binding domain comprises a light chain variable region comprising a sequence which has at least 70%, 80%, 90%, 91%, 92%, 93%, 94%, 95%, 96%, 97%, 98%, or 99% identity or similarity to the sequence given in SEQ ID NO:46 and/or a heavy chain variable region comprising a sequence which has at least 70%, 80%, 90%, 91%, 92%, 93%, 94%, 95%, 96%, 97%, 98%, or 99% identity or similarity to the sequence given in SEQ ID NO:47.

In some embodiments, the albumin-binding domain comprises a light chain variable region comprising a sequence which has at least 70%, 80%, 90%, 91%, 92%, 93%, 94%, 95%, 96%, 97%, 98%, or 99% identity or similarity to the sequence given in SEQ ID NO: 50 and/or a heavy chain variable region comprising a sequence which has at least 70%, 80%, 90%, 91%, 92%, 93%, 94%, 95%, 96%, 97%, 98%, or 99% identity or similarity to the sequence given in SEQ ID NO: 51.

In some embodiments, anti-albumin binding domain is a scFv comprising a sequence which has at least 70%, 80%, 90%, 91%, 92%, 93%, 94%, 95%, 96%, 97%, 98%, or 99% identity or similarity to the sequence given in SEQ ID NO:54 or a dsscFv comprising a sequence which has at least 70%, 80%, 90%, 91%, 92%, 93%, 94%, 95%, 96%, 97%, 98%, or 99% identity or similarity to the sequence given in SEQ ID NO: 56.

In some embodiments, the albumin-binding domain comprises CDR-L1/CDR-L2/CDR-L3/CDR-H1/CDR-H2/CDR-H3 sequences comprising SEQ ID NOs: 40/41/42/43/44/45 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO: 46 and 47 respectively.

In some embodiments, the albumin-binding domain comprises CDR-L1/CDR-L2/CDR-L3/CDR-H1/CDR-H2/CDR-H3 sequences comprising SEQ ID NOs: 40/41/42/43/44/45 respectively, and the remainder of the light chain and heavy chain variable regions have at least 70%, 80%, 90%, 91%, 92%, 93%, 94%, 95%, 96%, 97%, 98%, or 99% identity or similarity to SEQ ID NO: 50 and 51 respectively.

### Multi-Specific Antibody Variants

In certain embodiments, substitutions, insertions, or deletions may occur within one or more CDRs so long as such alterations do not substantially reduce the ability of the antibody to bind to IL22 and IL13. For example, conservative alterations that do not substantially reduce binding affinity may be made in CDRs. Such alterations may be outside of CDRs. In certain embodiments of the variant VH and VL sequences, each CDR either is unaltered, or contains no more than one, two or three amino acid substitutions.

Accordingly, the present invention provides a multi-specific antibody comprising CDRs as defined by the sequences given in SEQ ID NOs:8, 9, 10, 11, 12, 13, 22, 23, 24, 25, 26, 27, in which one or more amino acids in one or more of the CDRs has been substituted with another amino acid, for example a similar amino acid as defined herein below.

Additionally, the present invention provides a multi-specific antibody comprising CDRs as defined by the sequences given in SEQ ID NOs: 8, 9, 10, 11, 12, 13, 22, 23, 24, 25, 26, 27, 40, 41, 42, 43, 44, 45, in which one or more amino acids in one or more of the CDRs has been substituted with another amino acid, for example a similar amino acid as defined herein below.

In one embodiment, the CDRs of the multi-specific antibody comprise sequences which have at least 70%, 80%, 90%, 91%, 92%, 93%, 94%, 95%, 96%, 97%, 98%, or 99% identity or similarity to the sequences given in SEQ ID NOs: 8, 9, 10, 11, 12, 13, 22, 23, 24, 25, 26, 27, while retaining the ability to bind to IL13 and IL22.

In one embodiment, V_{L} comprises a sequence which has at least 70%, 80%, 90%, 95% or 98% identity or similarity to the sequence given in SEQ ID NO:14 and VH comprises a sequence which has at least 70%, 80%, 90%, 95% or 98% identity or similarity to the sequence given in SEQ ID NO:16.

In one embodiment, V₁ comprises a light chain variable region comprising a sequence which has at least 70%, 80%, 90%, 95% or 98% identity or similarity to the sequence given in SEQ ID NO: 46 and/or a heavy chain variable region comprising a sequence which has at least 70%, 80%, 90%, 95% or 98% identity or similarity to the sequence given in SEQ ID NO:47.

In one embodiment, V₁ comprises a light chain variable region comprising a sequence which has at least 70%, 80%, 90%, 95% or 98% identity or similarity to the sequence given in SEQ ID NO:50 and/or a heavy chain variable region comprising a sequence which has at least 70%, 80%, 90%, 95% or 98% identity or similarity to the sequence given in SEQ ID NO: 51.

In one embodiment, the light chain variable region and heavy chain variable region of V₁ in formulas (I), (Ia), (Ib), (Ic), or (Id) are connected by a linker, said linker comprising a sequence which has at least 70%, 80%, 90%, 91%, 92%, 93%, 94%, 95%, 96%, 97%, 98%, or 99% identity or similarity to the sequence given in SEQ ID NO:69.

In one embodiment, V₁ is a scFv comprising a sequence which has at least 70%, 80%, 90%, 95% or 98% identity or similarity to the sequence given in SEQ ID NO:54 or a dsscFv comprising a sequence which has at least 70%, 80%, 90%, 95% or 98% identity or similarity to the sequence given in SEQ ID NO:56.

In one embodiment, V₂ comprises a light chain variable region comprising a sequence which has at least 70%, 80%, 90%, 95% or 98% identity or similarity to the sequence given in SEQ ID NO: 28 and/or a heavy chain variable region comprising a sequence which has at least 70%, 80%, 90%, 95% or 98% identity or similarity to the sequence given in SEQ ID NO:29.

In one embodiment, V₂ comprises a light chain variable region comprising a sequence which has at least 70%, 80%, 90%, 95% or 98% identity or similarity to the sequence given in SEQ ID NO: 32 and/or a heavy chain variable region comprising a sequence which has at least 70%, 80%, 90%, 95% or 98% identity or similarity to the sequence given in SEQ ID NO: 33.

In one embodiment, the light chain variable region and heavy chain variable region of V₂ are connected by a linker, said linker comprising a sequence which has at least 70%, 80%, 90%, 91%, 92%, 93%, 94%, 95%, 96%, 97%, 98%, or 99% identity or similarity to the sequence given in SEQ ID NO:67.

In one embodiment, V₂ is a scFv comprising a sequence which has at least 70%, 80%, 90%, 95% or 98% identity or similarity to the sequence given in SEQ ID NO:36 or a dsscFv comprising a sequence which has at least 70%, 80%, 90%, 95% or 98% identity or similarity to the sequence given in SEQ ID NO:38.

In one embodiment, X in formulas I, la, Ib, Ic, or Id is a linker comprising a sequence which has at least 70%, 80%, 90%, 91%, 92%, 93%, 94%, 95%, 96%, 97%, 98%, or 99% identity or similarity to the sequence given in SEQ ID NO:68.

In one embodiment, Y is a linker comprising a sequence which has at least 70%, 80%, 90%, 91%, 92%, 93%, 94%, 95%, 96%, 97%, 98%, or 99% identity or similarity to the sequence given in SEQ ID NO:66.

In one embodiment, the polypeptide chain of formula (Ia) comprises a sequence which has at least 70%, 80%, 90%, 91%, 92%, 93%, 94%, 95%, 96%, 97%, 98%, or 99% identity or similarity to the sequence given in SEQ ID NO:58 or SEQ ID NO: 58 or 60.

In one embodiment, the polypeptide chain of formula (IIa) comprises a sequence which has at least 70%, 80%, 90%, 95% or 98% identity or similarity to the sequence given in SEQ ID NO:62 or SEQ ID NO: 64.

In one embodiment, the polypeptide chain of formula (Ia) comprises a sequence which has at least 70%, 80%, 90%, 91%, 92%, 93%, 94%, 95%, 96%, 97%, 98%, or 99% identity or similarity to the sequence given in SEQ ID NO:58 and the polypeptide chain of formula (IIa) comprises a sequence which has at least 70%, 80%, 90%, 91%, 92%, 93%, 94%, 95%, 96%, 97%, 98%, or 99% identity or similarity to the sequence given in SEQ ID NO: 62.

In one embodiment, the polypeptide chain of formula (Ia) comprises a sequence which has at least 70%, 80%, 90%, 91%, 92%, 93%, 94%, 95%, 96%, 97%, 98%, or 99% identity or similarity to the sequence given in SEQ ID NO:60 and the polypeptide chain of formula (IIa) comprises a sequence which has at least 70%, 80%, 90%, 91%, 92%, 93%, 94%, 95%, 96%, 97%, 98%, or 99% identity or similarity to the sequence given in SEQ ID NO: 64.

In some embodiments, a multi-specific antibody comprises CDRs as defined by the sequences given in SEQ ID NOs: 8, 9, 10, 11, 12, 13, 22, 23, 24, 25, 26, 27, 40, 41, 42, 43, 44, 45, the respective heavy and light chain variable regions as defined by the sequences provided herein, and the remainder of the polypeptide chains of formula (Ia) and (Ha) outside of variable regions has at least 70%, 80%, 90%, 91%, 92%, 93%, 94%, 95%, 96%, 97%, 98%, or 99% identity or similarity to the sequences as provided herein.

In some embodiments, a multi-specific antibody comprises CDRs as defined by the sequences given in SEQ ID NOs: 8, 9, 10, 11, 12, 13, 22, 23, 24, 25, 26, 27, the respective heavy and light chain variable regions as defined by the sequences provided herein, and the remainder of the polypeptide chains of formula (III), (IV), (V), and (VI) outside of variable regions has at least 70%, 80%, 90%, 91%, 92%, 93%, 94%, 95%, 96%, 97%, 98%, or 99% identity or similarity to the sequences as provided herein.

### Sequence Identity and Similarity

Degrees of identity and similarity between sequences can be readily calculated. The “% sequence identity” (or “% sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) amino-acids (e.g., identical amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to obtain the % sequence identity or percent sequence similarity.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). Polypeptide sequences also can be compared using FASTA using default or recommended parameters. FASTA (e.g., FASTA2 and FASTA3) provides alignments and percent sequence identity of the regions of the best overlap between the query and search sequences.

In certain embodiments, substitutions, insertions, or deletions may occur within one or more CDR so long as such alterations do not substantially reduce the ability of the antibody to bind the target.

For example, conservative alterations that do not substantially reduce binding affinity may be made in CDRs. Such alterations may be made outside of antigen contacting residues in the CDRs.

Conservative substitutions are shown in Table 8 together with more substantial “exemplary substitutions”.

TABLE 8
Examples of amino-acid substitutions

| Original | | Conservative |
|---|---|---|
| Residue | Exemplary Substitutions | Substitution |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys(C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |

Substantial modifications in the biological properties of an antibody variant can be accomplished by selecting substitutions that differ significantly in their effect on maintaining the structure of the polypeptide backbone in the area of the substitution, the charge or hydrophobicity of the molecule at the target site, or the bulk of the side chain. Amino acids may be grouped according to similarities in the properties of their side chains (in A. L. Lehninger, Biochemistry second ed., pp. 73-75, Worth Publishers, New York (1975))

One type of substitutional variant involves substituting one or more CDR region residues of a parent antibody (humanized or human antibody). Generally, the resulting variant(s) selected for further study will have changes in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207: 179-196 (2008)), and/or residues that contact antigen, with the resulting variant VH or VL being tested for binding affinity. Affinity maturation by constructing and reselecting from secondary libraries has been described, e.g., in Hoogenboom et al. m Methods in Molecular Biology 178: 1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity.

One of the methods that can be used for identification of residues or regions of an antibody that may be targeted for mutagenesis is alanine scanning mutagenesis (Cunningham and Wells (1989) Science, 244: 1081-1085). In this method, a residue or a number of target residues are identified and replaced by alanine to determine whether the interaction of the antibody with antigen is affected. Alternatively, or additionally, a X-ray structure of an antigen-antibody complex can be used to identify contact points between the antibody and its antigen. Variants may be screened to determine whether they contain the desired properties.

### Constant Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

Antibodies with increased half-lives and improved binding to the neonatal Fc receptor (FcRn) are described in US2005/0014934. Those antibodies comprise an Fc region with one or more substitutions therein which improve binding of the Fc region to FcRn.

In certain embodiments, an antibody variant comprises an Fc region with one or more amino acid substitutions which improve ADCC, e.g., substitutions at positions 298, 333, and/or 334 of the Fc region (EU numbering of residues).

Antibodies with reduced effector function include those with substitution of one or more of Fc region residues 234, 235, 237, 238, 265, 269, 270, 297, 327 and 329 (see, e.g., U.S. Pat. No. 6,737,056). Such Fc mutants include Fc mutants with substitutions at two or more of amino acid positions 265, 269, 270, 297 and 327 wherein the amino acid residue is numbered according to the EU numbering system

In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcRI, FcγRII and FcγRIII. FcR expression on hematopoietic cells is summarized in Ravetch and Kinet, Annu. Rev. Immunol. 9:457-492 (1991). Non-limiting examples of in vitro assays to assess ADCC activity of a molecule of interest is described in U.S. Pat. Nos. 5,500,362; 5,821,337. Alternatively, or additionally, ADCC activity of the molecule of interest may be assessed in vivo, e.g., in an animal model such as that disclosed in Clynes et al. Proc. Nat 1 Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al, J. Immunol. Methods 202: 163 (1996); Cragg, M. S. et al, Blood 101: 1045-1052 (2003); and Cragg, M. S. and M. I Glennie, Blood 103:2738-2743 (2004)). FcRn binding and in vivo clearance/half-life determinations can also be performed using methods known in the art (see, e.g., Petkova, S. B. et al, Intl. Immunol. 18(12): 1759-1769 (2006)).

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

### Effector Molecules

If desired an antibody may be conjugated to one or more effector molecule(s). In one embodiment the antibody is not attached an effector molecule.

It will be appreciated that the effector molecule may comprise a single effector molecule or two or more such molecules so linked as to form a single moiety that can be attached to the multi-specific antibodies of the present invention. Where it is desired to obtain an antibody linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as 111In and 90Y, Lu177, Bismuth213, Californium252, Iridium192 and Tungsten 188/Rhenium188; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO2005/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment, the polymer is albumin or a fragment thereof, such as human serum albumin or a fragment thereof.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumor, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example, the antibody are attached to poly(ethyleneglycol) (PEG) moieties. In one particular embodiment, the antigen-binding fragment according to the present invention and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, AL, USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody comprises a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington DC and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

In one embodiment, the present invention provides an antibody molecule which comprises a modified Fab′ fragment having at the C-terminal end of its heavy chain a modified hinge region containing at least one cysteine residue to which an effector molecule is attached. Suitably the effector molecule is PEG and is attached using the methods described in (WO 98/25971 and WO 2004072116 or in WO 2007/003898. Effector molecules may be attached to antibody fragments using the methods described in International patent applications WO 2005/003169, WO 2005/003170 and WO 2005/003171.

In one embodiment the antibody is not attached an effector molecule.

### Polynucleotides and Vectors

The present invention also provides an isolated polynucleotide encoding the antibodies or a component thereof according to the present invention. The isolated polynucleotide according to the present invention may comprise synthetic DNA, for instance produced by chemical processing, cDNA, genomic DNA or any combination thereof.

TABLE 9
Amino-acid sequences of the antibodies of the present invention and their
corresponding nucleic acid sequences.

| | | |
|---|---|---|
| | Amino-acid | Nucleic acid |
| Antibody sequence | SEQ ID NO | SEQ ID NO |

IL22

| | | |
|---|---|---|
| 11041 light chain V region | 14 | 15 |
| 11041 heavy chain V region | 16 | 17 |
| 11041 light chain Fab | 18 | 19 |
| 11041 heavy chain Fab | 20 | 21 |
| 11070 light chain V region | 76 | 77 |
| 11070 heavy chain V region | 78 | 79 |
| 11070 light chain Fab | 80 | 81 |
| 11070 heavy chain Fab | 82 | 83 |

IL13

| | | |
|---|---|---|
| 650 light chain V region (unmutated) | 28 | 30 |
| 650 heavy chain V region (unmutated) | 29 | 31 |
| 650 light chain V region (mutated) | 32 | 34 |
| 650 heavy chain V region (mutated) | 33 | 35 |
| 650 scFv (VH/VL) gH9gL8 (unmutated) | 36 | 37 |
| 650 dsscFv (VH/VL) gH9gL8 (mutated) | 38 | 39 |

Albumin

| | | |
|---|---|---|
| 645 light chain V region (unmutated) | 46 | 48 |
| 645 heavy chain V region (unmutated) | 47 | 49 |
| 645 light chain V region (mutated) | 50 | 52 |
| 645 heavy chain V region (mutated) | 51 | 53 |
| 645 scFv (VH/VL) (unmutated) | 54 | 55 |
| 645 dsscFv (VH/VL) (mutated) | 56 | 57 |

IL13/IL22 TrYbe

| | | |
|---|---|---|
| 11041gH14 HC- 645 (VH/VL) scFv | 58 | 59 |
| (unmutated) | | |
| 11041gH14 HC- 645 (VH/VL) dsscFv | 60 | 61 |
| (mutated) | | |
| 11041gL13 LC- 650 scFv (unmutated) | 62 | 63 |
| 11041gL13 LC- 650 dsscFv (mutated) | 64 | 65 |

IL13/IL22 KiH

| | | |
|---|---|---|
| 11041 knob light chain | 142 | 143 |
| 11041 knob heavy chain | 144 | 145 |
| 11041 Hole light chain | 142 | 143 |
| 11041 Hole heavy chain | 146 | 147 |
| 650 knob light chain | 148 | 149 |
| 650 knob heavy chain | 150 | 151 |
| 650 Hole light chain | 148 | 149 |
| 650 Hole heavy chain | 152 | 153 |

Examples of suitable sequences are provided herein. Thus in one embodiment the present invention provides an isolated polynucleotide encoding an antibody, antigen-binding domain, or a part thereof comprising one or more sequence given in SEQ ID NOs 15, 17, 19, 21, 30, 31, 34, 35, 37, 39, 48, 49, 52, 53, 55, 57, 59, 61, 63, 65, 143, 145, 147, 149, 151, 153, 77, 79, 81, 83.

In one embodiment, the present invention provides an isolated polynucleotide encoding the multi-specific antibody of the present invention which comprises the sequence given in SEQ ID NO: 59, 61, 63, 65.

In one embodiment, the present invention provides an isolated polynucleotide encoding the multi-specific antibody of the present invention which comprises the sequence given in SEQ ID NO: 143, 145, 147, 149, 151, 153.

The present invention also provides for a cloning or expression vector comprising one or more polynucleotides described herein. In one example, the cloning or expression vector according to the present invention comprises one or more isolated polynucleotides comprising a sequence selected from SEQ ID NO: 15, 17, 19, 21, 30, 31, 34, 35, 37, 39, 48, 49, 52, 53, 55, 57, 59, 61, 63, 65, 143, 145, 147, 149, 151, 153, 77, 79, 81, 83.

Standard techniques of molecular biology may be used to prepare DNA sequences coding for the antibody or antigen-binding fragment thereof of the present invention. Desired DNA sequences may be synthesized completely or in part using oligonucleotide synthesis techniques. Site-directed mutagenesis and polymerase chain reaction (PCR) techniques may be used as appropriate.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

### Host Cells for Production of the Multi-Specific Antibodies

Also provided is a host cell comprising one or more isolated polynucleotide sequences according to the invention or one or more cloning or expression vectors comprising one or more isolated polynucleotide sequences encoding an antibody or antigen-binding fragment thereof of the present invention. Any suitable host cell/vector system may be used for expression of the polynucleotide sequences encoding the antibody or antigen-binding fragment thereof of the present invention. Bacterial, for example E. coli, and other microbial systems may be used or eukaryotic, for example mammalian, host cell expression systems may also be used. Suitable mammalian host cells include CHO, myeloma or hybridoma cells.

In a further embodiment, a host cell comprising such nucleic acid(s) or vector(s) is provided. In one such embodiment, a host cell comprises (e.g., has been transformed with): (1) a vector comprising a nucleic acid that encodes an amino acid sequence comprising the VL of the anti-IL13 antibody and an amino acid sequence comprising the VH of the anti-IL13 antibody, or (2) a first vector comprising a nucleic acid that encodes an amino acid sequence comprising the VL of the anti-IL22 antibody and a second vector comprising a nucleic acid that encodes an amino acid sequence comprising the VH of the anti-IL22 antibody. In one embodiment, the host cell is eukaryotic, e.g. a Chinese Hamster Ovary (CHO) cell or lymphoid cell (e.g., Y0, NS0, Sp20 cell). In one embodiment, the host cell is prokaryotic, e.g. an E. coli cell. In one embodiment, a method of making an anti-X antibody is provided, wherein the method comprises culturing a host cell comprising a nucleic acid encoding the antibody, as provided above, under conditions suitable for expression of the antibody, and optionally recovering the antibody from the host cell (or host cell culture medium).

Suitable host cells for cloning or expression of vectors encoding antibodies or components thereof include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See for example Charlton, Methods in Molecular Biology, Vol. 248, B. K. C. Lo, ed., Humana Press, Totowa, N J, 2003, pp. 245-254),). After expression, the antibody may be isolated and can be further purified.

Eukaryotic microbes such as fungi or yeast are suitable cloning and/or expression hosts for antibody-encoding vectors, including fungi and yeast strains whose glycosylation pathways have been “humanized,” resulting in the production of an antibody with a partially or fully human glycosylation pattern. (Gerngross, Nat. Biotech. 22: 1409-1414 (2004), and Li et al., Nat. Biotech. 24:210-215 (2006)).

Suitable types of Chinese Hamster Ovary (CHO cells) for use in the present invention may include CHO and CHO-K1 cells including dhfr-CHO cells, such as CHO-DG44 cells and CHO-DXB11 cells and which may be used with a DHFR selectable marker or CHOK1-SV cells which may be used with a glutamine synthetase selectable marker. Other cell types of use in expressing antibodies include lymphocytic cell lines, e.g., NS0 myeloma cells and SP2 cells, COS cells. The host cell may be stably transformed or transfected with the isolated polynucleotide sequences or the expression vectors according to the present invention.

Protein A Protein A is a 42 kDa surface protein originally found in the cell wall of the bacteria Staphylococcus aureus. Protein A has been widely used to detect, quantify and purify immunoglobulins. Protein A has been reported to bind the Fab portion derived from the VH3 family antibodies, and the Fc gamma region in the constant region portion of IgG (between the CH2 and CH3 domains). The crystal structure of the complex formed by protein A and the Fab has been described for example in Graille et al., 2000, PNAS, 97(10): 5399-5404. In the context of the present disclosure, protein A encompasses natural protein A and any variant or derivative thereof, to the extent that the protein A variant or derivative maintains its ability to bind VH3 domains and/or Fc gamma domains.

The polypeptide chain of formula (I) of the present invention comprises a protein A binding domain. In one embodiment, the polypeptide chain of formula (I) comprises one, two or three protein A binding domains.

A Protein A binding domain may refer to a VH3 domain or a portion of a VH3 domain which binds to protein A, i.e. which comprises a protein A binding interface. The portion of a VH3 domain which binds to protein A does not comprise the CDRs of the VH3 domain, i.e. the protein A binding interface of the VH3 does not involve the CDRs; consequently, it will be understood that a protein A binding domain does not compete with an antigen binding domain as disclosed in the present application.

In one embodiment, the polypeptide chain of formula (I) comprises a protein A binding domain which is present in V_{H} and/or CH2-CH3 and/or V₁. In one embodiment, the polypeptide chain of formula (I) comprises one, two or three protein A binding domains, which is/are present in V_{H} and/or CH2-CH3 and/or V₁. In one embodiment, the polypeptide chain of formula (I) comprises only one protein A binding domain which is present in V_{H} or V₁. In one embodiment, s is 0, t is 0 and the polypeptide chain of formula (I) comprises only one protein A binding domain which is present in V_{H} or V₁. In one embodiment, the polypeptide chain of formula (I) comprises only one protein A binding domain which is present in V_{H}. In one embodiment, s is 0, t is 0, p is 0, and the polypeptide chain of formula (I) comprises only one protein A binding domain which is present in V_{H}. In one embodiment, the polypeptide chain of formula (I) comprises only one protein A binding domain which is present in V₁. In one embodiment, s is 0, t is 0, p is 1, and the polypeptide chain of formula (I) comprises only one protein A binding domain which is present in V₁.

In one embodiment, the polypeptide chain of formula (I) comprises two protein A binding domains. In one embodiment, the polypeptide chain of formula (I) comprises two protein A binding domains which are present in VH and CH2-CH3 respectively. In another embodiment, the polypeptide chain of formula (I) comprises two protein A binding domains which are present in V_{H} and V₁ respectively. In another embodiment, the polypeptide chain of formula (I) comprises two protein A binding domains which are present in CH2-CH3 and V₁ respectively.

In one embodiment, the polypeptide chain of formula (I) comprises three protein A binding domains, each one being present in V_{H}, CH2-CH3 and V₁.

Natural protein A can interact in particular with the Fc gamma region, in the constant region portion of IgG. More particularly, protein A can interact with a binding domain between the CH2 and the CH3. In one embodiment when s is 1, t is 1, both CH2 and CH3 are naturally occurring domains of the IgG class.

In some embodiments, the protein A binding domain(s) comprise(s) or consist(s) of a VH3 domain or variant thereof which binds to protein A. In some embodiments, the protein A binding domain(s) comprise(s) or consist(s) of a naturally occurring VH3 domain. In some embodiments, a variant of a VH3 domain which binds to protein A is a variant of a naturally occurring VH3 domain, said naturally occurring VH3 domain being unable to bind protein A.

The polypeptide chain of formula (II) of the present disclosure does not bind protein A. In one embodiment, the binding domain of V₂ does not bind protein A. In one embodiment, the binding domain of V₃ does not bind protein A. In one embodiment, both V₂ and V₃ do not bind protein A.

In some embodiments, V₂ and/or V₃ comprise(s) or consist(s) of a VH1 and/or a VH2 and/or a VH4 and/or a VH5 and/or a VH6 and do(es) not comprise a VH3 domain. In some embodiments, V2 and/or V3, comprise(s) or consist(s) of a VH3 domain or variant thereof which does not bind protein A. In some embodiments, V2 and/or V3, comprise(s) or consist(s) of a naturally occurring VH3 domain being unable to bind protein A. In some embodiments, a variant of a VH3 domain which does not bind protein A is a variant of a naturally occurring VH3, said naturally occurring VH3 domain being able to bind protein A.

Human VH3 germline genes and VH3 domains (or frameworks) have been well characterized. Many of the naturally occurring VH3 domains have the capacity to bind protein A but certain naturally occurring VH3 domains do not have the capacity to bind protein A (see Roben et al., 1995, J Immunol.; 154(12):6437-6445).

A VH3 domain for use in the present disclosure can be obtained by several methods. In one embodiment, a VH3 domain for use in the present disclosure is a naturally occurring VH3 domain, selected for its ability or inability to bind protein A, depending on its position within the polypeptide (I) and/or (II) of the disclosure. For example, a panel of antibodies may be generated against an antigen of interest by immunization of a non-human animal, then humanized, and the humanized antibodies may be screened and selected based on their ability or inability to bind protein A via the humanized VH3 domain, for example against a protein A affinity column. Alternatively, display technologies (e.g. phage display, yeast display, ribosome display, bacterial display, mammalian cell surface display, mRNA display, DNA display) may be used to screen antibody libraries and select antibodies comprising a VH3 domain which binds to, notably via a protein A binding interface which does not involve the CDRs, or does not bind protein A.

Alternatively, a VH3 domain for use in the present disclosure is a variant of a naturally occurring VH3. In one embodiment, a VH3 variant comprises a sequence of a naturally occurring VH3 able to bind protein A, and further comprising at least one amino acid mutation, which abolishes its ability to bind protein A. In one embodiment, a VH3 variant which binds to protein A comprises a sequence of a naturally occurring VH3 unable to bind protein A, and further comprises at least one amino acid mutation. In such embodiment, the mutation(s) is/are responsible for the VH3 domain to gain the ability to bind protein A, i.e. the mutation(s) contribute(s) to the generation of a protein A binding domain which was not naturally present.

In one embodiment, a VH3 variant comprises 1, 2, 3, 4, 5, 6, 7, 8, 9, 10, 11, or 12 amino acid mutations. In one embodiment, a VH3 variant comprises a mutation at the position 15, 17, 19, 57, 59, 64, 65, 66, 68, 70, 81 or 82 on the VH3, numbering according to Kabat and as described for example in Graille et al., 2000, PNAS, 97(10): 5399-5404). The mutation may be a substitution, a deletion, or an insertion. In one embodiment, the VH3 variant comprises a substitution at the position 15, 17, 19, 57, 59, 64, 65, 66, 68, 70, 81 or 82 on the VH3, numbering according to Kabat.

Naturally occurring VH1, VH2, VH4, VH5 and VH6 do not bind protein A. In one embodiment, a VH domain which does not bind protein A is a VH1. In one embodiment, a VH domain which does not bind protein A is a VH2. In one embodiment, a VH domain which does not bind protein A is a VH4. In one embodiment, a VH domain which does not bind protein A is a VH5. In one embodiment, a VH domain which does not bind protein A is a VH6.

### Production of Multi-Specific Antibodies

There are a number of approaches for generating multi-specific, notably bispecific antibodies. Morrison et al (Coloma and Morrison 1997, Nat Biotechnol. 15, 159-163) describes the fusion of single chain variable fragments (scFv) to whole antibodies, e.g. IgG. Schoonjans et al., 2000, Journal of Immunology, 165, 7050-7057, describes the fusion of scFv to antibody Fab fragments. WO2015/197772 describes the fusion of disulphide stabilised scFv (dsscFv) to Fab fragments.

Standard approaches described in the prior art comprise the expression in a host cell of at least two polypeptides, each one coding for a heavy chain (HC) or a light chain (LC) of a whole antibody or an antibody fragment e.g. a Fab, to which an additional antigen binding fragment of an antibody can be fused to the N- and/or C-terminal position of the heavy chain and/or the light chain. When trying to recombinantly produce such multi-specific antibodies by expressing two (one light chain and one heavy chain to form an appended Fab) or four polypeptides (two light chains and two heavy chains to form an appended IgG), it usually requires expressing the light chain in excess over the heavy chain, in order to ensure the proper folding of the heavy chain upon assembly with its corresponding light chain. In particular, the CH1 (domain 1 of the heavy chain constant region) is prevented from folding on itself by BIP proteins, which can be displaced by a corresponding LC; therefore, the correct folding of the CH1/HC is dependent on the availability of its corresponding LC (Lee et al., 1999, Molecular Biology of the Cell, Vol. 10, 2209-2219).

The methods of expressing multi-specific antibodies may result in the production of the light chain in excess over the heavy chain, which remains in the host cell harvest, and that the excess of light chain tends to form dimeric complexes (or “LC dimers”) which are present as a by-product of the production process with the desired multi-specific antibody, notably monomeric, and thus need to be purified away.

Importantly, the technical problem associated with the formation of dimers of light chains, when fused on N- and/or C-terminal to additional antigen binding fragment(s), has not been identified so far, and the commonly used analytical methods have not allowed the detection and quantification of those appended LC dimers amongst the heterogenous products of the production process. This may result in a significant bias when estimating the amount of the products using standard analytical methods.

Thus, there is a need to improve multi-specific antibodies and methods of production thereof, which allow the isolation and removal of the appended LC dimers easily and efficiently at the earliest steps of the production process, and thus improve the yield of the protein of interest for use in therapy, which is the multi-specific antibody, in particular in its monomeric form.

The multi-specific antibodies of the present invention have been engineered to provide improved multi-specific antibodies with equivalent functionality and stability, whilst increasing the yield of “multi-specific antibody” material, notably monomeric, obtained after purification, notably after a one-step purification comprising a protein A affinity chromatography.

Advantageously, the multi-specific antibodies of the present disclosure can be more efficiently purified with a purification method which is improved over the methods commonly used in the prior art, notably in that the improved method comprises fewer steps, which is cost and time effective at the industrial scale. In particular, the multi-specific antibodies of the present disclosure maximize the quantity of proteins of interest (i.e, the correct multi-specific antibody format) obtained after a one-step purification method comprising a protein A affinity chromatography, whereby the purification of the multi-specific antibodies of interest and the removal of the appended LC dimers occur concurrently. Advantageously, the methods of production and purification of the multi-specific antibodies of the present disclosure do not require an additional purification step to capture the free, unbound light chains in excess, notably the appended LC dimers.

The present invention provides a process for the production of a multi-specific antibody or of an antigen-binding domain according to the present invention comprising culturing a host cell according to the present invention under conditions suitable for producing the multi-specific antibody or the antigen-binding domain according to the invention, and isolating the multi-specific antibody or antigen-binding domain.

The multi-specific antibody or antigen-binding domain may comprise only a heavy or light chain polypeptide, in which case only a heavy chain or light chain polypeptide coding sequence needs to be used to transfect the host cells. For production of antibodies or antigen-binding domains comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Thus, there is provided a process for culturing a host cell and expressing a multi-specific antibody or an antigen-binding domain, isolating the latter and optionally purifying the same to provide an isolated multi-specific antibody or an antigen-binding domain. In one embodiment, the process further comprises the step of conjugating an effector molecule to the isolated antibody or fragment.

The present invention also provides a process for the production of an antibody molecule according to the present invention comprising culturing a host cell containing a vector of the present invention under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

The antibodies and antigen-binding fragments according to the present invention are expressed at good levels from host cells. Thus the properties of the antibodies and/or fragments appear to be optimized and conducive to commercial processing.

### Purified Antibodies

In one embodiment there is provided a purified antibody, for example a humanized antibody, in particular an antibody according to the invention, in substantially purified from, in particular free or substantially free of endotoxin and/or host cell protein or DNA.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 pg per mg of antibody product or less such as 100 μg per mg or less, in particular 20 μg per mg, as appropriate.

### In Vitro and Ex Vivo Use of the Multi-Specific Antibodies

The present invention also provides an in vitro or ex vivo method of inhibiting IL22-induced STAT3 phosphorylation, the method comprising contacting and incubating keratinocyte cells with a multi-specific antibody of the invention. Any keratinocyte cells and their derivatives can be used, including, for example, HaCaT cells.

The present invention further provides an in vitro or ex vivo method of inhibiting IL22 induced IL-10 release, the method comprising contacting and incubating epithelial cells with an antibody comprising an IL22-binding domain according to the present invention. More specifically COLO205 cells may be used.

Also is provided an in vitro or ex vivo method of inhibiting IL22 induced S100A7 release, the method comprising contacting and incubating keratinocytes with an antibody comprising an IL22-binding domain according to the present invention.

Also is provided an in vitro or ex vivo method of inhibiting IL22 induced epidermal thickening associated with aberrant keratinocyte differentiation and parakeratosis, the method comprising contacting and incubating a reconstituted epithelium consisting of keratinocytes and dermal fibroblasts with an antibody according to the present invention. In particular the aberrant keratinocyte proliferation and differentiation demonstrated by epidermal thickening and parakeratosis induced by IL22 is inhibited.

The cells are generally incubated for the time sufficient to allow the antibody or an antigen-binding fragment thereof to bind to the target and cause the biological effect.

The methods involving multi-specific antibodies can be used to achieve biological effects as described in the Examples herein.

### Therapeutic Use of the Multi-Specific Antibodies

The multi-specific antibodies of the invention, formulations, or pharmaceutical compositions thereof may be administered for prophylactic and/or therapeutic treatments.

The present invention provides a multi-specific antibody of the invention or pharmaceutical composition thereof for use as a medicament.

In prophylactic applications, multi-specific antibodies, formulations, or compositions are administered to a subject at risk of a disorder or condition as described herein, in an amount sufficient to prevent or reduce the subsequent effects of the condition or one or more of its symptoms.

In therapeutic applications, the multi-specific antibodies are administered to a subject already suffering from a disorder or condition as described herein, in an amount sufficient to cure, alleviate or partially arrest the condition or one or more of its symptoms. Such therapeutic treatment may result in a decrease in severity of disease symptoms, or an increase in frequency or duration of symptom-free periods.

The subjects to be treated can be animals. Preferably, the pharmaceutical compositions according to the present invention are adapted for administration to human subjects.

The present invention provides a method of treating a disorder or condition as described herein in a subject in need thereof, the method comprising administering to the subject a multi-specific antibody according to the present invention. The multi-specific antibody is administered in a therapeutically effective amount.

The present invention also provides a multi-specific antibody of the invention for use in the treatment of a disorder or condition as described herein.

### Therapeutic Use of a Combination of Antibodies Binding to IL22 and IL13

The present invention also provides a therapeutic use of a combination of an antibody that binds to IL13 and an antibody that binds to IL22. Such combination might be in a form of a composition comprising an antibody that binds to IL13 and an a composition comprising an antibody that binds to IL22, or in a form of two separate antibodies.

The antibody combination, the composition, their formulations, or pharmaceutical compositions thereof may be administered for prophylactic and/or therapeutic treatments.

In prophylactic applications, the combination of the antibodies, formulations, or compositions thereof are administered to a subject at risk of a disorder or condition as described herein, in an amount sufficient to prevent or reduce the subsequent effects of the condition or one or more of its symptoms.

In therapeutic applications, the antibodies are administered to a subject already suffering from a disorder or condition as described herein, in an amount sufficient to cure, alleviate or partially arrest the condition or one or more of its symptoms. Such therapeutic treatment may result in a decrease in severity of disease symptoms, or an increase in frequency or duration of symptom-free periods.

The subjects to be treated can be animals. Preferably, the pharmaceutical compositions comprising the combination of the antibodies are adapted for administration to human subjects.

The present invention provides a method of treating a disorder or condition as described herein in a subject in need thereof, the method comprising administering to the subject a combination of an antibody that binds to IL13 and an antibody that binds to IL22. Such antibodies are administered in a therapeutically effective amount.

The present invention provides a method of treating a disorder or condition as described herein in a subject in need thereof, the method comprising administering to the subject a composition comprising an antibody that binds to IL13 and an antibody that binds to IL22. Such antibodies are administered in a therapeutically effective amount.

The combination attenuates impaired barrier function of the skin, and/or parakeratosis, and/or the release of antimicrobial peptides

In the context of the combination, the anti-IL13 antibody and the anti-IL22 antibody might be administered either simultaneously or subsequently.

The present invention also provides a combination of an antibody that binds to IL13 and an antibody that binds to IL22 for use in the treatment of a disorder or condition as described herein.

The present invention also provides a composition comprising an antibody that binds to IL13 and an antibody that binds to IL22 for use in the treatment of a disorder or condition as described herein.

Each antibody of the combination or the composition can be independently selected from a full length antibody, Fab, scFv, Fv, dsFv and dsscFv as described above.

Each antibody of the combination can be independently selected from a monoclonal, humanized, human, and chimeric.

### Therapeutic Indications

The multi-specific antibodies, the combinations, and the compositions of the antibodies of present invention may be used in treating, preventing or ameliorating inflammatory skin conditions that are associated with IL22, IL22R1, IL13, or IL13RA1 activity; for example, any condition which results in whole or in part from signaling through IL22R1, IL13RA1, IL-13R2 and/or IL-22BP.

IL22 is mainly produced by lymphoid cells such as T helper 1 (Th1) cells, Th17 cells, and Th22 cells, γδ T cells, Natural Killer (NK) cells and innate lymphoid cells (ILCs) 3 as well as non-lymphoid cells such as fibroblasts, neutrophils, macrophages and mast cells. High levels of IL22 have been found in human psoriatic plaques (Boniface et al., Clin Exp Immunol. 150: 407-415 (2007)) and the involvement of this cytokine in the pathogenesis of psoriasis has been demonstrated in mouse models of skin inflammation (Van Belle et al. J Immunol. January 1; 188(1):462⁻⁹ (2012)). The ligands, such as IL22, that signal via the IL22R1 have been implicated in a number of diseases and since IL22R1 is expressed on skin and epithelial cells, the key diseases are those affecting skin and epithelia. IL13 is a pleiotropic cytokine involved in immune response conditions, such as atopy, asthma, allergy, and inflammatory response. The role of IL13 in immune response is facilitated by its effect on cell-signaling pathways. IL13 has been shown to play role in epidermal thickening.

The antibodies and compositions of the present invention can be used to treat inflammatory skin conditions. In certain embodiments, inflammatory skin conditions are selected from psoriasis, psoriatic arthritis, contact dermatitis, chronic hand eczema or atopic dermatitis. More specifically, the skin inflammatory disease is atopic dermatitis.

In particular, as demonstrated by the Examples, the antibodies and compositions of the invention inhibit epidermal thickening associated with aberrant keratinocyte differentiation and parakeratosis in a subject diagnosed with an inflammatory skin condition.

Consequently, the invention provides a method of attenuating impaired barrier function of the skin, and/or parakeratosis, and/or the release of cytokines and/or antimicrobial peptides, such as, for example, S100A7 in a subject diagnosed with a skin inflammatory disease, the method comprising administering to said subject an antibody as provided in the present invention.

In yet another embodiment the present invention provides an antibody of the invention for use in attenuating epidermal thickening, impaired barrier function of the skin, and/or parakeratosis, and/or the release of cytokines and/or antimicrobial peptides, such as, for example, S100A7, and/or Eotaxin-3 release in a subject diagnosed with an inflammatory skin disease.

In yet another embodiment the present invention provides use of an antibody of the invention for the manufacture of a medicament for attenuating epidermal thickening, impaired barrier function of the skin, and/or parakeratosis, and/or the release of cytokines and/or antimicrobial peptides, such as, for example, S100A7 in a subject diagnosed with a skin inflammatory disease.

In particular, such attenuation of impaired barrier function of the skin is achieved by reducing aberrant IL22 mediated keratinocyte proliferation and differentiation.

### Diagnostic Use of the Antibodies

Also part of the present invention is the use of the antibodies as diagnostically active agents or in diagnostic assays, for example for diagnosing inflammatory skin disease.

The diagnosis may preferably be performed on biological samples. A “biological sample” encompasses a variety of sample types obtained from an individual and can be used in a diagnostic or monitoring assay. The definition encompasses cerebrospinal fluid, blood such as plasma and serum, and other liquid samples of biological origin such as urine and saliva, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides.

Diagnostic testing may preferably be performed on biological samples which are not in contact with the human or animal body. Such diagnostic testing is also referred to as in vitro testing. In vitro diagnostic testing may rely on an in vitro method of detection of a marker in a biological sample which has been obtained from an individual.

### Pharmaceutical and Diagnostic Compositions

An antibody or a composition of antibodies may be provided as a pharmaceutical composition. The pharmaceutical composition will normally be sterile and will typically include a pharmaceutically acceptable carrier and/or adjuvant. A pharmaceutical composition of the present invention may additionally comprise a pharmaceutically acceptable adjuvant and/or carrier.

As the antibodies of the present invention are useful in the treatment, diagnosis and/or prophylaxis of a disorder or condition as described herein, the present invention also provides for a pharmaceutical or diagnostic composition comprising an antibody or antigen-binding fragment thereof according to the present invention in combination with one or more of a pharmaceutically acceptable carrier, excipient or diluent.

In particular the antibody or antigen-binding fragment thereof is provided as a pharmaceutical composition comprising one or more of a pharmaceutically acceptable excipient, diluent or carrier.

These compositions may comprise, in addition to the therapeutically active ingredient(s), a pharmaceutically acceptable excipient, carrier, diluent, buffer, stabilizer or other materials well known to those skilled in the art. Such materials should be non-toxic and should not interfere with the efficacy of the active ingredient.

Also provided are compositions, including pharmaceutical formulations, comprising an antibody of the present invention, or polynucleotides comprising sequences encoding such antibody. In certain embodiments, compositions comprise one or more antibodies that bind to IL13 and IL22 or one or more polynucleotides comprising sequences encoding one or more antibodies that bind to IL13 and IL22. These compositions may further comprise suitable carriers, such as pharmaceutically acceptable excipients and/or adjuvants including buffers, which are well known in the art.

Pharmaceutical compositions of an antibody as described herein are prepared by mixing such antibody having the desired degree of purity with one or more optional pharmaceutically acceptable carriers in the form of lyophilized formulations or aqueous solutions.

Examples of the techniques and protocols mentioned above can be found in Remington's Pharmaceutical Sciences, 20th Edition, 2000, pub. Lippincott, Williams & Wilkins.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Exemplary lyophilized antibody formulations are described in U.S. Pat. No. 6,267,958. Aqueous antibody formulations include those described in U.S. Pat. No. 6,171,586 and WO2006/044908.

The pharmaceutical compositions of the invention may include one or more pharmaceutically acceptable salts.

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Pharmaceutical compositions typically must be sterile and stable under the conditions of manufacture and storage. The composition can be formulated as a solution, microemulsion, liposome, or other ordered structure suitable to high drug concentration.

In one embodiment, the antibody or antigen-binding fragment thereof according to the present invention is the sole active ingredient. In another embodiment, the antibody or antigen-binding fragment thereof according to the present invention is in combination with one or more additional active ingredients. Alternatively, the pharmaceutical compositions comprise the antibody or antigen-binding fragment thereof according to the present invention which is the sole active ingredient and it may be administered individually to a patient in combination (e.g. simultaneously, sequentially or separately) with other agents, drugs or hormones.

The precise nature of the carrier or other material may depend on the route of administration, e.g. oral, intravenous, cutaneous or subcutaneous, nasal, intramuscular and intraperitoneal routes. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Solutions for intravenous administration or infusion may contain as carrier, for example, sterile water or preferably they may be in the form of sterile, aqueous, isotonic saline solutions.

Preferably, the pharmaceutical or diagnostic composition comprises a humanized antibody according to the present invention.

### Therapeutically Effective Amount and Dosage

The antibodies and pharmaceutical compositions may be administered suitably to a patient to identify the therapeutically effective amount required. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Compositions may be conveniently presented in unit dose forms containing a predetermined amount of an active agent of the disclosure per dose. Dose ranges and regimens for any of the embodiments described herein include, but are not limited to, dosages ranging from 1 mg-1000 mg unit doses.

A suitable dosage of an antibody/modulatory agent or pharmaceutical composition of the invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.01 μg/kg to about 1000 mg/kg body weight, typically from about 0.1 μg/kg to about 100 mg/kg body weight, of the patient to be treated.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

### Administration of Pharmaceutical Compositions or Formulations

The antibodies or formulations or compositions thereof may be administered for prophylactic and/or therapeutic treatments.

An antibody or pharmaceutical composition may be administered via one or more routes of administration using one or more of a variety of methods known in the art. As will be appreciated by the skilled artisan, the route and/or mode of administration will vary depending upon the desired results. Examples of routes of administration for compounds or pharmaceutical compositions of the invention include intravenous, intramuscular, intradermal, intraocular, intraperitoneal, subcutaneous, spinal or other parenteral routes of administration, for example by injection or infusion. Alternatively, antibody/modulatory agent or pharmaceutical composition of the invention can be administered via a non-parenteral route, such as a topical, epidermal or mucosal route of administration. The antibody/modulatory agent or pharmaceutical composition of the invention may be for oral administration.

Suitable forms for administration include forms suitable for parenteral administration, e.g. by injection or infusion, for example by bolus injection or continuous infusion, in intravenous, inhalable or sub-cutaneous form. Where the product is for injection or infusion, it may take the form of a suspension, solution or emulsion in an oily or aqueous vehicle and it may contain additional agents, such as suspending, preservative, stabilizing and/or dispersing agents. Alternatively, the antibody or antigen-binding fragment thereof according to the invention may be in dry form, for reconstitution before use with an appropriate sterile liquid. Solid forms suitable for solution in, or suspension in, liquid vehicles prior to injection may also be prepared.

Once formulated, the compositions of the invention can be administered directly to the subject. Accordingly, provided herein is the use of an antibody or an antigen-binding fragment thereof according to the invention for the manufacture of a medicament.

### Articles of Manufacture and Kits

The present disclosure also provides kits comprising the antibodies of the present invention and instructions for use. The kit may further contain one or more additional reagents, such as an additional therapeutic or prophylactic agent as discussed above.

The present invention provides use of a multi-specific antibody according to the invention or pharmaceutical composition thereof for the manufacture of a medicament.

The present invention also provides use of a multi-specific antibody of the present invention for the manufacture of a medicament for the treatment of a disorder or condition as described herein.

The present invention also provides use of a combination of an antibody that binds to IL22 and

an antibody that binds to IL13 or pharmaceutical composition thereof for the manufacture of a medicament for the treatment of a disorder or condition as described herein.

The present invention also provides use of a composition comprising an antibody that binds to IL22 and an antibody that binds to IL13 or pharmaceutical composition thereof for the manufacture of a medicament for the treatment of a disorder or condition as described herein.

In certain embodiments, the article of manufacture or kit comprises a container containing one or more of the antibodies of the invention, or the compositions described herein. In certain embodiments, the article of manufacture or kit comprises a container containing nucleic acids(s) encoding one (or more) of the antibodies or the compositions described herein. In some embodiments, the kit includes a cell or a cell line that produces an antibody as described herein.

In certain embodiments, the article of manufacture or kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treatment, prevention and/or diagnosis and may have a sterile access port. At least one agent in the composition is an antibody of the present invention. The label or package insert indicates that the composition is used for the treatment of a skin inflammatory condition, more specifically atopic dermatitis.

It should be noted that the above-mentioned embodiments illustrate rather than limit the invention, and that those skilled in the art will be able to design many alternative embodiments without departing from the scope of the claims. I

The sequences included in the present invention are shown in Tables 10-17

TABLE 10
Sequences of IL22, IL13 and albumin related
proteins

| | | SEQ |
|---|---|---|
| | | ID |
| Name | Sequence | NO |
| IL22 | MAALQKSVSSFLMGTLATSCLLLLALLVQGGAAAPISS | 1 |
| | HCRLDKSNFQQPYITNRTFMLAKEASLADNNTDVRLIG | |
| | EKLFHGVSMSERCYLMKQVLNFTLEEVLFPQSDRFQPY | |
| | MQEVVPFLARLSNRLSTCHIEGDDLHIQRNVQKLKDTV | |
| | KKLGESGEIKAIGELDLLEMSLRNACI | |
| IL22 | APISSHCRLDKSNFQQPYITNRTFMLAKEASLADNNTD | 2 |
| (34-179) | VRLIGEKLFHGVSMSERCYLMKQVLNFTLEEVLFPQSD | |
| Without | RFQPYMQEVVPFLARLSNRLSTCHIEGDDLHIQRNVQK | |
| signal | LKDTVKKLGESGEIKAIGELDLLFMSLRNACI | |
| peptide | | |
| His- | MGSSHHHHHHSSGENLYFQGSQGGAAAPISSHCRLDKS | 3 |
| tagged | NFQQPYITNRTFMLAKEASLADNNTDVRLIGEKLFHGV | |
| IL22 | SMSERCYLMKQVLNFTLEEVLFPQSDRFQPYMQEVVPF | |
| | LARLSNRLSTCHIEGDDLHIQRNVQKLKDTVKKLGESG | |
| | EIKAIGELDLLFMSLRNACI | |
| Cleaved | GSQGGAAAPISSHCRLDKSNFQQPYITNRTFMLAKEAS | 4 |
| IL22 | LADNNTDVRLIGEKLFHGVSMSERCYLMKQVLNFTLEE | |
| | VLFPQSDRFQPYMQEVVPFLARLSNRLSTCHIEGDDLH | |
| | IQRNVQKLKDTVKKLGESGEIKAIGELDLLFMSLRNAC | |
| | I | |
| IL13 | MHPLLNPLLLALGLMALLLTTVIALTCLGGFASPGPVP | 5 |
| | PSTALRELIEELVNITQNQKAPLCNGSMVWSINLTAGM | |
| | YCAALESLINVSGCSAIEKTQRMLSGFCPHKVSAGQFS | |
| | SLHVRDTKIEVAQFVKDLLLHLKKLFREGRFN | |
| IL13 | LTCLGGFASPGPVPPSTALRELIEELVNITQNQKAPLC | 6 |
| mature | NGSMVWSINLTAGMYCAALESLINVSGCSAIEKTQRML | |
| | SGFCPHKVSAGQFSSLHVRDTKIEVAQFVKDLLLHLKK | |
| | LFREGRFN | |
| Human | MKWVTFISLLFLFSSAYSRGVFRRDAHKSEVAHRFKDL | 7 |
| Albumin | GEENFKALVLIAFAQYLQQCPFEDHVKLVNEVTEFAKT | |
| | CVADESAENCDKSLHTLFGDKLCTVATLRETYGEMADC | |
| | CAKQEPERNECFLQHKDDNPNLPRLVRPEVDVMCTAFH | |
| | DNEETFLKKYLYEIARRHPYFYAPELLFFAKRYKAAFT | |
| | ECCQAADKAACLLPKLDELRDEGKASSAKQRLKCASLQ | |
| | KFGERAFKAWAVARLSQRFPKAEFAEVSKLVTDLTKVH | |
| | TECCHGDLLECADDRADLAKYICENQDSISSKLKECCE | |
| | KPLLEKSHCIAEVENDEMPADLPSLAADFVESKDVCKN | |
| | YAEAKDVFLGMFLYEYARRHPDYSVVLLLRLAKTYETT | |
| | LEKCCAAADPHECYAKVFDEFKPLVEEPQNLIKQNCEL | |
| | FEQLGEYKFQNALLVRYTKKVPQVSTPTLVEVSRNLGK | |
| | VGSKCCKHPEAKRMPCAEDYLSVVLNQLCVLHEKTPVS | |
| | DRVTKCCTESLVNRRPCFSALEVDETYVPKEFNAETFT | |
| | FHADICTLSEKERQIKKQTALVELVKHKPKATKEQLKA | |
| | VMDDFAAFVEKCCKADDKETCFAEEGKKLVAASQAALG | |
| | L | |

TABLE 11
CDRs of IL13-, IL22- and albumin-binding

| domains and IL13/IL22 TrYbe sequences | | |
|---|---|---|
| Name | Sequence | SEQ ID NO |
| 11041 CDRL1 | QASEDIYTNLA | 8 |
| 11041 CDRL2 | WASTLAS | 9 |
| 11041 CDRL3 | QASVYGNAADSRYT | 10 |
| 11041 CDRH1 | GFSLSSYAMI | 11 |
| 11041 CDRH2 | IIDIEGSTYYASWAKG | 12 |
| 11041 CDRH3 | DRFVGVDIFDP | 13 |
| 11041gL13 V-region | AVQLTQSPSSLSASVGDRVTITCQASEDIYTNLAWYQQKPGKAPKLLI | 14 |
| | YWASTLASGVPSRFSGSGSGTDFTLTISSLQPEDFATYYCQASVYGNA | |
| | ADSRYTFGGGTKVEIK | |
| 11041gL13 V-region | gccgtccaactgactcagtccccgagctcactttccgcgagcgtggga | 15 |
| | gatcgcgtgaccattacgtgccaggcctcggaggacatctacaccaac | |
| | ctcgcctggtatcaacagaagcctggcaaagctcccaagctgttgatc | |
| | tactgggcctccactctggcctccggagtgccttcgcggttctccggt | |
| | tctggatcaggcaccgacttcaccctgacaatcagcagcctccagccg | |
| | gaagattttgccacttactactgccaagcatccgtctacgggaacgca | |
| | gcggactccagatataccttcggcgggggaaccaaagtggagattaag | |
| | cgtacg | |
| 11041gH14 V-region | EVQLVESGGGLVQPGGSLRLSCAVSGFSLSSYAMIWVRQAPGKGLEWI | 16 |
| | GIIDIEGSTYYASWAKGRFTISRDNSKNTVYLQMNSLRAEDTAVYYCA | |
| | RDRFVGVDIFDPWGQGTLVTVSS | |
| 11041gH14 V-region | gaggtgcagctcgtggaaagcggaggaggactggtgcagccaggaggg | 17 |
| | tccttgcggcttagctgtgccgtgtccggcttctccctgtcctcctac | |
| | gccatgatctgggtccgccaagctcctgggaagggcctcgaatggatt | |
| | ggtattatcgacatcgagggatcaacctactacgcctcgtgggccaag | |
| | ggacggttcaccatctcgcgggacaactccaagaacactgtgtatctg | |
| | cagatgaacagcctgagggcagaagataccgccgtgtactactgcgcg | |
| | agagatcgcttcgtgggcgtggacatctttgacccgtggggtcaaggc | |
| | accctggtcactgtctcgagc | |
| Light chain (VL- | AVQLTQSPSSLSASVGDRVTITCQASEDIYTNLAWYQQKPGKAPKLLI | 18 |
| CL) 11041gL13 | YWASTLASGVPSRFSGSGSGTDFTLTISSLQPEDFATYYCQASVYGNA | |
| | ADSRYTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYE | |
| | KHKVYACEVTHQGLSSPVTKSENRGEC | |
| Light chain (VL- | gccgtccaactgactcagtccccgagctcactttccgcgagcgtggga | 19 |
| CL) 11041gL13 | gatcgcgtgaccattacgtgccaggcctcggaggacatctacaccaac | |
| | ctcgcctggtatcaacagaagcctggcaaagctcccaagctgttgatc | |
| | tactgggcctccactctggcctccggagtgccttcgcggttctccggt | |
| | tctggatcaggcaccgacttcaccctgacaatcagcagcctccagccg | |
| | gaagattttgccacttactactgccaagcatccgtctacgggaacgca | |
| | gcggactccagatataccttcggcgggggaaccaaagtggagattaag | |
| | cgtacggtggccgctccctccgtgttcatcttcccaccctccgacgag | |
| | cagctgaagtccggcaccgcctccgtcgtgtgcctgctgaacaacttc | |
| | tacccccgcgaggccaaggtgcagtggaaggtggacaacgccctgcag | |
| | tccggcaactcccaggaatccgtcaccgagcaggactccaaggacagc | |
| | acctactccctgtcctccaccctgaccctgtccaaggccgactacgag | |
| | aagcacaaggtgtacgcctgcgaagtgacccaccagggcctgtccagc | |
| | cccgtgaccaagtccttcaaccggggcgagtgc | |
| Heavy chain (VH- | EVQLVESGGGLVQPGGSLRLSCAVSGFSLSSYAMIWVRQAPGKGLEWI | 20 |
| CH1) 11041gH14 | GIIDIEGSTYYASWAKGRFTISRDNSKNTVYLQMNSLRAEDTAVYYCA | |
| | RDRFVGVDIFDPWGQGTLVTVSSASTKGPSVFPLAPSSKSTSGGTAAL | |
| | GCLVKDYFPEPVTVSWNSGALTSGVHTFPAVLQSSGLYSLSSVVTVPS | |
| | SSLGTQTYICNVNHKPSNTKVDKKVEPKSC | |
| Heavy chain (VH- | gaggtgcagctcgtggaaagcggaggaggactggtgcagccaggaggg | 21 |
| CH1) 11041gH14 | tccttgcggcttagctgtgccgtgtccggcttctccctgtcctcctac | |
| | gccatgatctgggtccgccaagctcctgggaagggcctcgaatggatt | |
| | ggtattatcgacatcgagggatcaacctactacgcctcgtgggccaag | |
| | ggacggttcaccatctcgcgggacaactccaagaacactgtgtatctg | |
| | cagatgaacagcctgagggcagaagataccgccgtgtactactgcgcg | |
| | agagatcgcttcgtgggcgtggacatctttgacccgtggggtcaaggc | |
| | accctggtcactgtctcgagcgcgtccacaaagggcccatcggtcttc | |
| | cccctggcaccctcctccaagagcacctctgggggcacagcggccctg | |
| | ggctgcctggtcaaggactacttccccgaaccagtgacggtgtcgtgg | |
| | aactcaggtgccctgaccagcggcgttcacaccttcccggctgtccta | |
| | cagtcttcaggactctactccctgagcagcgtggtgaccgtgccctcc | |
| | agcagcttgggcacccagacctacatctgcaacgtgaatcacaagccc | |
| | agcaacaccaaggtcgataagaaagttgagcccaaatcttgt | |
| 650 CDRL1 | KASQNINENLD | 22 |
| 650 CDRL2 | YTDILQT | 23 |
| 650 CDRL3 | YQYYSGYT | 24 |
| 650 CDRH1 | GYSFTSYYIH | 25 |
| 650 CDRH2 | RIGPGSGDINYNEKFKG | 26 |
| 650 CDRH3 | FHYDGAD | 27 |
| 650 gL8 V-region | DIQMTQSPSSLSASVGDRVTITCKASQNINENLDWYQQKPGKAPKLLI | 28 |
| (unmutated*) | YYTDILQTGIPSRFSGSGSGTDYTLTISSLQPEDFATYYCYQYYSGYT | |
| | FGQGTKLEIK | |
| 650 gH9 V-region | EVQLVQSGAEVKKPGSSVKVSCKASGYSFTSYYIHWVRQAPGQGLEWM | 29 |
| (unmutated*) | GRIGPGSGDINYNEKFKGRATFTVDKSTSTAYMELSSLRSEDTAVYYC | |
| | ARFHYDGADWGQGTLVTVSS | |
| 650 gL8 V-region | gacatccagatgacccagtccccctcctccctgtccgcctccgtgggc | 30 |
| (unmutated*) | gacagggtgaccatcacctgcaaggcctcccagaacatcaacgagaac | |
| | ctggactggtaccagcagaagcccggcaaggcccccaagctgctgatc | |
| | tactacaccgacatcctgcagaccggcatcccctccaggttctccggc | |
| | tccggctccggcaccgactacaccctgaccatctcctccctgcagccc | |
| | gaggacttcgccacctactactgctaccagtactactccggctacacc | |
| | ttcggccagggcaccaagctggagatcaag | |
| 650 gH9 V-region | gaggtgcagctggtgcagtccggcgccgaggtgaagaagcccggctcc | 31 |
| (unmutated*) | tccgtgaaggtgtcctgcaaggcctccggctactccttcacctcctac | |
| | tacatccactgggtgaggcaggcccccggccagggcctggagtggatg | |
| | ggcaggatcggccccggctccggcgacatcaactacaacgagaagttc | |
| | aagggcagggccaccttcaccgtggacaagtccacctccaccgcctac | |
| | atggagctgtcctccctgaggtccgaggacaccgccgtgtactactgc | |
| | gccaggttccactacgacggcgccgactggggccagggcaccctggtg | |
| | accgtgtcctcc | |
| 650 gL8 V-region | DIQMTQSPSSLSASVGDRVTITCKASQNINENLDWYQQKPGKAPKLLI | 32 |
| (mutated**) | YYTDILQTGIPSRFSGSGSGTDYTLTISSLQPEDFATYYCYQYYSGYT | |
| | FGCGTKLEIK | |
| 650 gH9 V-region | EVQLVQSGAEVKKPGSSVKVSCKASGYSFTSYYIHWVRQAPGQCLEWM | 33 |
| (mutated**) | GRIGPGSGDINYNEKFKGRATFTVDKSTSTAYMELSSLRSEDTAVYYC | |
| | ARFHYDGADWGQGTLVTVSS | |
| 650 gL8 V-region | gacatccagatgacccagtccccctcctccctgtccgcctccgtgggc | 34 |
| (mutated**) | gacagggtgaccatcacctgcaaggcctcccagaacatcaacgagaac | |
| | ctggactggtaccagcagaagcccggcaaggcccccaagctgctgatc | |
| | tactacaccgacatcctgcagaccggcatcccctccaggttctccggc | |
| | tccggctccggcaccgactacaccctgaccatctcctccctgcagccc | |
| | gaggacttcgccacctactactgctaccagtactactccggctacacc | |
| | ttcggctgcggcaccaagctggagatcaag | |
| 650 gH9 V-region | gaggtgcagctggtgcagtccggcgccgaggtgaagaagcccggctcc | 35 |
| (mutated**) | tccgtgaaggtgtcctgcaaggcctccggctactccttcacctcctac | |
| | tacatccactgggtgaggcaggcccccggccagtgcctggagtggatg | |
| | ggcaggatcggccccggctccggcgacatcaactacaacgagaagttc | |
| | aagggcagggccaccttcaccgtggacaagtccacctccaccgcctac | |
| | atggagctgtcctccctgaggtccgaggacaccgccgtgtactactgc | |
| | gccaggttccactacgacggcgccgactggggccagggcaccctggtg | |
| | accgtgtcctcc | |
| 650 scFv (VH/VL) | EVQLVQSGAEVKKPGSSVKVSCKASGYSFTSYYIHWVRQAPGQGLEWM | 36 |
| gH9gL8 | GRIGPGSGDINYNEKFKGRATFTVDKSTSTAYMELSSLRSEDTAVYYC | |
| (unmutated*) | ARFHYDGADWGQGTLVTVSSGGGGSGGGGSGGGGSGGGGSDIQMTQSP | |
| | SSLSASVGDRVTITCKASQNINENLDWYQQKPGKAPKLLIYYTDILQT | |
| | GIPSRFSGSGSGTDYTLTISSLQPEDFATYYCYQYYSGYTFGQGTKLE | |
| | IK | |
| 650 scFv (VH/VL) | gaggtgcagctggtgcagtccggcgccgaggtgaagaagcccggctcc | 37 |
| gH9gL8 | tccgtgaaggtgtcctgcaaggcctccggctactccttcacctcctac | |
| (unmutated*) | tacatccactgggtgaggcaggcccccggccagggcctggagtggatg | |
| | ggcaggatcggccccggctccggcgacatcaactacaacgagaagttc | |
| | aagggcagggccaccttcaccgtggacaagtccacctccaccgcctac | |
| | atggagctgtcctccctgaggtccgaggacaccgccgtgtactactgc | |
| | gccaggttccactacgacggcgccgactggggccagggcaccctggtg | |
| | accgtgtcctccggaggtggcggttctggcggtggcggttccggtggc | |
| | ggtggatcgggaggtggcggttctgacatccagatgacccagtccccc | |
| | tcctccctgtccgcctccgtgggcgacagggtgaccatcacctgcaag | |
| | gcctcccagaacatcaacgagaacctggactggtaccagcagaagccc | |
| | ggcaaggcccccaagctgctgatctactacaccgacatcctgcagacc | |
| | ggcatcccctccaggttctccggctccggctccggcaccgactacacc | |
| | ctgaccatctcctccctgcagcccgaggacttcgccacctactactgc | |
| | taccagtactactccggctacaccttcggccagggcaccaagctggag | |
| | atcaag | |
| 650 dsscFv (VH/VL) | EVQLVQSGAEVKKPGSSVKVSCKASGYSFTSYYIHWVRQAPGQCLEWM | 38 |
| gH9gL8 (mutated**) | GRIGPGSGDINYNEKFKGRATFTVDKSTSTAYMELSSLRSEDTAVYYC | |
| | ARFHYDGADWGQGTLVTVSSGGGGSGGGGSGGGGSGGGGSDIQMTQSP | |
| | SSLSASVGDRVTITCKASQNINENLDWYQQKPGKAPKLLIYYTDILQT | |
| | GIPSRFSGSGSGTDYTLTISSLQPEDFATYYCYQYYSGYTFGCGTKLE | |
| | IK | |
| 650 dsscFv (VH/VL) | gaggtgcagctggtgcagtccggcgccgaggtgaagaagcccggctcc | 39 |
| gH9gL8 (mutated**) | tccgtgaaggtgtcctgcaaggcctccggctactccttcacctcctac | |
| | tacatccactgggtgaggcaggcccccggccagtgcctggagtggatg | |
| | ggcaggatcggccccggctccggcgacatcaactacaacgagaagttc | |
| | aagggcagggccaccttcaccgtggacaagtccacctccaccgcctac | |
| | atggagctgtcctccctgaggtccgaggacaccgccgtgtactactgc | |
| | gccaggttccactacgacggcgccgactggggccagggcaccctggtg | |
| | accgtgtcctccggaggtggcggttctggcggtggcggttccggtggc | |
| | ggtggatcgggaggtggcggttctgacatccagatgacccagtccccc | |
| | tcctccctgtccgcctccgtgggcgacagggtgaccatcacctgcaag | |
| | gcctcccagaacatcaacgagaacctggactggtaccagcagaagccc | |
| | ggcaaggcccccaagctgctgatctactacaccgacatcctgcagacc | |
| | ggcatcccctccaggttctccggctccggctccggcaccgactacacc | |
| | ctgaccatctcctccctgcagcccgaggacttcgccacctactactgc | |
| | taccagtactactccggctacaccttcggctgcggcaccaagctggag | |
| | atcaag | |
| 645 CDRL1 | QSSPSVWSNFLS | 40 |
| 645 CDRL2 | EASKLTS | 41 |
| 645 CDRL3 | GGGYSSISDTT | 42 |
| 645 CDRH1 | GIDLSNYAIN | 43 |
| 645 CDRH2 | IIWASGTTFYATWAKG | 44 |
| 645 CDRH3 | TVPGYSTAPYFDL | 45 |
| 645 VL-region | DIQMTQSPSSVSASVGDRVTITCQSSPSVWSNFLSWYQQKPGKAPKLL | 46 |
| (unmutated*) | IYEASKLTSGVPSRFSGSGSGTDFTLTISSLQPEDFATYYCGGGYSSI | |
| | SDTTFGGGTKVEIK | |
| 645 VH-region | EVQLLESGGGLVQPGGSLRLSCAVSGIDLSNYAINWVRQAPGKGLEWI | 47 |
| (unmutated*) | GIIWASGTTFYATWAKGRFTISRDNSKNTVYLQMNSLRAEDTAVYYCA | |
| | RTVPGYSTAPYFDLWGQGTLVTVSS | |
| 645 VL-region | gacatacaaatgactcagtctccttcatcggtatccgcgtccgttggc | 48 |
| (unmutated*) | gatagggtgactattacatgtcaaagctctcctagcgtctggagcaat | |
| | tttctatcctggtatcaacagaaaccggggaaggctccaaaacttctg | |
| | atttatgaagcctcgaaactcaccagtggagttccgtcaagattcagt | |
| | ggctctggatcagggacagacttcacgttgacaatcagttcgctgcaa | |
| | ccagaggactttgcgacctactattgtggtggaggttacagtagcata | |
| | agtgatacgacatttgggggcggtactaaggtggaaatcaaa | |
| 645 VH-region | gaggttcaactgcttgagtctggaggaggcctagtccagcctggaggg | 49 |
| (unmutated*) | agcctgcgtctctcttgtgcagtaagcggcatcgacctgagcaattac | |
| | gccatcaactgggtgagacaagctccggggaagggtttagaatggatc | |
| | ggtataatatgggccagtgggacgaccttttatgctacatgggcgaaa | |
| | ggaaggtttacaattagccgggacaatagcaaaaacaccgtgtatctc | |
| | caaatgaactccttgcgagcagaggacacggcggtgtactattgtgct | |
| | cgcactgtcccaggttatagcactgcaccctacttcgatctgtgggga | |
| | caagggaccctggtgactgtttcaagt | |
| 645 VL-region | DIQMTQSPSSVSASVGDRVTITCQSSPSVWSNFLSWYQQKPGKAPKLL | 50 |
| (mutated**) | IYEASKLTSGVPSRFSGSGSGTDFTLTISSLQPEDFATYYCGGGYSSI | |
| | SDTTFGCGTKVEIK | |
| 645 VH-region | EVQLLESGGGLVQPGGSLRLSCAVSGIDLSNYAINWVRQAPGKCLEWI | 51 |
| (mutated**) | GIIWASGTTFYATWAKGRFTISRDNSKNTVYLQMNSLRAEDTAVYYCA | |
| | RTVPGYSTAPYFDLWGQGTLVTVSS | |
| 645 VL-region | gacatacaaatgactcagtctccttcatcggtatccgcgtccgttggc | 52 |
| (mutated**) | gatagggtgactattacatgtcaaagctctcctagcgtctggagcaat | |
| | tttctatcctggtatcaacagaaaccggggaaggctccaaaacttctg | |
| | atttatgaagcctcgaaactcaccagtggagttccgtcaagattcagt | |
| | ggctctggatcagggacagacttcacgttgacaatcagttcgctgcaa | |
| | ccagaggactttgcgacctactattgtggtggaggttacagtagcata | |
| | agtgatacgacatttgggtgcggtactaaggtggaaatcaaa | |
| 645 VH-region | gaggttcaactgcttgagtctggaggaggcctagtccagcctggaggg | 53 |
| (mutated**) | agcctgcgtctctcttgtgcagtaagcggcatcgacctgagcaattac | |
| | gccatcaactgggtgagacaagctccggggaagtgtttagaatggatc | |
| | ggtataatatgggccagtgggacgaccttttatgctacatgggcgaaa | |
| | ggaaggtttacaattagccgggacaatagcaaaaacaccgtgtatctc | |
| | caaatgaactccttgcgagcagaggacacggcggtgtactattgtgct | |
| | cgcactgtcccaggttatagcactgcaccctacttcgatctgtgggga | |
| | caagggaccctggtgactgtttcaagt | |
| 645 scFv (VH/VL) | EVQLLESGGGLVQPGGSLRLSCAVSGIDLSNYAINWVRQAPGKGLEWI | 54 |
| (unmutated*) | GIIWASGTTFYATWAKGRFTISRDNSKNTVYLQMNSLRAEDTAVYYCA | |
| | RTVPGYSTAPYFDLWGQGTLVTVSSGGGGSGGGGSGGGGSGGGGSDIQ | |
| | MTQSPSSVSASVGDRVTITCQSSPSVWSNFLSWYQQKPGKAPKLLIYE | |
| | ASKLTSGVPSRFSGSGSGTDFTLTISSLQPEDFATYYCGGGYSSISDT | |
| | TFGGGTKVEIK | |
| 645 scFv (VH/VL) | gaggttcaactgcttgagtctggaggaggcctagtccagcctggaggg | 55 |
| (unmutated*) | agcctgcgtctctcttgtgcagtaagcggcatcgacctgagcaattac | |
| | gccatcaactgggtgagacaagctccggggaagggtttagaatggatc | |
| | ggtataatatgggccagtgggacgaccttttatgctacatgggcgaaa | |
| | ggaaggtttacaattagccgggacaatagcaaaaacaccgtgtatctc | |
| | caaatgaactccttgcgagcagaggacacggcggtgtactattgtgct | |
| | cgcactgtcccaggttatagcactgcaccctacttcgatctgtgggga | |
| | caagggaccctggtgactgtttcaagtggaggtggcggttctggcggt | |
| | ggcggttccggtggcggtggatcgggaggtggcggttctgacatacaa | |
| | atgactcagtctccttcatcggtatccgcgtccgttggcgatagggtg | |
| | actattacatgtcaaagctctcctagcgtctggagcaattttctatcc | |
| | tggtatcaacagaaaccggggaaggctccaaaacttctgatttatgaa | |
| | gcctcgaaactcaccagtggagttccgtcaagattcagtggctctgga | |
| | tcagggacagacttcacgttgacaatcagttcgctgcaaccagaggac | |
| | tttgcgacctactattgtggtggaggttacagtagcataagtgatacg | |
| | acatttgggggcggtactaaggtggaaatcaaa | |
| 645 dsscFv (VH/VL) | EVQLLESGGGLVQPGGSLRLSCAVSGIDLSNYAINWVRQAPGKCLEWI | 56 |
| (mutated**) | GIIWASGTTFYATWAKGRFTISRDNSKNTVYLQMNSLRAEDTAVYYCA | |
| | RTVPGYSTAPYFDLWGQGTLVTVSSGGGGSGGGGSGGGGSGGGGSDIQ | |
| | MTQSPSSVSASVGDRVTITCQSSPSVWSNFLSWYQQKPGKAPKLLIYE | |
| | ASKLTSGVPSRFSGSGSGTDFTLTISSLQPEDFATYYCGGGYSSISDT | |
| | TFGCGTKVEIK | |
| 645 dsscFv (VH/VL) | gaggttcaactgcttgagtctggaggaggcctagtccagcctggaggg | 57 |
| (mutated**) | agcctgcgtctctcttgtgcagtaagcggcatcgacctgagcaattac | |
| | gccatcaactgggtgagacaagctccggggaagtgtttagaatggatc | |
| | ggtataatatgggccagtgggacgaccttttatgctacatgggcgaaa | |
| | ggaaggtttacaattagccgggacaatagcaaaaacaccgtgtatctc | |
| | caaatgaactccttgcgagcagaggacacggcggtgtactattgtgct | |
| | cgcactgtcccaggttatagcactgcaccctacttcgatctgtgggga | |
| | caagggaccctggtgactgtttcaagtggaggtggcggttctggcggt | |
| | ggcggttccggtggcggtggatcgggaggtggcggttctgacatacaa | |
| | atgactcagtctccttcatcggtatccgcgtccgttggcgatagggtg | |
| | actattacatgtcaaagctctcctagcgtctggagcaattttctatcc | |
| | tggtatcaacagaaaccggggaaggctccaaaacttctgatttatgaa | |
| | gcctcgaaactcaccagtggagttccgtcaagattcagtggctctgga | |
| | tcagggacagacttcacgttgacaatcagttcgctgcaaccagaggac | |
| | tttgcgacctactattgtggtggaggttacagtagcataagtgatacg | |
| | acatttgggtgcggtactaaggtggaaatcaaa | |
| 11041gH14 HC- 645 | EVQLVESGGGLVQPGGSLRLSCAVSGFSLSSYAMIWVRQAPGKGLEWI | 58 |
| (VH/VL) scFv | GIIDIEGSTYYASWAKGRFTISRDNSKNTVYLQMNSLRAEDTAVYYCA | |
| (unmutated*) | RDRFVGVDIFDPWGQGTLVTVSSASTKGPSVFPLAPSSKSTSGGTAAL | |
| | GCLVKDYFPEPVTVSWNSGALTSGVHTFPAVLQSSGLYSLSSVVTVPS | |
| | SSLGTQTYICNVNHKPSNTKVDKKVEPKSCSGGGGSGGGGSEVQLLES | |
| | GGGLVQPGGSLRLSCAVSGIDLSNYAINWVRQAPGKGLEWIGIIWASG | |
| | TTFYATWAKGRFTISRDNSKNTVYLQMNSLRAEDTAVYYCARTVPGYS | |
| | TAPYFDLWGQGTLVTVSSGGGGSGGGGSGGGGSGGGGSDIQMTQSPSS | |
| | VSASVGDRVTITCQSSPSVWSNFLSWYQQKPGKAPKLLIYEASKLTSG | |
| | VPSRFSGSGSGTDFTLTISSLQPEDFATYYCGGGYSSISDTTFGGGTK | |
| | VEIKRT | |
| 11041gH14 HC- 645 | gaggtgcagctcgtggaaagcggaggaggactggtgcagccaggaggg | 59 |
| (VH/VL) scFv | tccttgcggcttagctgtgccgtgtccggcttctccctgtcctcctac | |
| (unmutated*) | gccatgatctgggtccgccaagctcctgggaagggcctcgaatggatt | |
| | ggtattatcgacatcgagggatcaacctactacgcctcgtgggccaag | |
| | ggacggttcaccatctcgcgggacaactccaagaacactgtgtatctg | |
| | cagatgaacagcctgagggcagaagataccgccgtgtactactgcgcg | |
| | agagatcgcttcgtgggcgtggacatctttgacccgtggggtcaaggc | |
| | accctggtcactgtctcgagcgcgtccacaaagggcccatcggtcttc | |
| | cccctggcaccctcctccaagagcacctctgggggcacagcggccctg | |
| | ggctgcctggtcaaggactacttccccgaaccagtgacggtgtcgtgg | |
| | aactcaggtgccctgaccagcggcgttcacaccttcccggctgtccta | |
| | cagtcttcaggactctactccctgagcagcgtggtgaccgtgccctcc | |
| | agcagcttgggcacccagacctacatctgcaacgtgaatcacaagccc | |
| | agcaacaccaaggtcgataagaaagttgagcccaaatcttgtagcggt | |
| | ggcggtggctccggaggtggcggttcagaggttcaactgcttgagtct | |
| | ggaggaggcctagtccagcctggagggagcctgcgtctctcttgtgca | |
| | gtaagcggcatcgacctgagcaattacgccatcaactgggtgagacaa | |
| | gctccggggaagggtttagaatggatcggtataatatgggccagtggg | |
| | acgaccttttatgctacatgggcgaaaggaaggtttacaattagccgg | |
| | gacaatagcaaaaacaccgtgtatctccaaatgaactccttgcgagca | |
| | gaggacacggcggtgtactattgtgctcgcactgtcccaggttatagc | |
| | actgcaccctacttcgatctgtggggacaagggaccctggtgactgtt | |
| | tcaagtggaggtggcggttctggcggtggcggttccggtggcggtgga | |
| | tcgggaggtggcggttctgacatacaaatgactcagtctccttcatcg | |
| | gtatccgcgtccgttggcgatagggtgactattacatgtcaaagctct | |
| | cctagcgtctggagcaattttctatcctggtatcaacagaaaccgggg | |
| | aaggctccaaaacttctgatttatgaagcctcgaaactcaccagtgga | |
| | gttccgtcaagattcagtggctctggatcagggacagacttcacgttg | |
| | acaatcagttcgctgcaaccagaggactttgcgacctactattgtggt | |
| | ggaggttacagtagcataagtgatacgacatttgggggcggtactaag | |
| | gtggaaatcaaacgtacc | |
| 11041gH14 HC- 645 | EVQLVESGGGLVQPGGSLRLSCAVSGFSLSSYAMIWVRQAPGKGLEWI | 60 |
| (VH/VL) dsscFv | GIIDIEGSTYYASWAKGRFTISRDNSKNTVYLQMNSLRAEDTAVYYCA | |
| (mutated**) | RDRFVGVDIFDPWGQGTLVTVSSASTKGPSVFPLAPSSKSTSGGTAAL | |
| | GCLVKDYFPEPVTVSWNSGALTSGVHTFPAVLQSSGLYSLSSVVTVPS | |
| | SSLGTQTYICNVNHKPSNTKVDKKVEPKSCSGGGGSGGGGSEVQLLES | |
| | GGGLVQPGGSLRLSCAVSGIDLSNYAINWVRQAPGKCLEWIGIIWASG | |
| | TTFYATWAKGRFTISRDNSKNTVYLQMNSLRAEDTAVYYCARTVPGYS | |
| | TAPYFDLWGQGTLVTVSSGGGGSGGGGSGGGGSGGGGSDIQMTQSPSS | |
| | VSASVGDRVTITCQSSPSVWSNFLSWYQQKPGKAPKLLIYEASKLTSG | |
| | VPSRFSGSGSGTDFTLTISSLQPEDFATYYCGGGYSSISDTTFGCGTK | |
| | VEIKRT | |
| 11041gH14 HC- 645 | gaggtgcagctcgtggaaagcggaggaggactggtgcagccaggaggg | 61 |
| (VH/VL) dsscFv | tccttgcggcttagctgtgccgtgtccggcttctccctgtcctcctac | |
| (mutated**) | gccatgatctgggtccgccaagctcctgggaagggcctcgaatggatt | |
| | ggtattatcgacatcgagggatcaacctactacgcctcgtgggccaag | |
| | ggacggttcaccatctcgcgggacaactccaagaacactgtgtatctg | |
| | cagatgaacagcctgagggcagaagataccgccgtgtactactgcgcg | |
| | agagatcgcttcgtgggcgtggacatctttgacccgtggggtcaaggc | |
| | accctggtcactgtctcgagcgcgtccacaaagggcccatcggtcttc | |
| | cccctggcaccctcctccaagagcacctctgggggcacagcggccctg | |
| | ggctgcctggtcaaggactacttccccgaaccagtgacggtgtcgtgg | |
| | aactcaggtgccctgaccagcggcgttcacaccttcccggctgtccta | |
| | cagtcttcaggactctactccctgagcagcgtggtgaccgtgccctcc | |
| | agcagcttgggcacccagacctacatctgcaacgtgaatcacaagccc | |
| | agcaacaccaaggtcgataagaaagttgagcccaaatcttgtagcggt | |
| | ggcggtggctccggaggtggcggttcagaggttcaactgcttgagtct | |
| | ggaggaggcctagtccagcctggagggagcctgcgtctctcttgtgca | |
| | gtaagcggcatcgacctgagcaattacgccatcaactgggtgagacaa | |
| | gctccggggaagtgtttagaatggatcggtataatatgggccagtggg | |
| | acgaccttttatgctacatgggcgaaaggaaggtttacaattagccgg | |
| | gacaatagcaaaaacaccgtgtatctccaaatgaactccttgcgagca | |
| | gaggacacggcggtgtactattgtgctcgcactgtcccaggttatagc | |
| | actgcaccctacttcgatctgtggggacaagggaccctggtgactgtt | |
| | tcaagtggaggtggcggttctggcggtggcggttccggtggcggtgga | |
| | tcgggaggtggcggttctgacatacaaatgactcagtctccttcatcg | |
| | gtatccgcgtccgttggcgatagggtgactattacatgtcaaagctct | |
| | cctagcgtctggagcaattttctatcctggtatcaacagaaaccgggg | |
| | aaggctccaaaacttctgatttatgaagcctcgaaactcaccagtgga | |
| | gttccgtcaagattcagtggctctggatcagggacagacttcacgttg | |
| | acaatcagttcgctgcaaccagaggactttgcgacctactattgtggt | |
| | ggaggttacagtagcataagtgatacgacatttgggtgcggtactaag | |
| | gtggaaatcaaacgtacc | |
| 11041gL13 LC- 650 | AVQLTQSPSSLSASVGDRVTITCQASEDIYTNLAWYQQKPGKAPKLLI | 62 |
| scFv (unmutated*) | YWASTLASGVPSRFSGSGSGTDFTLTISSLQPEDFATYYCQASVYGNA | |
| | ADSRYTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSENRGECSGGGGSGGGGSEVQLVQSGAE | |
| | VKKPGSSVKVSCKASGYSFTSYYIHWVRQAPGQGLEWMGRIGPGSGDI | |
| | NYNEKFKGRATFTVDKSTSTAYMELSSLRSEDTAVYYCARFHYDGADW | |
| | GQGTLVTVSSGGGGSGGGGSGGGGSGGGGSDIQMTQSPSSLSASVGDR | |
| | VTITCKASQNINENLDWYQQKPGKAPKLLIYYTDILQTGIPSRFSGSG | |
| | SGTDYTLTISSLQPEDFATYYCYQYYSGYTFGQGTKLEIKRT | |
| 11041gL13 LC- 650 | gccgtccaactgactcagtccccgagctcactttccgcgagcgtggga | 63 |
| scFv (unmutated*) | gatcgcgtgaccattacgtgccaggcctcggaggacatctacaccaac | |
| | ctcgcctggtatcaacagaagcctggcaaagctcccaagctgttgatc | |
| | tactgggcctccactctggcctccggagtgccttcgcggttctccggt | |
| | tctggatcaggcaccgacttcaccctgacaatcagcagcctccagccg | |
| | gaagattttgccacttactactgccaagcatccgtctacgggaacgca | |
| | gcggactccagatataccttcggcgggggaaccaaagtggagattaag | |
| | cgtacggtggccgctccctccgtgttcatcttcccaccctccgacgag | |
| | cagctgaagtccggcaccgcctccgtcgtgtgcctgctgaacaacttc | |
| | tacccccgcgaggccaaggtgcagtggaaggtggacaacgccctgcag | |
| | tccggcaactcccaggaatccgtcaccgagcaggactccaaggacagc | |
| | acctactccctgtcctccaccctgaccctgtccaaggccgactacgag | |
| | aagcacaaggtgtacgcctgcgaagtgacccaccagggcctgtccagc | |
| | cccgtgaccaagtccttcaaccggggcgagtgcagcggtggcggtggc | |
| | tccggaggtggcggttcagaggtgcagctggtgcagtccggcgccgag | |
| | gtgaagaagcccggctcctccgtgaaggtgtcctgcaaggcctccggc | |
| | tactccttcacctcctactacatccactgggtgaggcaggcccccggc | |
| | cagggcctggagtggatgggcaggatcggccccggctccggcgacatc | |
| | aactacaacgagaagttcaagggcagggccaccttcaccgtggacaag | |
| | tccacctccaccgcctacatggagctgtcctccctgaggtccgaggac | |
| | accgccgtgtactactgcgccaggttccactacgacggcgccgactgg | |
| | ggccagggcaccctggtgaccgtgtcctccggaggtggcggttctggc | |
| | ggtggcggttccggtggcggtggatcgggaggtggcggttctgacatc | |
| | cagatgacccagtccccctcctccctgtccgcctccgtgggcgacagg | |
| | gtgaccatcacctgcaaggcctcccagaacatcaacgagaacctggac | |
| | tggtaccagcagaagcccggcaaggcccccaagctgctgatctactac | |
| | accgacatcctgcagaccggcatcccctccaggttctccggctccggc | |
| | tccggcaccgactacaccctgaccatctcctccctgcagcccgaggac | |
| | ttcgccacctactactgctaccagtactactccggctacaccttcggc | |
| | cagggcaccaagctggagatcaagcgtacc | |
| 11041gL13 LC- 650 | AVQLTQSPSSLSASVGDRVTITCQASEDIYTNLAWYQQKPGKAPKLLI | 64 |
| dsscFv (mutated**) | YWASTLASGVPSRFSGSGSGTDFTLTISSLQPEDFATYYCQASVYGNA | |
| | ADSRYTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSENRGECSGGGGSGGGGSEVQLVQSGAE | |
| | VKKPGSSVKVSCKASGYSFTSYYIHWVRQAPGQCLEWMGRIGPGSGDI | |
| | NYNEKFKGRATFTVDKSTSTAYMELSSLRSEDTAVYYCARFHYDGADW | |
| | GQGTLVTVSSGGGGSGGGGSGGGGSGGGGSDIQMTQSPSSLSASVGDR | |
| | VTITCKASQNINENLDWYQQKPGKAPKLLIYYTDILQTGIPSRESGSG | |
| | SGTDYTLTISSLQPEDFATYYCYQYYSGYTFGCGTKLEIKRT | |
| 11041gL13 LC- 650 | gccgtccaactgactcagtccccgagctcactttccgcgagcgtggga | 65 |
| dsscFv (mutated**) | gatcgcgtgaccattacgtgccaggcctcggaggacatctacaccaac | |
| | ctcgcctggtatcaacagaagcctggcaaagctcccaagctgttgatc | |
| | tactgggcctccactctggcctccggagtgccttcgcggttctccggt | |
| | tctggatcaggcaccgacttcaccctgacaatcagcagcctccagccg | |
| | gaagattttgccacttactactgccaagcatccgtctacgggaacgca | |
| | gcggactccagatataccttcggcgggggaaccaaagtggagattaag | |
| | cgtacggtggccgctccctccgtgttcatcttcccaccctccgacgag | |
| | cagctgaagtccggcaccgcctccgtcgtgtgcctgctgaacaacttc | |
| | tacccccgcgaggccaaggtgcagtggaaggtggacaacgccctgcag | |
| | tccggcaactcccaggaatccgtcaccgagcaggactccaaggacagc | |
| | acctactccctgtcctccaccctgaccctgtccaaggccgactacgag | |
| | aagcacaaggtgtacgcctgcgaagtgacccaccagggcctgtccagc | |
| | cccgtgaccaagtccttcaaccggggcgagtgcagcggtggcggtggc | |
| | tccggaggtggcggttcagaggtgcagctggtgcagtccggcgccgag | |
| | gtgaagaagcccggctcctccgtgaaggtgtcctgcaaggcctccggc | |
| | tactccttcacctcctactacatccactgggtgaggcaggcccccggc | |
| | cagtgcctggagtggatgggcaggatcggccccggctccggcgacatc | |
| | aactacaacgagaagttcaagggcagggccaccttcaccgtggacaag | |
| | tccacctccaccgcctacatggagctgtcctccctgaggtccgaggac | |
| | accgccgtgtactactgcgccaggttccactacgacggcgccgactgg | |
| | ggccagggcaccctggtgaccgtgtcctccggaggtggcggttctggc | |
| | ggtggcggttccggtggcggtggatcgggaggtggcggttctgacatc | |
| | cagatgacccagtccccctcctccctgtccgcctccgtgggcgacagg | |
| | gtgaccatcacctgcaaggcctcccagaacatcaacgagaacctggac | |
| | tggtaccagcagaagcccggcaaggcccccaagctgctgatctactac | |
| | accgacatcctgcagaccggcatcccctccaggttctccggctccggc | |
| | tccggcaccgactacaccctgaccatctcctccctgcagcccgaggac | |
| | ttcgccacctactactgctaccagtactactccggctacaccttcggc | |
| | tgcggcaccaagctggagatcaagcgtacc | |
| Light chain linker | SGGGGSGGGGS | 66 |
| between kappa | | |
| constant region | | |
| and 650 VH of | | |
| scFv/dssFv (Y) | | |
| Light chain linker | GGGGSGGGGSGGGGSGGGGS | 67 |
| between VH and VL | | |
| of 650 scFv/dsscFv | | |
| Heavy chain linker | SGGGGSGGGGS | 68 |
| between CH1 | | |
| constant region | | |
| and 645 VH of | | |
| scFv/dssFv (X) | | |
| Heavy chain linker | GGGGSGGGGSGGGGSGGGGS | 69 |
| between VH and VL | | |
| of 645 scFv/dsscFv | | |

*ie without cysteines engineered for a disulphide bond
**ie with cysteines engineered for a disulphide bond

TABLE 12
11070 IL22-binding domain sequences

| Name | Sequence | SEQ ID NO |
|---|---|---|
| 11070 CDRL1 | KASKTISKYLA | 70 |
| 11070 CDRL2 | SGSTLQS | 71 |
| 11070 CDRL3 | QQHNEYPLT | 72 |
| 11070 CDRH1 | GFSLTSYSVH | 73 |
| 11070 CDRH2 | RMWSDGDTSYNTAFTS | 74 |
| 11070 CDRH3 | SLDFYYDTTLAF | 75 |
| 11070gL7 V-region | DIQMTQSPSSVSASVGDRVTITCKASKTISKYLAWYQQKPGKANKLLI | 76 |
| | YSGSTLQSGVPSRFSGSGSGTDFTLTISSLQPEDFATYYCQQHNEYPL | |
| | TFGQGTKLEIK | |
| 11070gL7 V-region | gacattcagatgactcagtcgccttcgtccgtgagcgccagcgtcgga | 77 |
| | gacagagtgacaatcacctgtaaagcgtccaagaccatctccaagtac | |
| | ctggcttggtatcagcagaaaccggggaaggccaacaagttgcttatc | |
| | tactccggttctactctccaatcgggagtgccaagccggttttccggg | |
| | tccggatcaggcaccgacttcaccctcaccatctcatccctgcaaccg | |
| | gaggatttcgccacgtactactgccagcagcacaacgaataccccctg | |
| | accttcggccaaggaactaagctggaaattaag | |
| 11070gH16 V-region | EVQLQESGPGLVKPSQTLSLTCTVSGFSLTSYSVHWVRQHPGKGLEWI | 78 |
| | GRMWSDGDTSYNTAFTSRLTISRDTSKNQVSLKLSSVTAADTAVYYCA | |
| | RSLDFYYDTTLAFWGQGTTVTVSS | |
| 11070gH16 V-region | gaggtgcagctgcaagaatccggtcctggcctcgtgaagccgtcgcag | 79 |
| | accttgagcctgacctgtactgtgtccggattcagcctcacatcctac | |
| | tcggtgcactgggtcagacagcatcccggaaaaggcctggaatggatt | |
| | gggaggatgtggtctgatggagacacctcctacaacacggcgttcacc | |
| | agccggctgaccatctcccgcgacacctccaagaaccaagtgtcgctt | |
| | aagctgtcctcagtcactgccgccgataccgcagtgtattactgcgct | |
| | cggtcactggacttttactacgacaccaccctggccttctggggacag | |
| | gggactactgtgactgtctcgagc | |
| 11070gL7 Light | DIQMTQSPSSVSASVGDRVTITCKASKTISKYLAWYQQKPGKANKLLI | 80 |
| chain | YSGSTLQSGVPSRFSGSGSGTDFTLTISSLQPEDFATYYCQQHNEYPL | |
| | TFGQGTKLEIKRTVAAPSVFIFPPSDEQLKSGTASVVCLLNNFYPREA | |
| | KVQWKVDNALQSGNSQESVTEQDSKDSTYSLSSTLTLSKADYEKHKVY | |
| | ACEVTHQGLSSPVTKSENRGEC | |
| 11070gL7 Light | gacattcagatgactcagtcgccttcgtccgtgagcgccagcgtcgga | 81 |
| chain | gacagagtgacaatcacctgtaaagcgtccaagaccatctccaagtac | |
| | ctggcttggtatcagcagaaaccggggaaggccaacaagttgcttatc | |
| | tactccggttctactctccaatcgggagtgccaagccggttttccggg | |
| | tccggatcaggcaccgacttcaccctcaccatctcatccctgcaaccg | |
| | gaggatttcgccacgtactactgccagcagcacaacgaataccccctg | |
| | accttcggccaaggaactaagctggaaattaagcgtacggtggccgct | |
| | ccctccgtgttcatcttcccaccctccgacgagcagctgaagtccggc | |
| | accgcctccgtcgtgtgcctgctgaacaacttctacccccgcgaggcc | |
| | aaggtgcagtggaaggtggacaacgccctgcagtccggcaactcccag | |
| | gaatccgtcaccgagcaggactccaaggacagcacctactccctgtcc | |
| | tccaccctgaccctgtccaaggccgactacgagaagcacaaggtgtac | |
| | gcctgcgaagtgacccaccagggcctgtccagccccgtgaccaagtcc | |
| | ttcaaccggggcgagtgc | |
| 11070gH16 Fab | EVQLQESGPGLVKPSQTLSLTCTVSGFSLTSYSVHWVRQHPGKGLEWI | 82 |
| Heavy chain | GRMWSDGDTSYNTAFTSRLTISRDTSKNQVSLKLSSVTAADTAVYYCA | |
| | RSLDFYYDTTLAFWGQGTTVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKKVEPKSC | |
| 11070gH16 Fab | gaggtgcagctgcaagaatccggtcctggcctcgtgaagccgtcgcag | 83 |
| Heavy chain | accttgagcctgacctgtactgtgtccggattcagcctcacatcctac | |
| | tcggtgcactgggtcagacagcatcccggaaaaggcctggaatggatt | |
| | gggaggatgtggtctgatggagacacctcctacaacacggcgttcacc | |
| | agccggctgaccatctcccgcgacacctccaagaaccaagtgtcgctt | |
| | aagctgtcctcagtcactgccgccgataccgcagtgtattactgcgct | |
| | cggtcactggacttttactacgacaccaccctggccttctggggacag | |
| | gggactactgtgactgtctcgagcgcgtccacaaagggcccatcggtc | |
| | ttccccctggcaccctcctccaagagcacctctgggggcacagcggcc | |
| | ctgggctgcctggtcaaggactacttccccgaaccagtgacggtgtcg | |
| | tggaactcaggtgccctgaccagcggcgttcacaccttcccggctgtc | |
| | ctacagtcttcaggactctactccctgagcagcgtggtgaccgtgccc | |
| | tccagcagcttgggcacccagacctacatctgcaacgtgaatcacaag | |
| | cccagcaacaccaaggtcgataagaaagttgagcccaaatcttgt | |

TABLE 13
Other 11041 sequences (IL22-binding domain)

| Name | Sequence | SEQ ID NO |
|---|---|---|
| 11041 CDRL1 | Same as SEQ ID NO 8 | 8 |
| 11041 CDRL2 | Same as SEQ ID NO 9 | 9 |
| 11041 CDRL3 (not | QACVYGNSADSRYT | 84 |
| mutated) | | |
| 11041 CDRL3 C91S | QASVYGNSADSRYT | 85 |
| 11041 CDRL3 C91V | QAVVYGNSADSRYT | 86 |
| 11041 CDRL3 S96A | QACVYGNAADSRYT | 87 |
| 11041 CDRL3 C91S | Same as SEQ ID NO 10 | 10 |
| S96A | | |
| 11041 CDRL3 C91V | QAVVYGNAADSRYT | 88 |
| S96A | | |
| 11041 CDRL3 N95D | QACVYGDSADSRYT | 89 |
| 11041 CDRL3 C91S | QASVYGDSADSRYT | 90 |
| N95D | | |
| 11041 CDRL3 C91V | QAVVYGDSADSRYT | 91 |
| N95D | | |
| 11041 CDRH1 | Same as SEQ ID NO 11 | 11 |
| 11041 CDRH2 (not | IIDIDGSTYYASWAKG | 92 |
| mutated) | | |
| 11041 CDRH2 G55A | IIDIDASTYYASWAKG | 93 |
| 11041 CDRH2 D54E | Same as SEQ ID NO 12 | 12 |
| 11041 CDRH3 | Same as SEQ ID NO 13 | 13 |
| 11041 CDRH3 D107E | DRFVGVDIFEP | 94 |
| Rabbit 11041 VL- | AVVLTQTASPVSAPVGGTVTIKCQASEDIYTNLAWYQQKPGQPPKLLI | 95 |
| region | YWASTLASGVPSRFKGSGSGTEFTLTISDLECADAATYYCQACVYGNS | |
| | ADSRYTFGGGTEVVVK | |
| Rabbit 11041 VL- | gccgtcgtgctgacccagactgcatcccccgtgtctgcacctgtggga | 96 |
| region | ggcacagtcaccatcaagtgccaggccagtgaggacatttacaccaat | |
| | ttagcctggtatcaacagaaaccaggacagcctcccaagctcctgatc | |
| | tactgggcatccactctggcatctggggtcccatcgcggttcaaaggc | |
| | agtggatctgggacagagttcactctcaccatcagcgacctggagtgt | |
| | gccgatgctgccacttactactgtcaagcctgtgtttatggcaatagt | |
| | gctgatagtcggtatactttcggcggagggaccgaggtggtggtcaaa | |
| Rabbit 11041 VH- | QSVEESGGRLVTPGTPLTLTCTVSGFSLSSYAMIWVRQAPGEGLEWIG | 97 |
| region | IIDIDGSTYYASWAKGRFTISRTSTTVDLKITSPTTGDTATYFCARDR | |
| | FVGVDIFDPWGPGTLVTVSS | |
| Rabbit 11041 VH- | cagtcggtggaggagtccgggggtcgcctggtcacgcctgggacaccc | 98 |
| region | ctgacactcacctgcaccgtctctggattctccctcagtagctatgca | |
| | atgatctgggtccgccaggctccaggggaggggctggaatggatcgga | |
| | atcattgatattgatgggagcacatactacgcgagctgggcgaaaggc | |
| | cgattcaccatctccagaacctcgaccacggtggatctgaaaatcacc | |
| | agtccgacaaccggggacacggccacctatttctgtgccagagatcgt | |
| | tttgttggtgttgatatttttgatccctggggcccaggcaccctggtc | |
| | accgtctcgagc | |
| 11041gL1 V-region | AVVLTQSPSSLSASVGDRVTITCQASEDIYTNLAWYQQKPGKAPKLLI | 99 |
| | YWASTLASGVPSRFSGSGSGTDFTLTISSLQPEDFATYYCQACVYGNS | |
| | ADSRYTFGGGTKVEIK | |
| 11041 gL1 C91S V- | AVVLTQSPSSLSASVGDRVTITCQASEDIYTNLAWYQQKPGKAPKLLI | 100 |
| region (gL2) | YWASTLASGVPSRFSGSGSGTDFTLTISSLQPEDFATYYCQASVYGNS | |
| | ADSRYTFGGGTKVEIK | |
| 11041 gL1 C91V V- | AVVLTQSPSSLSASVGDRVTITCQASEDIYTNLAWYQQKPGKAPKLLI | 101 |
| region (gL3) | YWASTLASGVPSRFSGSGSGTDFTLTISSLQPEDFATYYCQAVVYGNS | |
| | ADSRYTFGGGTKVEIK | |
| 11041gL6 V-region | AVQLTQSPSSLSASVGDRVTITCQASEDIYTNLAWYQQKPGKAPKLLI | 102 |
| | YWASTLASGVPSRFSGSGSGTDFTLTISSLQPEDFATYYCQACVYGNS | |
| | ADSRYTFGGGTKVEIK | |
| 11041gL7 V-region | AIQLTQSPSSLSASVGDRVTITCQASEDIYTNLAWYQQKPGKAPKLLI | 103 |
| | YWASTLASGVPSRFSGSGSGTDFTLTISSLQPEDFATYYCQACVYGNS | |
| | ADSRYTFGGGTKVEIK | |
| 11041 gL1 N95D V- | AVVLTQSPSSLSASVGDRVTITCQASEDIYTNLAWYQQKPGKAPKLLI | 104 |
| region (gL8) | YWASTLASGVPSRFSGSGSGTDFTLTISSLQPEDFATYYCQACVYGDS | |
| | ADSRYTFGGGTKVEIK | |
| 11041 gL1 S96A V- | AVVLTQSPSSLSASVGDRVTITCQASEDIYTNLAWYQQKPGKAPKLLI | 105 |
| region (gL9) | YWASTLASGVPSRFSGSGSGTDFTLTISSLQPEDFATYYCQACVYGNA | |
| | ADSRYTFGGGTKVEIK | |
| 11041 gL1 C91S | AVVLTQSPSSLSASVGDRVTITCQASEDIYTNLAWYQQKPGKAPKLLI | 106 |
| S96A V-region | YWASTLASGVPSRFSGSGSGTDFTLTISSLQPEDFATYYCQASVYGNA | |
| (gL10) | ADSRYTFGGGTKVEIK | |
| 11041 gL6 C91S V- | AVQLTQSPSSLSASVGDRVTITCQASEDIYTNLAWYQQKPGKAPKLLI | 107 |
| region (gL11) | YWASTLASGVPSRFSGSGSGTDFTLTISSLQPEDFATYYCQASVYGNS | |
| | ADSRYTFGGGTKVEIK | |
| 11041 gL7 C91S V- | AIQLTQSPSSLSASVGDRVTITCQASEDIYTNLAWYQQKPGKAPKLLI | 108 |
| region (gL12) | YWASTLASGVPSRFSGSGSGTDFTLTISSLQPEDFATYYCQASVYGNS | |
| | ADSRYTFGGGTKVEIK | |
| 11041 gL7 C91S | AIQLTQSPSSLSASVGDRVTITCQASEDIYTNLAWYQQKPGKAPKLLI | 109 |
| S96A V-region | YWASTLASGVPSRFSGSGSGTDFTLTISSLQPEDFATYYCQASVYGNA | |
| (gL14) | ADSRYTFGGGTKVEIK | |
| 11041gH1 V-region | EVQLVESGGGLVQPGGSLRLSCAVSGESLSSYAMIWVRQAPGKGLEWI | 110 |
| | GIIDIDGSTYYASWAKGRFTISRDSSKNTVYLQMNSLRAEDTAVYYCA | |
| | RDRFVGVDIFDPWGQGTLVTVSS | |
| 11041 gH1 G55A V- | EVQLVESGGGLVQPGGSLRLSCAVSGFSLSSYAMIWVRQAPGKGLEWI | 111 |
| region (gH2) | GIIDIDASTYYASWAKGRFTISRDSSKNTVYLQMNSLRAEDTAVYYCA | |
| | RDRFVGVDIFDPWGQGTLVTVSS | |
| 11041 gH1 D54E V- | EVQLVESGGGLVQPGGSLRLSCAVSGESLSSYAMIWVRQAPGKGLEWI | 112 |
| region (gH3) | GIIDIEGSTYYASWAKGRFTISRDSSKNTVYLQMNSLRAEDTAVYYCA | |
| | RDRFVGVDIFDPWGQGTLVTVSS | |
| 11041 gH1 D107E V- | EVQLVESGGGLVQPGGSLRLSCAVSGFSLSSYAMIWVRQAPGKGLEWI | 113 |
| region (gH4) | GIIDIDGSTYYASWAKGRFTISRDSSKNTVYLQMNSLRAEDTAVYYCA | |
| | RDRFVGVDIFEPWGQGTLVTVSS | |
| 11041gH5 V-region | EVQLVESGGGLVQPGGSLRLSCAVSGFSLSSYAMIWVRQAPGKGLEWI | 114 |
| | GIIDIDGSTYYASWAKGRFTISRDNSKNTVYLQMNSLRAEDTAVYYCA | |
| | RDRFVGVDIFDPWGQGTLVTVSS | |
| 11041gH8 V-region | EVQLVESGGGLVQPGGSLRLSCAVSGFSLSSYAMIWVRQAPGKGLEWI | 115 |
| | GIIDIDGSTYYASWAKGRFTISRDSSKNTLYLQMNSLRAEDTAVYYCA | |
| | RDRFVGVDIFDPWGQGTLVTVSS | |
| 11041gH9 V-region | EVQLVESGGGLVQPGGSLRLSCAASGFSLSSYAMIWVRQAPGKGLEWI | 116 |
| | GIIDIDGSTYYASWAKGRFTISRDSSKNTVYLQMNSLRAEDTAVYYCA | |
| | RDRFVGVDIFDPWGQGTLVTVSS | |
| 11041gH11 V-region | EVQLVESGGGLVQPGGSLRLSCAVSGFSLSSYAMIWVRQAPGKGLEWV | 117 |
| | GIIDIDGSTYYASWAKGRFTISRDNSKNTLYLQMNSLRAEDTAVYYCA | |
| | RDRFVGVDIFDPWGQGTLVTVSS | |
| 11041gH12 V-region | EVQLVESGGGLVQPGGSLRLSCAVSGFSLSSYAMIWVRQAPGKGLEWI | 118 |
| | SIIDIDGSTYYASWAKGRFTISRDNSKNTLYLQMNSLRAEDTAVYYCA | |
| | RDRFVGVDIFDPWGQGTLVTVSS | |
| 11041 gH8 D54E V- | EVQLVESGGGLVQPGGSLRLSCAVSGFSLSSYAMIWVRQAPGKGLEWI | 119 |
| region (gH15) | GIIDIEGSTYYASWAKGRFTISRDSSKNTLYLQMNSLRAEDTAVYYCA | |
| | RDRFVGVDIFDPWGQGTLVTVSS | |
| 11041 gH11 D54E V- | EVQLVESGGGLVQPGGSLRLSCAVSGFSLSSYAMIWVRQAPGKGLEWV | 120 |
| region (gH17) | GIIDIEGSTYYASWAKGRFTISRDNSKNTLYLQMNSLRAEDTAVYYCA | |
| | RDRFVGVDIFDPWGQGTLVTVSS | |
| 11041 gH12 D54E V- | EVQLVESGGGLVQPGGSLRLSCAVSGFSLSSYAMIWVRQAPGKGLEWI | 121 |
| region (gH18) | SIIDIEGSTYYASWAKGRFTISRDNSKNTLYLQMNSLRAEDTAVYYCA | |
| | RDRFVGVDIFDPWGQGTLVTVSS | |
| 11041 gL6 C91S | Same as SEQ ID NO 18 | 18 |
| S96A (gL13) light | | |
| chain | | |
| 11041 gH5 D54E | Same as SEQ ID NO 20 | 20 |
| (gH14) heavy chain | | |

TABLE 14
Other 11070 sequences (IL22-binding domain)

| Name | Sequence | SEQ ID NO |
|---|---|---|
| 11070 CDRH2 (not | RMWSDGDTSYNSAFTS | 122 |
| mutated) | | |
| 11070 CDRH2 S61T | Same as SEQ ID NO 74 | 74 |
| Rat Ab 11070 VL- | DIVMTQTPSNLAASPGESVSINCKASKTISKYLAWYQQKPGKANKLLI | 123 |
| region | YSGSTLQSGTPSRFSGSGSSTDFTLTIRNLEPEDFGLYYCQQHNEYPL | |
| | TFGSGTKLEIK | |
| Rat Ab 11070 VL- | gatattgtgatgacacagactccatctaatcttgctgcctctcctgga | 124 |
| region | gaaagtgtttccatcaattgcaaggcaagtaagaccattagcaagtat | |
| | ttagcctggtatcaacagaaacctgggaaagcaaataagcttcttatc | |
| | tattctgggtcaactttgcaatctggaactccatcgaggttcagtggc | |
| | agtggatctagtacagatttcactctcaccatcagaaacctggagcct | |
| | gaagattttggactctattactgtcaacagcataatgaatacccgctc | |
| | acgttcggttctgggaccaagttggaaataaaa | |
| Rat Ab 11070 VH- | EVQLQESGPGLVQPSQTLSPTCTVSGFSLTSYSVHWVRQHSGKSLEWM | 125 |
| region | GRMWSDGDTSYNSAFTSRLSITRDTSKSQVFLKMNSLQTEDTGTYYCA | |
| | RSLDFYYDTTLAFWGPGTTVTVSS | |
| Rat Ab 11070 VH- | gaggtgcagctgcaggagtcaggacctgggctggtgcagccctcacag | 126 |
| region | accctgtcccccacctgcactgtctctgggttctcactaactagttac | |
| | agtgtacactgggttcgccagcattcaggaaagagtctggaatggatg | |
| | ggaagaatgtggagtgatggagacacatcatataattcagcgttcaca | |
| | tcccgattgagcatcactagggacacctccaagagccaagttttctta | |
| | aaaatgaacagtctgcaaactgaagacacaggcacttactactgtgcc | |
| | agaagtctcgatttttactatgatactactcttgccttctggggccca | |
| | ggaaccacggtcaccgtctcgagt | |
| 11070gL1 V-region | DIVMTQSPSSVSASVGDRVTITCKASKTISKYLAWYQQKPGKANKLLI | 127 |
| | YSGSTLQSGTPSRFSGSGSSTDFTLTISSLQPEDFATYYCQQHNEYPL | |
| | TFGQGTKLEIK | |
| 11070gH1 V-region | EVQLQESGPGLVKPSQTLSLTCTVSGFSLTSYSVHWVRQHSGKGLEWM | 128 |
| | GRMWSDGDTSYNSAFTSRLTISRDTSKSQVSLKLSSVTAADTAVYYCA | |
| | RSLDFYYDTTLAFWGQGTTVTVSS | |
| 11070gH13 V-region | EVQLQESGPGLVKPSQTLSLTCTVSGFSLTSYSVHWVRQHSGKGLEWM | 129 |
| (gH1 S61T) | GRMWSDGDTSYNTAFTSRLTISRDTSKSQVSLKLSSVTAADTAVYYCA | |
| | RSLDFYYDTTLAFWGQGTTVTVSS | |

TABLE 15
Other IL13-binding domain sequences

| Name | Sequence | SEQ ID NO |
|---|---|---|
| Rat Ab 650 (1539) | DIQMTQSPPVLSASVGDRVTLSCKASQNINENLDWYHQKHGEAPKLLI | 130 |
| VL-region | YYTDILQTGIPSRESGSGSGTDYTLTISSLQPEDVATYYCYQYYSGYT | |
| | FGPGTKLEIK | |
| Rat Ab 650 (1539) | gacatccagatgacccagtctcctccagtcctgtctgcatctgtggga | 131 |
| VL-region | gacagagtcactctcagttgcaaagcaagtcagaatattaatgagaac | |
| | ttagactggtatcatcaaaagcatggcgaagctccaaaactcctgata | |
| | tattatacagacattttgcaaacgggcatcccatcaaggttcagtggc | |
| | agtggatctggtacagattacacactcaccatcagcagcctgcagcct | |
| | gaagatgttgccacatattactgctatcagtattacagtgggtacacg | |
| | tttggacctgggaccaagctggaaataaaa | |
| Rat Ab 650 (1539) | QVQLQQSGAELVKPGSSVKMSCKASGYSFTSYYIHWIKQRPGQGLEWI | 132 |
| VH-region | GRIGPGSGDINYNEKFKGKATFTVDKYFSTAYMQLSSLSPEDTAVFYC | |
| | ARFHYDGADWGQGTLVTVSS | |
| Rat Ab 650 (1539) | caggtacaactgcagcagtctggagctgagttggtgaagcctgggtct | 133 |
| VH-region | tcagtgaagatgtcctgcaaggcttctggctacagtttcaccagctac | |
| | tacatacactggataaagcagaggcctggacagggccttgagtggatt | |
| | gggcgtattggtcctggaagtggagatattaattacaatgagaagttc | |
| | aagggcaaggccacatttactgtggacaaatatttcagcacagcctac | |
| | atgcaactcagcagcctgtcacctgaggacactgcggtcttttactgt | |
| | gcaagatttcactatgatggggctgactggggccaaggcactctggtc | |
| | acagtctcgagc | |

TABLE 16
Human acceptor frameworks used for IL22-binding domains

| Name | Sequence | SEQ ID NO |
|---|---|---|
| Human IGKV1D-13 | AIQLTQSPSSLSASVGDRVTITCRASQGISSALAWYQQKPGKAPKLLI | 134 |
| IGKJ4 acceptor | YDASSLESGVPSRFSGSGSGTDFTLTISSLQPEDFATYYCQQFNSYPL | |
| framework | TFGGGTKVEIK | |
| Human IGKV1D-13 | gccatccagttgacccagtctccatcctccctgtctgcatctgtagga | 135 |
| IGKJ4 acceptor | gacagagtcaccatcacttgccgggcaagtcagggcattagcagtgct | |
| framework | ttagcctggtatcagcagaaaccagggaaagctcctaagctcctgatc | |
| | tatgatgcctccagtttggaaagtggggtcccatcaaggttcagcggc | |
| | agtggatctgggacagatttcactctcaccatcagcagcctgcagcct | |
| | gaagattttgcaacttattactgtcaacagtttaatagttaccctctc | |
| | actttcggcggagggaccaaggtggagatcaaa | |
| Human IGHV3-66 | EVQLVESGGGLVQPGGSLRLSCAASGFTVSSNYMSWVRQAPGKGLEWV | 136 |
| IGHJ4 acceptor | SVIYSGGSTYYADSVKGRFTISRDNSKNTLYLQMNSLRAEDTAVYYCA | |
| framework | RYFDYWGQGTLVTVSS | |
| Human IGHV3-66 | gaggtgcagctggtggagtctgggggaggcttggtccagcctgggggg | 137 |
| IGHJ4 acceptor | tccctgagactctcctgtgcagcctctggattcaccgtcagtagcaac | |
| framework | tacatgagctgggtccgccaggctccagggaaggggctggagtgggtc | |
| | tcagttatttatagcggtggtagcacatactacgcagactccgtgaag | |
| | ggcagattcaccatctccagagacaattccaagaacacgctgtatctt | |
| | caaatgaacagcctgagagccgaggacacggctgtgtattactgtgcg | |
| | agatactttgactactggggccaaggaaccctggtcaccgtctcctca | |
| Human IGKV1-12 | DIQMTQSPSSVSASVGDRVTITCRASQGISSWLAWYQQKPGKAPKLLI | 138 |
| IGKJ2 acceptor | YAASSLQSGVPSRFSGSGSGTDFTLTISSLQPEDFATYYCQQANSFPY | |
| framework | TFGQGTKLEIK | |
| Human IGKV1-12 | gacatccagatgacccagtctccatcttccgtgtctgcatctgtagga | 139 |
| IGKJ2 acceptor | gacagagtcaccatcacttgtcgggcgagtcagggtattagcagctgg | |
| framework | ttagcctggtatcagcagaaaccagggaaagcccctaagctcctgatc | |
| | tatgctgcatccagtttgcaaagtggggtcccatcaaggttcagcggc | |
| | agtggatctgggacagatttcactctcaccatcagcagcctgcagcct | |
| | gaagattttgcaacttactattgtcaacaggctaacagtttcccttac | |
| | acttttggccaggggaccaagctggagatcaaa | |
| Human IGHV4-31 | QVQLQESGPGLVKPSQTLSLTCTVSGGSISSGGYYWSWIRQHPGKGLE | 140 |
| IGHJ6 acceptor | WIGYIYYSGSTYYNPSLKSRVTISVDTSKNQFSLKLSSVTAADTAVYY | |
| framework | CARYYYYYGMDVWGQGTTVTVSS | |
| Human IGHV4-31 | caggtgcagctgcaggagtcgggcccaggactggtgaagccttcacag | 141 |
| IGHJ6 acceptor | accctgtccctcacctgcactgtctctggtggctccatcagcagtggt | |
| framework | ggttactactggagctggatccgccagcacccagggaagggcctggag | |
| | tggattgggtacatctattacagtgggagcacctactacaacccgtcc | |
| | ctcaagagtcgagttaccatatcagtagacacgtctaagaaccagttc | |
| | tccctgaagctgagctctgtgactgccgcggacacggccgtgtattac | |
| | tgtgcgagatactactactactacggtatggacgtctgggggcaaggg | |
| | accacggtcaccgtctcctca | |

TABLE 17
Sequences of IL/IL22 bi-specific KiH molecules

| Name | Sequence | SEQ ID NO |
|---|---|---|
| IL22 knob Light | AVQLTQSPSSLSASVGDRVTITCQASEDIYTNLAWYQQKPGKAPKLLI | 142 |
| chain | YWASTLASGVPSRFSGSGSGTDFTLTISSLQPEDFATYYCQASVYGNA | |
| | ADSRYTFGGGTKVEIKRTVAAPSVFIFPPSDEQLKSGTASVVCLLNNE | |
| | YPREAKVQWKVDNALQSGNSQESVTEQDSKDSTYSLSSTLTLSKADYE | |
| | KHKVYACEVTHQGLSSPVTKSENRGEC | |
| IL22 knob Light | gcagtgcagctgactcagtccccgtcctccctgtcggcctcagtggga | 143 |
| chain | gatcgcgtgaccattacctgtcaagccagcgaagatatctacaccaac | |
| | ctcgcctggtaccagcagaaacccgggaaggctccgaagctgctcatc | |
| | tattgggccagcaccttggcgtctggcgtgccatcccggttttccggt | |
| | tcgggaagcggaaccgacttcacgcttaccatttcctccctgcaacct | |
| | gaggacttcgccacttactactgccaagcctccgtctacgggaacgcc | |
| | gcggactcaagatacactttcggcggcggaaccaaggtcgaaatcaag | |
| | cgtacggtagcggccccatctgtcttcatcttcccgccatctgatgag | |
| | cagttgaaatctggaactgcctctgttgtgtgcctgctgaataacttc | |
| | tatcccagagaggccaaagtacagtggaaggtggataacgccctccaa | |
| | tcgggtaactcccaggagagtgtcacagagcaggacagcaaggacagc | |
| | acctacagcctcagcagcaccctgacgctgagcaaagcagactacgag | |
| | aaacacaaagtctacgcctgcgaagtcacccatcagggcctgagctcg | |
| | cccgtcacaaagagcttcaacaggggagagtgt | |
| IL22 knob Heavy | EVQLVESGGGLVQPGGSLRLSCAVSGESLSSYAMIWVRQAPGKGLEWI | 144 |
| chain | GIIDIEGSTYYASWAKGRFTISRDNSKNTVYLQMNSLRAEDTAVYYCA | |
| | RDRFVGVDIFDPWGQGTLVTVSSASTKGPSVFPLAPCSRSTSESTAAL | |
| | GCLVKDYFPEPVTVSWNSGALTSGVHTFPAVLQSSGLYSLSSVVTVPS | |
| | SSLGTKTYTCNVDHKPSNTKVDKRVESKYGPPCPPCPAPEFLGGPSVF | |
| | LFPPKPKDTLMISRTPEVTCVVVDVSQEDPEVQFNWYVDGVEVHNAKT | |
| | KPREEQFNSTYRVVSVLTVLHQDWLNGKEYKCKVSNKGLPSSIEKTIS | |
| | KAKGQPREPQVYTLPPSQEEMTKNQVSLWCLVKGFYPSDIAVEWESNG | |
| | QPENNYKTTPPVLDSDGSFFLYSRLTVDKSRWQEGNVFSCSVMHEALH | |
| | NHYTQKSLSLSLGK | |
| IL22 knob Heavy | gaagtgcagctcgtggagtcggggggaggactggtgcagcccggaggt | 145 |
| chain | tccctgcgcttgagctgtgcagtgtcaggcttttccctgtcctcctac | |
| | gccatgatctgggtccgccaagctcctggaaaggggctggaatggatc | |
| | ggaatcatcgacatcgagggctccacctactacgcctcatgggccaag | |
| | ggccggttcaccatttcccgggataacagcaagaacactgtgtacctc | |
| | cagatgaactcgctgagggccgaggacactgccgtgtattactgcgcg | |
| | cgggacagattcgtcggggtggacattttcgacccgtggggtcaaggc | |
| | acccttgtgaccgtctcgagcgcttctacaaagggcccatccgtcttc | |
| | cccctggcgccctgctccaggagcacctccgagagcacagccgccctg | |
| | ggctgcctggtcaaggactacttccccgaaccggtgacggtgtcgtgg | |
| | aactcaggcgccctgaccagcggcgtgcacaccttcccggctgtccta | |
| | cagtcctcaggactctactccctcagcagcgtggtgaccgtgccctcc | |
| | agcagcttgggcacgaagacctacacctgcaacgtagatcacaagccc | |
| | agcaacaccaaggtggacaagagagttgagtccaaatatggtccccca | |
| | tgcccaccatgcccagcacctgagttcctggggggaccatcagtcttc | |
| | ctgttccccccaaaacccaaggacactctcatgatctcccggacccct | |
| | gaggtcacgtgcgtggtggtggacgtgagccaggaagaccccgaggtc | |
| | cagttcaactggtacgtggatggcgtggaggtgcataatgccaagaca | |
| | aagccgcgggaggagcagttcaacagcacgtaccgtgtggtcagcgtc | |
| | ctcaccgtcctgcaccaggactggctgaacggcaaggagtacaagtgc | |
| | aaggtatccaacaaaggcctcccgtcctccatcgagaaaaccatctcc | |
| | aaagccaaagggcagccccgagagccacaggtgtacaccctgccccca | |
| | tcccaggaggagatgaccaagaaccaggtcagcctgtggtgcctggtc | |
| | aaaggcttctaccccagcgacatcgccgtggagtgggagagcaatggg | |
| | cagccggagaacaactacaagaccacgcctcccgtgctggactccgac | |
| | ggctccttcttcctctacagcaggctaaccgtggacaagagcaggtgg | |
| | caggaggggaatgtcttctcatgctccgtgatgcatgaggctctgcac | |
| | aaccactacacacagaagagcctctccctgtctctgggtaaa | |
| IL22 Hole light | Same as SEQ ID NO 142 | 142 |
| chain | | |
| IL22 Hole light | Same as SEQ ID NO 143 | 143 |
| chain | | |
| IL22 Hole heavy | EVQLVESGGGLVQPGGSLRLSCAVSGFSLSSYAMIWVRQAPGKGLEWI | 146 |
| chain | GIIDIEGSTYYASWAKGRFTISRDNSKNTVYLQMNSLRAEDTAVYYCA | |
| | RDRFVGVDIFDPWGQGTLVTVSSASTKGPSVFPLAPCSRSTSESTAAL | |
| | GCLVKDYFPEPVTVSWNSGALTSGVHTFPAVLQSSGLYSLSSVVTVPS | |
| | SSLGTKTYTCNVDHKPSNTKVDKRVESKYGPPCPPCPAPEFLGGPSVF | |
| | LFPPKPKDTLMISRTPEVTCVVVDVSQEDPEVQFNWYVDGVEVHNAKT | |
| | KPREEQFNSTYRVVSVLTVLHQDWLNGKEYKCKVSNKGLPSSIEKTIS | |
| | KAKGQPREPQVYTLPPSQEEMTKNQVSLSCAVKGFYPSDIAVEWESNG | |
| | QPENNYKTTPPVLDSDGSFFLVSRLTVDKSRWQEGNVFSCSVMHEALH | |
| | NHYTQKSLSLSLGK | |
| IL22 Hole heavy | gaagtgcagctcgtggagtcggggggaggactggtgcagcccggaggt | 147 |
| chain | tccctgcgcttgagctgtgcagtgtcaggcttttccctgtcctcctac | |
| | gccatgatctgggtccgccaagctcctggaaaggggctggaatggatc | |
| | ggaatcatcgacatcgagggctccacctactacgcctcatgggccaag | |
| | ggccggttcaccatttcccgggataacagcaagaacactgtgtacctc | |
| | cagatgaactcgctgagggccgaggacactgccgtgtattactgcgcg | |
| | cgggacagattcgtcggggtggacattttcgacccgtggggtcaaggc | |
| | acccttgtgaccgtctcgagcgcttctacaaagggcccatccgtcttc | |
| | cccctggcgccctgctccaggagcacctccgagagcacagccgccctg | |
| | ggctgcctggtcaaggactacttccccgaaccggtgacggtgtcgtgg | |
| | aactcaggcgccctgaccagcggcgtgcacaccttcccggctgtccta | |
| | cagtcctcaggactctactccctcagcagcgtggtgaccgtgccctcc | |
| | agcagcttgggcacgaagacctacacctgcaacgtagatcacaagccc | |
| | agcaacaccaaggtggacaagagagttgagtccaaatatggtccccca | |
| | tgcccaccatgcccagcacctgagttcctggggggaccatcagtcttc | |
| | ctgttccccccaaaacccaaggacactctcatgatctcccggacccct | |
| | gaggtcacgtgcgtggtggtggacgtgagccaggaagaccccgaggtc | |
| | cagttcaactggtacgtggatggcgtggaggtgcataatgccaagaca | |
| | aagccgcgggaggagcagttcaacagcacgtaccgtgtggtcagcgtc | |
| | ctcaccgtcctgcaccaggactggctgaacggcaaggagtacaagtgc | |
| | aaggtatccaacaaaggcctcccgtcctccatcgagaaaaccatctcc | |
| | aaagccaaagggcagccccgagagccacaggtgtacaccctgccccca | |
| | tcccaggaggagatgaccaagaaccaggtcagcctgagctgcgcggtc | |
| | aaaggcttctaccccagcgacatcgccgtggagtgggagagcaatggg | |
| | cagccggagaacaactacaagaccacgcctcccgtgctggactccgac | |
| | ggctccttcttcctcgtcagcaggctaaccgtggacaagagcaggtgg | |
| | caggaggggaatgtcttctcatgctccgtgatgcatgaggctctgcac | |
| | aaccactacacacagaagagcctctccctgtctctgggtaaa | |
| IL13 knob light | DIQMTQSPSSLSASVGDRVTITCKASQNINENLDWYQQKPGKAPKLLI | 148 |
| chain | YYTDILQTGIPSRFSGSGSGTDYTLTISSLQPEDFATYYCYQYYSGYT | |
| | FGQGTKLEIKRTVAAPSVFIFPPSDEQLKSGTASVVCLLNNFYPREAK | |
| | VQWKVDNALQSGNSQESVTEQDSKDSTYSLSSTLTLSKADYEKHKVYA | |
| | CEVTHQGLSSPVTKSENRGEC | |
| IL13 knob light | gacatccagatgacccagtccccctcctccctgtccgcctccgtgggc | 149 |
| chain | gacagggtgaccatcacctgcaaggcctcccagaacatcaacgagaac | |
| | ctggactggtaccagcagaagcccggcaaggcccccaagctgctgatc | |
| | tactacaccgacatcctgcagaccggcatcccctccaggttctccggc | |
| | tccggctccggcaccgactacaccctgaccatctcctccctgcagccc | |
| | gaggacttcgccacctactactgctaccagtactactccggctacacc | |
| | ttcggccagggcaccaagctggagatcaagcgtacggtagcggcccca | |
| | tctgtcttcatcttcccgccatctgatgagcagttgaaatctggaact | |
| | gcctctgttgtgtgcctgctgaataacttctatcccagagaggccaaa | |
| | gtacagtggaaggtggataacgccctccaatcgggtaactcccaggag | |
| | agtgtcacagagcaggacagcaaggacagcacctacagcctcagcagc | |
| | accctgacgctgagcaaagcagactacgagaaacacaaagtctacgcc | |
| | tgcgaagtcacccatcagggcctgagctcgcccgtcacaaagagcttc | |
| | aacaggggagagtgt | |
| IL13 knob heavy | EVQLVQSGAEVKKPGSSVKVSCKASGYSFTSYYIHWVRQAPGQGLEWM | 150 |
| chain | GRIGPGSGDINYNEKFKGRATFTVDKSTSTAYMELSSLRSEDTAVYYC | |
| | ARFHYDGADWGQGTLVTVSSASTKGPSVFPLAPCSRSTSESTAALGCL | |
| | VKDYFPEPVTVSWNSGALTSGVHTFPAVLQSSGLYSLSSVVTVPSSSL | |
| | GTKTYTCNVDHKPSNTKVDKRVESKYGPPCPPCPAPEFLGGPSVFLFP | |
| | PKPKDTLMISRTPEVTCVVVDVSQEDPEVQFNWYVDGVEVHNAKTKPR | |
| | EEQFNSTYRVVSVLTVLHQDWLNGKEYKCKVSNKGLPSSIEKTISKAK | |
| | GQPREPQVYTLPPSQEEMTKNQVSLWCLVKGFYPSDIAVEWESNGQPE | |
| | NNYKTTPPVLDSDGSFFLYSRLTVDKSRWQEGNVFSCSVMHEALHNHY | |
| | TQKSLSLSLGK | |
| IL13 knob heavy | gaggtgcagctggtgcagtccggcgccgaggtgaagaagcccggctcc | 151 |
| chain | tccgtgaaggtgtcctgcaaggcctccggctactccttcacctcctac | |
| | tacatccactgggtgaggcaggcccccggccagggcctggagtggatg | |
| | ggcaggatcggccccggctccggcgacatcaactacaacgagaagttc | |
| | aagggcagggccaccttcaccgtggacaagtccacctccaccgcctac | |
| | atggagctgtcctccctgaggtccgaggacaccgccgtgtactactgc | |
| | gccaggttccactacgacggcgccgactggggccagggcaccctggtg | |
| | accgtctcgagcgcttctacaaagggcccatccgtcttccccctggcg | |
| | ccctgctccaggagcacctccgagagcacagccgccctgggctgcctg | |
| | gtcaaggactacttccccgaaccggtgacggtgtcgtggaactcaggc | |
| | gccctgaccagcggcgtgcacaccttcccggctgtcctacagtcctca | |
| | ggactctactccctcagcagcgtggtgaccgtgccctccagcagcttg | |
| | ggcacgaagacctacacctgcaacgtagatcacaagcccagcaacacc | |
| | aaggtggacaagagagttgagtccaaatatggtcccccatgcccacca | |
| | tgcccagcacctgagttcctggggggaccatcagtcttcctgttcccc | |
| | ccaaaacccaaggacactctcatgatctcccggacccctgaggtcacg | |
| | tgcgtggtggtggacgtgagccaggaagaccccgaggtccagttcaac | |
| | tggtacgtggatggcgtggaggtgcataatgccaagacaaagccgcgg | |
| | gaggagcagttcaacagcacgtaccgtgtggtcagcgtcctcaccgtc | |
| | ctgcaccaggactggctgaacggcaaggagtacaagtgcaaggtatcc | |
| | aacaaaggcctcccgtcctccatcgagaaaaccatctccaaagccaaa | |
| | gggcagccccgagagccacaggtgtacaccctgcccccatcccaggag | |
| | gagatgaccaagaaccaggtcagcctgtggtgcctggtcaaaggcttc | |
| | taccccagcgacatcgccgtggagtgggagagcaatgggcagccggag | |
| | aacaactacaagaccacgcctcccgtgctggactccgacggctccttc | |
| | ttcctctacagcaggctaaccgtggacaagagcaggtggcaggagggg | |
| | aatgtcttctcatgctccgtgatgcatgaggctctgcacaaccactac | |
| | acacagaagagcctctccctgtctctgggtaaa | |
| IL13 Hole light | Same as SEQ ID NO 148 | 148 |
| chain | | |
| IL13 Hole light | Same as SEQ ID NO 149 | 149 |
| chain | | |
| IL13 Hole heavy | EVQLVQSGAEVKKPGSSVKVSCKASGYSFTSYYIHWVRQAPGQGLEWM | 152 |
| chain | GRIGPGSGDINYNEKFKGRATFTVDKSTSTAYMELSSLRSEDTAVYYC | |
| | ARFHYDGADWGQGTLVTVSSASTKGPSVFPLAPCSRSTSESTAALGCL | |
| | VKDYFPEPVTVSWNSGALTSGVHTFPAVLQSSGLYSLSSVVTVPSSSL | |
| | GTKTYTCNVDHKPSNTKVDKRVESKYGPPCPPCPAPEFLGGPSVFLFP | |
| | PKPKDTLMISRTPEVTCVVVDVSQEDPEVQFNWYVDGVEVHNAKTKPR | |
| | EEQFNSTYRVVSVLTVLHQDWLNGKEYKCKVSNKGLPSSIEKTISKAK | |
| | GQPREPQVYTLPPSQEEMTKNQVSLSCAVKGFYPSDIAVEWESNGQPE | |
| | NNYKTTPPVLDSDGSFFLVSRLTVDKSRWQEGNVFSCSVMHEALHNHY | |
| | TQKSLSLSLGK | |
| IL13 Hole heavy | gaggtgcagctggtgcagtccggcgccgaggtgaagaagcccggctcc | 153 |
| chain | tccgtgaaggtgtcctgcaaggcctccggctactccttcacctcctac | |
| | tacatccactgggtgaggcaggcccccggccagggcctggagtggatg | |
| | ggcaggatcggccccggctccggcgacatcaactacaacgagaagttc | |
| | aagggcagggccaccttcaccgtggacaagtccacctccaccgcctac | |
| | atggagctgtcctccctgaggtccgaggacaccgccgtgtactactgc | |
| | gccaggttccactacgacggcgccgactggggccagggcaccctggtg | |
| | accgtctcgagcgcttctacaaagggcccatccgtcttccccctggcg | |
| | ccctgctccaggagcacctccgagagcacagccgccctgggctgcctg | |
| | gtcaaggactacttccccgaaccggtgacggtgtcgtggaactcaggc | |
| | gccctgaccagcggcgtgcacaccttcccggctgtcctacagtcctca | |
| | ggactctactccctcagcagcgtggtgaccgtgccctccagcagcttg | |
| | ggcacgaagacctacacctgcaacgtagatcacaagcccagcaacacc | |
| | aaggtggacaagagagttgagtccaaatatggtcccccatgcccacca | |
| | tgcccagcacctgagttcctggggggaccatcagtcttcctgttcccc | |
| | ccaaaacccaaggacactctcatgatctcccggacccctgaggtcacg | |
| | tgcgtggtggtggacgtgagccaggaagaccccgaggtccagttcaac | |
| | tggtacgtggatggcgtggaggtgcataatgccaagacaaagccgcgg | |
| | gaggagcagttcaacagcacgtaccgtgtggtcagcgtcctcaccgtc | |
| | ctgcaccaggactggctgaacggcaaggagtacaagtgcaaggtatcc | |
| | aacaaaggcctcccgtcctccatcgagaaaaccatctccaaagccaaa | |
| | gggcagccccgagagccacaggtgtacaccctgcccccatcccaggag | |
| | gagatgaccaagaaccaggtcagcctgagctgcgcggtcaaaggcttc | |
| | taccccagcgacatcgccgtggagtgggagagcaatgggcagccggag | |
| | aacaactacaagaccacgcctcccgtgctggactccgacggctccttc | |
| | ttcctcgtcagcaggctaaccgtggacaagagcaggtggcaggagggg | |
| | aatgtcttctcatgctccgtgatgcatgaggctctgcacaaccactac | |
| | acacagaagagcctctccctgtctctgggtaaa | |

### EXAMPLES

### Example 1. Generation and Selection of Therapeutic Anti-IL13 Antibody CA650

Rats were immunized with either purified human IL13 (Peprotech) or rat fibroblasts expressing human IL13 (expressing approx 1 μg/ml in culture supernatant), or in some cases, a combination of the two. Following 3 to 6 shots, animals were sacrificed and PBMC, spleen, bone marrow and lymph nodes harvested. Sera was monitored for binding to human IL13 in ELISA and also for the ability to neutralize hIL13 in the HEK-293 IL13R-STAT-6 reporter cell assay (HEK-Blue assay, Invivogen).

B cell cultures were set up and supernatants were first screened for their ability to bind hIL13 in a bead-based assay in an Applied Biosystems FMAT assay. This was a homogeneous assay using biotinylated human IL13 coated onto streptavidin beads and a goat anti-rat Fc-Cy5 conjugate as a reveal agent. Positives from this assay were then progressed into a HEK-293 IL13R-STAT-6 reporter cell assay (HEK-Blue assay, Invivogen) to identify neutralisers. Neutralising supernatants were then profiled in the Biacore to estimate off-rate and also to characterize the mode of action of neutralization. Neutralization was categorized as either bin 1 or bin 2. Bin 1 represents an antibody that binds to human IL13 and prevents binding of IL13Rα1 and as a result also blocks IL-4R from binding. Bin 2 represents an antibody that binds human IL13 in such a way that allows binding to IL13Rα1 but prevents recruitment of IL-4R into the complex. Bin 1 antibodies were selected.

Approx. 7500 IL13-specific positives were identified in the primary FMAT screen from a total of 27×100-plate SLAM experiments. 800 wells demonstrated neutralization in the HEK-blue assay. 170 wells had desirable Biacore profiles, i.e. bin 1 antibodies with off-rates <5×10-4 s-1. Variable region cloning from these 170 wells was attempted and 160 successfully yielded fluorescent foci. 100 wells generated heavy and light chain variable region gene pairs following reverse transcription (RT)-PCR. These V-region genes were cloned as mouse IgG1 full-length antibodies and re-expressed in a HEK-293 transient expression system. Sequence analysis revealed that there were 27 unique families of anti-human IL13 antibody. These recombinant antibodies were then retested for their ability to block recombinant hIL13 (E. coli-derived and mammalian-derived), recombinant variant hIL13 (R130Q) (E. coli-derived), natural wild type and variant hIL13 (human donor-derived) and cynomolgus IL13 (mammalian-derived) in the cell-based assay. Recombinant antibodies were also tested for their ability to bind variant human IL13 (R130Q) and cynomolgus IL13 in the Biacore. Following this characterization, 5 antibody families fulfilled our criteria, i.e. sub-100 pM antibody with minimal drop-off in potency and affinity for all human and cynomolgus IL13 preparations.

Based on neutralization potency, affinity and donor content in humanized grafts (see below), humanized CA650 was selected for further progression.

### Example 2. Antibody CA650 Humanization

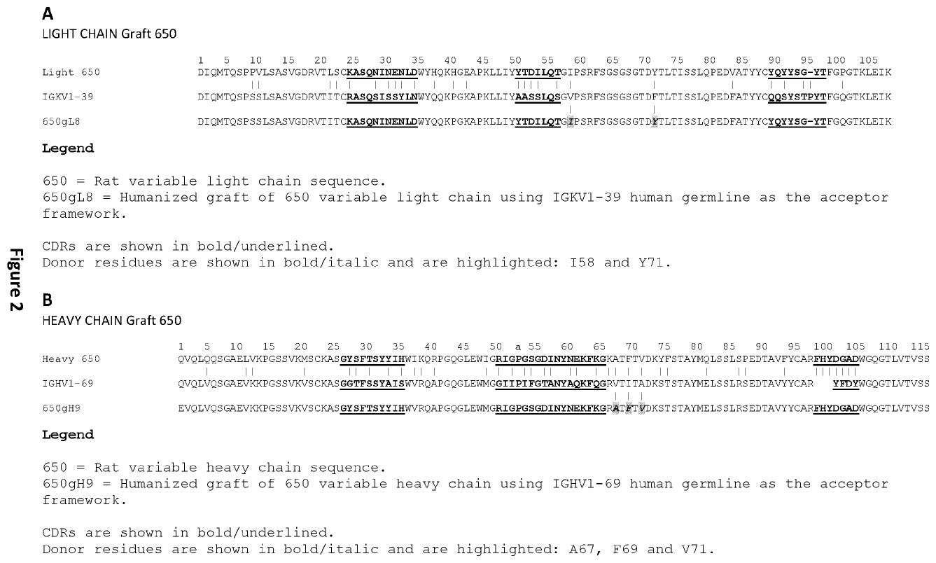

Antibody 650 was humanized by grafting the CDRs from the rat V-region onto human germline antibody V-region frameworks. In order to recover the activity of the antibody, a number of framework residues from the rat V-region were also retained in the humanized sequence. These residues were selected using the protocol outlined by Adair et al. (1991) (Humanized antibodies. WO91/09967). Alignments of the rat antibody (donor) V-region sequences with the human germline (acceptor) V-region sequences are shown in FIG. 2, together with the designed humanized sequences. (FIG. 2(A) light chain graft 650 and FIG. 2(B) heavy chain graft 650). The CDRs grafted from the donor to the acceptor sequence are as defined by Kabat (Kabat et al., 1987), with the exception of CDR-H1 where the combined Chothia/Kabat definition is used (see Adair et al., 1991 Humanized antibodies. WO91/09967).

Genes encoding initial V-region sequences were designed and constructed by an automated synthesis approach by Entelechon GmbH, and modified to generate the grafted versions gL8 and gH9 by oligonucleotide directed mutagenesis. The gL8 sequence was sub-cloned into the UCB Celltech human light chain expression vector pVhCK, which contains DNA encoding the human C-Kappa constant region (Km3 allotype). The gH9 sequence was sub-cloned into pVhg1Fab, which contains DNA encoding human heavy chain gamma-1 CH1 constant region.

Human V-region IGKV1-39 plus JK2 J-region (International Immunogenetics Information System® IMGT, http://www.imgt.org) was chosen as the acceptor for antibody 650 light chain CDRs. The light chain framework residues in graft gL8 are all from the human germline gene, with the exception of residues 58 and 71 (numbering according to Kabat), where the donor residues Isoleucine (158) and Tyrosine (Y71) were retained, respectively. Retention of residues I58 and Y71 was essential for full potency of the humanized antibody.

Human V-region IGHV1-69 plus JH4 J-region (IMGT, http://www.imgt.org) was chosen as the acceptor for the heavy chain CDRs of antibody 650. The heavy chain framework residues in grafts gH9 are all from the human germline gene, with the exception of residues 67, 69 and 71 (numbering according to Kabat, residues 68, 70 and 72 with reference to SEQ ID NO:29), where the donor residues Alanine (A67), Phenylalanine (F69) and Valine (V71) were retained, respectively. Retention of residues A67, F69 and V71 was essential for full potency of the humanized antibody. The Glutamine residue at position 1 of the human framework was replaced with Glutamic acid (E1) to afford the expression and purification of a homogeneous product: the conversion of Glutamine to pyroGlutamate at the N-terminus of antibodies and antibody fragments is widely reported. The final selected variable graft sequences gL8 and gH9 are shown in FIG. 2(A) and FIG. 2(B) respectively (1539gL8gH9).

The amino acid and DNA sequences encoding the CDRs, heavy and light variable regions, scFv and dsscFV formats of antibody 650 are shown in FIG. 2.

### Example 3. Generation of Anti-Human Albumin Antibody 645

The production of the anti-human albumin antibody 645 has been previously described in WO2013/068571. The amino acid and DNA sequences encoding the CDRs, heavy and light variable regions, scFv and dsscFV formats of antibody 645 are listed in Table 11.

### Example 4. Generation and Selection of Therapeutic Anti-IL22 Antibodies 11041 and 11070

A number of animals across different species (including mice, rats and rabbits) were immunized with either, purified in-house produced or commercially available human IL22 (R&D systems). Following 3-5 shots, the animals were sacrificed and PBMC, spleen, bone marrow and lymph nodes harvested. Sera was monitored for binding to human and cynomolgus IL22 in ELISA.

In case of 11041, memory B cell cultures were set up and supernatants were first screened for their ability to bind human and cynomolgus IL22 in a bead-based assay on the TTP Labtech Mirrorball system. This was a homogeneous multiplex assay using biotinylated human IL22 and biotinylated cynomolgus IL22 coated onto Sol-R streptavidin beads (TTP Labtech) and a goat anti-rabbit Fc-FITC conjugate as a reveal agent.

Approx. 4500 IL22-specific positive hits were identified in the primary Mirrorball screens from a total of 12×(164-400)-plate B culture experiments. Positive supernatants from this assay were then progressed for further characterization by:
ELISA, to confirm binding to human and cynomolgus monkey IL-22,
progression into an IL22 dependent HACAT phospho STAT-3 HTRF cell assay (CisBio) to identify neutralizers and,
profiling in BIAcore to estimate off-rate and to characterize the mode of action of neutralization.

Neutralization was categorized as either bin 1 or bin 2. Bin 1 represents an antibody that binds to human IL22 and prevents binding of IL22R1. Bin 2 represents an antibody that binds human IL22 but allows IL22R1 binding. Bin 1 antibodies were selected. Wells demonstrating neutralization in the phospho STAT-3 HTRF assay and/or wells with desirable BIAcore profiles were progressed for V region recovery using the fluorescent foci method.

Plasma cells from bone marrow were also directly screened for their ability to bind human IL22 using the fluorescent foci method (relevant for 11070). Here, B cells secreting IL22 specific antibodies were picked on biotinylated human IL22 immobilized on streptavidin beads using a goat anti-rat Fc-FITC conjugate reveal reagent. Approx. 300 direct foci were picked.

Following reverse transcription (RT) and PCR of the picked cells, ‘transcriptionally active PCR’ (TAP) products encoding the antibodies' V regions were generated and used to transiently transfect HEK-293 cells. The resultant TAP supernatants, containing recombinant antibody, were tested for their ability to; bind human and cynomolgus IL22, block IL22R1 binding in the BIAcore and neutralize IL22 in the HACAT phospho STAT-3 HTRF cell assay.

Heavy and light chain variable region gene pairs from interesting TAP products were then cloned as either rabbit or mouse Fab antibodies and re-expressed in a HEK-293 transient expression system. In total 131 V regions were cloned and sequenced. Recombinant cloned antibodies were then retested for their ability to bind human and cynomolgus IL22, block IL22R1 binding in the BIAcore and neutralize IL22 dependent IL-10 release in the COLO205 IL-10 HTRF cell-based assay (CisBio). Following this characterization, 2 antibodies fulfilled the criteria i.e. rabbit derived 11041 and rat derived 11070.

Based on neutralization potency, affinity to both human and cynomolgus IL22, donor content in humanized grafts (see below) and expression data, rabbit derived 11041 was selected for further progression.

### Example 5. Binding of Rabbit 11041 Fab to Human, Cynomolgus, and Mouse IL22

The affinity of purified 11041 rabbit Fab to human, cynomolgus and mouse IL22 was assessed using a Biacore T200 instrument (GE Healthcare) by capturing the rabbit 11041 Fab to an immobilized anti rabbit IgG F(ab′)2 followed by titration of IL22 from each species Affinipure Goat anti-Rabbit IgG-F(ab′)2 fragment specific (Jackson ImmunoResearch) was immobilized on a CM5 Sensor Chip via amine coupling chemistry to a capture level of 5000 response units (RUs). HBS-EP+ buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of 11041 Fab at 0.5 μg/mL was used for capture by the immobilized Goat Anti-Rabbit Fab. Human IL22, Cyno IL22 and mouse IL22 were titrated over the captured 11041 Fab (PB0006661) (at 0 nM, 0.6 nM, 1.8 nM, 5.5 nM, 16.6 nM and 50 nM) at a flow rate of 30 μL/min to assess affinity. Blocking of human IL22R1 was assessed by injecting 100 nM IL-22 (for 180 seconds at 30 μL/min) followed by injecting human IL22R1 (at 50 nM for 180s).

The surface was generated by 2×10 μL injection of 50 mM HCl, interspersed by a 10 μL injection of 5 mM NaOH at flowrate of 10 μL/min. Background subtraction binding curves were analyzed using the Biacore T200 evaluation software following standard procedures. Kinetic parameters were determined from the fitting algorithm. The kinetic parameters of purified 11041 binding to human, cynomolgus and mouse IL22 are shown in Table 18.

TABLE 18
Kinetic parameters of rabbit 11041 binding to human, cynomolgus and mouse IL22

| Human IL22 | | | Cynomolgus IL22 | | | Mouse IL22 | | |
|---|---|---|---|---|---|---|---|---|
| ka | | | ka | | | ka | | |
| (1/Ms) | kd (1/s) | KD (M) | (1/Ms) | kd (1/s) | KD (M) | (1/Ms) | kd (1/s) | KD (M) |
| 8.70E+05 | 2.90E−04 | 3.40E−10 | 2.60E+05 | 2.80E−04 | 1.10E−09 | 3.70E+07 | 1.10E−01 | 3.00E−09 |

### Example 6. Humanization of 11041

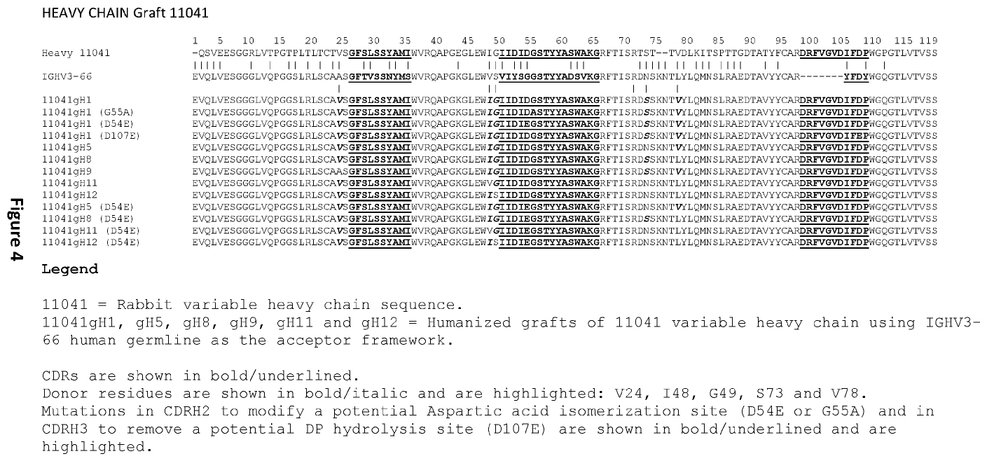

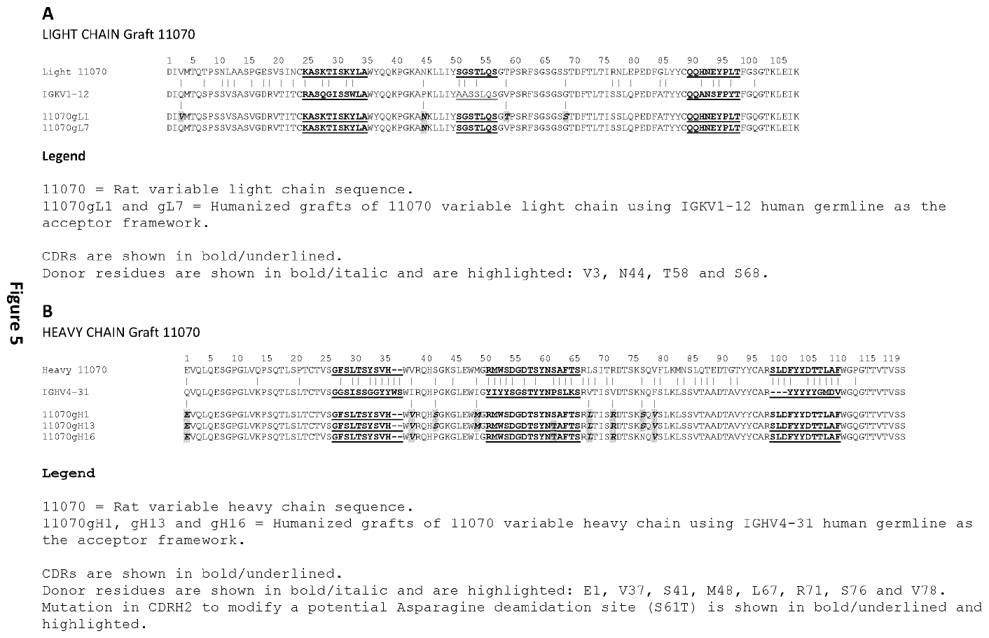

Antibody 11041 was humanized by grafting the CDRs from the rabbit V-region onto human germline antibody V-region frameworks. In order to recover the activity of the antibody, a number of framework residues from the rabbit V-region were also retained in the humanized sequence. These residues were selected using the protocol outlined by Adair et al. (1991) (WO91/09967). Alignments of the rabbit antibody (donor) V-region sequences with the human germline (acceptor) V-region sequences are shown in FIGS. 4 and 5, together with the designed humanized sequences. The CDRs grafted from the donor to the acceptor sequence are as defined by Kabat (Kabat et al., 1987), with the exception of CDR-H1 where the combined Chothia/Kabat definition is used (see Adair et al., WO91/09967).

Human V-region IGKV1D-13 plus IGKJ4 J-region (IMGT, http://www.imgt.org/) was chosen as the acceptor for antibody 11041 light chain CDRs. The light chain framework residues in the humanized graft variants are all from the human germline gene, with the exception of none, one or two residues from the group comprising residues 2 and 3 (with reference to SEQ ID NO:99 gL1), where the donor residues Valine (V2) and Valine (V3) were retained, respectively. In some humanized graft variants, an unpaired/free Cysteine residue at position 91 in CDRL3 was removed by mutation to either Valine (C91V) or Serine (C91S): free cysteine residues can be subject to post-translational modification, such as cysteinylation, and may contribute to covalent aggregation and poor stability. Mutation of this residue resulted in an unexpected 15- to 50-fold increase in binding affinity, respectively, as measured by surface plasmon resonance (Table 19, gL1gH1 (642 pM) compared to gL1 (C91V)gH1 (41.9 pM) or gL1(C91S)gH1 (12.4 pM)). In some humanized graft variants, a potential Asparagine deamidation site in CDRL3 was modified by either replacing the Asparagine residue at position 95 with Aspartic acid (N95D) or by replacing the Serine residue at position 96 with Alanine (S96A). Modification of the deamidation site by S96A mutation significantly reduced the basal level of deamidation.

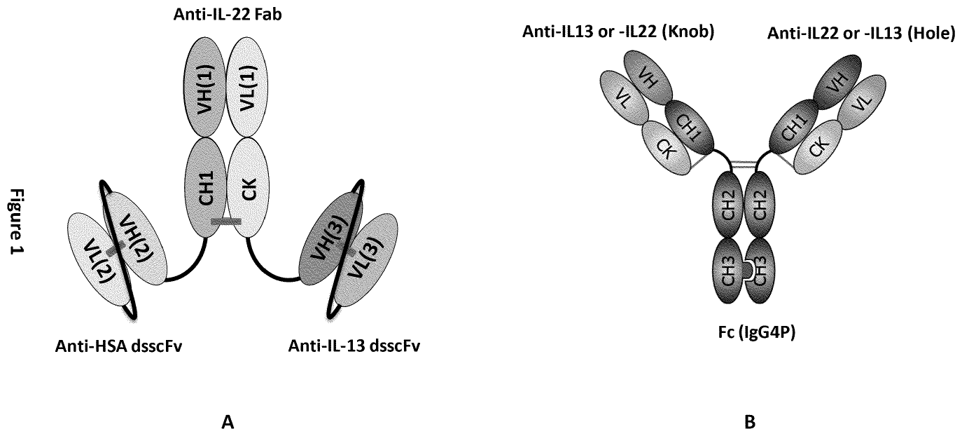

Human V-region IGHV3-66 plus IGHJ4 J-region (IMGT, http://www.imgt.org/) was chosen as the acceptor for the heavy chain CDRs of antibody 11041. In common with many rabbit antibodies, the VH gene of antibody 11041 is shorter than the selected human acceptor. When aligned with the human acceptor sequence, framework 1 of the VH region of antibody 11041 lacks the N-terminal residue, which is retained in the humanized antibody (FIG. 4). Framework 3 of the 11041 rabbit VH region also lacks two residues (75 and 76, with reference to SEQ ID NO:110 gH1) in the loop between beta sheet strands D and E: in the humanized graft variants the gap is filled with the corresponding residues (Lysine 75, K75; Asparagine 76, N76) from the selected human acceptor sequence (FIG. 1). The heavy chain framework residues in the humanized graft variants are all from the human germline gene, with the exception of the residues 24, 48, 49, 73 and 78 (with reference to SEQ ID NO: 110 gH1), where the donor residues Valine (V24), Isoleucine (I48), Glycine (G49), Serine (S73) and Valine (V78) were retained, respectively. Retention of donor residues V24, I48, G49 and V78 was essential for the highest affinity binding to human IL22, as measured by surface plasmon resonance. In some humanized graft variants, a potential Aspartic acid isomerization site in CDRH2 was modified by either replacing the Aspartic acid residue at position 54 with Glutamic acid (D54E), or by replacing the Glycine residue at position 55 with Alanine (G55A). In some humanized graft variants, a potential hydrolysis site in CDRH3 was modified by replacing the Aspartic acid residue at position 107 with glutamic acid (D107E).

TABLE 19
Binding affinity of different generated variants

| | Light | | | | |
|---|---|---|---|---|---|
| | chain | Light | | Heavy | |
| | Donor | chain | Heavy chain | chain | Affinity |
| Antibody variant | residues | Mutation | Donor residues | Mutation | (KD) pM |
| 11041 (parental) | — | | — | | 569 |
| 11041gL1gH1 | V2, V3 | | V24, I48, G49, | | 642 |
| | | | S73, V78 | | |
| 11041gL1 C91S gH1 | V2, V3 | C91S | V24, I48, G49, | | 12.4 |
| | | | S73, V78 | | |
| 11041gL1 C91V gH1 | V2, V3 | C91V | V24, I48, G49, | | 41.9 |
| | | | S73, V78 | | |
| 11041gL1 N95D gH1 | V2, V3 | N95D | V24, I48, G49, | | 128.6 |
| | | | S73, V78 | | |
| 11041gL1 S96A gH1 | V2, V3 | S96A | V24, I48, G49, | | 200 |
| | | | S73, V78 | | |
| 11041gL6gH1 | V2 | | V24, I48, G49, | | 369 |
| | | | S73, V78 | | |
| 11041gL7gH1 | — | | V24, I48, G49, | | 446 |
| | | | S73, V78 | | |
| 11041gL1gH1 G55A | V2, V3 | | V24, I48, G49, | G55A | 627 |
| | | | S73, V78 | | |
| 11041gL1gH1 D54E | V2, V3 | | V24, I48, G49, | D54E | 166 |
| | | | S73, V78 | | |
| 11041gL1gH1 D107E | V2, V3 | | V24, I48, G49, | D107E | 657 |
| | | | S73, V78 | | |
| 11041gL1gH5 | V2, V3 | | V24, I48, G49, | | 378 |
| | | | V78 | | |
| 11041gL1gH8 | V2, V3 | | V24, I48, G49, | | 274 |
| | | | S73 | | |
| 11041gL1 C91S gH1 D54E | V2, V3 | C91S | V24, I48, G49, | D54E | 28.1 |
| | | | S73, V78 | | |
| 11041gL1 S96A gH1 D54E | V2, V3 | | V24, I48, G49, | D54E | 88.8 |
| | | | S73, V78 | | |
| 11041gL1 C91S gH9 | V2, V3 | C91S | I48, G49, S73, | | 11.4 |
| | | | V78 | | |
| 11041gL1 C91S S96A gH5 D54E | V2, V3 | C91S, | V24, I48, G49, | D54E | 23.3 |
| | | S96A | V78 | | |
| 11041gL1 C91S S96AgH15 | V2, V3 | C91S, | V24, I48, G49, | D54E | 79 |
| | | S96A | S73 | | |
| 11041gL1 C91S S96AgH17 | V2, V3 | C91S, | V24, G49 | D54E | 39.8 |
| | | S96A | | | |
| 11041gL1 C91S S96AgH18 | V2, V3 | C91S, | V24, I48 | D54E | 45.4 |
| | | S96A | | | |
| 11041gL6 C91S gH5 D54E | V2 | C91S | V24, I48, G49, | D54E | 16.1 |
| | | | V78 | | |
| 11041gL6 C91S gH8 D54E | V2 | C91S | V24, I48, G49, | D54E | 105.8 |
| | | | S73 | | |
| 11041gL6 C91S gH11 D54E | V2 | C91S | V24, G49 | D54E | 51.6 |
| 11041gL6 C91S gH12 D54E | V2 | C91S | V24, I48 | D54E | 44.4 |
| 11041gL7 C91S gH5 D54E | — | C91S | V24, I48, G49, | D54E | 29.3 |
| | | | V78 | | |
| 11041gL7 C91S gH8 D54E | — | C91S | V24, I48, G49, | D54E | 114.6 |
| | | | S73 | | |
| 11041gL7 C91S gH11 D54E | — | C91S | V24, G49 | D54E | 66.6 |
| 11041gL7 C91S gH12 D54E | — | C91S | V24, I48 | D54E | 73.9 |
| 11041gL6 C91S S96A gH5 D54E | V2 | C91S, | V24, I48, G49, | D54E | 12.5 |
| | | S96A | V78 | | |
| 11041gL6 C91S S96A gH8 D54E | V2 | C91S, | V24, I48, G49, | D54E | 84.9 |
| | | S96A | S73 | | |
| 11041gL6 C91S S96A gH11 D54E | V2 | C91S, | V24, G49 | D54E | 52.4 |
| | | S96A | | | |
| 11041gL6 C91S S96A gH12 D54E | V2 | C91S, | V24, I48 | D54E | 51.7 |
| | | S96A | | | |
| 11041gL7 C91S S96A gH5 D54E | — | C91S, | V24, I48, G49, | D54E | 26.6 |
| | | S96A | V78 | | |
| 11041gL7 C91S S96A gH8 D54E | — | C91S, | V24, I48, G49, | D54E | 103.8 |
| | | S96A | S73 | | |
| 11041gL7 C91S S96A gH11 D54E | — | C91S, | V24, G49 | D54E | 61.7 |
| | | S96A | | | |
| 11041gL7 C91S S96A gH12 D54E | — | C91S, | V24, I48 | D54E | 67.6 |
| | | S96A | | | |

### Example 7. Humanization of 11070

Antibody 11070 was humanized by grafting the CDRs from the rat V-region onto human germline antibody V-region frameworks. In order to recover the activity of the antibody, a number of framework residues from the rat V-region were also retained in the humanized sequence. These residues were selected using the protocol outlined by Adair et al. (1991) (WO91/09967). Alignments of the rat antibody (donor) V-region sequences with the human germline (acceptor) V-region sequences are shown in FIGS. 5A and 5B, together with the designed humanized sequences. The CDRs grafted from the donor to the acceptor sequence are as defined by Kabat (Kabat et al., 1987), with the exception of CDR-H1 where the combined Chothia/Kabat definition is used (see Adair et al., WO91/09967).

Human V-region IGKV1-12 plus IGKJ2 J-region (IMGT, http://www.imgt.org/) was chosen as the acceptor for antibody 11070 light chain CDRs. The light chain framework residues in the humanized graft variants are all from the human germline gene, with the exception of one or more residues from the group comprising residues 3, 44, 58 and 68 (with reference to SEQ ID NO:127, gL1), where the donor residues Valine (V3), Asparagine (N44), Threonine (T58) and Serine (S68) were retained, respectively. Retention of donor residue N44 was essential for the highest affinity binding to human IL22, as measured by surface plasmon resonance (Table 20).

Human V-region IGHV4-31 plus IGHJ6 J-region (IMGT, http://www.imgt.org/) was chosen as the acceptor for the heavy chain CDRs of antibody 11070. The heavy chain framework residues in the humanized graft variants are all from the human germline gene, with the exception of one or more residues from the group comprising residues 37, 41, 48, 67, 71, 76 and 78 (with reference to SEQ ID NO: 128, gH1), where the donor residues Valine (V37), Serine (S41), Methionine (M48), Leucine (L67), Arginine (R71), Serine (S76) and Valine (V78) were retained, respectively. The Glutamine residue at position 1 of the human framework was replaced with Glutamic acid (E1) to afford the expression and purification of a homogeneous product: the conversion of Glutamine to pyroGlutamate at the N-terminus of antibodies and antibody fragments is widely reported. Retention of donor residues V37, L67, R71 and V78 was essential for the highest affinity binding to human IL-22, as measured by surface plasmon resonance (Table 20). In some humanized graft variants, a potential Asparagine deamidation site in CDRH2 was modified by replacing the Serine residue at position 61 with Threonine (S61T).

TABLE 20
Binding affinity of different generated variants of 11070 antibody

| Antibody 11070 | Light chain Donor | Heavy chain Donor | Heavy chain | Affinity (KD) |
|---|---|---|---|---|
| variant | residues | residues | Mutation | pM |
| 11070 | — | — | — | 49 |
| 11070gL1gH1 | V3, N44, T58, S68 | E1, V37, S41, M48, | — | 36.8 |
| | | L67, R71, S76, V78 | | |
| 11070gL1gH13 | V3, N44, T58, S68 | E1, V37, S41, M48, | S61T | 31.7 |
| | | L67, R71, S76, V78 | | |
| 11070gL7gH16 | N44 | E1, V37, L67, R71, | S61T | 25.7 |
| | | V78 | | |

### Example 8. Cloning and Production of Variants

Genes encoding different variants of heavy and light chain V-region sequences were designed and constructed by an automated synthesis approach by ATUM (Newark, CA). Further variants of heavy and light chain V-regions were created by modifying the VH and VK genes by oligonucleotide-directed mutagenesis, including, in some cases, mutations within CDRs. For transient expression in mammalian cells, the humanized light chain V-region genes were cloned into the UCB light chain expression vector pMhCK, which contains DNA encoding the human Kappa chain constant region (Km3 allotype). The humanized heavy chain V-region genes were cloned into the UCB human gamma-1 Fab heavy chain expression vector pMhFabnh, which contains DNA encoding the human gamma-1 CH1-hinge domain. Co-transfection of the resulting heavy and light chain vectors into Expi293™ suspension cells gave expression of the humanized, recombinant antibodies in the human Fab format. The variant humanized Fab antibodies were assessed for their binding affinity for human IL22 relative to the parent antibody, their potency in in vitro assays, their biophysical properties and suitability for downstream processing.

### Example 9. Binding Properties of Humanized 11041 Fab Antibody

Humanized samples for 11041 antibody were tested by capturing the samples on immobilized anti human IgG-F(ab′)₂ then titration of Human IL22 over the captured surface. The assay was run on Biacore 8K instrument (GE Healthcare) and BIA (Biomolecular Interaction Analysis) was performed using Biacore 8000 evaluation software Affinipure Goat anti-human IgG-F(ab′)2 fragment specific (Jackson ImmunoResearch) was immobilized on a CM5 Sensor Chip via amine coupling chemistry to a capture level of ˜5000 response units (RUs). HBS-EP+ buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of humanized samples of 11041 antibody at 0.5 μg/mL was used for capture by the immobilized Goat anti-human Fab IgG. Human IL22 (50 nM, 16.7 nM, 5.6 nM, 1.9 nM and 617 pM) and were titrated over the captured 11041 antibodies at a flow rate of 30 μL/min.

The surface was generated by 2×10 μL injections of 50 mM HCl, interspersed by a 5 μL injection of 5 mM NaOH at flowrate of 10 μL/min. Background subtraction binding curves were analyzed using the Insight evaluation software following standard procedures. Kinetic parameters were determined from the fitting algorithm. The IL22 affinity determined from a single experiment is shown in Table 21 and was shown to be less than 100 pM.

TABLE 21
Binding affinity between humanized 11041 Fab and IL22

| Sample | ka | kd | KD (M) | KD (pM) |
|---|---|---|---|---|
| 11041gL13gH14 Fab | 1.01E+06 | 1.26E−05 | 1.25E−11 | 12.48 |

Values determined from a single experiment

### Example 10. Assessment of Blocking of IL22BP Binding Site on IL22 by Humanized 11041 Antibody

Surface plasmon resonance (Biacore T200) was used to assess whether 11041gL13gH14 Fab (as a part of a bispecific antibody) or Fezakinumab are able to block the IL22BP binding site of IL22.

A goat anti-human IgG Fab specific antibody (Jackson ImmunoResearch) was immobilized on a CM5 Sensorchip via amine coupling chemistry to a level of approximately 6000 RU.

Each analysis cycle consisted of capture of 11041gL13gH14 Fab or Fezakinumab molecules to the anti Fab surface, injection of IL22 at 20 nM (prepared in house) followed by injection of IL22BP at 100 nM, with each injected for 180s at 30 μl/min. At the end of each cycle the surface was regenerated at a flowrate of 10 μL/min using a 60s injection of 50 mM HCl followed by a 30s injection of 5 mM NaOH and a final 60s injection of 50 mM HCl. Background binding and drift were subtracted using control cycles consisting of buffer capture, or buffer analyte injections.

TABLE 22
The IL22 and IL22BP binding responses

| | | IL22 Binding | IL22BP Binding |
|---|---|---|---|
| | Capture | at 20 nM | at 100 nM |
| Sample | (RU) | (RU) | (RU) |
| 11041gL13gH14 Fab | 280 | 40 | 0 |
| Fezakinumab | 202 | 37 | 59 |

When IL22 was bound to surface-captured 11041gL13gH14 Fab, IL22BP was unable to bind IL22. When IL22 was bound to surface-captured Fezakinumab IL22BP was still capable of binding IL22. In conclusion, 11041gL13gH14 Fab (as a part of a bispecific antibody) blocks the IL22BP binding site of IL22, while Fezakinumab does not.

### Example 11. Purification of IL22

A his-tagged version of IL22 was purified largely as described by Nagem et al [Nagem et al Structure. 2002 August; 10(8):1051-62.]. The BL21(DE3) E. coli strain was transformed by heat shock with an expression construct encoding His-tagged IL22.

The encoded protein sequence is:

(SEQ ID NO: 3)
MGSSHHHHHHSSGENLYFQGSQGGAAAPISSHCRLDKSNFQQPYITNRTF
MLAKEASLADNNTDVRLIGEKLFHGVSMSERCYLMKQVLNFTLEEVLFPQ
SDRFQPYMQEVVPFLARLSNRLSTCHIEGDDLHIQRNVQKLKDTVKKLGE
SGEIKAIGELDLLFMSLRNACI

IL22 protein sequence after TEV cleavage (see below):

(SEQ ID NO: 4)
GSQGGAAAPISSHCRLDKSNFQQPYITNRTFMLAKEASLADNNTDVRLIG
EKLFHGVSMSERCYLMKQVLNFTLEEVLFPQSDRFQPYMQEVVPFLARLS
NRLSTCHIEGDDLHIQRNVQKLKDTVKKLGESGEIKAIGELDLLFMSLRN
ACI

Cells were grown in the presence of 100 μg/ml ampicillin, and protein expression was induced by adding IPTG to a concentration of 1 mM when the cells reached an optical density (measured at 600 nM) of 1. After 4 hours, the cells were harvested by centrifugation. After cell lysis with a high-pressure cell homogenizer, the inclusion bodies containing IL22 were collected by high speed centrifugation. The inclusion bodies were washed with 50 mM Tris-HCl, 100 mM NaCl, 1 mM EDTA, 1 mM DTT, and 0.5% (w/v) DOC (pH 8) and then again with the same buffer without detergent. The washed inclusion bodies were solubilized overnight at 4° C. in a buffer containing 50 mM MES, 10 mM EDTA, 1 mM DTT and 8M urea. Insoluble material was separated by centrifugation and IL22 in the soluble fraction was refolded by dilution to 0.1 mg/ml in 100 mM Tris-HCl, 2 mM EDTA, 0.5M Arginine, 1 mM reduced glutathione and 0.1 mM oxidized glutathione, with a final pH of 8.0. After 72 hours of incubation at 4° C., the protein was concentrated, and purified by size-exclusion chromatography on a HiLoad 26/600 Superdex 75 pg column, equilibrated with 25 mM MES pH 5.4 and 150 mM NaCl. The protein was then frozen at −80° C. until further use.

The his-tag was removed by overnight incubation of the IL22 protein with TEV protease, at 4° C. After diluting the protein in PBS containing 25 mM imidazole, the cleaved protein was passed over a 5 ml HisTrap™ High Performance column (GE Healthcare) and collected in the flow-through.

### Example 12. HDX-MS of IL22 in the Presence of 11041gL13gH14 Fab and 11070gL7gH16 Fab

Hydrogen Deuterium Exchange Mass Spectrometry (HDX-MS) was used for epitope mapping of IL22 against 11041gL13gH14 Fab and 11070gL7gH16 Fab.

### Sample Preparation and Data Acquisition

For HDX-MS analysis, 30 μM of IL22 (prepared as described in Example 11) and 30 μM of IL22 complexed with 90 μM of either 11041gL13gH14 Fab or 11070gL7gH16 Fab were prepared and incubated for 1 hour at 4° C. 4 μl of IL22, IL22/11041gL13gH14 Fab or IL22/11070gL7gH16 Fab complexes were diluted into 57 μL of 10 mM phosphate in H₂O (pH 7.0), or into 10 mM phosphate in D₂O (pD 7.0) at 25° C. The deuterated samples were then incubated for 0.5, 2, 15 and 60 min at 25° C. After the reaction, all samples were quenched by mixing at 1:1 with a quench buffer (4 M Guanadine Hydrochloride, 250 mM Tris(2-carboxyethyl)phosphine hydrochloride (TCEP), 100 mM phosphate) at 1° C. The mixed solution was at a final pH 2.5. The mixture was immediately injected into the nanoAcquity HDX module (Waters Corp.) for peptic digest. Peptide digestion was then performed on-line using a Enzymatic online digestion column (Waters) in 0.2% formic acid in water at 20° C. and with a flow rate of 100 μL/min. All deuterated time points and un-deuterated controls were carried out in triplicate with blanks run between each data-point.

Peptide fragments were then trapped using an Acquity BEH C18 1.7 μM VANGUARD chilled pre-column for 3 min. Peptides were then eluted into a chilled Acquity UPLC BEH C18 1.7 μM 1.0×100 using the following gradient: 0 min, 5% B; 6 min, 35% B; 7 min, 40% B; 8 min, 95% B, 11 min, 5% B; 12 min, 95% B; 13 min, 5% B; 14 min, 95% B; 15 min, 5% B (A: 0.2% HCOOH in H2O, B: 0.2% HCOOH in acetonitrile. Peptide fragments were ionized by positive electrospray into a Synapt G2-Si mass spectrometer (Waters). Data acquisition was run in ToF-only mode over an m/z range of 50-2000 Th, using an MSe method (low collision energy, 4V; high collision energy: ramp from 18V to 40V). Glu-1-Fibrinopeptide B peptide was used for internal lock mass correction.

### HDX-MS Data Processing

MSE data from un-deuterated controls samples of IL22, IL22/11041gL13gH14 Fab or IL22/11070gL7gH16 Fab complexes were used for sequence identification using the Waters Protein Lynx Global Server 2.5.1 (PLGS). Peptide search was performed against a database of the IL22 sequence only, with precursor intensity threshold of 500 counts and 3 matched product ions required for assignment. Ion accounting files for the 3 control samples were combined into a peptide list imported into Dynamx v3.0 software.

Peptides were subjected to further filtering in DynamX. Filtering parameters used were a minimum and maximum peptide sequence length of 4 and 25, respectively, minimum intensity of 1000, minimum MS/MS products of 2, minimum products per amino acid of 0.2, and a maximum MH+error threshold of 10 ppm. DynamX v3.0 was used to quantify the isotopic envelopes resulting from deuterium uptake for each peptide at each time-point. Furthermore, all the spectra were examined and checked visually to ensure correct assignment of m/z peaks and only peptides with a high signal to noise ratios were used for HDX-MS analysis.

Following manual filtration in Dynamx, statistical analysis and filtration were performed using Deuteros (https://academic.oup.com/bioinformatics/article/35/17/3171/5288775) that uses statistical analysis published by Houde et al., 2011 (https://www.ncbi.nlm.nih.gov/pubmed/21491437). Deuteros generates a woods plot that displays peptide length, start and end residues, global coverage and a y-axis metric which is the absolute uptake (in Daltons). It is the difference in uptake in the presence of a ligand (bound) and the apo form. Woods plots first apply confidence filtering to all peptides in each timepoint. Peptides with differential deuteration outside of the selected confidence limits are non-significant and are shown in light grey. The significant peptides are shown as dark grey and black. An in-house algorithm was used to filter the results and identify an epitope. Data presented is after 0.5 minutes of deuteration incubation.

### Coverage Map of IL22

HDX analysis of IL22 with 11041gL13gH14 Fab and 11070gL7gH16 Fab was performed in a single experiment. A total of 91.3% coverage was obtained for the HDX-MS experiment from 47 peptides. The peptide redundancy following filtering and analysis was 3.48.

HDX-MS of IL22 in the Presence of 11041gL13gH14 Fab

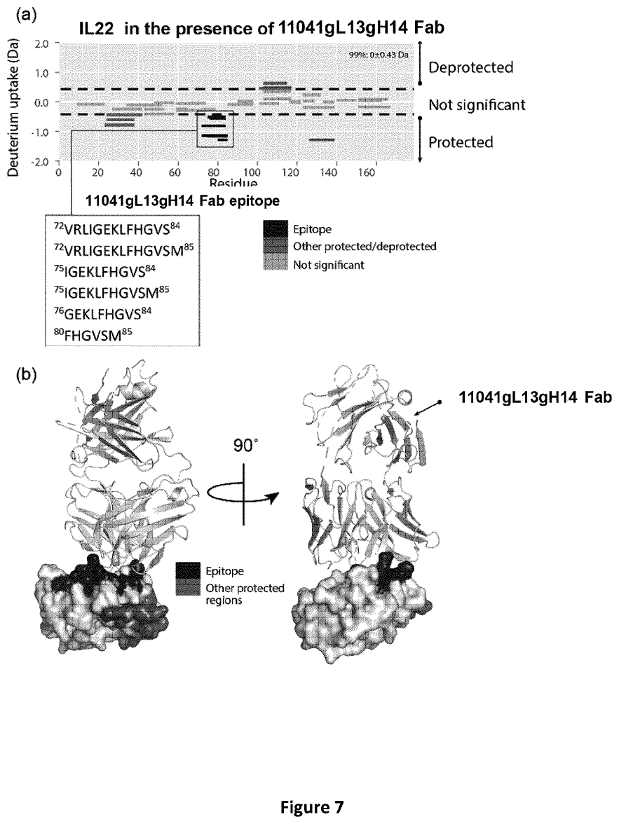

Seven peptides showing statistically significant reduced deuterium incorporation upon antibody binding (i.e. potential epitope) were observed, six of which agreed with the analysis (highlighted in black on the woods plot in FIG. 7, A): 72VRLIGEKLFHGVS84, 72VRLIGEKLFHGVSM85, 75IGEKLFHGVS84, 75IGEKLFHGVSM85, 76GEKLFHGVS84 and 80FHGVSM85. An increase in deuterium uptake (i.e. potential conformational change) was observed in three peptides: 101EEVLFPQSDRF111, 103VLFPQSDRFQPYM115 and 103VLFPQSDRFQPYMQE117. The 11041gL13gH14 Fab epitope was projected onto the structure of IL22 (FIG. 7, B). Other regions that were protected or deprotected upon antibody binding due to conformational change are highlighted in dark grey.

In conclusion, the protected region which represents the epitope region of 11041gL13gH14 Fab is residues 72-85 (VRLIGEKLFHGVSM).

HDX-MS of IL22 in the Presence of 11070gL7gH16 Fab

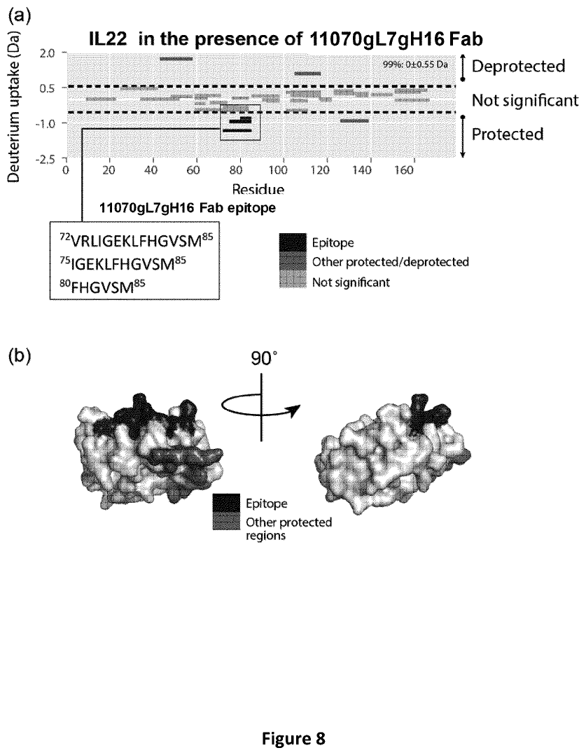

Four peptides showing statistically significant reduced deuterium incorporation upon antibody binding (i.e. potential epitope) were observed, three of which agreed with the SPEED analysis (highlighted in black on the woods plot in FIG. 8, A): 72VRLIGEKLFHGVSM85, 75IGEKLFHGVSM85 and 80FHGVSM85. An increase in deuterium uptake (i.e. potential conformational change) was observed in two peptides: 43DKSNFQQPYITNRTFM58 and 105FPQSDRFQPYMQE117. The 11070gL7gH16 Fab epitope was projected onto the structure of IL22 (FIG. 8, B). Other regions that were protected or deprotected upon antibody binding due to conformational change are highlighted in dark grey.

In conclusion, the protected region which represents the epitope region of 11070gL7gH16 Fab is residues 72-85 (VRLIGEKLFHGVSM).

TABLE 23
List of peptides that show significant change upon
antibody binding measured by HDX-MS.

| | | | | |
|---|---|---|---|---|
| | | | SEQ | |
| Start | End | Peptide sequence | ID NO | Deuterium Uptake |

HDX-MS of IL22 in the presence
of 11041gL13gH14 Fab

| | | | | |
|---|---|---|---|---|
| 72 | 84 | VRLIGEKLFHGVS | 154 | Protected/epitope |
| 72 | 85 | VRLIGEKLFHGVSM | 155 | Protected/epitope |
| 75 | 84 | IGEKLFHGVS | 156 | Protected/epitope |
| 75 | 85 | IGEKLFHGVSM | 157 | Protected/epitope |
| 76 | 84 | GEKLFHGVS | 158 | Protected/epitope |
| 80 | 85 | FHGVSM | 159 | Protected/epitope |
| 126 | 139 | SNRLSTCHIEGDDL | 160 | Protected |
| 101 | 111 | EEVLFPQSDRF | 161 | Deprotected |
| 103 | 115 | VLFPQSDRFQPYM | 162 | Deprotected |

HDX-MS of IL22 in the presence
of 11070gL7gH16 Fab

| | | | | |
|---|---|---|---|---|
| 103 | 117 | VLFPQSDRFQPYMQE | 163 | Deprotected |
| 72 | 85 | VRLIGEKLFHGVSM | 155 | Protected/epitope |
| 75 | 85 | IGEKLFHGVSM | 157 | Protected/epitope |
| 80 | 85 | FHGVSM | 159 | Protected/epitope |
| 126 | 139 | SNRLSTCHIEGDDL | 160 | Protected |
| 43 | 58 | DKSNFQQPYITNRTFM | 164 | Deprotected |
| 105 | 117 | FPQSDRFQPYMQE | 165 | Deprotected |

### Example 13. Purification and Structural Analysis of the IL22/11041gL13gH14 Complex

IL22 was purified as described in Example 11.

Cleaved IL22 was mixed with 11041gL13gH14 Fab and purified by size-exclusion chromatography on a HiLoad® 26/600 Superdex® 75 pg column (GE Healthcare), equilibrated with 10 mM Tris pH7.4 and 150 mM NaCl.

The IL22/11041gL13gH14 Fab complex was concentrated to 10.1 mg/ml. Crystallization conditions for the complex were identified using several commercially available crystallisation screens. These were carried out in sitting drop format, using Swissci 96-well 2-drop MRC Crystallization plates (sourced from Molecular Dimensions, Cat No. MD11-00-100). First, the reservoirs were filled with 75 μL of each crystallization condition in the screens using a Microlab STAR liquid handling system (Hamilton). Then, 300 nL of the IL22/Fab complex and 300 nL of the reservoir solutions were dispensed in the wells of the crystallisation plates using a Mosquito liquid handler (TTP LabTech). An initial crystallisation condition was identified in condition 59 of the Nextal Tubes JCSG+ screen (Qiagen Cat No: 130720), containing 0.16 M Calcium acetate hexahydrate, 0.08 M sodium cacodylate pH6.5, 14.4% PEG8000 and 20% glycerol. This condition will be further referred to as JCSG+59. Optimized crystals were obtained by adding Yttrium(III) Chloride hexahydrate—which is included in the Additive Screen (Hampton Research Cat No HR2-138)—at 0.01 M to JCSG+59 which was sourced from Molecular Dimensions (Cat No. MDSR-37-E11). The optimized crystals were grown in MRC Maxi 48 Well Crystallisation Plates (Swissci), using a reservoir volume of 250 μL and a drop consisting of 2 μL reservoir solution mixed with 2 μL of the IL22/Fab complex. Before flash freezing in liquid nitrogen, the crystals were transferred to a 4 μL drop of cryoprotection solution. This solution was prepared by mixing 40 μL of the optimized reservoir solution with 10 μL of solution CryoMixes™ 7, included with the CryoProtX™ kit (Molecular Dimensions MD1-61). CryoMixes™ 7 contains 12.5% v/v Diethylene glycol, 12.5% v/v Ethylene glycol, 25% v/v 1,2-Propanediol, 12.5% v/v Dimethyl sulfoxide and 12.5% v/v Glycerol.

Diffraction data were collected at beamline I04(Diamond Light Source, UK). The data was indexed and integrated using XDS [Kabsch, W. Acta Cryst. D66, 125-132 (2010)], followed by scaling using AIMLESS [Evans et al Acta Crystallogr D Biol Crystallogr. 2013; 69(Pt 7):1204-1214]. The IL22/Fab structure was solved by molecular replacement using Phaser [McCoy et al J. Appl. Cryst. (2007). 40, 658-674] in the Phenix software suite [Adams et al Methods. 2011; 55(1):94-106]. In this procedure, IL22 structure 1YKB [Xu et al Acta Crystallogr D Biol Crystallogr. 2005 July; 61(Pt 7):942-50] and Fab structure 5BVJ [Rondeau et al MAbs. 2015; 7(6):1151-60] were used as molecular replacement templates. Coot [P. Emsley et al (2010). Acta Crystallographica. D66: 486-501] and phenix.refine [P. V. Afonine et al Acta Crystallogr D Biol Crystallogr 68, 352-67 (2012)] were used in the following cycles of manual model completion and refinement. MolProbity [Williams et al. (2018) Protein Science 27: 293-315] was used to analyze the quality of the final model.

Three IL22/11041gL13gH14 Fab complexes are observed in the crystal asymmetric unit.

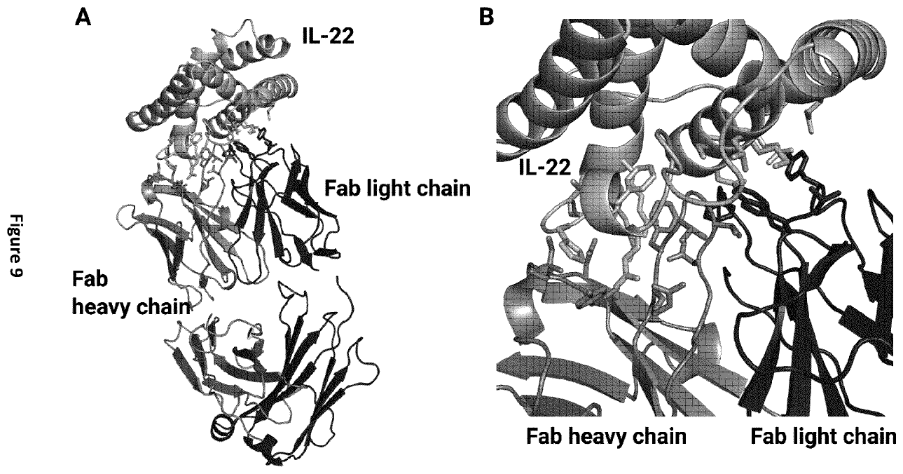

FIG. 9A shows the interaction of 11041gL13gH14 Fab with IL22, with a detailed view on the interaction interface (FIG. 9B). NCONT in the CCP4 software suite [Winn M D et al Acta Crystallogr D Biol Crystallogr. 2011 April; 67(Pt 4):235-42] was used to determine the epitope on IL22, recognized by the Fab molecule. The IL22 amino acid numbering is based on UnitProtKB entry Q9GZX6.

At a <4 Å contact distance with the Fab molecule, the IL22 epitope is composed of residues: Gln48, Glu77, Phe80, His81, Gly82, Val83, Ser84, Met85, Arg88, Leu169, Met172, Ser173, Arg175, Asn176 and Ile179.

At a <5 Å contact distance with the Fab molecule, the IL22 epitope is composed of residues: Lys44, Phe47, Gln48, Ile75, Gly76, Glu77, Phe80, His81, Gly82, Val83, Ser84, Met85, Ser86, Arg88, Leu169, Met172, Ser173, Arg175, Asn176 and Ile179.

TABLE 24
Amino acids of the light chain of 11041gL13gH14 Fab and their
corresponding contacts on IL22. Residues in bold are involved in
contacts between 4 and 5 Å. Other residues have ≤4 Å contact distance
between the antibody and IL22. Sequential numbering is used.

| light chain | IL22 residue |
|---|---|
| Tyr30 (CDR1) | Gln48 |
| | Arg175 |
| | Lys44 |
| | Phe47 |
| | Gln48 |
| | Ile179 |
| Thr31 (CDR1) | Met172 |
| | Arg175 |
| Asn32 (CDR1) | Arg175 |
| | Asn176 |
| Trp50 (CDR2) | Leu169 |
| | Met172 |
| | Arg175 |
| | Asn176 |
| | Ser173 |
| Tyr93 (CDR3) | Asn176 |
| | Ile179 |
| | Arg88 |
| Gly94 (CDR3) | Ile179 |
| Tyr101 (CDR3) | Met85 |

TABLE 25
Amino acids of the heavy chain of 11041gL13gH14 Fab and their
corresponding contacts on IL22. Residues in bold are involved in
contacts between 4 and 5 Å. Other residues have ≤4 Å contact distance
between the antibody and IL22. Sequential numbering is used.

| heavy chain | IL22 residue |
|---|---|
| Ser30 (CDR1) | Gly82 |
| Ser31 (CDR1) | Gly82 |
| | Phe80 |
| | His81 |
| Tyr32 (CDR1) | Glu77 |
| | His81 |
| | Glu77 |
| Ala33 (CDR1) | Met85 |
| Ile50 (CDR2) | Met85 |
| Asp52 (CDR2) | Ser84 |
| | Met85 |
| | Val83 |
| | Ser86 |
| Ile53 (CDR2) | Gly82 |
| | Val83 |
| | Ser84 |
| Glu54 (CDR2) | Ser84 |
| Tyr58 (CDR2) | Met85 |
| Arg97 (CDR3) | Glu77 |
| Asp98 (CDR3) | Met85 |
| | Arg88 |
| Arg99 (CDR3) | Glu77 |
| Phe100 (CDR3) | Glu77 |
| | Phe80 |
| | Arg88 |
| | Gly76 |
| | Glu77 |
| Val101 (CDR3) | Glu77 |
| | Ile75 |
| | Gly76 |
| | Glu77 |
| | Phe80 |
| | Ser173 |
| Gly102 (CDR3) | Phe80 |
| | Met172 |
| | Ser173 |
| | Asn176 |
| Val103 (CDR3) | Asn176 |
| Asp104 (CDR3) | Arg88 |
| | Met85 |

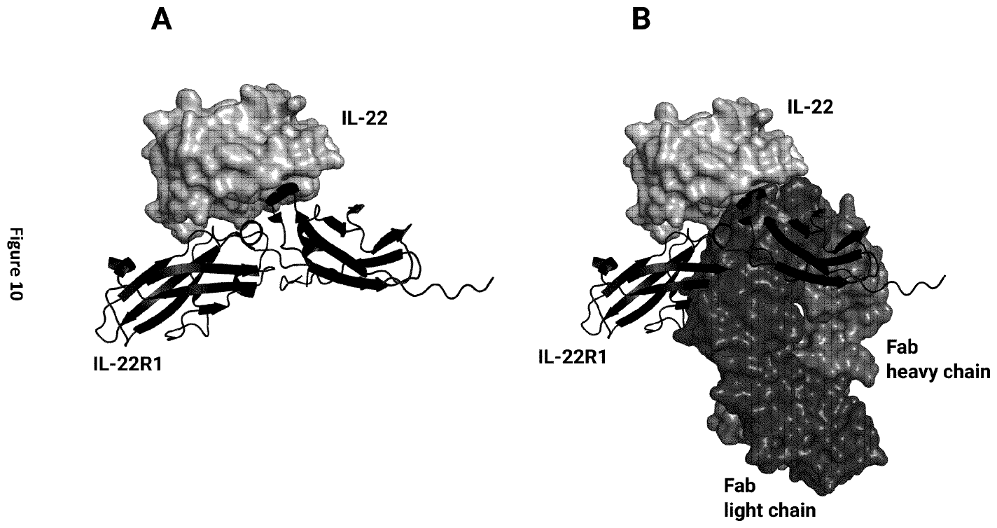

The 11041gL13gH14 Fab molecule prevents the interaction of IL22 with the IL22R1 receptor, as the Fab light chain binds to the same epitope on IL22. (FIG. 10)

### Example 14. Structure Determination of IL-22 in Complex with Fezakinumab and 11070gL7gH16 Fab by Cryo-EM

IL-22 was expressed using Expi293 cells, fused to an N-terminal human Fc tag. After clearing the cells by centrifugation, the supernatant was loaded on a 5 ml HiTrap Protein A column (Cytiva). The protein was eluted with a buffer gradient from PBS to 0.1 M Sodium Citrate at pH 2.0. The hFc tag was cleaved using TEV protease and IL-22 was separated from the cleaved tag by another pass by gravity flow over 4 ml packed protein A resin. After elution from the resin, IL-22 was further purified on a HiLoad 26/600 Superdex75 pg column (Cytiva), equilibrated in PBS.

70 microliters 11070gL7gH16 Fab at 12.1 mg/ml, 153 microliters Fezakinumab Fab at 11.5 mg/ml and 153 microliter IL-22 at 1.36 mg/ml were mixed. 55 microliters were injected onto a Superdex200 5/150 column equilibrated in 10 mM Hepes pH 7.4 and 150 mM NaCl. A fraction containing the IL-22+11070+Fezakinumab complex at 1.7 mg/ml was collected and used for preparing cryo-EM grids.

Quantifoil® R 1.2/1.3 holey carbon grids (SPT Labtech) were glow-discharged in a Pelco easyGlow™ for 45s at 22 mA immediately before use. The IL22 with 11070gL7gH16 Fab and Fezakinumab Fab after gel filtration was applied to the freshly glow-discharged grid in a Vitrobot Mark IV (Thermo Fisher Scientific) for 2 s in the chamber at 100% humidity and 4° C. The grid was then blotted on fresh filter paper for 4 s at force 7 and plunged in liquid ethane. The grid was first screened for ice thickness and particle distribution in the in-house Glacios operated at 200 keV and equipped with a Falcon 3 camera. The data was then collected on the Krios2 of the Cambridge consortium equipped with a Falcon 4 and operated at 300 keV acceleration voltage. The 5700 movies were collected automatically using the EPU software, in counting mode at a defocus range of −1 to -2.5 μm, at a pixel size of 0.67 Å with a 12.2 s exposure for a final electron flux of 49.36 e⁻/Å² distributed over 42 fractions. All subsequent data analysis was performed on Cryosparc, version 2.15 (Structura Biotechnology Inc). The movies were aligned using Patch Motion, contrast transfer function parameters (CTF) were estimated using Patch CTF and particles were initially picked with the blob picker and resulted in a total of 5.5 M particles. The picked particles were binned 2× to a box size of 300 pixels and subjected to a first round of 2D classification that resulted in the selection of 488,000 particles with distinct features. Five ab initio models were generated, 2 of which differed from one another at the glycosylation sites of IL22. These two classes, for a total of 240,000 particles, were pooled together and a non-uniform refinement yielded a resolution estimation of 3.4 Å using the gold-standard FSC 0.143 criterion.

Two Fab molecules and the IL-22 structure were fitted in the cryo-EM density using UCSF Chimera [Pettersen, et al J. Comput. Chem. 25(13):1605-1612 (2004).]. After further manual model building using Coot [Emsley et al (2010) Acta Crystallographica. D66: 486-501.], the map was sharpened using the Autosharpen [Terwilliger, (2018). Acta Cryst. D74, 545-559.] tool in Phenix [Liebschner et al. Acta Cryst. D75, 861-877 (2019)] and the model was further refined using the Real-space refinement [Afonine et al Acta Cryst. D74, 531-544 (2018)] tool in Phenix.

NCONT in the CCP4 software suite [Winn et al Acta Crystallogr D Biol Crystallogr. 2011 April; 67(Pt 4):235-42] was used to determine the epitope on IL-22, recognized by the 11070 and Fezakinumab Fab molecules. The IL-22 amino acid numbering below is based on UnitProtKB entry

Q9GZX6.

At a <4 Å contact distance with the 11070gL7gH16 Fab molecule, the IL-22 epitope is composed of residues: Glu77, Lys78, His81, Ser84, Met85, Ser86, Arg88, Asn176, Ala177

At a <5 Å contact distance with the 11070gL7gH16 Fab molecule, the IL-22 epitope is composed of residues: Ile75, Gly76, Glu77, Lys78, Phe80, His81, Ser84, Met85, Ser86, Arg88, Leu169, Met172, Ser173, Asn176, Ala177

At a <4 Å contact distance with the Fezakinumab Fab molecule, the IL-22 epitope is composed of residues: Gln49, Tyr51, Phe105, Ser108, Asp109, Gln112, Pro113, Tyr114, Gln116, Glu117, Pro120, Ala123, Arg124

At a <5 Å contact distance with the Fezakinumab Fab molecule, the IL-22 epitope is composed of residues: Gln49, Pro50, Tyr51, Ile52, Arg55, Phe105, Pro106, Ser108, Asp109, Gln112, Pro113, Tyr114, Gln116, Glu117, Val119, Pro120, Phe121, Ala123, Arg124

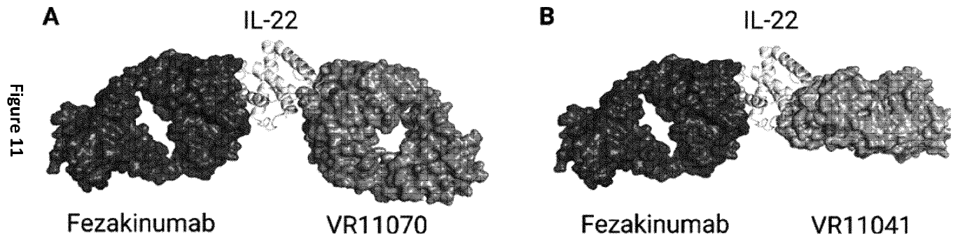

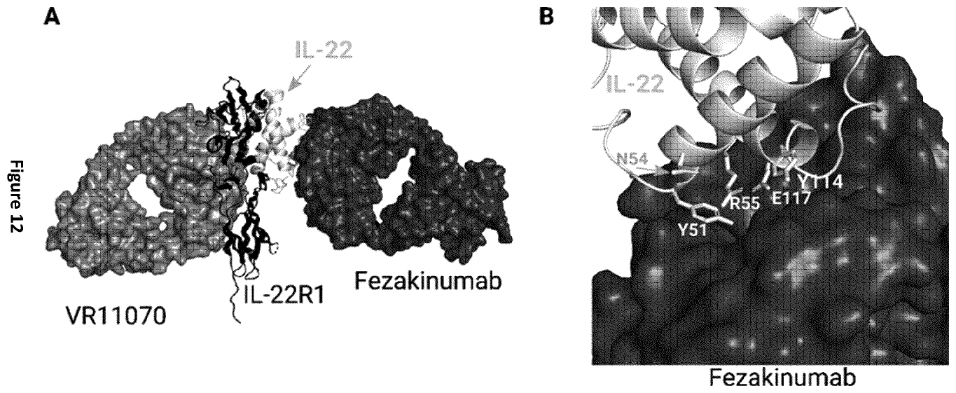

The structural analysis reveals that 11070gL7gH16 Fab has a different epitope on IL-22 then Fezakinumab. Also, the 11070gL7gH16 Fab is similar to the epitope of 11041gL13gH14 Fab on IL-22. (FIGS. 11A and 1B). Like 11041gL13gH14 Fab, 11070gL7gH16 Fab blocks IL-22 signaling by preventing the interaction with the IL22R1 receptor (FIG. 12A). In contrast, Fezakinumab blocks IL-22 signaling by preventing the interaction of IL22 with IL-10R2 (FIG. 12B).

### Example 15. Multi-Specific Antibody—Construction of Transient Plasmids and Expression in Cells

IL13/IL22 TrYbe antibodies were designed with the anti-IL22 V-region (11041gL13gH14) fixed in the Fab position; the anti-albumin V-region (645gL4gH5) and IL13 (1539gL8gH9) were reformatted into disulfide-linked scFv in the HL orientation (dsHL) and linked to the C-termini of the respective heavy and light chain constant regions via a 11-amino acid glycine-serine rich linkers.

The light chain and heavy chain genes were independently cloned into proprietary mammalian expression vectors for transient expression under the control of a hCMV promoter. The following light and heavy chain sequences were used:

Light chain:
(SEQ ID NO: 166)
gcggtgcagctgactcagtcaccgtcctcgctttccgcttccgtgggag
acagagtgaccatcacctgtcaagcctccgaagatatctacaccaacct
cgcctggtaccagcagaagcccggaaaggccccaaagctgttgatctac
tgggcgtctaccctcgcctccggggtgccgtcgcgctttagcggttcgg
gatccggcaccgacttcaccctgactattagcagcctgcagcctgagga
cttcgccacttattactgccaagcatccgtctacgggaacgccgccgat
tcacggtacaccttcggcggcggaacgaaagtcgagattaagcgtacgg
tagcggccccatctgtcttcatcttcccgccatctgatgagcagttgaa
atctggaactgcctctgttgtgtgcctgctgaataacttctatcccaga
gaggccaaagtacagtggaaggtggataacgccctccaatcgggtaact
cccaggagagtgtcacagagcaggacagcaaggacagcacctacagcct
gagcagcaccctgacgctgtctaaagcagactacgagaaacacaaagtg
tacgcctgcgaagtcacccatcagggcctgagctcaccagtaacaaaaa
gttttaatagaggggagtgtagcggtggcggtggctccggtggtggcgg
ttcagaggtgcagctggtgcagtccggcgccgaggtgaagaagcccggc
tcctccgtgaaggtgtcctgcaaggcctccggctactccttcacctcct
actacatccactgggtgaggcaggcccccggccagtgcctggagtggat
gggcaggatcggccccggctccggcgacatcaactacaacgagaagttc
aagggcagggccaccttcaccgtggacaagtccacctccaccgcctaca
tggagctgtcctccctgaggtccgaggacaccgccgtgtactactgcgc
caggttccactacgacggcgccgactggggccagggcaccctggtgacc
gtgtcctccggaggtggcggttctggcggtggcggttccggtggcggtg
gatcgggaggtggcggttctgacatccagatgacccagtccccctcctc
cctgtccgcctccgtgggcgacagggtgaccatcacctgcaaggcctcc
cagaacatcaacgagaacctggactggtaccagcagaagcccggcaagg
cccccaagctgctgatctactacaccgacatcctgcagaccggcatccc
ctccaggttctccggctccggctccggcaccgactacaccctgaccatc
tcctccctgcagcccgaggacttcgccacctactactgctaccagtact
actccggctacaccttcggctgcggcaccaagctggagatcaagcgtac
c
Heavy chain:
(SEQ ID NO: 167)
gaggtgcagctcgtggaatccggcggcggactggtgcagccgggcggat
ccctgcggctgtcctgcgccgtgtcgggtttttccctgtcctcatacgc
catgatctgggtcagacaggcacctgggaagggtctggagtggattggc
atcatcgacatcgaagggtcgacctactacgcgagctgggccaagggaa
ggttcaccattagccgggacaacagcaagaacaccgtgtaccttcaaat
gaactccctccgggccgaagataccgccgtgtattactgtgctcgcgac
cgcttcgtgggagtggacatcttcgatccctggggacagggaactttgg
tcactgtctcgagcgcgtccacaaagggcccatcggtcttccccctggc
accctcctccaagagcacctctgggggcacagcggccctgggctgcctg
gtcaaggactacttccccgaaccagtgacggtgtcgtggaactcaggtg
ccctgaccagcggcgttcacaccttcccggctgtcctacagtcttcagg
actctactccctgagcagcgtggtgaccgtgccctccagcagcttgggc
acccagacctacatctgcaacgtgaatcacaagcccagcaacaccaagg
tcgataagaaagttgagcccaaatcttgtagcggtggcggtggctccgg
tggtggcggttcagaagtgcagttgctggagtcaggtggagggctggtg
cagcccggaggatcgctgcggttgtcatgcgcggtgtccggtattgatt
tgtccaattacgccatcaattgggtacgccaagcgccagggaagtgcct
tgagtggattggcatcatctgggcgtcggggacgaccttttatgctact
tgggccaaaggaagattcacaatctcccgagacaactcgaagaacaccg
tgtatcttcaaatgaactcgctcagggccgaggacacggcggtctacta
ctgtgcacggacagtgccgggttattcaacggcaccttactttgatctt
tggggccaggggaccctcgtgactgtctcaagtggaggtggcggttctg
gcggtggcggttccggtggcggtggatcgggaggtggcggttctgatat
tcagatgacgcaatcaccttcgagcgtatccgcctcggtgggagacagg
gtgacaatcacttgtcagtcatccccctcagtctggagcaactttttgt
catggtatcagcagaagcccggaaaggctccgaaattgctgatctacga
ggcatcgaagttgacgagcggtgtaccaagcagattctccggttcgggg
tcgggaactgacttcaccettacgatctcatcgctgcagccggaggatt
ttgcgacctactactgtgggggtgggtattcgtcgatttccgacacaac
attcgggtgcggcacgaaagtggaaatcaagcgtacc

Equal ratios of both plasmids were transfected into the CHO—S XE cell line (UCB) using the commercial ExpiCHO expifectamine transient expression kit (Thermo Scientific). The cultures were incubated in Corning roller bottles with vented caps at 37° C., 8.0% CO2, 190 rpm. After 18-22 h, the cultures were fed with the appropriate volumes of CHO enhancer and feeds for the HiTiter method as provided by the manufacturer. Cultures were re-incubated at 32° C., 8.0% CO₂, 190 rpm for an additional 10 to 12 days. The supernatant was harvested by centrifugation at 4000 rpm for 1 h at 4° C. prior to filter-sterilization through a 0.45 μm followed by a 0.2 μm filter. Expression titres were quantified by Protein G HPLC using a 1 ml GE HiTrap Protein G column (GE Healthcare) and Fab standards produced in-house.

### Example 16. Mammalian Cell Line Development

To demonstrate stable expression of IL13/IL22 TrYbe a stably expressing mammalian cell line was created. A CHO cell line was transfected with a vector containing 11041gL13gH14 Fab (IL22 binding domain), 650gH9gL8 dsscFv (IL13-binding domain), 645gH5gL4 dsscFv (albumin-binding domain) and a selectable marker. Sequences of SEQ IDs 61 and 65 were included in the vector used to generate the cell line. The cell lines were cloned and evaluated for fit to a suitable manufacturing process. To assess the quality and quantity of the protein and to ensure the optimal cell line was selected, the cell line was evaluated in a small scale model of a manufacturing fed-batch bioreactor. A CHO cell line was selected that expressed sufficient levels of IL13/IL22 TrYbe.

### Example 17. Purification of IL13/IL22 TrYbe Multi-Specific Antibody

The TrYbe protein was purified by native protein A capture step followed by a preparative size exclusion polishing step. Clarified supernatants from standard transient CHO expression were loaded onto a MabSelect (GE Healthcare) column giving a 12 min contact time and washed with binding buffer (200 mM Glycine pH7.4). Bound material was eluted with a 0.1M sodium citrate pH3.2 step elution and neutralised with 2M Tris/HCl pH8.5 and quantified by absorbance at 280 nm.

Size exclusion chromatography (SE-UPLC) was used to determine the purity status of the eluted product. The antibody (˜3 lag) was loaded on to a BEH200, 200 Å, 1.7 μm, 4.6 mm ID×300 mm column (Waters ACQUITY) and developed with an isocratic gradient of 0.2 M phosphate pH 7 at 0.35 mL/min. Continuous detection was by absorbance at 280 nm and multi-channel fluorescence (FLR) detector (Waters). The eluted TrYbe antibody was found to be 65% monomer.

The neutralised samples were concentrated using Amicon Ultra-15 concentrator (10 kDa molecular weight cut off membrane) and centrifugation at 4000×g in a swing out rotor. Concentrated samples were applied to a XK26/60 Superdex200 column (GE Healthcare) equilibrated in PBS, pH7.4 and developed with an isocratic gradient of PBS, pH7.4 at 2.5 ml/min. Fractions were collected and analysed by size exclusion chromatography on a BEH200, 200 Å, 1.7 μm, 4.6 mm ID×300 mm column (Aquity) and developed with an isocratic gradient of 0.2 M phosphate pH 7 at 0.35 mL/min, with detection by absorbance at 280 nm and multi-channel fluorescence (FLR) detector (Waters). Selected monomer fractions were pooled, 0.22 μm sterile filtered and final samples were assayed for concentration by A280 Scanning on Cary UV Spectrophotometer. Endotoxin level was less than 1.0 EU/mg as assessed by Charles River's EndoSafe® Portable Test System with Limulus Amebocyte Lysate (LAL) test cartridges.

Monomer status of the final TrYbe was determined by size exclusion chromatography on a BEH200, 200 Å, 1.7 μm, 4.6 mm ID×300 mm column (Aquity) and developed with an isocratic gradient of 0.2 M phosphate pH 7 at 0.35 mL/min, with detection by absorbance at 280 nm and multi-channel fluorescence (FLR) detector (Waters). The final TrYbe antibody was found to have less than 1% HMW species.

For analysis by sodium dodecyl sulfate-polyacrylamide gel electrophoresis (SDS-PAGE) samples were prepared by adding 4× Novex NuPAGE LDS sample buffer (Life Technologies) and either 10× NuPAGE sample reducing agent (Life Technologies) or 100 mM N-ethylmaleimide (Sigma-Aldrich) to ˜3 μg purified protein, and were heated to 100° C. for 3 min. The samples were loaded onto a 15 well Novex 4-20% Tris-glycine 1.0 mm SDS-polyacrylamide gel (Life Technologies) and separated at a constant voltage of 225 V for 40 min in Tris-glycine SDS running buffer (Life Technologies). Novex Mark12 wide-range protein standards (Life Technologies) were used as standards. The gel was stained with Coomassie Quick Stain (Generon) and destained in distilled water.

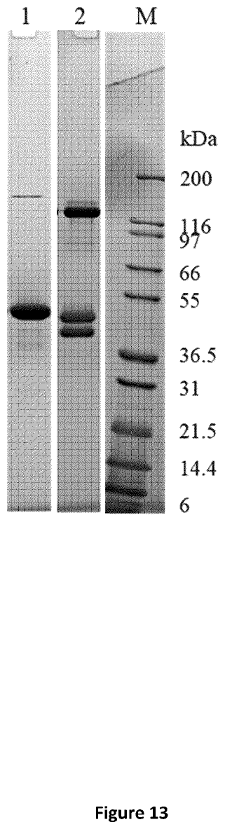

On non-reducing SDS-PAGE the TrYbe, theoretical molecular weight (MW) of ˜100 kDa, migrated to ˜120 kDa (FIG. 13). When the TrYbe protein was reduced, both chains migrated at a mobility rate approaching their respective theoretical MWs, ˜50 kDa. Additional bands on the non-reduced gel at ˜45-50 kDa are due to incomplete formation of the natural interchain disulphide (ds) bond between the CH1 and CK in a portion of the molecules, they do not migrate to the same position as the LC and HC in lane 2 as they are not fully reduced.

### Example 18. Production and Purification of KiH Antibodies Binding Human IL13 and IL22

Parental monoclonal antibodies (mAbs) containing either T366W (knob mutation) or L366S L368A, and Y407V (hole mutations) were expressed and purified by standard chromatography methods including a protein A capture step followed by preparative size exclusion chromatography (SEC). In order to generate the bispecific, parental mAbs were mixed at a 1:1 ratio in the presence of 50 mM beta-mercaptoethylamine for 18 hr at room temperature. High molecular weight species or parental mAbs were removed subsequently by a second round of preparative size exclusion using a S200 16/60 column equilibrated in PBS pH 7.4. Percentage bispecific was determined by analytical HIC chromatography and percentage purity was determined by analytical SEC and SDS-PAGE. Endotoxin levels were determined for all material and removed if necessary using High Capacity Endotoxin Removal Spin Columns (Pierce) to a final level of <1 EU/mg.

### Example 19. Mass Spectrometry—Sequence Identity of IL13/IL22 TrYbe Molecule

The sequence mass of IL13/IL22 TrYbe was confirmed by liquid chromatography-mass spectrometry (LC-MS). An aliquot of IL13/IL22 TrYbe at 0.25 mg/mL was reduced with 5 mM tris(2-carboxyethyl)phosphine (TCEP) in 150 mM ammonium acetate at 37° C. for 40 minutes, then centrifuged and analyzed. Data were acquired using a Waters ACQUITY UPLC System connected to a Waters Xevo G2 Q-ToF mass spectrometer operated with MassLynx™ and processed using OpenLynx™ software packages. LC conditions were as follows: BioResolveT RP mAb Polyphenyl, 450 Å, 2.7 μm column held at 80° C. with a flow rate of 0.6 mL/minute. The mobile phase buffers were; water/0.02% Trifluoroacetic acid (TFA)/0.08% formic acid (Solvent A) and 95% acetonitrile/5% water/0.02% TFA/0.08% formic acid (solvent B). A reverse phase gradient was run from 5% to 50% solvent B over 8.80 minutes with a 95% solvent B wash and re-equilibration. UV data were acquired at 280 nm. MS conditions were as follows: Ion mode: ESI positive ion, resolution mode, mass range: 400-5000 m/z and external calibration with NaI. The observed reduced masses were found to be consistent with the theoretical masses for each chain, that is, 50,427.8 Da for the light chain (50,422.6 Da theoretical) and 50,627.8 Da for the heavy chain (50,623.5 Da theoretical).

### Example 20. Thermal Stability of IL13/IL22 TrYbe

Thermal stability was performed to assess conformational stability of purified sample in pre-formulation storage buffer, PBS pH 7.4 and a commonly used formulation buffer, pH 5.5. The thermal stability was measured by a Fluorescence-based method (thermofluor).

The reaction mix contained 5 μL of 30×SYPRO™ Orange Protein Gel Stain (Thermofisher scientific), diluted from 5000× concentrate with test buffer. 45 μL of IL13/IL22 TrYbe at 0.2 mg/mL, in either buffer was added to the dye and mixed, 10 μL of this solution was dispensed in quadruplicate into a 384 PCR optical well plate and was run on a QuantStudio Real-Time PCR System (Thermofisher). The PCR system heating device was set at 20° C. and increased to 99° C. at a rate of 1.1° C./min. A charge-coupled device monitored fluorescence changes in the wells. Fluorescence intensity increases were plotted, the inflection point of the slope(s) was used to generate apparent midpoint temperatures (Tm).

IL13/IL22 TrYbe exhibited three unfolding transitions in PBS pH 7.4 of 58.3° C. (Tm1), 73.7° C. (Tm2) and 81.4° C. (Tm3) attributed to dsscFv 1539 gL8gH9 (CA650 anti IL13), dsscFv 645 gL4gH5 (anti HSA) and Fab 11041 gL13gH14 (anti IL22) respectively. (ii) Three transitions of 65.2° C. (Tm1), 73.2° C. (Tm2) and 81.3° C. (Tm3) were also seen in formulation buffer, pH 5.5 with the thermal stability of the dsscFv 1539 gL8gH9 (CA650 anti IL13) increasing from 58.3° C. to 65.2° C. as summarised in Table 26.

TABLE 26
Thermal Stability Data from the Thermofluor assay in two different buffers.

| Buffer | Tm1 (° C.) | Tm2 (° C.) | Tm3 (° C.) |
|---|---|---|---|
| PBS, pH7.4 | 58.3 | 73.7 | 81.4 |
| formulation buffer, pH 5.5 | 65.2 | 73.2 | 81.3 |

IL13/IL22 TrYbe exhibited a slightly lower first unfolding transition compared to an IgG4 molecule (˜65° C.; Ref 1) in PBS pH 7.4, however, unlike an IgG4 molecule this transition is stabilized in more acidic buffers.

### Example 21. Experimental pI (Isoelectric Point) of IL13/IL22 TrYbe Molecule

The experimental pI (isoelectric point) of IL13/IL22 TrYbe was obtained using whole-capillary imaged cIEF ICE3 system (ProteinSimple). Samples were prepared by mixing 30 μl sample (from a 1 mg/ml stock in HPLC grade water), 35 μl of 1% methylcellulose solution (Protein Simple), 4 μl pH3-10 ampholytes (Pharmalyte), 0.5 μl of 4.65 and 0.5 μl 9.77 synthetic pI markers (ProteinSimple), 12.5 μl of 8M urea solution (Sigma-Aldrich). HPLC grade water was used to make up the final volume to 100 The mixture was vortexed briefly to ensure complete mixing and centrifuged at 10,000 rpm for 3 minutes to remove air bubbles before analysis. Samples were focused for 1 min at 1.5 kV, followed by 5 min at 3 kV, and A280 images of the capillary were taken using the ProteinSimple software. markers). The calibrated electropherograms were then integrated using Empower software (Waters).

Two peaks were observed; an acidic peak of pI 8.77 and main species of pI 8.96. This was consistent with the theoretical value of 8.9 (non-reduced). The high pI allows for good manufacturability as well as low propensity for aggregation in usual formulation buffers (pH 5-6).

TABLE 27
pI determination by cIEF.

| Peak | pI | % |
|---|---|---|
| 1-Acidic | 8.77 | 32.8 |
| 2-Main species | 8.96 | 67.2 |

### Example 22. Hydrophobic Interaction Chromatography (HIC) of IL13/IL22 TrYbe Molecule

A Dionex ProPac HIC-10 column 100 mm×4.6 mm (ThermoFisher scientific) connected to an Agilent HP1260 HPLC with an on-line fluorescence detector was used to measure apparent hydrophobicity. The mobile phase was 0.8 M ammonium sulfate, 50 mM phosphate pH 7.4 (buffer A) and 50 mM phosphate pH 7.4 (buffer B). IL13/IL22 TrYbe (10 μg (10 μL) was injected onto the column; following a 5 min hold at 0% B, the protein was eluted using a linear gradient from 0 to 100% B over 45 min at a flow rate of 0.8 mL/minute. The separation was monitored by intrinsic fluorescence using excitation at 280 nm and emission at 340 nm.

TABLE 28
HIC Data for IL13/IL22 Tr Ybe

| | Retention Time (min) | Main Peak AUC (%) |
|---|---|---|
| IL13/IL22 TrYbe | 9.6 | 100 |

IL13/IL22 TrYbe exhibited a low apparent hydrophobicity as determined by this assay; that is a retention time of <10 minutes.

### Example 23. Polyethylene Glycol (PEG) Precipitation Assay

The PEG precipitation assay was performed to assess high concentration solubility in both PBS pH 7.4 and a common formulation buffer pH 5.5. PEG was used to reduce protein solubility in a quantitatively definable manner by increasing the concentrations of PEG (w/v) and measuring the amount of protein remaining in solution. This assay served to mimic the effect of high concentration without using conventional concentration methods.

Stock 40% PEG 3350 solutions (W/V) were prepared in PBS pH 7.4 or formulation buffer pH 5.5. A serial titration was performed by a Viaflo assist plus liquid handling robot (Integra), resulting in a PEG 3350 range of 40% to 15.4% PEG 3350. To minimize non-equilibrium precipitation, sample preparation consisted of mixing protein and PEG solutions at a 1:1 volume ratio. 35 μL of the PEG 3350 stock solutions was added to a 96 well v bottom PCR plate (A1 to H1) by a liquid handling robot. 35 μL of a 2 mg/mL sample solution (unless otherwise stated) was added to the PEG stock solutions resulting in a 1 mg/mL test concentration. This solution was mixed by automated slow repeat pipetting. After mixing, the sample plate was sealed and incubated at 37° C. for 0.5 h to re-dissolve any non-equilibrium aggregates. Samples were then incubated at 20° C. for 24 h. The sample plate was subsequently centrifuged at 4000×g for 1 h at 20° C. 50 μL of supernatant was dispensed into a UV-Star®, half area, 96 well, μClear®, microplate. The protein concentration was determined by UV spectrophotometry at 280 nm using a FLUOstar Omega® multi-detection microplate reader (BMG LABTECH). The resulting values were plotted against percent PEG using Graphpad prism and the PEG midpoint (PEGmdpnt) score was derived from the midpoint of the sigmoidal dose-response (variable slope) fit.

IL13/IL22 TrYbe exhibited a high PEGmdpnt in PBS pH 7.4 and is therefore expected to show low aggregation propensity. IL13/IL22 TrYbe exhibited an apparently increased PEGmdpnt in formulation buffer pH 5.5 compared to the sample tested in PBS pH 7.4 and so would be expected to show high concentration stability in a typical formulation buffer.

TABLE 29
PEG midpoint data for sample in PBS and formulation buffer pH 5.5.

| Buffer | PEG midpoint (%) |
|---|---|
| PBS pH 7.4 | 12.4 |
| formulation buffer, pH 5.5 | 17.6* |

*sample did not reach baseline at the highest test concentration of PEG 3350.The generated PEG_{mdpnt} is not accurate but does reflect the low aggregation propensity of this sample in formulation buffer, pH 5.5.

### Example 24. Effect of Stress at an Air-Liquid Interface (Agitation Assay) on IL13/IL22 TrYbe

Proteins tend to unfold when exposed to an air-liquid interface, where hydrophobic surfaces are presented to the hydrophobic environment (air) and hydrophilic surfaces to the hydrophilic environment (water). Agitation of protein solutions achieves a large air-liquid interface that can drive aggregation. This assay serves to mimic stresses that the molecule would be subjected to during manufacture (for example ultra-filtration) and potential transportation.

IL13/IL22 TrYbe samples in PBS pH 7.4 (typical storage buffer) and formulation buffer pH 5.5±Tween80 (typical formulation buffer) were stressed by vortexing using an Eppendorf Thermomixer Comfort™. The sample was buffer exchanged into the respective buffers using 7 mL Zeba™ desalting columns (Thermofisher) and the concentration adjusted to 1 mg/mL using the appropriate extinction coefficients (1.72 Abs 280 nm, 1 mg/mL, 1 cm path length). The absorbance at 280 nm and 595 nm was obtained using a Varian Cary® 50-Bio spectrophotometer to establish the time zero reading. The sample in each buffer was sub-aliquoted into 1.5 mL conical Eppendorf®-style capped tubes (4×250 μL) and subjected to vortexing at 1400 rpm at 25° C. Time dependent aggregation (turbidity) was monitored by measurement of the samples at 24h at 595 nm using a Varian Cary® 50-Bio spectrophotometer. The mean and SD of the triplicate readings was calculated and is summarized in Table 30.

TABLE 30
Mean turbidity measurements of IL13/IL22 TrYbe in PBS, pH7.4 and
formulation buffer, pH 5.5 +/− 0.03% Tween 80.

| Buffer | OD at 595 nm |
|---|---|
| PBS pH 7.4 | 0.66 ± 0.19 |
| formulation buffer, pH 5.5 | 0.0085 ± 0 |
| formulation buffer, pH 5.5 + 0.03% Tween80 | 0.0007 ± 0 |

The highest aggregation propensity was obtained in PBS, pH7.4. There was little aggregation observed (as judged by OD at 595 nm) when the samples were subjected to vortex in formulation buffer, pH 5.5±Tween 80. The samples would be expected to be stable for long term storage and transportation in a typical formulation buffer: formulation buffer, pH 5.5+/−0.03% Tween 80.

### Example 25. Deamidation and Asp Isomerization Stress Study

A stress study was set up to determine deamidation/Asp isomerization propensity of two identified sequence liabilities: Asn (95) Ala (deamidation) and Asp(98) Ser both in the light chain CDR3 of anti-IL22 domain of IL13/IL22 TrYbe. The propensity/rate of deamidation/Asp isomerization cannot be predicted as it is dependent on linear sequence and 3D structure as well as solution properties.

The basal deamidation/Asp isomerization levels were also obtained-low levels indicate low susceptibility but can change due to different manufacturing batches/conditions.

IL13/IL22 TrYbe was buffer exchanged into buffers (i) known to favor deamidation of Asn (N) residues (Tris, pH 8) and (ii) known to favor Asp(D) isomerization (acetate, pH 5). The final concentration was adjusted to approximately 5 mg/mL and then split into two aliquots where one was stored at 4° C. and one at 37° C. for up to 4 weeks. An aliquot was removed immediately (T0, non-stressed control) and 2 weeks and 4 weeks and stored at −20° C.

### Mass Spectrometry/Peptide Mapping

The 2-week samples were analyzed with liquid chromatography mass spectrometry (LCMS)/peptide mapping for chemical modification as follows. Both stressed and non-stressed samples (16 μL at 5 mg/mL) were incubated with 24 Dithiothreitol (DTT; 500 mM) and 60 μl 8M guanidine hydrochloride at 37° C. for 40 minutes and then capped with 64 iodoacetamide (IAM; 500 mM) for a further 30 minutes at room temperature. The samples were then buffer exchanged into digestion buffer (7.5 mM Tris hydrochloride/1.5 mM calcium chloride, pH 7.9) and immediately added to trypsin and incubated at 37° C. for 3 hours. A volume of 5 uL of 1% TFA (trifluoracetic acid) was used to quench the digestion before being analyzed by LCMS. This was performed on a Thermo Q Exactive Orbitrap, using a Waters C18 BEH 2.1 mm×150 mm, 1.7 μm column. Mobile phase A was 0.1% formic acid in water, mobile phase B was 0.1% formic acid in acetonitrile.

The results of the mass spectrometry and peptide mapping showed that the basal deamidation of Asn(95) in the light chain CDR3 was ˜10% and increased to >40% (40-60% derived from different data analysis methods) after 2 weeks at pH 8 and 37° C.

There was no evidence for chemical modification of Asp at D(98)Ser in either the non-stressed (basal) or stressed samples in any buffer conditions.

Isomerization of an aspartic acid usually results in earlier elution of the modified peptide than the corresponding unmodified sequence. A change in the elution profile of the tryptic peptide containing the Asp(98) Ser motif (residues spanning 62-100 of the light chain) was not observed, indicating that an isoAsp had not been formed or alternatively co-eluted with the unmodified and was therefore not detected at this site.

The propensity of chemical modification of the deamidation motif: Asn(95)Ala on the light chain CDR was shown to be high but can be controlled by careful monitoring and by avoiding prolonged exposure to high pH and formulating in a low pH buffer (<pH 5-6). No Asp isomerization at the Asp(98) Ser motif on the light chain CDR was observed.

### Surface Plasmon Resonance (SPR) Analysis

The effect of the chemical modification (deamidation) of the Asn(95)Ala motif in the light chain CDR3 on the affinity for IL22 was assessed. The binding kinetics and affinity of IL13/IL22 TrYbe was not changed. There would therefore be no impact on efficacy of this molecule as a consequence of chemical modification.

TABLE 31
Binding kinetics for T0 and 2 week samples pH8.0 (2weeks/37° C.);
KD = kd/ka.

| Condition | ka(1/Ms) | kd(1/s) | KD(pM) |
|---|---|---|---|
| T0 | 1.20E+06 | 3.10E−05 | 26 |
| 2 week/37° C. | 1.10E+06 | 3.20E−05 | 30 |

### Example 26. IL22R1 Cross-Blocking Experiment by IL13/IL22 TrYbe

A cross-blocking assay was performed on a Biacore T200 (GE Healthcare) to determine whether the binding of IL13/IL22 TrYbe to IL-22 prevent the binding of IL-22R1.

A CM5 Sensor chip was prepared by activating with a 7-minute injection (at 10 μL min-1) of a mixture of EDC/NHS (GE Healthcare) followed by a 7-minute injection of a human-Fab specific goat Fab′2 (Jackson Immuno Research) at 50 μg/ml in acetate buffer pH 5.0 (GE Healthcare) to achieve an immobilization level of approximately 5500 RU. Finally, a 7-minute injection (at 10 μL min-1) of 1M Ethanolamine hydrochloride-NaOH pH 8.5 was performed to deactivate the surface. A reference surface was prepared as above, omitting the human-Fc specific capture antibody.

Cross-blocking was performed at 25° C. in HBS-EP+ buffer (GE Healthcare). Each analysis cycle involved capturing of IL13/IL22 TrYbe on the chip surface followed by an injection of human IL-22 at 50 nM concentration for 300s and finally an injection of IL-22R1 at 50 nM for 300s. Binding responses were calculated after subtraction of buffer blank and no capture control samples. A positive response for the IL-22R1 would indicate that IL13/IL22 TrYbe binds to IL-22 on a different epitope from IL-22R1. A lack of response for the IL-22R1 would indicate that the binding site of IL13/IL22 TrYbe on IL-22 overlaps with IL-22R1 binding site. After each cycle the surface was regenerated by the following injections: 60s 50 mM HCl, 60s 5 mM NaOH and 60s HCl, all at 10 μL min-1.

As shown in table below, a clear binding response was observed when human IL-22 was injected on captured IL13/IL22 TrYbe but no response was seen for the IL-22R1 injection. This demonstrates that IL13/IL22 TrYbe and IL-22R1 have an overlapping binding site.

TABLE 32
Binding responses of IL22 and IL22R1 in the absence and in the
presence of IL13/IL22 TrYbe antibody.

| | | Human | Human | Human | Human |
|---|---|---|---|---|---|
| | Capture | IL-22 | IL-22 | IL-22R1 | IL-22R1 |
| | Level | concen- | binding | concen- | Binding |
| Capture | (RU) | tration | (RU) | tration | (RU) |
| Buffer | 0.0 | 50 nM | 1.9 | 50 nM | −0.1 |
| IL13/IL22 TrYbe | 301.1 | 0 nM | 1.3 | 0 nM | 0.6 |
| IL13/IL22 TrYbe | 298.1 | 50 nM | 54.0 | 0 nM | 0.2 |
| IL13/IL22 TrYbe | 297.2 | 0 nM | 1.2 | 50 nM | 0.5 |
| IL13/IL22 TrYbe | 296.4 | 50 nM | 53.5 | 50 nM | 0.2 |

### Example 27. Biacore Affinity and Simultaneous Binding of Antigen Targets of IL13/IL22 TrYbe

IL13/IL22 TrYbe was tested for affinity against human, cynomolgus and mouse IL22, IL13 and albumin according to the method described below:

The assay format was capture of the IL13/IL22 TrYbe by immobilized anti human IgG-F(ab′)₂ then titration of Human IL22, IL13 and albumin over the captured surface. BIA (Biomolecular Interaction Analysis) was performed using T200 (GE Healthcare). Affinipure IgG-F(ab′)₂ Fragment goat anti-human IgG, F(ab′)₂ fragment specific (Jackson ImmunoResearch) was immobilized on a CM5 Sensor Chip via amine coupling chemistry to a capture level of ˜5000 response units (RUs). HBS-EP+buffer (10 mM HEPES pH 7.4, 0.15 M NaCl, 3 mM EDTA, 0.05% Surfactant P20, GE Healthcare) was used as the running buffer with a flow rate of 10 μL/min. A 10 μL injection of IL13/IL22 TrYbe at 0.5 μg/mL was used for capture by the immobilized anti-human IgG-F(ab′)₂. Human, cynomolgus or mouse IL22, IL13 and albumin were titrated over the captured IL13/IL22 TrYbe at various concentrations (10 nM to 0.3125 nM, 10 nM to 0.3125 nM and 100 nM to 3 nM for IL22, IL13 and albumin respectively) at a flow rate of 30 μL/min.

The surface was generated by 2×10 μL injections of 50 mM HCl, interspersed by a 10 μL injection of 5 mM NaOH at flowrate of 10 μL/min. Background subtraction binding curves were analyzed using the T200 evaluation software (version 3.0) following standard procedures. Kinetic parameters were determined from the fitting algorithm. The results are shown in Table 33. Results for the affinity of the TrYbe molecule with 11070 gL7gH16 IL22 domain binding to human IL-22 are summarized in Table 34

TABLE 33
Binding affinity of IL13/IL22 TrYbe molecule for IL13, IL22 and albumin

| Sample | Mean KD | |
|---|---|---|
| Human IL-22 | Less than 100 | pM |
| Human IL-13 | Less than 100 | pM |
| Human Albumin | 1 to 3 | nM |
| Cyno IL-22 | Less than 100 | pM |
| Cyno IL-13 | Less than 250 | pM |
| Cyno Albumin | 1 to 3 | nM |
| Mouse IL-22 | 14 to 25 | nM |
| Mouse IL-13 | No Binding | |
| Mouse Albumin | 5 to 6 | nM |

TABLE 34
Binding Affinity of TrYbe Molecule with 11070 gL 7gH16 IL22
domain binding to human IL22

| Sample | Mean KD |
|---|---|
| Human IL-22 | Less than 100 pM |

The simultaneous binding of IL22, IL13 and albumin to IL13/IL22 TrYbe was assessed by SPR using a Biacore T200 (GE Healthcare). The IL13/IL22 TrYbe construct was captured to the sensor chip by immobilised anti-human IgG-F(ab′)2 then human or cynomolgus IL22 (10 nM), IL13(10 nM) and albumin (100 nM) alone or a mixed solution with a final concentration of 10 nM IL22, 10 nM IL13 and 100 nM albumin were injected over the captured IL13/IL22 TrYbe.

For both human and cynomolgus analytes, the binding response for the combined IL22, IL13 and albumin solution was equivalent to the sum of the response of the independent injections, as shown in Tables 35 and 36. This confirms that IL13/IL22 TrYbe is capable of simultaneous binding to either human or cynomolgus IL22, IL13 and albumin.

TABLE 35
Simultaneous Binding of IL13/IL22 TrYbe to Human IL22, IL13 and
Albumin (Experiment I)

| | Binding, | Binding, | Binding, | |
|---|---|---|---|---|
| | n = 1 | n = 2 | n = 3 | Mean |
| Analyte | (RU) | (RU) | (RU) | (%) |
| Human IL13 | 10.0 | 10.3 | 10.7 | |
| Human IL22 | 20.1 | 16.5 | 18.8 | |
| Human Albumin | 45.0 | 33.8 | 47.7 | |
| IL13 + IL22 + Albumin Mixture | 74.9 | 61.5 | 81.4 | |
| Sum of Individual Binding | 75.1 | 60.6 | 77.2 | |
| Responses | | | | |
| Binding of Mixture as a | 99.7 | 101.5 | 105.4 | 102.2 |
| Percentage of Individual | | | | |
| Binding Responses (%) | | | | |

TABLE 36
Simultaneous Binding of IL13/IL22 TrYbe to cynomolgus IL22, IL13,
and albumin (Experiment II)

| | Binding, | Binding, | Binding, | |
|---|---|---|---|---|
| | n = 1 | n = 2 | n = 3 | Mean |
| Analyte | (RU) | (RU) | (RU) | (%) |
| Human IL13 | 15.0 | 14.9 | 16.1 | |
| Human IL22 | 14.5 | 15.7 | 18.5 | |
| Human Albumin | 37.2 | 38.4 | 41.9 | |
| IL13 + IL22 + Albumin Mixture | 65.0 | 65.8 | 72.3 | |
| Sum of Individual Binding | 66.7 | 69.0 | 76.5 | |
| Responses | | | | |
| Binding of Mixture as a | 97.5 | 95.4 | 94.5 | 95.8 |
| Percentage of Individual | | | | |
| Binding Responses (%) | | | | |

### Example 28. IL13/IL22 TrYbe and IL13/IL22 KiH Molecules in Primary Human Keratinocyte Assay

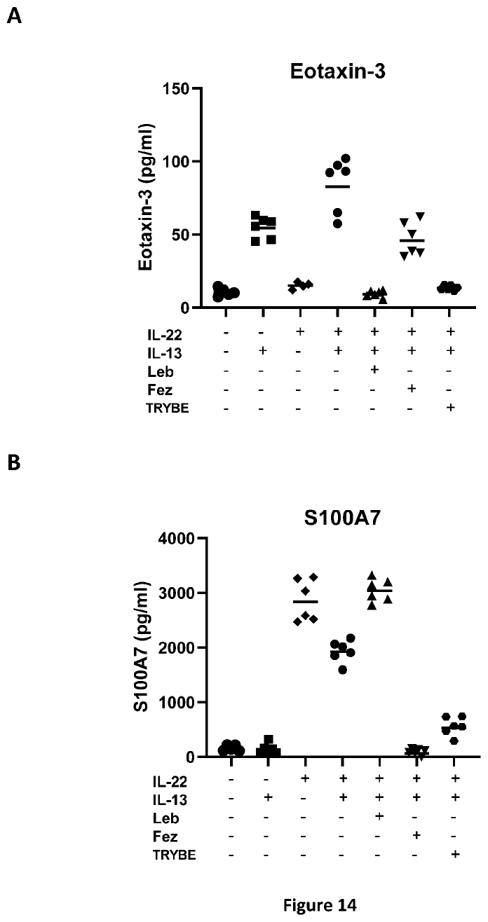

IL13/IL22 TrYbe multi-specific antibody and IL13/IL22 Knobs in Holes (KiH) bi-specific were tested in an in vitro cell assay against activity of human IL13 (R&D Systems, cat no #213-ILB-025) and IL22 (in house protein). The primary human neonatal epidermal keratinocytes from foreskin (NHEK) were ethically sourced from donors (Promocell, cat no #C-12001), expanded in culture and used in the assay. NHEK cells respond to IL13 stimulation and IL22 stimulation by secretion of soluble molecules that can be detected in cell supernatants. IL13 stimulation resulted in an increase of eotaxin-3 (CCL-26, FIG. 14A) and IL22 stimulation resulted in increase of S100A7 (psoriasin, FIG. 14B). Those biomarkers have been used in the assay to assess IL13/IL22 TrYbe activity.

NHEK cells from three donors at passage 2 or 3 were plated at 1×10⁴ cells per well in dermal basal media (LGC, cat no #ATCC-PCS-200-030) containing keratinocyte growth kit (LGC, ca no #ATCC-PCS-200-040) in 48-well plates (Corning, Costar® Clear TC-treated Plates, cat no #3548) pre-coated with extracellular matrix (TheromoFisher, cat no #R011K). Keratinocytes were cultured in standard conditions (37° C., 5% CO2, 100% humidity) until they reached confluence. On day 3 growth medium was aspirated from all wells and cells were washed with 200 IA basal dermal media to remove any dead cells and growth factors. IL13/IL22 TrYbe was preincubated with 100 ng/ml IL13 and IL22 at concentrations from 100 to 0.01 nM (10000-1 ng/ml, batch no #PB7916 and PB8056) in dermal basal media at 37° C. for 30 minutes. IL13/IL22 bi-specific molecules in KiH formats, IL13/22 (IL13 H/IL 22 K) and IL22/IL13 (IL13 K/IL 22 H), were preincubated with 100 ng/ml of IL13 and IL22 at concentrations from 100 to 0.01 nM (15000-1.5 ng/ml) in dermal basal media at 37° C. for 30 minutes. Fezakinumab (anti-IL22 antibody, produced in house, batch no #BSN.9787.hIgG4.801) and Lebrikizumab (anti-IL13 antibody produced in house, batch no #BSN.9874.hIgG4.983) at 100 nM (15000 ng/ml) were also preincubated with 100 ng/ml of IL13 and IL22 in dermal basal media at 37° C. for 30 minutes. After pre-incubation, the antibody/cytokine solution was transferred to cells. After 48 hrs of stimulation, supernatants were collected and levels of eotaxin-3 were measured using MSD (Meso Scale Diagnostics, cat no #K15067L-2) and S100A7 using ELISA (LSBio, cat no #LS-F50031).

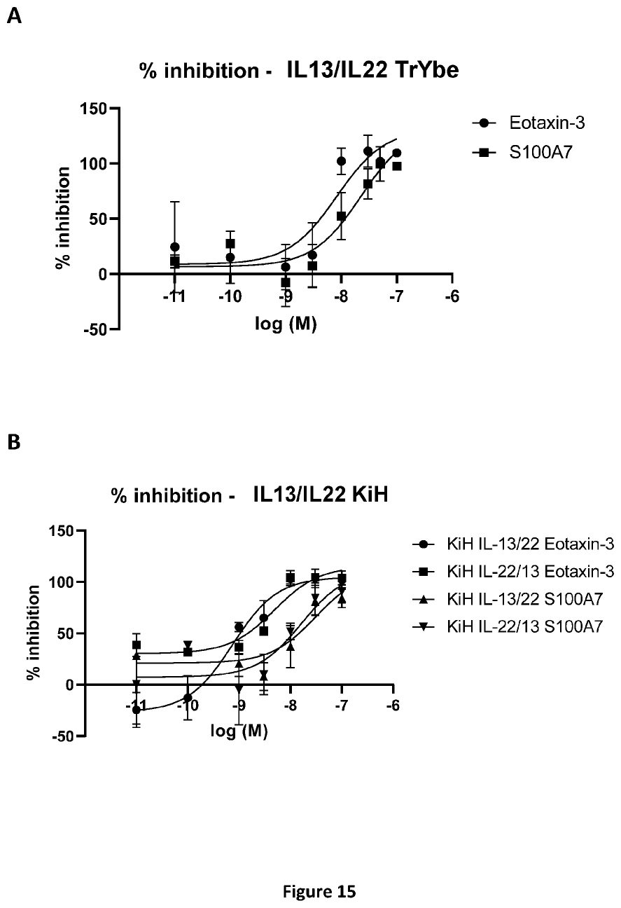

An increase in eotaxin-3 was measured post IL13 and IL13/IL22 stimulations (FIG. 14A). IL-22 stimulation alone did not induce Eotaxin-3 secretion. Lebrikizumab alone at 100 nM showed full inhibition of eotaxin-3 secretion induced by IL13/IL22 stimulation whereas Fezakinumab alone at 100 nM did not fully inhibit eotaxin-3 levels (FIG. 14A). This shows that Eotaxin-3 secretion in this assay in dependent on IL-13 stimulation only. IL13/IL22 TrYbe antibody at 25 nM showed full inhibition of eotaxin-3 (FIG. 14A). IL13/IL22 TrYbe also showed a concentration dependent inhibition of eotaxin-3 indicating that the anti-IL13 arm of IL13/IL22 TrYbe neutralizes IL13 activity (FIG. 15A). KiH molecules, in both IL13/22 and IL22/IL13 formats, also showed concentration dependent inhibition of eotaxin-3 (FIG. 15B) with efficacy comparable to IL13/IL22 TrYbe.

An increase in S100A7 was measured post IL22 and IL13/22 stimulations (FIG. 14B). IL-13 stimulation alone did not induce S100A7 secretion. Fezakinumab alone at 100 nM fully inhibited S100A7 secretion induced by IL13/IL22. Lebrikizumab alone at 100 nM did not inhibit S100A7 (FIG. 14B). IL13/IL22 TrYbe at 25 nM showed successful inhibition of IL13/IL22 induced S100A7 secretion (FIG. 14B). IL13/IL22 TrYbe showed a concentration dependent inhibition of S100A7 indicating that the anti-IL22 arm of IL13/IL22 TrYbe neutralizes IL22 activity (FIG. 15A). IL13/IL22 bi-specific KiH, in both IL13K/22H and IL22K/IL13H formats, showed concentration dependent inhibition of S100A7 (FIG. 15B) with efficacy equivalent to IL13/IL22 TrYbe.

In conclusion, both IL13/IL22 TrYbe and IL13/IL22 KiH bi-specific formats tested in the human primary keratinocyte assay showed simultaneous and concentration dependent inhibition of IL13 and IL22 activity. The results are summarized on FIGS. 14 and 15.

### Example 29. COLO205 IL-10 Release Assay for IL13/IL22 TrYbe

The antibody was tested in an in vitro cell assay for activity against human IL22. The COLO205 cell line is a human colorectal cancer epithelial cell line. IL22 binds to IL22R1 and IL-10R2 on the cell surface to induce STAT3 phosphorylation and downstream cytokine release (e.g. IL-10). In this assay, COLO205 cells were stimulated with IL22 with or without anti-IL22 antibodies. The resultant IL-10 response is then measured in the cell culture supernatant using a homogenous time-resolved FRET (HTRF) kit (Cisbio).

COLO205 cells were seeded at 25000 cells per well in tissue culture treated flat bottomed 96 well plates. Human IL22 (final assay concentration 30 pM) was pre-incubated with antibody (final assay concentration 3 nM-1.4 pM) at 37° C. for one hour. The antibody/cytokine complexes were then transferred to the COLO205 cells and incubated for 48 hours at 37° C., 5% CO2. Cell-free cell culture supernatants were then collected and stored at −80° C. Cell culture supernatants were defrosted on ice and the levels of IL-10 were determined by HTRF. All samples were run in duplicate in each repetition of the assay.

The results confirm that IL13/IL22 TrYbe inhibits the IL22-induced IL-10 response of COLO205 cells in the COLO205 IL-10 release assay. Two purifications of IL13/IL22 TrYbe were tested; PB8056 and PB7916. PB8056 had an IC50 of 36.6 pM and PB7916 had an IC50 of 34.0 pM, as determined by the geometric mean of 4 occasions of the assay (table 37). These measurements are deemed reliable as the range of the measured IC50 in each repetition of the assay was found to vary by less than threefold in each instance.

TABLE 37
IL13/IL22 TrYbe in the COLO205 IL-10 release assay

| | | | Maximum % inhibition | | | |
|---|---|---|---|---|---|---|
| | IC₅₀ (M) | | Arithmetic | | Hill Slope | |
| | Geomean | Range | mean | Range | Geomean | Range |
| IL13/IL22 TrYbe | 3.66e−11 | 3.42e−11 | 100.4 | 99.2 | 1.270 | 1.184 |
| PB8056 (N = 4) | | 4.15e−11 | | 101.2 | | 1.409 |
| IL13/IL22 TrYbe | 3.40e−11 | 2.44e−11 | 100.0 | 99.4 | 1.377 | 1.294 |
| PB7916 (N = 4) | | 4.60e−11 | | 100.6 | | 1.504 |

### Example 30. COLO205 IL-10 Release Assay for KiH Bi-Specific

The knob in hole bispecific antibodies were tested in an in vitro cell assay for activity against human IL22. COLO205 cell line is a human colorectal cancer epithelial cell line. IL22 binds to IL22R1 and IL-10R2 on the cell surface to induce STAT3 phosphorylation and downstream cytokine release (e.g. IL-10). In this assay, COLO205 cells were stimulated with IL22 with or without anti-IL22 antibodies. The resultant IL-10 response is then measured in the cell culture supernatant using a homogenous time-resolved FRET (HTRF) kit (Cisbio).

COLO205 cells were seeded at 25000 cells per well in tissue culture treated flat bottomed 96 well plates. Human IL22 (final assay concentration 30 pM) was pre-incubated with antibody (final assay concentration 3 nM-1.4 pM) at 37° C. for one hour. The antibody/cytokine complexes were then transferred to the COLO205 cells and incubated for 48 hours at 37° C., 5% CO2. Cell-free cell culture supernatants were then collected and stored at −80° C. Cell culture supernatants were defrosted on ice and the levels of IL-10 were determined by HTRF. Samples were run in duplicate.

2 batches of purifications for each KiH molecule were tested: IL13K/IL22H (PB8920 and PB8841) and IL13H/IL22K (PB8842 and PB8919).

### PB8919 Knob in Hole Bispecific Results

PB8919 knob in hole bispecific has anti-IL22 on the knob arm and anti-IL13 on the hole arm. The results confirm that PB8919 inhibits the IL22-induced IL-10 response of COLO205 cells in the COLO205 IL-10 release assay. PB8919 had an IC50 of 57.3 pM, as determined by the geometric mean of 2 occasions of the assay (Table 38). These measurements are deemed reliable as the range of the measured IC50 in each repetition of the assay was found to vary by less than threefold in each instance.

TABLE 38
PB8919 in the COLO205 IL-10 release assay.

| | | | Maximum % inhibition | | | |
|---|---|---|---|---|---|---|
| | IC₅₀ (M) | | Arithmetic | | Hill Slope | |
| | Geomean | Range | mean | Range | Geomean | Range |
| PB8919 | 5.73E−11 | 5.35E−11 | 101.0 | 98.2 | 1.509 | 1.199 |
| (N = 2) | | 6.14E−11 | | 103.8 | | 1.900 |

### PB8920 Knob in Hole Bispecific Results

PB8920 knob in hole bispecific has anti-IL13 on the knob arm and anti-IL22 on the hole arm. The results confirm that PB8920 inhibits the IL22-induced IL-10 response of COLO205 cells in the COLO205 IL-10 release assay. PB8920 had an IC50 of 63.8 pM, as determined by the geometric mean of 2 occasions of the assay (Table 39). These measurements are deemed reliable as the range of the measured IC50 in each repetition of the assay was found to vary by less than threefold in each instance.

TABLE 39
PB8920 in the COLO205 IL-10 release assay.

| | | | Maximum % inhibition | | | |
|---|---|---|---|---|---|---|
| | IC₅₀ (M) | | Arithmetic | | Hill Slope | |
| | Geomean | Range | mean | Range | Geomean | Range |
| PB8920 | 6.38E−11 | 5.85E−11 | 101.8 | 100.9 | 1.330 | 1.321 |
| (N = 2) | | 6.95E−11 | | 102.6 | | 1.339 |

### PB8841 Knob in Hole Bispecific Results

PB8841 knob in hole bispecific has anti-IL13 on the knob arm and anti-IL22 on the hole arm. The results confirm that PB8841 inhibits the IL22-induced IL-10 response of COLO205 cells in the COLO205 IL-10 release assay. PB8841 had an IC50 of 65.9 pM, as determined by the geometric mean of 2 occasions of the assay (Table 40). These measurements are deemed reliable as the range of the measured IC50 in each repetition of the assay was found to vary by less than threefold in each instance.

TABLE 40
PB8841 in the COLO205 IL-10 release assay.

| | | | Maximum % inhibition | | | |
|---|---|---|---|---|---|---|
| | IC₅₀ (M) | | Arithmetic | | Hill Slope | |
| | Geomean | Range | mean | Range | Geomean | Range |
| PB8841 | 6.59E−11 | 6.40E−11 | 99.8 | 97.4 | 1.593 | 1.371 |
| (N = 2) | | 6.79E−11 | | 102.2 | | 1.851 |

### PB8842 Knob in Hole Bispecific Results

PB8842 knob in hole bispecific has anti-IL22 on the knob arm and anti-IL13 on the hole arm. The results confirm that PB8842 inhibits the IL22-induced IL-10 response of COLO205 cells in the COLO205 IL-10 release assay. PB8842 had an IC50 of 71.3 pM, as determined by the geometric mean of 2 occasions of the assay (Table 41). These measurements are deemed reliable as the range of the measured IC50 in each repetition of the assay was found to vary by less than threefold in each instance.

TABLE 41
PB8842 in the COLO205 IL-10 release assay.

| | | | Maximum % inhibition | | | |
|---|---|---|---|---|---|---|
| | IC₅₀ (M) | | Arithmetic | | Hill Slope | |
| | Geomean | Range | mean | Range | Geomean | Range |
| PB8842 | 7.13E−11 | 6.89E−11 | 96.6 | 93.7 | 1.859 | 1.734 |
| (N = 2) | | 7.37E−11 | | 99.5 | | 1.994 |

### Example 30. STAT-6 Reporter Assay

The knob in hole bispecific antibodies IL13K/IL22H and IL13H/IL22K (2 protein purifications for each) were tested in an in vitro cell assay for activity against human IL13 response that is generated by stimulating HEK-Blue™ IL-4/IL13 cells with exogenously added human IL13.

HEK-Blue™ IL-4/IL13 cells allow the detection of bioactive IL-4/IL13 by monitoring the activation of the STAT-6 pathway induced by IL-4/IL13. These cells were generated by the stable transfection of HEK293 cells with the human STAT6 gene and a STAT6-inducible SEAP (secreted embryonic alkaline phosphatase) reporter gene. The secreted SEAP is measured using a QUANTI-Blue™ (InvivoGen) detection medium.

HEK-Blue™ IL-4/IL13 cells were seeded at a cell density of 5.0E+05 cells per well in a tissue culture treated flat bottomed 96 well plate and incubated for 24 hours at 37° C., 5% CO2.

Human IL13 (final assay concentration 20 pM) was pre-incubated with antibody (final assay concentration 1 nM-0.05 pM) at 37° C. for one hour. The antibody/cytokine complexes were then transferred to the HEK-Blue TM IL-4/IL13 cells and incubated for a further 24 hours at 37° C., 5% CO2. Cell-free cell culture supernatants were then collected in a tissue culture treated flat bottomed 96 well plate and add QUANTI-Blue™ (InvivoGen), and the levels of SEAP release were determined by reading the optical density at 630 nm absorbance settings using a BioTek® Synergy™ reader with Gen5™ software.

2 batches of purified proteins for each KiH molecule were tested: IL13K/IL22H (PB8920 and PB8841) and IL13H/IL22K (PB8842 and PB8919).

### PB8920 IL13K/IL22H Bispecific Results

The results confirm that IL13K/IL22H knob in hole bispecific format that has an anti IL13 on the knob and anti IL22 on the hole arm inhibits the IL13 response of HEK-Blue™ IL-4/IL13 cells in the STAT-6 reporter assay generating an IC50 of 2.9 pM as determined by the geometric mean of 2 occasions of the assay (table 42). These measurements are deemed reliable as the range of the measured IC50 in each repetition of the assay was found to vary by less than threefold in each instance.

TABLE 42
PB8920 IL13K/IL22H bispecific in the STAT-6 reporter assay.

| | IC₅₀ (pM) N = 2 | | Maximum % inhibition | | | |
|---|---|---|---|---|---|---|
| | | Concatenated | Arithmetic | | Hill Slope | |
| | Geomean | IC50 values | mean | Range | Geomean | Range |
| KiH | 2.9 | 2.1- | 107.6 | 106.3- | 1.8 | 1.6- |
| bispecific | | 4.0 | | 109.0 | | 2.1 |

### PB8841 IL13K/IL22H Bispecific Results

The results confirm that IL13K/IL22H knob in hole bispecific format that has an anti IL13 on the knob and anti IL22 on the hole arm inhibits the IL13 response of HEK-Blue™ IL-4/IL13 cells in the STAT-6 reporter assay generating an IC50 of 3.4 pM as determined by the geometric mean of 2 occasions of the assay (table 48). These measurements are deemed reliable as the range of the measured IC50 in each repetition of the assay was found to vary by less than threefold in each instance.

TABLE 43
PB8841 IL13K/IL22H bispecific in the STAT-6 reporter assay.

| | IC₅₀ (pM) N = 2 | | Maximum % inhibition | | | |
|---|---|---|---|---|---|---|
| | | Concatenated | Arithmetic | | Hill Slope | |
| | Geomean | IC50 values | mean | Range | Geomean | Range |
| KiH | 3.4 | 1.9- | 107.9 | 106.4- | 1.6 | 1.5- |
| bispecific | | 6.1 | | 109.4 | | 1.8 |

### PB8842 IL13H/IL22K Bispecific Results

IL13H/IL22K bispecific has anti-IL22 on the knob arm and anti-IL13 on the hole arm. The results confirm that IL13H/IL22K inhibits the IL13 response of HEK-Blue™ IL-4/IL13 cells in the STAT-6 reporter assay generating an IC50 of 4.9 pM as determined by the geometric mean of 2 occasions of the assay (table 44). These measurements are deemed reliable as the range of the measured IC50 in each repetition of the assay was found to vary by less than threefold in each instance.

TABLE 44
PB8842 IL13H/IL22K bispecific in the STAT-6 reporter assay.

| | IC₅₀ (pM) N = 2 | | Maximum % inhibition | | | |
|---|---|---|---|---|---|---|
| | | Concatenated | Arithmetic | | Hill Slope | |
| | Geomean | IC50 values | mean | Range | Geomean | Range |
| KiH | 4.9 | 4.6- | 106.4 | 104.5- | 2.8 | 1.7- |
| bispecific | | 5.2 | | 108.3 | | 4.7 |

### PB8919 IL13H/IL22K Bispecific Results

IL13H/IL22K bispecific has anti-IL22 on the knob arm and anti-IL13 on the hole arm. The results confirm that IL13H/IL22K inhibits the IL13 response of HEK-Blue™ IL-4/IL13 cells in the STAT-6 reporter assay generating an IC50 of 2.3 pM as determined by the geometric mean of 2 occasions of the assay (table 45). These measurements are deemed reliable as the range of the measured IC50 in each repetition of the assay was found to vary by less than threefold in each instance.

TABLE 45
Table of data for PB8919 IL13H/IL22K bispecific in the STAT-6 reporter assay.

| | IC₅₀ (pM) N = 2 | | Maximum % inhibition | | | |
|---|---|---|---|---|---|---|
| | | Concatenated | Arithmetic | | Hill Slope | |
| | Geomean | IC50 values | mean | Range | Geomean | Range |
| KiH | 2.3 | 2- | 109.3 | 106- | 1.2 | 0.9- |
| bispecific | | 2.7 | | 112.8 | | 1.6 |

### Example 31. Effects of a Combination of an Anti-IL13 and Anti-IL22 Antibodies, and IL13/IL22 TrYbe in a Full Thickness Reconstituted Skin Tissue Model

To evaluate the effect of IL13/IL22 TrYbe mediated dual blockade of IL13 and IL22 induced epidermal thickness and aberrant keratinocyte differentiation, a full thickness skin tissue model was used.

EpiDermFT™ (MatTek Corporation) full thickness reconstituted skin tissue was equilibrated in EFT-400-ASY assay medium (MatTek Corporation) at 37° C. 5% CO2 in a cell culture incubator overnight.

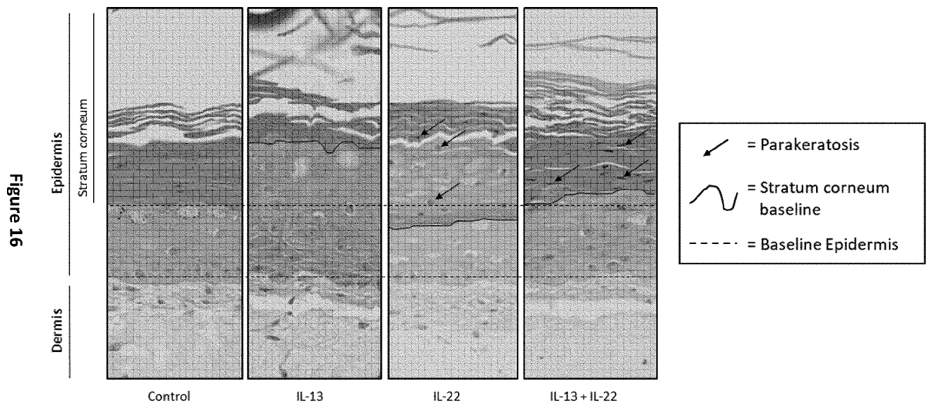

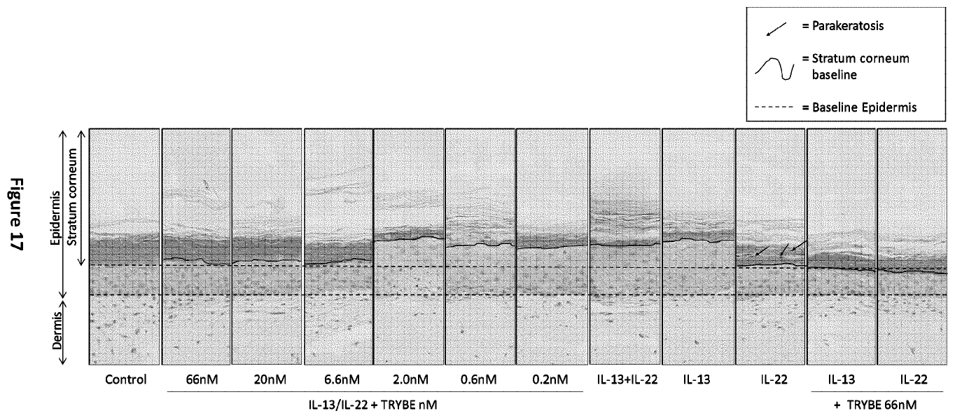

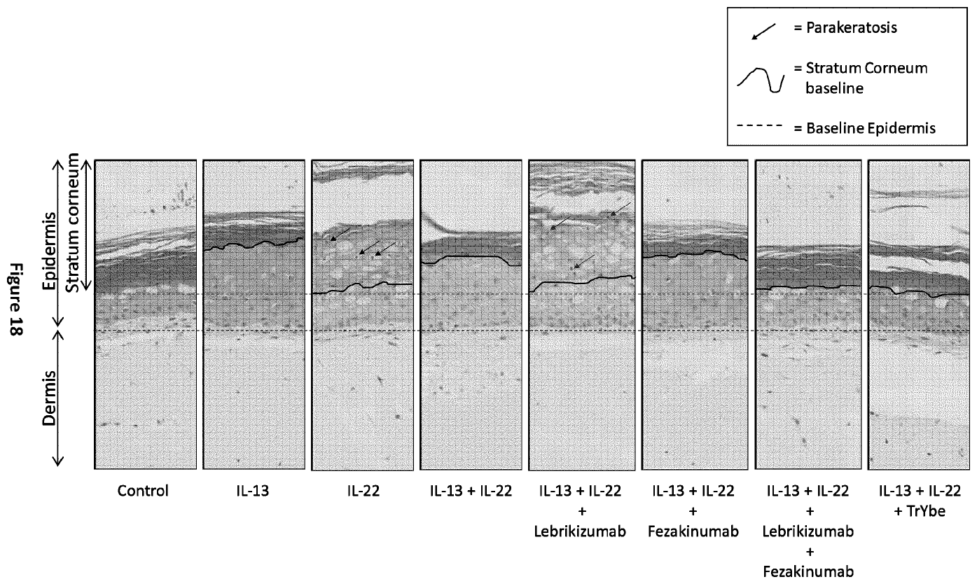

On Day 0, media was removed from wells and replaced with 2.5 ml of the following conditions into the respective wells and plates incubated at 37° C. 5% CO₂:
Media alone, IL13 (R & D systems) or IL22 (in house produced) or IL13/IL22 combination at 100 ng/ml final concentration in EFT-400ASY media (FIG. 16)
Media alone or IL13/IL22 combination at 100 ng/ml final concentration in EFT-400ASY media with a titration of IL13/IL22 TrYbe from 66 nM to 0.2 nM. IL13 or IL22 alone with/without 66 nM IL13/IL22 TrYbe. (FIG. 17)
Media alone, IL13 or IL22 or IL13/IL22 combination at 100 ng/ml final concentration in EFT-400ASY media. IL13/IL22 combination with 66 nM Lebrikizumab (anti-IL13 antibody) or Fezakinumab (anti-IL22 antibody) or Lebrikizumab/Fezakinumab combination or IL13/IL22 TrYbe alone (FIG. 18).

Conditions were refreshed every 2 days (0,2,4,6) and experiments stopped on day 7.

Tissues were removed from transwells, bisected using a scalpel on a sterile petri dish, and placed in 10% Neutral Buffered Formalin (Sigma) in preparation for Histological analysis with Haematoxylin and Eosin staining on 41,tm section.

The results show that IL13 and IL22 individually increase epidermal thickness (FIG. 16). Aberrant keratinocyte differentiation is also observed following IL22 treatment, illustrated by increased parakeratosis and thickening of the stratum corneum (the uppermost layer of the epidermis). The combined effect of IL13 and IL22 is greater than either cytokine alone, suggesting an additive or synergistic effect between IL13 and IL22 (FIG. 16).

The results demonstrate that IL13/IL22 TrYbe inhibits IL13 and IL22 induced changes and enables a normal skin phenotype to be maintained (FIG. 17). IL13/IL22 TrYbe also successfully inhibits epidermal thickening and aberrant keratinocyte differentiation as a result of combined IL13/IL22 stimulation in a concentration dependent manner (FIG. 17).

The results also demonstrate that inhibition of both IL13 and IL22 (by the combination of the anti-IL13 and anti-IL22 antibodies or IL13/IL22 multi-specific antibody) is required to ameliorate IL13/IL22 induced epidermal thickening and aberrant keratinocyte differentiation (FIG. 18), as inhibition of either cytokine alone was unable to maintain a normal skin phenotype when compared to control (media alone). The data shows that IL13/IL22 TrYbe was able to fully inhibit IL13/IL22 induced epidermal thickening and aberrant keratinocyte differentiation, demonstrating dual blockade of IL13 and IL22.

### Example 32. IL22 Phospho STAT3 Method

Hacat cells were added to a 96 well flat bottomed tissue culture plate at 150,000 cells per well in 100 μls of DMEM+10% FBS+2 mM L-glutamine per well and incubated at 37 degrees and 5% CO₂ overnight. An anti-IL22 antibody was diluted in mock supernatant media to a final assay concentration of 18.75 nM and 60 μls was added to columns 1 and 12 of a 96 well polypropylene V bottomed plate as the minimum signal control. 60 μls of mock supernatant media was added to cols 2 and 11 of the 96 well polypropylene V bottomed plate as the maximum signal controls. Samples were titrated 1:3 into mock supernatant media leaving a final volume of 60 μls in columns 3-10 of the 96 well polypropylene V bottomed plate. 30 μls IL22 solution was added to all wells giving a final assay concentration of 30 ng/ml FAC. The plates were preincubated for 1 hr at 37 degrees. 75 μls of the culture media from cell culture plates to leave 25 μls in the plate. 75 μls of sample titration/controls+il22 was transferred to the cell plate. These plates were Incubated at 37 degrees for 30 minutes. Supernatants were removed. Cisbio STAT3 Phospho Y705 kit was used to lyse the remaining cells and generate HTRF signals from the lysate for each well. The plates were sealed and incubated at room temperature on a shaker over night for 18 hours. The well signals were measured using a HTRF protocol on the Synergy Neo 2 plate reader. Both 11041 and 11070 antibodies demonstrated clear inhibition of IL22-induced STAT3 phosphorylation.

All references cited herein, including patents, patent applications, papers, textbooks and the like, and the references cited therein, to the extent that they are not already, are hereby incorporated herein by reference in their entirety.

1. A multi-specific antibody comprising at least two antigen-binding domains, wherein one antigen binding domain binds to IL13 (IL13-binding domain) and the second antigen-binding domain binds to IL22 (IL22-binding domain).

2. The multi-specific antibody according to claim 1, wherein IL13 is human and/or cynomolgus IL13, and IL22 is human and/or cynomolgus IL22.

3. The multi-specific antibody according to claim 1, wherein each antigen-binding domain comprises two antibody variable domains.

4. (canceled)

5. The multi-specific antibody according to claim 1 wherein the IL13-binding domain and the IL22-binding domain are independently selected from a Fab, scF, Fv, dsFv and dsscFv.

6. The multi-specific antibody according to claim 1, wherein said multi-specific antibody neutralizes one or more IL13 and/or IL22 activities.

7. The multi-specific antibody according to claim 6, wherein said antibody is capable of inhibiting or attenuating IL22 binding to IL22 receptor 1 (IL22R1).

8. The multi-specific antibody according to claim 6, wherein said antibody binds to a region on IL22 such that the binding sterically blocks the interaction between IL22 and IL22R1.

9. The multi-specific antibody according to claim 6, wherein said antibody is capable of inhibiting or attenuating IL22 binding to IL22 binding protein (IL22RA2).

10. The multi-specific antibody according to claim 6, wherein said antibody is capable of inhibiting or attenuating IL13 binding to IL13Ralpha1.

11-15. (canceled)

16. The multi-specific antibody according to claim 1 additionally comprising a third antigen binding domain that binds to serum albumin (albumin-binding domain).

17. The multi-specific antibody according to claim 16 comprising
a) a polypeptide chain of formula (Ia):
V_{H}—CH₁—X—V₁; and
b) a polypeptide chain of formula (IIa):
V_{L}—C_{L}—Y—V₂;
wherein:
V_{H} represents a heavy chain variable domain;
CH₁ represents domain 1 of a heavy chain constant region;
X represents a bond or linker;
Y represents a bond or linker;
V₁ represents a scFv, a dsscFv, or a dsFv;
V_{L} represents a light chain variable domain;
C_{L} represents a domain from a light chain constant region, such as Ckappa;
V₂ represents a scFv, a dsscFv or a dsFv;
wherein at least one of V1 or V2 is a dsscFv or a dsFv.

18. The multi-specific antibody according to claim 17, wherein:
V_{L} and V_{H} comprise the antigen-binding domain that binds to IL22,
V₂ comprises the antigen binding-domain that binds to IL13, and
V₁ comprises the antigen binding-domain that binds to serum albumin;

19. (canceled)

20. The multi-specific antibody according to claim 1, wherein the IL22-binding domain comprises a light chain variable region comprising:
a CDR-L1 comprising SEQ ID NO:8,
a CDR-L2 comprising SEQ ID NO:9, and
a CDR-L3 comprising SEQ ID NO:10;
and a heavy chain variable region comprising:
a CDR-H1 comprising SEQ ID NO:11,
a CDR-H2 comprising SEQ ID NO:12, and
a CDR-H3 comprising SEQ ID NO:13.

21. The multi-specific antibody according to claim 1, wherein the IL13-binding domain comprises a light chain variable region comprising:
a CDR-L1 comprising SEQ ID NO:22,
a CDR-L2 comprising SEQ ID NO:23, and
a CDR-L3 comprising SEQ ID NO:24;
and a heavy chain variable region comprising:
a CDR-H1 comprising SEQ ID NO:25,
a CDR-H2 comprising SEQ ID NO:26, and
a CDR-H3 comprising SEQ ID NO:27.

22. The multi-specific antibody according to claim 1, wherein the albumin-binding domain comprises a light chain variable region comprising:
a CDR-L1 comprising SEQ ID NO:40,
a CDR-L2 comprising SEQ ID NO:41, and
a CDR-L3 comprising SEQ ID NO:42;
and a heavy chain variable region comprising:
a CDR-H1 comprising SEQ ID NO:43,
a CDR-H2 comprising SEQ ID NO:44, and
a CDR-H3 comprising SEQ ID NO:45.

23. (canceled)

24. The multi-specific antibody according to claim 1, wherein the IL22-binding domain comprises a light chain variable region comprising the sequence given in SEQ ID NO:14 and a heavy chain variable region comprising the sequence given in SEQ ID NO:16.

25. (canceled)

26. The multi-specific antibody according to claim 1, wherein the IL22-binding domain is a Fab comprising a light chain comprising the sequence given in SEQ ID NO:18 and a heavy chain comprising the sequence given in SEQ ID NO:20.

27. The multi-specific antibody according to claim 1, wherein the IL13-binding domain comprises a light chain variable region comprising the sequence given in SEQ ID NO: 28 or 32 and a heavy chain variable region comprising the sequence given in SEQ ID NO:29 or 33.

28-31. (canceled)

32. The multi-specific antibody according to claim 1, wherein the IL13-binding domain is a scFv comprising the sequence given in SEQ ID NO:36 or a dsscFv comprising the sequence given in SEQ ID NO:38.

33. (canceled)

34. The multi-specific antibody according to claim 1, wherein
(i) the IL22-binding domain is a Fab comprising a light chain comprising the sequence given in SEQ ID NO:18 and a heavy chain comprising the sequence given in SEQ ID NO: 20; and
(ii) the IL13-binding domain is a scFv comprising the sequence given in SEQ ID NO:36 or a dsscFv comprising the sequence given in SEQ ID NO:38.

35-37. (canceled)

38. The multi-specific antibody according to claim 1, wherein the albumin-binding domain is a scFv comprising the sequence given in SEQ ID NO:54 or a dsscFv comprising the sequence given in SEQ ID NO:56.

39-42. (canceled)

43. The multi-specific antibody according to claim 17, comprising the sequence given in SEQ ID NO:60 and comprising the sequence given in SEQ ID NO: 64.

44. An isolated polynucleotide encoding the multi-specific antibody or a polypeptide chain thereof according to claim 1.

45. An expression vector carrying the polynucleotide of claim 44.

46. A host cell comprising the vector as defined in claim 45.

47. A method of producing the multi-specific antibody as defined in claim 1, comprising culturing a host cell comprising an expression vector carrying a polynucleotide encoding the multi-specific antibody under conditions permitting production of the antibody, and recovering the antibody produced.

48. A pharmaceutical composition comprising the multi-specific antibody as defined in claim 1 and a pharmaceutically acceptable adjuvant and/or carrier.

49-52. (canceled)

53. A method of treating or preventing an inflammatory skin condition, comprising administering a therapeutically effective amount of the multi-specific antibody according to claim 1 or a pharmaceutical composition comprising the multi-specific antibody and a pharmaceutically acceptable adjuvant and/or carrier to a patient in need thereof.

54. (canceled)

55. (canceled)

56. A pharmaceutical composition comprising an antibody that binds to IL13 and an antibody that binds to IL22.

57. The pharmaceutical composition according to claim 56, wherein the antibody that binds to IL13 neutralizes IL13, and the antibody that binds to IL22 neutralizes IL22.

58. The pharmaceutical composition according to claim 56, wherein each antibody comprises two antibody variable domains.

59-66. (canceled)

67. The pharmaceutical composition according to claim 56, wherein the antibody that binds to IL22 comprises
a light chain variable region comprising:
a CDR-L1 comprising SEQ ID NO:8,
a CDR-L2 comprising SEQ ID NO:9, and
a CDR-L3 comprising SEQ ID NO:10;
and a heavy chain variable region comprising:
a CDR-H1 comprising SEQ ID NO:11,
a CDR-H2 comprising SEQ ID NO:12, and
a CDR-H3 comprising SEQ ID NO:13.

68. The pharmaceutical composition according to claim 56, wherein the antibody that binds to IL13 comprises a light chain variable region comprising:
a CDR-L1 comprising SEQ ID NO:22,
a CDR-L2 comprising SEQ ID NO:23, and
a CDR-L3 comprising SEQ ID NO:24;
and a heavy chain variable region comprising:
a CDR-H1 comprising SEQ ID NO:25,
a CDR-H2 comprising SEQ ID NO:26, and
a CDR-H3 comprising SEQ ID NO:27.

69. (canceled)

70. The pharmaceutical composition according to claim 56, wherein the antibody that binds to IL22 comprises a light chain variable region comprising the sequence given in SEQ ID NO:14 and a heavy chain variable region comprising the sequence given in SEQ ID NO:16.

71. (canceled)

72. (canceled)

73. The pharmaceutical composition according to claim 56, wherein the antibody that binds to IL13 comprises a light chain variable region comprising the sequence given in SEQ ID NO: 28 and a heavy chain variable region comprising the sequence given in SEQ ID NO:29.

74-82. (canceled)

83. A method of treating or preventing an inflammatory skin condition, comprising administering a therapeutically effective amount of the pharmaceutical composition according to claim 56 to a patient in need thereof.

84-86. (canceled)

87. A method of treating an inflammatory skin condition in a subject in need thereof, the method comprising administering to the subject a therapeutically effective amount of a combination of an antibody that binds to and neutralizes IL13 and an antibody that binds to and neutralizes IL22.

88. (canceled)

89. (canceled)

90. The method of claim 87, wherein said inflammatory skin condition is psoriasis, psoriatic arthritis, contact dermatitis, chronic hand eczema or atopic dermatitis.

91. The method of claim 87, wherein each antibody of the combination is independently selected from a full length antibody, Fab, scFv, Fv, dsFv and dsscFv.

92. The method of claim 87, wherein each of the antibodies of the combination is provided as a pharmaceutical composition comprising one or more of a pharmaceutically acceptable excipient, diluent or carrier.


SEQUENCE LISTING (167 sequences)

SEQ ID NO: 1 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - protein, 146 aa, Homo sapiens
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 3 - protein, 172 aa, Artificial Sequence
  IL22 with His tag
MGSSHHHHHHSSGENLYFQGSQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 4 - protein, 153 aa, Artificial Sequence
  IL22 cleaved
GSQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 5 - protein, 146 aa, Homo sapiens [sequence omitted]

SEQ ID NO: 6 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 7 - protein, 609 aa, Homo sapiens [sequence omitted]

SEQ ID NO: 8 - protein, 11 aa, Artificial Sequence
  11041 CDRL1
QASEDIYTNLA

SEQ ID NO: 9 - protein, 7 aa, Artificial Sequence
  11041 CDRL2
WASTLAS

SEQ ID NO: 10 - protein, 14 aa, Artificial Sequence
  11041 CDRL3
QASVYGNAADSRYT

SEQ ID NO: 11 - protein, 10 aa, Artificial Sequence
  11041 CDRH1
GFSLSSYAMI

SEQ ID NO: 12 - protein, 16 aa, Artificial Sequence
  11041 CDRH2
IIDIEGSTYYASWAKG

SEQ ID NO: 13 - protein, 11 aa, Artificial Sequence
  11041 CDRH3
DRFVGVDIFDP

SEQ ID NO: 14 - protein, 112 aa, Artificial Sequence
  11041gL13 V-region
AVQLTQSPSSLSASVGDRVTITCQASEDIYTNLAWYQQKPGKAPKLLIYWASTLASGVPSRFSGSGSGTDFTLTISSLQPEDFATYYCQASVYGNAADSRYTFGGGTKVEIK

SEQ ID NO: 15 - DNA, 342 bp, Artificial Sequence
  11041gL13 V-region
gccgtccaactgactcagtccccgagctcactttccgcgagcgtgggagatcgcgtgaccattacgtgccaggcctcggaggacatctacaccaacctcgcctggtatcaacagaagcctggcaaagctcccaagctgttgatctactgggcctccactctggcctccggagtgccttcgcggttctccggttctggatcaggcaccgacttcaccctgacaatcagcagcctccagccggaagattttgccacttactactgccaagcatccgtctacgggaacgcagcggactccagatataccttcggcgggggaaccaaagtggagattaagcgtacg

SEQ ID NO: 16 - protein, 119 aa, Artificial Sequence
  11041gH14 V-region
EVQLVESGGGLVQPGGSLRLSCAVSGFSLSSYAMIWVRQAPGKGLEWIGIIDIEGSTYYASWAKGRFTISRDNSKNTVYLQMNSLRAEDTAVYYCARDRFVGVDIFDPWGQGTLVTVSS

SEQ ID NO: 17 - DNA, 357 bp, Artificial Sequence
  11041gH14 V-region
gaggtgcagctcgtggaaagcggaggaggactggtgcagccaggagggtccttgcggcttagctgtgccgtgtccggcttctccctgtcctcctacgccatgatctgggtccgccaagctcctgggaagggcctcgaatggattggtattatcgacatcgagggatcaacctactacgcctcgtgggccaagggacggttcaccatctcgcgggacaactccaagaacactgtgtatctgcagatgaacagcctgagggcagaagataccgccgtgtactactgcgcgagagatcgcttcgtgggcgtggacatctttgacccgtggggtcaaggcaccctggtcactgtctcgagc

SEQ ID NO: 18 - protein, 219 aa, Artificial Sequence
  Light chain (VL-CL) 11041gL13
AVQLTQSPSSLSASVGDRVTITCQASEDIYTNLAWYQQKPGKAPKLLIYWASTLASGVPSRFSGSGSGTDFTLTISSLQPEDFATYYCQASVYGNAADSRYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 657 bp, Artificial Sequence
  Light chain (VL-CL) 11041gL13
gccgtccaactgactcagtccccgagctcactttccgcgagcgtgggagatcgcgtgaccattacgtgccaggcctcggaggacatctacaccaacctcgcctggtatcaacagaagcctggcaaagctcccaagctgttgatctactgggcctccactctggcctccggagtgccttcgcggttctccggttctggatcaggcaccgacttcaccctgacaatcagcagcctccagccggaagattttgccacttactactgccaagcatccgtctacgggaacgcagcggactccagatataccttcggcgggggaaccaaagtggagattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 20 - protein, 222 aa, Artificial Sequence
  Heavy chain (VH-CH1) 11041gH14
EVQLVESGGGLVQPGGSLRLSCAVSGFSLSSYAMIWVRQAPGKGLEWIGIIDIEGSTYYASWAKGRFTISRDNSKNTVYLQMNSLRAEDTAVYYCARDRFVGVDIFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - DNA, 666 bp, Artificial Sequence
  Heavy chain (VH-CH1) 11041gH14
gaggtgcagctcgtggaaagcggaggaggactggtgcagccaggagggtccttgcggcttagctgtgccgtgtccggcttctccctgtcctcctacgccatgatctgggtccgccaagctcctgggaagggcctcgaatggattggtattatcgacatcgagggatcaacctactacgcctcgtgggccaagggacggttcaccatctcgcgggacaactccaagaacactgtgtatctgcagatgaacagcctgagggcagaagataccgccgtgtactactgcgcgagagatcgcttcgtgggcgtggacatctttgacccgtggggtcaaggcaccctggtcactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgt

SEQ ID NO: 22 - protein, 11 aa, Artificial Sequence
  650 CDRL1
KASQNINENLD

SEQ ID NO: 23 - protein, 7 aa, Artificial Sequence
  650 CDRL2
YTDILQT

SEQ ID NO: 24 - protein, 8 aa, Artificial Sequence
  650 CDRL3
YQYYSGYT

SEQ ID NO: 25 - protein, 10 aa, Artificial Sequence
  650 CDRH1
GYSFTSYYIH

SEQ ID NO: 26 - protein, 17 aa, Artificial Sequence
  650 CDRH2
RIGPGSGDINYNEKFKG

SEQ ID NO: 27 - protein, 7 aa, Artificial Sequence
  650 CDRH3
FHYDGAD

SEQ ID NO: 28 - protein, 106 aa, Artificial Sequence
  650 gL8 V-region (unmutated*)
DIQMTQSPSSLSASVGDRVTITCKASQNINENLDWYQQKPGKAPKLLIYYTDILQTGIPSRFSGSGSGTDYTLTISSLQPEDFATYYCYQYYSGYTFGQGTKLEIK

SEQ ID NO: 29 - protein, 116 aa, Artificial Sequence
  650 gH9 V-region (unmutated*)
EVQLVQSGAEVKKPGSSVKVSCKASGYSFTSYYIHWVRQAPGQGLEWMGRIGPGSGDINYNEKFKGRATFTVDKSTSTAYMELSSLRSEDTAVYYCARFHYDGADWGQGTLVTVSS

SEQ ID NO: 30 - DNA, 318 bp, Artificial Sequence
  650 gL8 V-region (unmutated*)
gacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggccagggcaccaagctggagatcaag

SEQ ID NO: 31 - DNA, 348 bp, Artificial Sequence
  650 gH9 V-region (unmutated*)
gaggtgcagctggtgcagtccggcgccgaggtgaagaagcccggctcctccgtgaaggtgtcctgcaaggcctccggctactccttcacctcctactacatccactgggtgaggcaggcccccggccagggcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctcc

SEQ ID NO: 32 - protein, 106 aa, Artificial Sequence
  650 gL8 V-region (mutated**)
DIQMTQSPSSLSASVGDRVTITCKASQNINENLDWYQQKPGKAPKLLIYYTDILQTGIPSRFSGSGSGTDYTLTISSLQPEDFATYYCYQYYSGYTFGCGTKLEIK

SEQ ID NO: 33 - protein, 116 aa, Artificial Sequence
  650 gH9 V-region (mutated**)
EVQLVQSGAEVKKPGSSVKVSCKASGYSFTSYYIHWVRQAPGQCLEWMGRIGPGSGDINYNEKFKGRATFTVDKSTSTAYMELSSLRSEDTAVYYCARFHYDGADWGQGTLVTVSS

SEQ ID NO: 34 - DNA, 318 bp, Artificial Sequence
  650 gL8 V-region (mutated**)
gacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggctgcggcaccaagctggagatcaag

SEQ ID NO: 35 - DNA, 348 bp, Artificial Sequence
  650 gH9 V-region (mutated**)
gaggtgcagctggtgcagtccggcgccgaggtgaagaagcccggctcctccgtgaaggtgtcctgcaaggcctccggctactccttcacctcctactacatccactgggtgaggcaggcccccggccagtgcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctcc

SEQ ID NO: 36 - protein, 242 aa, Artificial Sequence
  650 scFv (VH/VL) gH9gL8 (unmutated*)
EVQLVQSGAEVKKPGSSVKVSCKASGYSFTSYYIHWVRQAPGQGLEWMGRIGPGSGDINYNEKFKGRATFTVDKSTSTAYMELSSLRSEDTAVYYCARFHYDGADWGQGTLVTVSSGGGGSGGGGSGGGGSGGGGSDIQMTQSPSSLSASVGDRVTITCKASQNINENLDWYQQKPGKAPKLLIYYTDILQTGIPSRFSGSGSGTDYTLTISSLQPEDFATYYCYQYYSGYTFGQGTKLEIK

SEQ ID NO: 37 - DNA, 726 bp, Artificial Sequence
  650 scFv (VH/VL) gH9gL8 (unmutated*)
gaggtgcagctggtgcagtccggcgccgaggtgaagaagcccggctcctccgtgaaggtgtcctgcaaggcctccggctactccttcacctcctactacatccactgggtgaggcaggcccccggccagggcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctccggaggtggcggttctggcggtggcggttccggtggcggtggatcgggaggtggcggttctgacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggccagggcaccaagctggagatcaag

SEQ ID NO: 38 - protein, 242 aa, Artificial Sequence
  650 dsscFv (VH/VL) gH9gL8 (mutated**)
EVQLVQSGAEVKKPGSSVKVSCKASGYSFTSYYIHWVRQAPGQCLEWMGRIGPGSGDINYNEKFKGRATFTVDKSTSTAYMELSSLRSEDTAVYYCARFHYDGADWGQGTLVTVSSGGGGSGGGGSGGGGSGGGGSDIQMTQSPSSLSASVGDRVTITCKASQNINENLDWYQQKPGKAPKLLIYYTDILQTGIPSRFSGSGSGTDYTLTISSLQPEDFATYYCYQYYSGYTFGCGTKLEIK

SEQ ID NO: 39 - DNA, 726 bp, Artificial Sequence
  650 dsscFv (VH/VL) gH9gL8 (mutated**)
gaggtgcagctggtgcagtccggcgccgaggtgaagaagcccggctcctccgtgaaggtgtcctgcaaggcctccggctactccttcacctcctactacatccactgggtgaggcaggcccccggccagtgcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctccggaggtggcggttctggcggtggcggttccggtggcggtggatcgggaggtggcggttctgacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggctgcggcaccaagctggagatcaag

SEQ ID NO: 40 - protein, 12 aa, Artificial Sequence
  645 CDRL1
QSSPSVWSNFLS

SEQ ID NO: 41 - protein, 7 aa, Artificial Sequence
  645 CDRL2
EASKLTS

SEQ ID NO: 42 - protein, 11 aa, Artificial Sequence
  645 CDRL3
GGGYSSISDTT

SEQ ID NO: 43 - protein, 10 aa, Artificial Sequence
  645 CDRH1
GIDLSNYAIN

SEQ ID NO: 44 - protein, 16 aa, Artificial Sequence
  645 CDRH2
IIWASGTTFYATWAKG

SEQ ID NO: 45 - protein, 13 aa, Artificial Sequence
  645 CDRH3
TVPGYSTAPYFDL

SEQ ID NO: 46 - protein, 110 aa, Artificial Sequence
  645 VL-region (unmutated*)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 47 - protein, 121 aa, Artificial Sequence
  645 VH-region (unmutated*)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 48 - DNA, 330 bp, Artificial Sequence
  645 VL-region (unmutated*)
gacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggggcggtactaaggtggaaatcaaa

SEQ ID NO: 49 - DNA, 363 bp, Artificial Sequence
  645 VH-region (unmutated*)
gaggttcaactgcttgagtctggaggaggcctagtccagcctggagggagcctgcgtctctcttgtgcagtaagcggcatcgacctgagcaattacgccatcaactgggtgagacaagctccggggaagggtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagt

SEQ ID NO: 50 - protein, 110 aa, Artificial Sequence
  645 VL-region (mutated**)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK

SEQ ID NO: 51 - protein, 121 aa, Artificial Sequence
  645 VH-region (mutated**)
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 52 - DNA, 330 bp, Artificial Sequence
  645 VL-region (mutated**)
gacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaa

SEQ ID NO: 53 - DNA, 363 bp, Artificial Sequence
  645 VH-region (mutated**)
gaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagt

SEQ ID NO: 54 - protein, 251 aa, Artificial Sequence
  645 scFv (VH/VL) (unmutated*)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 55 - DNA, 753 bp, Artificial Sequence
  645 scFv (VH/VL) (unmutated*)
gaggttcaactgcttgagtctggaggaggcctagtccagcctggagggagcctgcgtctctcttgtgcagtaagcggcatcgacctgagcaattacgccatcaactgggtgagacaagctccggggaagggtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagtggaggtggcggttctggcggtggcggttccggtggcggtggatcgggaggtggcggttctgacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggggcggtactaaggtggaaatcaaa

SEQ ID NO: 56 - protein, 251 aa, Artificial Sequence
  645 dsscFv (VH/VL) (mutated**)
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK

SEQ ID NO: 57 - DNA, 753 bp, Artificial Sequence
  645 dsscFv (VH/VL) (mutated**)
gaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagtggaggtggcggttctggcggtggcggttccggtggcggtggatcgggaggtggcggttctgacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaa

SEQ ID NO: 58 - protein, 486 aa, Artificial Sequence
  11041gH14 HC- 645 (VH/VL) scFv (unmutated*)
EVQLVESGGGLVQPGGSLRLSCAVSGFSLSSYAMIWVRQAPGKGLEWIGIIDIEGSTYYASWAKGRFTISRDNSKNTVYLQMNSLRAEDTAVYYCARDRFVGVDIFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRT

SEQ ID NO: 59 - DNA, 1458 bp, Artificial Sequence
  11041gH14 HC- 645 (VH/VL) scFv (unmutated*)
gaggtgcagctcgtggaaagcggaggaggactggtgcagccaggagggtccttgcggcttagctgtgccgtgtccggcttctccctgtcctcctacgccatgatctgggtccgccaagctcctgggaagggcctcgaatggattggtattatcgacatcgagggatcaacctactacgcctcgtgggccaagggacggttcaccatctcgcgggacaactccaagaacactgtgtatctgcagatgaacagcctgagggcagaagataccgccgtgtactactgcgcgagagatcgcttcgtgggcgtggacatctttgacccgtggggtcaaggcaccctggtcactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagcggtggcggtggctccggaggtggcggttcagaggttcaactgcttgagtctggaggaggcctagtccagcctggagggagcctgcgtctctcttgtgcagtaagcggcatcgacctgagcaattacgccatcaactgggtgagacaagctccggggaagggtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagtggaggtggcggttctggcggtggcggttccggtggcggtggatcgggaggtggcggttctgacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggggcggtactaaggtggaaatcaaacgtacc

SEQ ID NO: 60 - protein, 486 aa, Artificial Sequence
  11041gH14 HC- 645 (VH/VL) dsscFv (mutated**)
EVQLVESGGGLVQPGGSLRLSCAVSGFSLSSYAMIWVRQAPGKGLEWIGIIDIEGSTYYASWAKGRFTISRDNSKNTVYLQMNSLRAEDTAVYYCARDRFVGVDIFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGGGGSEVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 61 - DNA, 1458 bp, Artificial Sequence
  11041gH14 HC- 645 (VH/VL) dsscFv (mutated**)
gaggtgcagctcgtggaaagcggaggaggactggtgcagccaggagggtccttgcggcttagctgtgccgtgtccggcttctccctgtcctcctacgccatgatctgggtccgccaagctcctgggaagggcctcgaatggattggtattatcgacatcgagggatcaacctactacgcctcgtgggccaagggacggttcaccatctcgcgggacaactccaagaacactgtgtatctgcagatgaacagcctgagggcagaagataccgccgtgtactactgcgcgagagatcgcttcgtgggcgtggacatctttgacccgtggggtcaaggcaccctggtcactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagcggtggcggtggctccggaggtggcggttcagaggttcaactgcttgagtctggaggaggcctagtccagcctggagggagcctgcgtctctcttgtgcagtaagcggcatcgacctgagcaattacgccatcaactgggtgagacaagctccggggaagtgtttagaatggatcggtataatatgggccagtgggacgaccttttatgctacatgggcgaaaggaaggtttacaattagccgggacaatagcaaaaacaccgtgtatctccaaatgaactccttgcgagcagaggacacggcggtgtactattgtgctcgcactgtcccaggttatagcactgcaccctacttcgatctgtggggacaagggaccctggtgactgtttcaagtggaggtggcggttctggcggtggcggttccggtggcggtggatcgggaggtggcggttctgacatacaaatgactcagtctccttcatcggtatccgcgtccgttggcgatagggtgactattacatgtcaaagctctcctagcgtctggagcaattttctatcctggtatcaacagaaaccggggaaggctccaaaacttctgatttatgaagcctcgaaactcaccagtggagttccgtcaagattcagtggctctggatcagggacagacttcacgttgacaatcagttcgctgcaaccagaggactttgcgacctactattgtggtggaggttacagtagcataagtgatacgacatttgggtgcggtactaaggtggaaatcaaacgtacc

SEQ ID NO: 62 - protein, 474 aa, Artificial Sequence
  11041gL13 LC- 650 scFv (unmutated*)
AVQLTQSPSSLSASVGDRVTITCQASEDIYTNLAWYQQKPGKAPKLLIYWASTLASGVPSRFSGSGSGTDFTLTISSLQPEDFATYYCQASVYGNAADSRYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSEVQLVQSGAEVKKPGSSVKVSCKASGYSFTSYYIHWVRQAPGQGLEWMGRIGPGSGDINYNEKFKGRATFTVDKSTSTAYMELSSLRSEDTAVYYCARFHYDGADWGQGTLVTVSSGGGGSGGGGSGGGGSGGGGSDIQMTQSPSSLSASVGDRVTITCKASQNINENLDWYQQKPGKAPKLLIYYTDILQTGIPSRFSGSGSGTDYTLTISSLQPEDFATYYCYQYYSGYTFGQGTKLEIKRT

SEQ ID NO: 63 - DNA, 1422 bp, Artificial Sequence
  11041gL13 LC- 650 scFv (unmutated*)
gccgtccaactgactcagtccccgagctcactttccgcgagcgtgggagatcgcgtgaccattacgtgccaggcctcggaggacatctacaccaacctcgcctggtatcaacagaagcctggcaaagctcccaagctgttgatctactgggcctccactctggcctccggagtgccttcgcggttctccggttctggatcaggcaccgacttcaccctgacaatcagcagcctccagccggaagattttgccacttactactgccaagcatccgtctacgggaacgcagcggactccagatataccttcggcgggggaaccaaagtggagattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgcagcggtggcggtggctccggaggtggcggttcagaggtgcagctggtgcagtccggcgccgaggtgaagaagcccggctcctccgtgaaggtgtcctgcaaggcctccggctactccttcacctcctactacatccactgggtgaggcaggcccccggccagggcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctccggaggtggcggttctggcggtggcggttccggtggcggtggatcgggaggtggcggttctgacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggccagggcaccaagctggagatcaagcgtacc

SEQ ID NO: 64 - protein, 474 aa, Artificial Sequence
  11041gL13 LC- 650 dsscFv (mutated**)
AVQLTQSPSSLSASVGDRVTITCQASEDIYTNLAWYQQKPGKAPKLLIYWASTLASGVPSRFSGSGSGTDFTLTISSLQPEDFATYYCQASVYGNAADSRYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSEVQLVQSGAEVKKPGSSVKVSCKASGYSFTSYYIHWVRQAPGQCLEWMGRIGPGSGDINYNEKFKGRATFTVDKSTSTAYMELSSLRSEDTAVYYCARFHYDGADWGQGTLVTVSSGGGGSGGGGSGGGGSGGGGSDIQMTQSPSSLSASVGDRVTITCKASQNINENLDWYQQKPGKAPKLLIYYTDILQTGIPSRFSGSGSGTDYTLTISSLQPEDFATYYCYQYYSGYTFGCGTKLEIKRT

SEQ ID NO: 65 - DNA, 1422 bp, Artificial Sequence
  11041gL13 LC- 650 dsscFv (mutated**)
gccgtccaactgactcagtccccgagctcactttccgcgagcgtgggagatcgcgtgaccattacgtgccaggcctcggaggacatctacaccaacctcgcctggtatcaacagaagcctggcaaagctcccaagctgttgatctactgggcctccactctggcctccggagtgccttcgcggttctccggttctggatcaggcaccgacttcaccctgacaatcagcagcctccagccggaagattttgccacttactactgccaagcatccgtctacgggaacgcagcggactccagatataccttcggcgggggaaccaaagtggagattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgcagcggtggcggtggctccggaggtggcggttcagaggtgcagctggtgcagtccggcgccgaggtgaagaagcccggctcctccgtgaaggtgtcctgcaaggcctccggctactccttcacctcctactacatccactgggtgaggcaggcccccggccagtgcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctccggaggtggcggttctggcggtggcggttccggtggcggtggatcgggaggtggcggttctgacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggctgcggcaccaagctggagatcaagcgtacc

SEQ ID NO: 66 - protein, 11 aa, Artificial Sequence
  Light chain linker between kappa constant region and 650 VH of scFv/dssFv (Y)
SGGGGSGGGGS

SEQ ID NO: 67 - protein, 20 aa, Artificial Sequence
  Light chain linker between VH and VL of 650 scFv/dsscFv
GGGGSGGGGSGGGGSGGGGS

SEQ ID NO: 68 - protein, 11 aa, Artificial Sequence
  Heavy chain linker between CH1 constant region and 645 VH of scFv/dssFv (X)
SGGGGSGGGGS

SEQ ID NO: 69 - protein, 20 aa, Artificial Sequence
  Heavy chain linker between VH and VL of 645 scFv/dsscFv
GGGGSGGGGSGGGGSGGGGS

SEQ ID NO: 70 - protein, 11 aa, Artificial Sequence
  11070 CDRL1
KASKTISKYLA

SEQ ID NO: 71 - protein, 7 aa, Artificial Sequence
  11070 CDRL2
SGSTLQS

SEQ ID NO: 72 - protein, 9 aa, Artificial Sequence
  11070 CDRL3
QQHNEYPLT

SEQ ID NO: 73 - protein, 10 aa, Artificial Sequence
  11070 CDRH1
GFSLTSYSVH

SEQ ID NO: 74 - protein, 16 aa, Artificial Sequence
  11070 CDRH2
RMWSDGDTSYNTAFTS

SEQ ID NO: 75 - protein, 12 aa, Artificial Sequence
  11070 CDRH3
SLDFYYDTTLAF

SEQ ID NO: 76 - protein, 107 aa, Artificial Sequence
  11070gL7 V-region
DIQMTQSPSSVSASVGDRVTITCKASKTISKYLAWYQQKPGKANKLLIYSGSTLQSGVPSRFSGSGSGTDFTLTISSLQPEDFATYYCQQHNEYPLTFGQGTKLEIK

SEQ ID NO: 77 - DNA, 321 bp, Artificial Sequence
  11070gL7 V-region
gacattcagatgactcagtcgccttcgtccgtgagcgccagcgtcggagacagagtgacaatcacctgtaaagcgtccaagaccatctccaagtacctggcttggtatcagcagaaaccggggaaggccaacaagttgcttatctactccggttctactctccaatcgggagtgccaagccggttttccgggtccggatcaggcaccgacttcaccctcaccatctcatccctgcaaccggaggatttcgccacgtactactgccagcagcacaacgaataccccctgaccttcggccaaggaactaagctggaaattaag

SEQ ID NO: 78 - protein, 120 aa, Artificial Sequence
  11070gH16 V-region
EVQLQESGPGLVKPSQTLSLTCTVSGFSLTSYSVHWVRQHPGKGLEWIGRMWSDGDTSYNTAFTSRLTISRDTSKNQVSLKLSSVTAADTAVYYCARSLDFYYDTTLAFWGQGTTVTVSS

SEQ ID NO: 79 - DNA, 360 bp, Artificial Sequence
  11070gH16 V-region
gaggtgcagctgcaagaatccggtcctggcctcgtgaagccgtcgcagaccttgagcctgacctgtactgtgtccggattcagcctcacatcctactcggtgcactgggtcagacagcatcccggaaaaggcctggaatggattgggaggatgtggtctgatggagacacctcctacaacacggcgttcaccagccggctgaccatctcccgcgacacctccaagaaccaagtgtcgcttaagctgtcctcagtcactgccgccgataccgcagtgtattactgcgctcggtcactggacttttactacgacaccaccctggccttctggggacaggggactactgtgactgtctcgagc

SEQ ID NO: 80 - protein, 214 aa, Artificial Sequence
  11070gL7 Light chain
DIQMTQSPSSVSASVGDRVTITCKASKTISKYLAWYQQKPGKANKLLIYSGSTLQSGVPSRFSGSGSGTDFTLTISSLQPEDFATYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 642 bp, Artificial Sequence
  11070gL7 Light chain
gacattcagatgactcagtcgccttcgtccgtgagcgccagcgtcggagacagagtgacaatcacctgtaaagcgtccaagaccatctccaagtacctggcttggtatcagcagaaaccggggaaggccaacaagttgcttatctactccggttctactctccaatcgggagtgccaagccggttttccgggtccggatcaggcaccgacttcaccctcaccatctcatccctgcaaccggaggatttcgccacgtactactgccagcagcacaacgaataccccctgaccttcggccaaggaactaagctggaaattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 82 - protein, 223 aa, Artificial Sequence
  11070gH16 Fab Heavy chain
EVQLQESGPGLVKPSQTLSLTCTVSGFSLTSYSVHWVRQHPGKGLEWIGRMWSDGDTSYNTAFTSRLTISRDTSKNQVSLKLSSVTAADTAVYYCARSLDFYYDTTLAFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 83 - DNA, 669 bp, Artificial Sequence
  11070gH16 Fab Heavy chain
gaggtgcagctgcaagaatccggtcctggcctcgtgaagccgtcgcagaccttgagcctgacctgtactgtgtccggattcagcctcacatcctactcggtgcactgggtcagacagcatcccggaaaaggcctggaatggattgggaggatgtggtctgatggagacacctcctacaacacggcgttcaccagccggctgaccatctcccgcgacacctccaagaaccaagtgtcgcttaagctgtcctcagtcactgccgccgataccgcagtgtattactgcgctcggtcactggacttttactacgacaccaccctggccttctggggacaggggactactgtgactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgt

SEQ ID NO: 84 - protein, 14 aa, Artificial Sequence
  11041 CDRL3 (not mutated)
QACVYGNSADSRYT

SEQ ID NO: 85 - protein, 14 aa, Artificial Sequence
  11041 CDRL3 C91S
QASVYGNSADSRYT

SEQ ID NO: 86 - protein, 14 aa, Artificial Sequence
  11041 CDRL3 C91V
QAVVYGNSADSRYT

SEQ ID NO: 87 - protein, 14 aa, Artificial Sequence
  11041 CDRL3 S96A
QACVYGNAADSRYT

SEQ ID NO: 88 - protein, 14 aa, Artificial Sequence
  11041 CDRL3 C91V S96A
QAVVYGNAADSRYT

SEQ ID NO: 89 - protein, 14 aa, Artificial Sequence
  11041 CDRL3 N95D
QACVYGDSADSRYT

SEQ ID NO: 90 - protein, 14 aa, Artificial Sequence
  11041 CDRL3 C91S N95D
QASVYGDSADSRYT

SEQ ID NO: 91 - protein, 14 aa, Artificial Sequence
  11041 CDRL3 C91V N95D
QAVVYGDSADSRYT

SEQ ID NO: 92 - protein, 16 aa, Artificial Sequence
  11041 CDRH2 (not mutated)
IIDIDGSTYYASWAKG

SEQ ID NO: 93 - protein, 16 aa, Artificial Sequence
  11041 CDRH2 G55A
IIDIDASTYYASWAKG

SEQ ID NO: 94 - protein, 11 aa, Artificial Sequence
  11041 CDRH3 D107E
DRFVGVDIFEP

SEQ ID NO: 95 - protein, 112 aa, Artificial Sequence
  Rabbit 11041 VL-region
AVVLTQTASPVSAPVGGTVTIKCQASEDIYTNLAWYQQKPGQPPKLLIYWASTLASGVPSRFKGSGSGTEFTLTISDLECADAATYYCQACVYGNSADSRYTFGGGTEVVVK

SEQ ID NO: 96 - DNA, 336 bp, Artificial Sequence
  Rabbit 11041 VL-region
gccgtcgtgctgacccagactgcatcccccgtgtctgcacctgtgggaggcacagtcaccatcaagtgccaggccagtgaggacatttacaccaatttagcctggtatcaacagaaaccaggacagcctcccaagctcctgatctactgggcatccactctggcatctggggtcccatcgcggttcaaaggcagtggatctgggacagagttcactctcaccatcagcgacctggagtgtgccgatgctgccacttactactgtcaagcctgtgtttatggcaatagtgctgatagtcggtatactttcggcggagggaccgaggtggtggtcaaa

SEQ ID NO: 97 - protein, 116 aa, Artificial Sequence
  Rabbit 11041 VH-region
QSVEESGGRLVTPGTPLTLTCTVSGFSLSSYAMIWVRQAPGEGLEWIGIIDIDGSTYYASWAKGRFTISRTSTTVDLKITSPTTGDTATYFCARDRFVGVDIFDPWGPGTLVTVSS

SEQ ID NO: 98 - DNA, 348 bp, Artificial Sequence
  Rabbit 11041 VH-region
cagtcggtggaggagtccgggggtcgcctggtcacgcctgggacacccctgacactcacctgcaccgtctctggattctccctcagtagctatgcaatgatctgggtccgccaggctccaggggaggggctggaatggatcggaatcattgatattgatgggagcacatactacgcgagctgggcgaaaggccgattcaccatctccagaacctcgaccacggtggatctgaaaatcaccagtccgacaaccggggacacggccacctatttctgtgccagagatcgttttgttggtgttgatatttttgatccctggggcccaggcaccctggtcaccgtctcgagc

SEQ ID NO: 99 - protein, 112 aa, Artificial Sequence
  11041gL1 V-region
AVVLTQSPSSLSASVGDRVTITCQASEDIYTNLAWYQQKPGKAPKLLIYWASTLASGVPSRFSGSGSGTDFTLTISSLQPEDFATYYCQACVYGNSADSRYTFGGGTKVEIK

SEQ ID NO: 100 - protein, 112 aa, Artificial Sequence
  11041 gL1 C91S V-region (gL2)
AVVLTQSPSSLSASVGDRVTITCQASEDIYTNLAWYQQKPGKAPKLLIYWASTLASGVPSRFSGSGSGTDFTLTISSLQPEDFATYYCQASVYGNSADSRYTFGGGTKVEIK

SEQ ID NO: 101 - protein, 112 aa, Artificial Sequence
  11041 gL1 C91V V-region (gL3)
AVVLTQSPSSLSASVGDRVTITCQASEDIYTNLAWYQQKPGKAPKLLIYWASTLASGVPSRFSGSGSGTDFTLTISSLQPEDFATYYCQAVVYGNSADSRYTFGGGTKVEIK

SEQ ID NO: 102 - protein, 112 aa, Artificial Sequence
  11041gL6 V-region
AVQLTQSPSSLSASVGDRVTITCQASEDIYTNLAWYQQKPGKAPKLLIYWASTLASGVPSRFSGSGSGTDFTLTISSLQPEDFATYYCQACVYGNSADSRYTFGGGTKVEIK

SEQ ID NO: 103 - protein, 112 aa, Artificial Sequence
  11041gL7 V-region
AIQLTQSPSSLSASVGDRVTITCQASEDIYTNLAWYQQKPGKAPKLLIYWASTLASGVPSRFSGSGSGTDFTLTISSLQPEDFATYYCQACVYGNSADSRYTFGGGTKVEIK

SEQ ID NO: 104 - protein, 112 aa, Artificial Sequence
  11041 gL1 N95D V-region (gL8)
AVVLTQSPSSLSASVGDRVTITCQASEDIYTNLAWYQQKPGKAPKLLIYWASTLASGVPSRFSGSGSGTDFTLTISSLQPEDFATYYCQACVYGDSADSRYTFGGGTKVEIK

SEQ ID NO: 105 - protein, 112 aa, Artificial Sequence
  11041 gL1 S96A V-region (gL9)
AVVLTQSPSSLSASVGDRVTITCQASEDIYTNLAWYQQKPGKAPKLLIYWASTLASGVPSRFSGSGSGTDFTLTISSLQPEDFATYYCQACVYGNAADSRYTFGGGTKVEIK

SEQ ID NO: 106 - protein, 112 aa, Artificial Sequence
  11041 gL1 C91S S96A V-region (gL10)
AVVLTQSPSSLSASVGDRVTITCQASEDIYTNLAWYQQKPGKAPKLLIYWASTLASGVPSRFSGSGSGTDFTLTISSLQPEDFATYYCQASVYGNAADSRYTFGGGTKVEIK

SEQ ID NO: 107 - protein, 112 aa, Artificial Sequence
  11041 gL6 C91S V-region (gL11)
AVQLTQSPSSLSASVGDRVTITCQASEDIYTNLAWYQQKPGKAPKLLIYWASTLASGVPSRFSGSGSGTDFTLTISSLQPEDFATYYCQASVYGNSADSRYTFGGGTKVEIK

SEQ ID NO: 108 - protein, 112 aa, Artificial Sequence
  11041 gL7 C91S V-region (gL12)
AIQLTQSPSSLSASVGDRVTITCQASEDIYTNLAWYQQKPGKAPKLLIYWASTLASGVPSRFSGSGSGTDFTLTISSLQPEDFATYYCQASVYGNSADSRYTFGGGTKVEIK

SEQ ID NO: 109 - protein, 112 aa, Artificial Sequence
  11041 gL7 C91S S96A V-region (gL14)
AIQLTQSPSSLSASVGDRVTITCQASEDIYTNLAWYQQKPGKAPKLLIYWASTLASGVPSRFSGSGSGTDFTLTISSLQPEDFATYYCQASVYGNAADSRYTFGGGTKVEIK

SEQ ID NO: 110 - protein, 119 aa, Artificial Sequence
  11041gH1 V-region
EVQLVESGGGLVQPGGSLRLSCAVSGFSLSSYAMIWVRQAPGKGLEWIGIIDIDGSTYYASWAKGRFTISRDSSKNTVYLQMNSLRAEDTAVYYCARDRFVGVDIFDPWGQGTLVTVSS

SEQ ID NO: 111 - protein, 119 aa, Artificial Sequence
  11041 gH1 G55A V-region (gH2)
EVQLVESGGGLVQPGGSLRLSCAVSGFSLSSYAMIWVRQAPGKGLEWIGIIDIDASTYYASWAKGRFTISRDSSKNTVYLQMNSLRAEDTAVYYCARDRFVGVDIFDPWGQGTLVTVSS

SEQ ID NO: 112 - protein, 119 aa, Artificial Sequence
  11041 gH1 D54E V-region (gH3)
EVQLVESGGGLVQPGGSLRLSCAVSGFSLSSYAMIWVRQAPGKGLEWIGIIDIEGSTYYASWAKGRFTISRDSSKNTVYLQMNSLRAEDTAVYYCARDRFVGVDIFDPWGQGTLVTVSS

SEQ ID NO: 113 - protein, 119 aa, Artificial Sequence
  11041 gH1 D107E V-region (gH4)
EVQLVESGGGLVQPGGSLRLSCAVSGFSLSSYAMIWVRQAPGKGLEWIGIIDIDGSTYYASWAKGRFTISRDSSKNTVYLQMNSLRAEDTAVYYCARDRFVGVDIFEPWGQGTLVTVSS

SEQ ID NO: 114 - protein, 119 aa, Artificial Sequence
  11041gH5 V-region
EVQLVESGGGLVQPGGSLRLSCAVSGFSLSSYAMIWVRQAPGKGLEWIGIIDIDGSTYYASWAKGRFTISRDNSKNTVYLQMNSLRAEDTAVYYCARDRFVGVDIFDPWGQGTLVTVSS

SEQ ID NO: 115 - protein, 119 aa, Artificial Sequence
  11041gH8 V-region
EVQLVESGGGLVQPGGSLRLSCAVSGFSLSSYAMIWVRQAPGKGLEWIGIIDIDGSTYYASWAKGRFTISRDSSKNTLYLQMNSLRAEDTAVYYCARDRFVGVDIFDPWGQGTLVTVSS

SEQ ID NO: 116 - protein, 119 aa, Artificial Sequence
  11041gH9 V-region
EVQLVESGGGLVQPGGSLRLSCAASGFSLSSYAMIWVRQAPGKGLEWIGIIDIDGSTYYASWAKGRFTISRDSSKNTVYLQMNSLRAEDTAVYYCARDRFVGVDIFDPWGQGTLVTVSS

SEQ ID NO: 117 - protein, 119 aa, Artificial Sequence
  11041gH11 V-region
EVQLVESGGGLVQPGGSLRLSCAVSGFSLSSYAMIWVRQAPGKGLEWVGIIDIDGSTYYASWAKGRFTISRDNSKNTLYLQMNSLRAEDTAVYYCARDRFVGVDIFDPWGQGTLVTVSS

SEQ ID NO: 118 - protein, 119 aa, Artificial Sequence
  11041gH12 V-region
EVQLVESGGGLVQPGGSLRLSCAVSGFSLSSYAMIWVRQAPGKGLEWISIIDIDGSTYYASWAKGRFTISRDNSKNTLYLQMNSLRAEDTAVYYCARDRFVGVDIFDPWGQGTLVTVSS

SEQ ID NO: 119 - protein, 119 aa, Artificial Sequence
  11041 gH8 D54E V-region (gH15)
EVQLVESGGGLVQPGGSLRLSCAVSGFSLSSYAMIWVRQAPGKGLEWIGIIDIEGSTYYASWAKGRFTISRDSSKNTLYLQMNSLRAEDTAVYYCARDRFVGVDIFDPWGQGTLVTVSS

SEQ ID NO: 120 - protein, 119 aa, Artificial Sequence
  11041 gH11 D54E V-region (gH17)
EVQLVESGGGLVQPGGSLRLSCAVSGFSLSSYAMIWVRQAPGKGLEWVGIIDIEGSTYYASWAKGRFTISRDNSKNTLYLQMNSLRAEDTAVYYCARDRFVGVDIFDPWGQGTLVTVSS

SEQ ID NO: 121 - protein, 119 aa, Artificial Sequence
  11041 gH12 D54E V-region (gH18)
EVQLVESGGGLVQPGGSLRLSCAVSGFSLSSYAMIWVRQAPGKGLEWISIIDIEGSTYYASWAKGRFTISRDNSKNTLYLQMNSLRAEDTAVYYCARDRFVGVDIFDPWGQGTLVTVSS

SEQ ID NO: 122 - protein, 16 aa, Artificial Sequence
  11070 CDRH2 (not mutated)
RMWSDGDTSYNSAFTS

SEQ ID NO: 123 - protein, 107 aa, Artificial Sequence
  Rat Ab 11070 VL-region
DIVMTQTPSNLAASPGESVSINCKASKTISKYLAWYQQKPGKANKLLIYSGSTLQSGTPSRFSGSGSSTDFTLTIRNLEPEDFGLYYCQQHNEYPLTFGSGTKLEIK

SEQ ID NO: 124 - DNA, 321 bp, Artificial Sequence
  Rat Ab 11070 VL-region
gatattgtgatgacacagactccatctaatcttgctgcctctcctggagaaagtgtttccatcaattgcaaggcaagtaagaccattagcaagtatttagcctggtatcaacagaaacctgggaaagcaaataagcttcttatctattctgggtcaactttgcaatctggaactccatcgaggttcagtggcagtggatctagtacagatttcactctcaccatcagaaacctggagcctgaagattttggactctattactgtcaacagcataatgaatacccgctcacgttcggttctgggaccaagttggaaataaaa

SEQ ID NO: 125 - protein, 120 aa, Artificial Sequence
  Rat Ab 11070 VH-region
EVQLQESGPGLVQPSQTLSPTCTVSGFSLTSYSVHWVRQHSGKSLEWMGRMWSDGDTSYNSAFTSRLSITRDTSKSQVFLKMNSLQTEDTGTYYCARSLDFYYDTTLAFWGPGTTVTVSS

SEQ ID NO: 126 - DNA, 360 bp, Artificial Sequence
  Rat Ab 11070 VH-region
gaggtgcagctgcaggagtcaggacctgggctggtgcagccctcacagaccctgtcccccacctgcactgtctctgggttctcactaactagttacagtgtacactgggttcgccagcattcaggaaagagtctggaatggatgggaagaatgtggagtgatggagacacatcatataattcagcgttcacatcccgattgagcatcactagggacacctccaagagccaagttttcttaaaaatgaacagtctgcaaactgaagacacaggcacttactactgtgccagaagtctcgatttttactatgatactactcttgccttctggggcccaggaaccacggtcaccgtctcgagt

SEQ ID NO: 127 - protein, 107 aa, Artificial Sequence
  11070gL1 V-region
DIVMTQSPSSVSASVGDRVTITCKASKTISKYLAWYQQKPGKANKLLIYSGSTLQSGTPSRFSGSGSSTDFTLTISSLQPEDFATYYCQQHNEYPLTFGQGTKLEIK

SEQ ID NO: 128 - protein, 120 aa, Artificial Sequence
  11070gH1 V-region
EVQLQESGPGLVKPSQTLSLTCTVSGFSLTSYSVHWVRQHSGKGLEWMGRMWSDGDTSYNSAFTSRLTISRDTSKSQVSLKLSSVTAADTAVYYCARSLDFYYDTTLAFWGQGTTVTVSS

SEQ ID NO: 129 - protein, 120 aa, Artificial Sequence
  11070gH13 V-region (gH1 S61T)
EVQLQESGPGLVKPSQTLSLTCTVSGFSLTSYSVHWVRQHSGKGLEWMGRMWSDGDTSYNTAFTSRLTISRDTSKSQVSLKLSSVTAADTAVYYCARSLDFYYDTTLAFWGQGTTVTVSS

SEQ ID NO: 130 - protein, 106 aa, Artificial Sequence
  Rat Ab 650 (1539) VL-region
DIQMTQSPPVLSASVGDRVTLSCKASQNINENLDWYHQKHGEAPKLLIYYTDILQTGIPSRFSGSGSGTDYTLTISSLQPEDVATYYCYQYYSGYTFGPGTKLEIK

SEQ ID NO: 131 - DNA, 318 bp, Artificial Sequence
  Rat Ab 650 (1539) VL-region
gacatccagatgacccagtctcctccagtcctgtctgcatctgtgggagacagagtcactctcagttgcaaagcaagtcagaatattaatgagaacttagactggtatcatcaaaagcatggcgaagctccaaaactcctgatatattatacagacattttgcaaacgggcatcccatcaaggttcagtggcagtggatctggtacagattacacactcaccatcagcagcctgcagcctgaagatgttgccacatattactgctatcagtattacagtgggtacacgtttggacctgggaccaagctggaaataaaa

SEQ ID NO: 132 - protein, 116 aa, Artificial Sequence
  Rat Ab 650 (1539) VH-region
QVQLQQSGAELVKPGSSVKMSCKASGYSFTSYYIHWIKQRPGQGLEWIGRIGPGSGDINYNEKFKGKATFTVDKYFSTAYMQLSSLSPEDTAVFYCARFHYDGADWGQGTLVTVSS

SEQ ID NO: 133 - DNA, 348 bp, Artificial Sequence
  Rat Ab 650 (1539) VH-region
caggtacaactgcagcagtctggagctgagttggtgaagcctgggtcttcagtgaagatgtcctgcaaggcttctggctacagtttcaccagctactacatacactggataaagcagaggcctggacagggccttgagtggattgggcgtattggtcctggaagtggagatattaattacaatgagaagttcaagggcaaggccacatttactgtggacaaatatttcagcacagcctacatgcaactcagcagcctgtcacctgaggacactgcggtcttttactgtgcaagatttcactatgatggggctgactggggccaaggcactctggtcacagtctcgagc

SEQ ID NO: 134 - protein, 107 aa, Artificial Sequence [sequence omitted]

SEQ ID NO: 135 - DNA, 321 bp, Artificial Sequence
  Human IGKV1D-13 IGKJ4 acceptor framework
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctctcactttcggcggagggaccaaggtggagatcaaa

SEQ ID NO: 136 - protein, 112 aa, Artificial Sequence
  Human IGHV3-66 IGHJ4 acceptor framework
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARYFDYWGQGTLVTVSS

SEQ ID NO: 137 - DNA, 336 bp, Artificial Sequence
  Human IGHV3-66 IGHJ4 acceptor framework
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagatactttgactactggggccaaggaaccctggtcaccgtctcctca

SEQ ID NO: 138 - protein, 107 aa, Artificial Sequence
  Human IGKV1-12 IGKJ2 acceptor framework
DIQMTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKLEIK

SEQ ID NO: 139 - DNA, 321 bp, Artificial Sequence
  Human IGKV1-12 IGKJ2 acceptor framework
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccttacacttttggccaggggaccaagctggagatcaaa

SEQ ID NO: 140 - protein, 119 aa, Artificial Sequence
  Human IGHV4-31 IGHJ6 acceptor framework
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARYYYYYGMDVWGQGTTVTVSS

SEQ ID NO: 141 - DNA, 357 bp, Artificial Sequence
  Human IGHV4-31 IGHJ6 acceptor framework
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagatactactactactacggtatggacgtctgggggcaagggaccacggtcaccgtctcctca

SEQ ID NO: 142 - protein, 219 aa, Artificial Sequence; identical to SEQ ID NO: 18
  IL22 knob Light chain

SEQ ID NO: 143 - DNA, 657 bp, Artificial Sequence
  IL22 knob Light chain
gcagtgcagctgactcagtccccgtcctccctgtcggcctcagtgggagatcgcgtgaccattacctgtcaagccagcgaagatatctacaccaacctcgcctggtaccagcagaaacccgggaaggctccgaagctgctcatctattgggccagcaccttggcgtctggcgtgccatcccggttttccggttcgggaagcggaaccgacttcacgcttaccatttcctccctgcaacctgaggacttcgccacttactactgccaagcctccgtctacgggaacgccgcggactcaagatacactttcggcggcggaaccaaggtcgaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 144 - protein, 446 aa, Artificial Sequence
  IL22 knob Heavy chain
EVQLVESGGGLVQPGGSLRLSCAVSGFSLSSYAMIWVRQAPGKGLEWIGIIDIEGSTYYASWAKGRFTISRDNSKNTVYLQMNSLRAEDTAVYYCARDRFVGVDIFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 145 - DNA, 1338 bp, Artificial Sequence
  IL22 knob Heavy chain
gaagtgcagctcgtggagtcggggggaggactggtgcagcccggaggttccctgcgcttgagctgtgcagtgtcaggcttttccctgtcctcctacgccatgatctgggtccgccaagctcctggaaaggggctggaatggatcggaatcatcgacatcgagggctccacctactacgcctcatgggccaagggccggttcaccatttcccgggataacagcaagaacactgtgtacctccagatgaactcgctgagggccgaggacactgccgtgtattactgcgcgcgggacagattcgtcggggtggacattttcgacccgtggggtcaaggcacccttgtgaccgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtatccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 146 - protein, 446 aa, Artificial Sequence
  IL22 Hole heavy chain
EVQLVESGGGLVQPGGSLRLSCAVSGFSLSSYAMIWVRQAPGKGLEWIGIIDIEGSTYYASWAKGRFTISRDNSKNTVYLQMNSLRAEDTAVYYCARDRFVGVDIFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 147 - DNA, 1338 bp, Artificial Sequence
  IL22 Hole heavy chain
gaagtgcagctcgtggagtcggggggaggactggtgcagcccggaggttccctgcgcttgagctgtgcagtgtcaggcttttccctgtcctcctacgccatgatctgggtccgccaagctcctggaaaggggctggaatggatcggaatcatcgacatcgagggctccacctactacgcctcatgggccaagggccggttcaccatttcccgggataacagcaagaacactgtgtacctccagatgaactcgctgagggccgaggacactgccgtgtattactgcgcgcgggacagattcgtcggggtggacattttcgacccgtggggtcaaggcacccttgtgaccgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtatccaacaaaggcctcccgtcctccatcgagaaaaccatctccaaagccaaagggcagccccgagagccacaggtgtacaccctgcccccatcccaggaggagatgaccaagaaccaggtcagcctgagctgcgcggtcaaaggcttctaccccagcgacatcgccgtggagtgggagagcaatgggcagccggagaacaactacaagaccacgcctcccgtgctggactccgacggctccttcttcctcgtcagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 148 - protein, 213 aa, Artificial Sequence
  IL13 knob light chain
DIQMTQSPSSLSASVGDRVTITCKASQNINENLDWYQQKPGKAPKLLIYYTDILQTGIPSRFSGSGSGTDYTLTISSLQPEDFATYYCYQYYSGYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - DNA, 639 bp, Artificial Sequence
  IL13 knob light chain
gacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggccagggcaccaagctggagatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 150 - protein, 443 aa, Artificial Sequence
  IL13 knob heavy chain
EVQLVQSGAEVKKPGSSVKVSCKASGYSFTSYYIHWVRQAPGQGLEWMGRIGPGSGDINYNEKFKGRATFTVDKSTSTAYMELSSLRSEDTAVYYCARFHYDGADWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 151 - DNA, 1329 bp, Artificial Sequence
  IL13 knob heavy chain
gaggtgcagctggtgcagtccggcgccgaggtgaagaagcccggctcctccgtgaaggtgtcctgcaaggcctccggctactccttcacctcctactacatccactgggtgaggcaggcccccggccagggcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtatccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 152 - protein, 443 aa, Artificial Sequence
  IL13 Hole heavy chain
EVQLVQSGAEVKKPGSSVKVSCKASGYSFTSYYIHWVRQAPGQGLEWMGRIGPGSGDINYNEKFKGRATFTVDKSTSTAYMELSSLRSEDTAVYYCARFHYDGADWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 153 - DNA, 1329 bp, Artificial Sequence
  IL13 Hole heavy chain
gaggtgcagctggtgcagtccggcgccgaggtgaagaagcccggctcctccgtgaaggtgtcctgcaaggcctccggctactccttcacctcctactacatccactgggtgaggcaggcccccggccagggcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtctcgagcgcttctacaaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtatccaacaaaggcctcccgtcctccatcgagaaaaccatctccaaagccaaagggcagccccgagagccacaggtgtacaccctgcccccatcccaggaggagatgaccaagaaccaggtcagcctgagctgcgcggtcaaaggcttctaccccagcgacatcgccgtggagtgggagagcaatgggcagccggagaacaactacaagaccacgcctcccgtgctggactccgacggctccttcttcctcgtcagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 154 - protein, 13 aa, Artificial Sequence
  IL22 peptide 72-84
VRLIGEKLFHGVS

SEQ ID NO: 155 - protein, 14 aa, Artificial Sequence
  IL22 peptide 72-85
VRLIGEKLFHGVSM

SEQ ID NO: 156 - protein, 10 aa, Artificial Sequence
  IL22 peptide 75-84
IGEKLFHGVS

SEQ ID NO: 157 - protein, 11 aa, Artificial Sequence
  IL22 peptide 75-85
IGEKLFHGVSM

SEQ ID NO: 158 - protein, 9 aa, Artificial Sequence
  IL22 peptide 76-84
GEKLFHGVS

SEQ ID NO: 159 - protein, 6 aa, Artificial Sequence
  IL22 peptide 80-85
FHGVSM

SEQ ID NO: 160 - protein, 14 aa, Artificial Sequence
  IL22 peptide 126-139
SNRLSTCHIEGDDL

SEQ ID NO: 161 - protein, 11 aa, Artificial Sequence
  IL22 peptide 101-111
EEVLFPQSDRF

SEQ ID NO: 162 - protein, 13 aa, Artificial Sequence
  IL22 peptide 103-115
VLFPQSDRFQPYM

SEQ ID NO: 163 - protein, 15 aa, Artificial Sequence
  IL22 peptide 103-117
VLFPQSDRFQPYMQE

SEQ ID NO: 164 - protein, 16 aa, Artificial Sequence
  IL22 peptide 43-58
DKSNFQQPYITNRTFM

SEQ ID NO: 165 - protein, 13 aa, Artificial Sequence
  IL22 peptide 105-117
FPQSDRFQPYMQE

SEQ ID NO: 166 - DNA, 1422 bp, Artificial Sequence
  light chain for transient expression
gcggtgcagctgactcagtcaccgtcctcgctttccgcttccgtgggagacagagtgaccatcacctgtcaagcctccgaagatatctacaccaacctcgcctggtaccagcagaagcccggaaaggccccaaagctgttgatctactgggcgtctaccctcgcctccggggtgccgtcgcgctttagcggttcgggatccggcaccgacttcaccctgactattagcagcctgcagcctgaggacttcgccacttattactgccaagcatccgtctacgggaacgccgccgattcacggtacaccttcggcggcggaacgaaagtcgagattaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctgagcagcaccctgacgctgtctaaagcagactacgagaaacacaaagtgtacgcctgcgaagtcacccatcagggcctgagctcaccagtaacaaaaagttttaatagaggggagtgtagcggtggcggtggctccggtggtggcggttcagaggtgcagctggtgcagtccggcgccgaggtgaagaagcccggctcctccgtgaaggtgtcctgcaaggcctccggctactccttcacctcctactacatccactgggtgaggcaggcccccggccagtgcctggagtggatgggcaggatcggccccggctccggcgacatcaactacaacgagaagttcaagggcagggccaccttcaccgtggacaagtccacctccaccgcctacatggagctgtcctccctgaggtccgaggacaccgccgtgtactactgcgccaggttccactacgacggcgccgactggggccagggcaccctggtgaccgtgtcctccggaggtggcggttctggcggtggcggttccggtggcggtggatcgggaggtggcggttctgacatccagatgacccagtccccctcctccctgtccgcctccgtgggcgacagggtgaccatcacctgcaaggcctcccagaacatcaacgagaacctggactggtaccagcagaagcccggcaaggcccccaagctgctgatctactacaccgacatcctgcagaccggcatcccctccaggttctccggctccggctccggcaccgactacaccctgaccatctcctccctgcagcccgaggacttcgccacctactactgctaccagtactactccggctacaccttcggctgcggcaccaagctggagatcaagcgtacc

SEQ ID NO: 167 - DNA, 1458 bp, Artificial Sequence
  heavy chain for transient expression
gaggtgcagctcgtggaatccggcggcggactggtgcagccgggcggatccctgcggctgtcctgcgccgtgtcgggtttttccctgtcctcatacgccatgatctgggtcagacaggcacctgggaagggtctggagtggattggcatcatcgacatcgaagggtcgacctactacgcgagctgggccaagggaaggttcaccattagccgggacaacagcaagaacaccgtgtaccttcaaatgaactccctccgggccgaagataccgccgtgtattactgtgctcgcgaccgcttcgtgggagtggacatcttcgatccctggggacagggaactttggtcactgtctcgagcgcgtccacaaagggcccatcggtcttccccctggcaccctcctccaagagcacctctgggggcacagcggccctgggctgcctggtcaaggactacttccccgaaccagtgacggtgtcgtggaactcaggtgccctgaccagcggcgttcacaccttcccggctgtcctacagtcttcaggactctactccctgagcagcgtggtgaccgtgccctccagcagcttgggcacccagacctacatctgcaacgtgaatcacaagcccagcaacaccaaggtcgataagaaagttgagcccaaatcttgtagcggtggcggtggctccggtggtggcggttcagaagtgcagttgctggagtcaggtggagggctggtgcagcccggaggatcgctgcggttgtcatgcgcggtgtccggtattgatttgtccaattacgccatcaattgggtacgccaagcgccagggaagtgccttgagtggattggcatcatctgggcgtcggggacgaccttttatgctacttgggccaaaggaagattcacaatctcccgagacaactcgaagaacaccgtgtatcttcaaatgaactcgctcagggccgaggacacggcggtctactactgtgcacggacagtgccgggttattcaacggcaccttactttgatctttggggccaggggaccctcgtgactgtctcaagtggaggtggcggttctggcggtggcggttccggtggcggtggatcgggaggtggcggttctgatattcagatgacgcaatcaccttcgagcgtatccgcctcggtgggagacagggtgacaatcacttgtcagtcatccccctcagtctggagcaactttttgtcatggtatcagcagaagcccggaaaggctccgaaattgctgatctacgaggcatcgaagttgacgagcggtgtaccaagcagattctccggttcggggtcgggaactgacttcacccttacgatctcatcgctgcagccggaggattttgcgacctactactgtgggggtgggtattcgtcgatttccgacacaacattcgggtgcggcacgaaagtggaaatcaagcgtacc